(19) United States
(12) Patent Application Publication
(10) Pub. No.: US 2022/0193343 A1
(43) Pub. Date: Jun. 23, 2022

(54) AUTO-INJECTOR AND RELATED METHODS OF USE

(71) Applicant: Regeneron Pharmaceuticals, Inc., Tarrytown, NY (US)

(72) Inventors: Bart BURGESS, Bernville, PA (US); Bryan GRYGUS, Clifton Park, NY (US); Daniel HALBIG, Ballston Lake, NY (US); Ross KENYON, Saratoga Springs, NY (US); Trevor LANGLEY, Rensselaer, NY (US); Jeremy MCNAMARA, Albany, NY (US); Matthew PAUSLEY, Gansevoort, NY (US); Andrew DUMONT, Rensselaer, NY (US); Paige WAECHTER, Cohoes, NY (US); John BURKE, Ickleton (GB); Martin BONTOFT, Ickleton (GB); Craig MCGARRELL, Whitburn (GB); Scott MARTIN, Edinburgh (GB); James Nicholas MOWER, Edinburgh (GB); James Cunningham GLENCROSS, Edinburgh (GB); James Donald MCLUSKY, Edinburgh (GB); Andrew LABAT-ROCHECOUSTE, Keynsham (GB); Tim QUIGG, Ashley Down (GB); Matt HILL, Llanishen (GB); Matthew PHILLIPPO, Bath (GB); Kirsty WYNNE, Sheffield (GB)

(73) Assignee: Regeneron Pharmaceuticals, Inc., Tarrytown, NY (US)

(21) Appl. No.: 17/565,104

(22) Filed: Dec. 29, 2021

Related U.S. Application Data
(63) Continuation of application No. PCT/US2020/040729, filed on Jul. 2, 2020.
(60) Provisional application No. 62/869,851, filed on Jul. 2, 2019.
(60) Provisional application No. 62/869,777, filed on Jul. 2, 2019.
(60) Provisional application No. 62/932,786, filed on Nov. 8, 2019.
(60) Provisional application No. 62/932,934, filed on Nov. 8, 2019.

(51) Int. Cl. A61M 5/20; A61M 5/24; A61M 5/315

(52) U.S. Cl. CPC A61M 5/2053; A61M 5/2459; A61M 5/2425; A61M 5/3158; A61M 2005/3128; A61M 2205/582; A61M 2205/584; A61M 2005/206; A61M 2205/583

(57) ABSTRACT

An auto-injector may include a housing having a longitudinal axis and a transverse axis, the housing having a shorter dimension along the transverse axis than along the longitudinal axis, wherein the transverse axis is perpendicular to the longitudinal axis; a flowpath having a first end and a second end; and a container enclosing a first fluid, the container extending from a first end toward a second end along or parallel to the longitudinal axis and being movable from a first position to a second position along or parallel to the longitudinal axis, the container being fluidly isolated from the flowpath in the first position and fluidly connected to the flowpath in the second position.

### CROSS-REFERENCE TO RELATED APPLICATIONS

This application claims priority to U.S. Provisional Application No. 62/869,851, filed on Jul. 2, 2019, U.S. Provisional Application No. 62/869,777, filed on Jul. 2, 2019, U.S. Provisional Application No. 62/932,786, filed on Nov. 8, 2019, and U.S. Provisional Application No. 62/932,934, filed on Nov. 8, 2019, the entireties of each of which is incorporated by reference herein.

### TECHNICAL FIELD

This disclosure is directed to an auto-injector and related methods of use.

### INTRODUCTION

In various available auto-injectors, upon activation by a user, a needle is deployed, and fluid is delivered from the needle into the user. After completion of fluid delivery, the needle may be retracted for user comfort, needle safety, and positive perception of the product. However, many auto-injectors require separate user actions for both inserting and removing the needle. In addition, many available auto-injectors have a high profile. For example, existing pen-type injectors that align a medicament container along the axis of injection show a high profile relative to the skin of the patient. Patients may respond to such auto-injectors with anxiety, especially because the high profile is often perceived by patients to correspond to a long needle length, whereas the actual needle length may be relatively short. Additionally, many auto-injectors must be secured to the user for extended periods of time, which may be an inconvenience for the user.

### SUMMARY OF THE DISCLOSURE

In one aspect, the disclosure is directed to an auto-injector, comprising a housing having a longitudinal axis and a transverse axis, the housing having a shorter dimension along the transverse axis than along the longitudinal axis, wherein the transverse axis is perpendicular to the longitudinal axis; a flowpath having a first end and a second end; and a container enclosing a first fluid, the container extending from a first end toward a second end along or parallel to the longitudinal axis and being movable from a first position to a second position along or parallel to the longitudinal axis, the container being fluidly isolated from the flowpath in the first position and fluidly connected to the flowpath in the second position, the container further including a plunger configured to move from the first end toward the second end of the container to expel the first fluid from the container into the flowpath; and wherein the first end of the flowpath is insertable into the container and the second end of the flowpath is extendable from the housing in a direction along or parallel to the transverse axis through an opening in the housing.

The auto-injector further includes a fluid source configured to release a pressurized second fluid, wherein the container is movable from the first position to the second position by the release of the pressurized second fluid from the fluid source; and release of the pressurized second fluid from the fluid source urges the plunger from the first end toward the second end of the container to expel the first fluid from the container into the flowpath. The container includes a seal at the second end of the container; and in the first position, a gap is disposed between the seal and the first end of the flowpath. The first end of the flowpath pierces through the seal and enters the container upon movement of the container into the second position. The container is movable from a second position to a third position, upon loss of pressure from the pressurized second fluid to the container. The third position is the same as the first position. The third position is different than the first position. The auto-injector includes a first resilient member coupled to the container, wherein movement of the container from the first position to the second position compresses the resilient member; and the compressed resilient member expands to move the container to the third position, upon loss of pressure from the pressurized second fluid. The auto-injector includes a carrier, a driver coupled to the second end of the flowpath, the driver being slidable relative to the carrier between a retracted configuration and a deployed configuration; a shuttle configured to move the driver between the retracted configuration and the deployed configuration; and a stop configured to move from a first configuration to a second configuration, wherein the stop is configured to maintain the driver in the deployed configuration, and movement of the stop from the first configuration to the second configuration allows the shuttle to move the driver from the deployed configuration to the retracted configuration. Before activation, the driver is in contact with an impediment, and is prevented from moving out of the retracted configuration by the impediment. The impediment is coupled to the container. Movement of the container from the first position to the second position moves the impediment out of contact with the driver, allowing the driver to move from the retracted configuration to the deployed configuration.

In another aspect, the disclosure is directed to an auto-injector comprising a body housing a conduit; a fluid source configured to provide pressurized fluid into the conduit; a container fluidly connected to the conduit, the container housing a medicament and a plunger, wherein the container is configured to expel the medicament upon application of pressure from the pressurized fluid to the plunger; a pressure restrictor configured to restrict flow of the pressurized fluid in the conduit, the pressure restrictor defining a high pressure flow area and a low pressure flow area of the conduit; a valve including a valve inlet and a valve outlet, wherein the valve inlet is fluidly coupled to the conduit, and wherein the valve is configured to regulate flow of the pressurized fluid from the conduit to the valve outlet; and a flowpath extendable from the body and configured to deliver the medicament from the container to a patient, wherein a direction in which the container expels the medicament is offset from a direction in which the flowpath extends from the body.

The pressurized fluid is a gas. The medicament includes a monoclonal antibody. The pressure restrictor includes one of a porous material or a serpentine channel. A direction in which the container expels the medicament is approximately perpendicular to a direction in which the flowpath extends from the body. The container is fluidly connected to the low pressure flow area of the conduit, and the high pressure flow area of the conduit is fluidly connected to the valve inlet. The container is movable from a first container position to a second container position, and further comprising a spring mechanism configured to extend the flowpath from the body when the container is in the second container position. The valve is configured to allow flow of the pressurized fluid from the conduit to the valve outlet after the container expels at least a portion of the medicament, and wherein application of pressure from pressurized fluid flowing to the valve outlet is configured to actuate an additional mechanism of the auto-injector. The additional mechanism is a flowpath retraction mechanism. The flowpath retraction mechanism includes a rod movable by the pressurized fluid flowing through the valve outlet, wherein the rod is configured to cause the flowpath to retract after being moved by a first distance. The auto-injector may include a piston disposed in the valve outlet, and movable from a first position to a second position; and a secondary channel coupled to the fluid source and to the valve outlet, wherein the secondary channel is sealed from the valve outlet by the piston when the piston is in the first position; and the secondary channel is fluidly connected to the valve outlet when the piston is in the second position, such that pressurized fluid flows from the fluid source, through the secondary channel, and through the valve outlet. The valve is configured to prevent flow of the pressurized fluid from the conduit to the valve outlet while the container is expelling medicament.

In yet another aspect, the present disclosure is directed to an auto-injector comprising a conduit; a fluid source configured to provide pressurized fluid into the conduit; a container fluidly connected to the conduit, the container housing a plunger, wherein the plunger is movable from a first position to a second position upon application of pressure from the pressurized fluid; a pressure restrictor configured to restrict flow of the pressurized fluid through the conduit, the pressure restrictor defining a high pressure flow area and a low pressure flow area of the conduit; and a valve, including a first valve inlet fluidly coupling the high pressure flow area of the conduit to a first valve cavity; a second valve inlet fluidly coupling the low pressure flow area of the conduit to a second valve cavity; and a valve outlet, wherein the valve is configured to regulate flow of the pressurized fluid from the low pressure flow area of the conduit to the valve outlet.

The first valve cavity and the second valve cavity are separated by one of a diaphragm or a piston. The first valve cavity and the second valve cavity are separated by a diaphragm held in a stretched configuration, and wherein the diaphragm is held in place by at least one of a clamp or a groove. The valve is configured to allow flow of the pressurized fluid from the low pressure flow area of the conduit to the valve outlet when a fluid pressure in the low pressure flow area of the conduit is within a threshold range of a fluid pressure in the high pressure flow area of the conduit. The valve outlet is fluidly connected to a flowpath retraction mechanism configured to be actuated by pressurized fluid flowing through the valve outlet. The valve outlet is fluidly connected to a ventilation aperture.

The auto-injector further includes a fluid source configured to expel a pressurized fluid, wherein expulsion of the pressurized fluid from the fluid source moves an entirety of the container from the first position to the second position in a direction along or parallel to the longitudinal axis of the housing. The auto-injector further includes a dispensing chamber coupled to the fluid source and a sliding seal coupled to an outer surface of the container and to an inner surface of the dispensing chamber, wherein expulsion of the pressurized fluid from the fluid source into the dispensing chamber urges the entirety of the container and the sliding seal to move relative to the dispensing chamber along or parallel to the longitudinal axis. The container expels the treatment fluid into the flowpath along or parallel to the longitudinal axis. Expulsion of the pressurized fluid is activated only after the shroud has collapsed or retracted. Expulsion of the pressurized fluid cannot be stopped after its initiation. Alternately, expulsion of the pressurized fluid is ceased after its initiation. In some cases, however, expansion of the shroud or retraction of the flowpath through the opening of the shroud stops expulsion of the pressurized fluid from the fluid source.

The container includes a seal at the second end, and movement of the container into the second position causes the first end of the flowpath to pierce the seal. A second end of the flowpath is extendable from the housing only after the shroud is collapsed or retracted. Entireties of the container and the flowpath move along the transverse axis during collapse or retraction of the shroud. The flowpath of this auto-injector is nonlinear. The auto-injector further includes an actuator coupled to the fluid source, wherein activation of the actuator by a user initiates expulsion of the pressurized fluid, the actuator comprising a button, switch, trigger mechanism, or a combination thereof. Deactivation of the actuator stops expulsion of the pressurized fluid from the fluid source. The auto-injector may be a handheld auto-injector configured to complete an injection procedure in 30 seconds or less. The auto-injector further includes a power source configured to move the plunger from the first end toward the second end of the container. Activation of the power source causes the container to move from a first position along the longitudinal axis, to a second position along the longitudinal axis. The power source includes a spring, a resilient member, a motor, or a pressurized fluid source.

In another aspect, the present disclosure is directed to an auto-injector comprising a housing having a longitudinal axis and a transverse axis, the housing having a shorter dimension along the transverse axis than along the longitudinal axis, wherein the transverse axis is perpendicular to the longitudinal axis, and the housing contains a shroud configured to collapse or retract along the transverse axis; a power source; a flowpath having a first end and a second end; and a container containing a treatment fluid and a plunger, the container extending from a first end toward a second end along or parallel to the longitudinal axis, wherein, activation of the power source moves the plunger from the first end toward the second end of the container to expel the treatment fluid out of the container and into the flowpath, wherein the second end of the flowpath is extendable from the housing in a direction along or parallel to the transverse axis through an opening in the shroud when the shroud is collapsed or retracted; and wherein the auto-injector is a handheld auto-injector configured to complete an injection procedure in 30 seconds or less. The power source is configured to be activated after the shroud is collapsed or retracted.

In another aspect, the present disclosure is directed to an injection device that includes a collapsible housing movable between an expanded configuration and a collapsed or retracted configuration, a fluid source configured to release a pressurized fluid, and a flowpath having a first end and a second end, the flowpath being entirely contained within the collapsible housing in the expanded configuration. The second end of the flowpath is configured to extend out of the collapsible housing in the collapsed or retracted configuration, wherein the first end of the flowpath and the second end of the flowpath extend along axes that are offset from one another. The injection device also includes a container containing a treatment fluid, the container extending from a first end toward a second end along or parallel to a longitudinal axis of the container, and the container is movable from a first position to a second position by a flow of the pressurized fluid from the fluid source, the container being fluidly isolated from the flowpath when the collapsible housing is in the expanded configuration, and the container is in fluid communication with the flowpath when the collapsible housing in the compressed configuration and after the container is moved to the second position, the container further including a plunger, wherein, after the container is moved to the second position, further release of the pressurized fluid from the fluid source urges the plunger from the first end toward the second end of the container to expel the treatment fluid from the container into the first end of the flowpath, and out of the second end of the flowpath, wherein the auto-injector is a handheld auto-injector configured to complete an injection procedure in 30 seconds or less.

Movement of the collapsible housing to the collapsed or retracted configuration automatically causes the release of the pressurized fluid from the fluid source. The collapsible housing is configured to compress by application of a force to an outer surface of the collapsible housing, and is configured to expand upon release of the force to the outer surface. Alternatively, the collapsible housing is configured to compress by application of a force to an outer surface of the collapsible housing, and is configured to remain in the collapsed or retracted configuration upon release of the force to the outer surface.

### BRIEF DESCRIPTION OF THE FIGURES

The accompanying drawings, which are incorporated in and constitute a part of this specification, illustrate various examples and together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect nor embodiment thereof, nor to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIGS. 1 and 1A are perspective views of auto-injectors, according to examples of the disclosure.

FIG. 2 is an illustration of an auto-injector.

FIGS. 3A-3C are schematic views of features of an auto-injector.

FIG. 3D is an illustration of a sliding seal disposed within an auto-injector.

FIGS. 3E-G illustrate details of an auto-injector with a plurality of containers.

FIGS. 4A and 4B are schematic and cross-sectional views of an exemplary valve used with an auto-injector.

FIG. 5 is a schematic and cross-sectional view of another exemplary valve used with an auto-injector.

FIGS. 6, 7A, and 7B, illustrate exemplary flow restrictors used with an auto-injector.

FIGS. 7C-7F illustrate additional exemplary valves used with an auto-injector.

FIGS. 7G and 7H illustrate an additional exemplary valve used in an auto-injector.

FIGS. 7I-N illustrate additional details of a diaphragm.

FIG. 7O illustrates a partially exploded view of another exemplary valve.

FIGS. 8A-8D illustrate an exemplary venting system.

FIGS. 9A-9H illustrate another exemplary venting system.

FIGS. 9I-9K illustrate yet another exemplary venting system.

FIGS. 10A-F illustrate yet another exemplary venting system.

FIGS. 11 and 11A-11H, 12A-12C, 13A-13D, 14A, 14B, 15A, 15B, and 16A-16E, show various venting mechanisms according to the disclosure.

FIG. 17 is a schematic view of features of an auto-injector.

FIG. 18A is an exploded view of a needle mechanism.

FIGS. 18B-D are schematic illustrations of portions of a needle mechanism.

FIGS. 19-22 are side views of the needle mechanism.

FIG. 23 is a view of a portion of the needle mechanism.

FIGS. 23A-L illustrate various mechanisms for initiating needle insertion and/or retraction.

FIG. 23M is a schematic view of an auto-injector, according to another exemplary embodiment.

FIG. 23N is a schematic view of another alternative auto-injector, according to another embodiment.

FIGS. 23O-Q illustrate another mechanism for initiating needle insertion and/or retraction.

FIGS. 23R-U are schematic views of additional features of an auto-injector, according to examples of the disclosure.

FIG. 24 is a schematic view of an auto-injector, according to another exemplary embodiment.

FIGS. 25A and 25B are illustrations of a drive system used with an auto-injector.

FIGS. 26A and 26B show an alternative mechanism for sealing a container.

FIGS. 27A, 27B, 28A, and 28B show various mechanisms for establishing fluid communication between a container and a fluid conduit.

FIGS. 29A and 29B show various mechanisms for sealing a first end of a container.

FIGS. 30A, 30B, 31A, 31B, 32A, and 32B show various mechanisms for activating a fluid source.

FIGS. 32C-V show various additional mechanisms for activating a fluid source.

FIGS. 33A and 33B show an auto-injector having a retractable shroud.

FIGS. 34A-B, 35A-B, 36A-B, 37A-B, 38A-B, 39A-B, 40A-B, 41A-E, 42A-C, 43A-D, 44A-D, 45A-B, 46A-E, 47A-D, 48A-I, and 49A-F illustrate various exemplary transverse auto-injectors of the present disclosure.

FIGS. 50A-J illustrate various surface modifications for auto-injectors of the present disclosure.

FIGS. 51A-D illustrate various locations for labels on auto-injectors of the present disclosure.

FIGS. 52A-C illustrate a peel-off seal and contact switch of the present disclosure.

FIGS. 53A and 53B illustrate various indicators for auto-injectors of the present disclosure.

FIGS. 54A-N illustrate the use of various indicator flags in auto-injectors of the present disclosure.

FIGS. 55A-G illustrate the use of window tinting or covers in auto-injectors of the present disclosure.

FIGS. 56A-E illustrate various locations for labels on auto-injectors of the present disclosure.

FIGS. 57A-E illustrate various features for providing visual indication of a needle insertion depth, according to various embodiments.

FIGS. 58A-H illustrate various features for providing visual indication of the stage and/or progress of injection, according to various embodiments of another auto-injector.

FIGS. 59A-R illustrate various features for restricting flow of gas or fluid, according to various embodiments of another auto-injector.

FIG. 60A is a perspective view of an auto-injector in an initial, unactuated state, according to an example of the disclosure.

FIG. 60B is a perspective view of a fluid-actuated auto-injector in an initial, unactuated state, according to an example of the disclosure.

FIG. 61 is a perspective view of the auto-injector of FIG. 60B in an intermediate state.

FIG. 62 is a perspective view of the auto-injector of FIG. 60B showing coupling of a medicament cartridge with a flowpath.

FIG. 63 is a perspective view of the auto-injector of FIG. 60B during injection,

FIG. 64 is a perspective view of the auto-injector of FIG. 60B after completion of an injection.

FIGS. 65A-H illustrate a sterile connector, according to another embodiment of the disclosure.

Again, there are many embodiments described and illustrated herein. The present disclosure is neither limited to any single aspect nor embodiment thereof, nor to any combinations and/or permutations of such aspects and/or embodiments. Each of the aspects of the present disclosure, and/or embodiments thereof, may be employed alone or in combination with one or more of the other aspects of the present disclosure and/or embodiments thereof. For the sake of brevity, many of those combinations and permutations are not discussed separately herein.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the cross-sectional views are not drawn to scale and should not be viewed as representing proportional relationships between different components. The cross-sectional views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. In the discussion that follows, relative terms such as “about,” “substantially,” “approximately,” etc. are used to indicate a possible variation of ±10% in a stated numeric value.

As described above, existing auto-injectors often require multiple user interactions to self-administer a drug, including, e.g., separate user interactions for deploying a needle and subsequently retracting the needle after drug delivery. These additional steps can increase complexity of self-administration of drugs, introduce user errors, and cause user discomfort. Accordingly, the present disclosure is directed to various embodiments of an injection device (e.g., auto-injector) that simplifies self-administration of drugs, or other therapeutic agents, by a user. Specifically, according to certain embodiments, the auto-injector may not require any additional user interaction to withdraw a needle once the needle is subcutaneously inserted into the user. Thus, auto-injectors of the present disclosure are simplified to help prevent misuse or user error.

As described above, existing auto-injectors often require multiple components and user operations to administer a drug, including, various spring or motor mechanisms. These additional components can increase complexity of manufacture and introduce mechanical faults or user error. Accordingly, the present disclosure is directed to various embodiments of an injection device (e.g., auto-injector) that simplifies and refines administration of drugs, or other therapeutic agents.

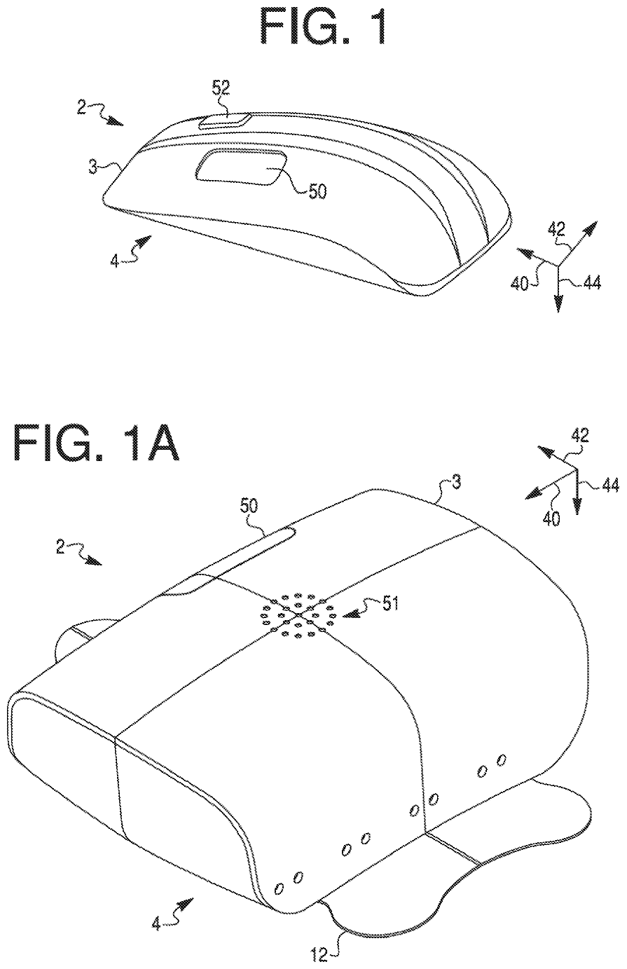

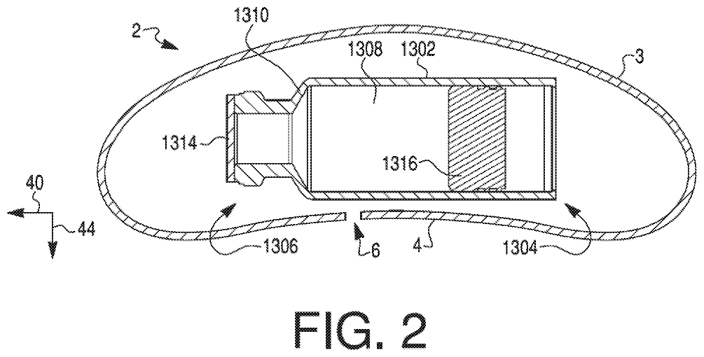

An example of such an auto-injector 2 is shown in FIGS. 1 and 2. Auto-injector 2 may include a housing 3 having a tissue-engaging (e.g., bottom) surface 4 through which a needle may be deployed and retracted via an opening 6 (FIG. 2). Housing 3 may include a transparent window 50 to enable a viewer to visualize a container disposed within housing 3. Housing 3 also may include an actuator or button 52 configured to actuate a drive mechanism for delivering medicament (treatment fluid) contained within the auto-injector 2 into a patient (e.g., fluid source 1366 described in further detail below). In some embodiments, it is contemplated that auto-injector 2 will not include any electrical components. In other embodiments, one or more displays or LEDs (not shown) may be disposed within housing 3, and/or housing 3 may include a plurality of openings 51 (see alternative embodiment of FIG. 1A) configured to facilitate the travel of sound generated within housing 3 (by, e.g., a speaker). Auto-injector 2 may have any suitable dimensions suitable to enable portability and self-attachment by a user. Auto-injector 2, for example, may have a length from about 0.5 inches to about 5.0 inches, a width of about 0.5 inches to about 3.0 inches, and a height from 0.5 inches to about 2.0 inches. Auto-injector 2 also may include a grippy or tacky coating such that the outer surface of auto-injector 2 is a non-slip surface.

Auto-injector 2 may be oriented about a longitudinal axis 40 (e.g., an X axis), a lateral axis 42 (e.g., a Y axis) that is substantially perpendicular to longitudinal axis 40, and a transverse axis 44 (e.g., a Z axis) that is substantially perpendicular to both longitudinal axis 40 and lateral axis 42. Transverse auto-injectors of the present disclosure, in some embodiments, may have a long dimension along longitudinal axis 40 than along transverse axis 44.

In certain embodiments of auto-injector 2, such as when auto-injector 2 is a wearable auto-injector, auto-injector 2 may include an adhesive patch 12 as shown in FIG. 1A. Adhesive patch 12 may be coupled to tissue-engaging surface 4 to help secure auto-injector 2 to a user's body (e.g., skin). Adhesive patch 12 may be formed from fabric or any other suitable material, and may include an adhesive. The adhesive may be an aqueous or solvent-based adhesive, or may be a hot melt adhesive, for example. Suitable adhesives also include acrylic based, dextrin based, and urethane based adhesives as well as natural and synthetic elastomers. In some examples, the adhesive provided on patch 12 may be activated upon contact with a user's skin. In yet another example, patch 12 may include a non-woven polyester substrate and an acrylic or silicone adhesive. Patch 12 may be joined to housing 3 by, e.g., a double-sided adhesive, or by other mechanisms like ultrasonic welding. Patch 12 may have a length dimension (e.g., a dimension parallel to longitudinal axis 40) greater than a width (e.g., a dimension parallel to lateral axis 42) of auto-injector 2.

In other embodiments of the disclosure, auto-injector 2 does not include an adhesive patch. For example, auto-injector 2 may be a handheld auto-injector e.g., FIG. 1), as opposed to a wearable auto-injector (e.g., FIG. 1A). In at least some embodiments, a handheld auto-injector may require a user to hold the auto-injector against the user's skin for the entirety of an injection procedure, whereas, a wearable injector may include features for securing the wearable auto-injector to the skin. For example, a wearable auto-injector may include one or more features, such as, e.g., an adhesive patch (e.g., adhesive patch 12), straps, or the like, for securing to the user. In some embodiments, a handheld auto-injector according to this disclosure may be configured to deliver a medicament volume of less than 3.5 mL (or a medicament volume from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL), whereas a wearable auto-injector may be configured to deliver a medicament volume of greater than 3.5 mL, greater than 4.0 mL, or greater than 5.0 mL.

Furthermore, handheld auto-injectors according to the present disclosure may be configured to complete an injection procedure, as measured from 1) a point at which that the user places the auto-injector onto the skin to 2) a point at which the user removes the auto-injector from the skin after completion of an injection, in less than about 30 seconds, less than about 25 seconds, less than about 20 seconds, less than about 15 seconds, or less than about 10 seconds. A wearable auto-injector may or will take longer than 30 seconds to complete the same steps 1) and 2) discussed above, i.e., from 1) the point in time at which the auto-injector is placed onto a user's skin, to 2) the point in time at which the auto-injector is removed from the skin.

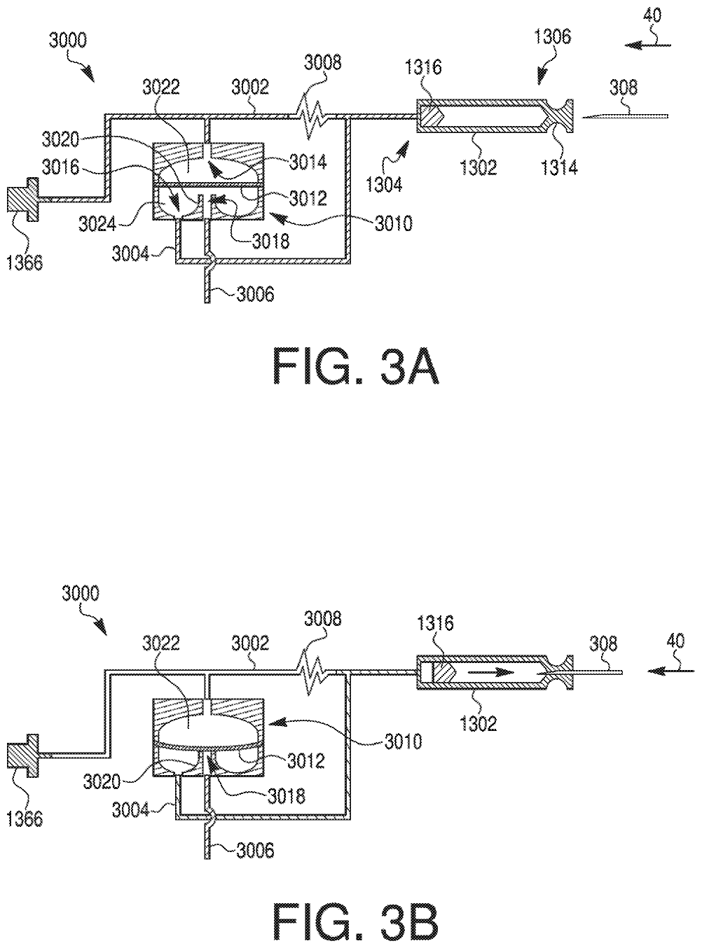

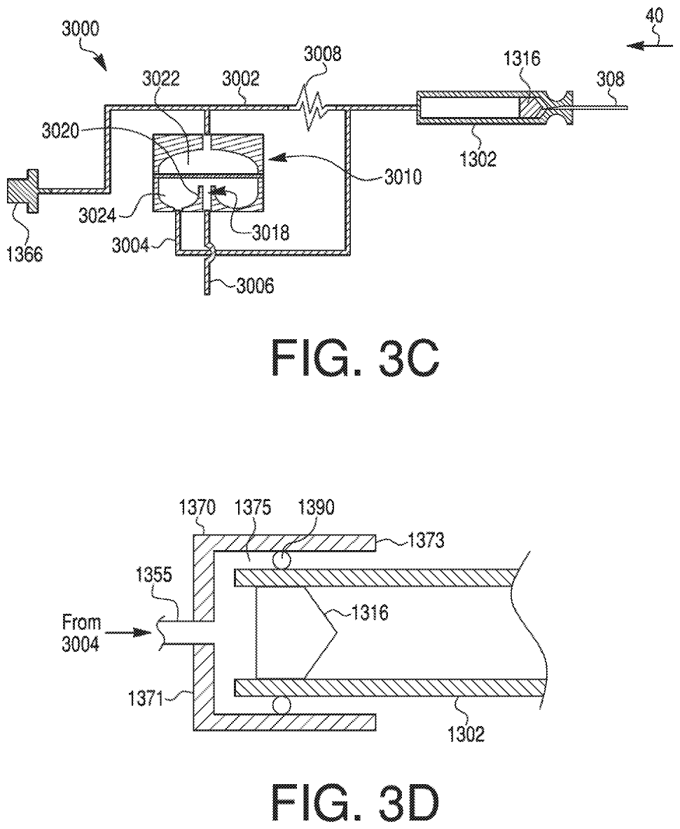

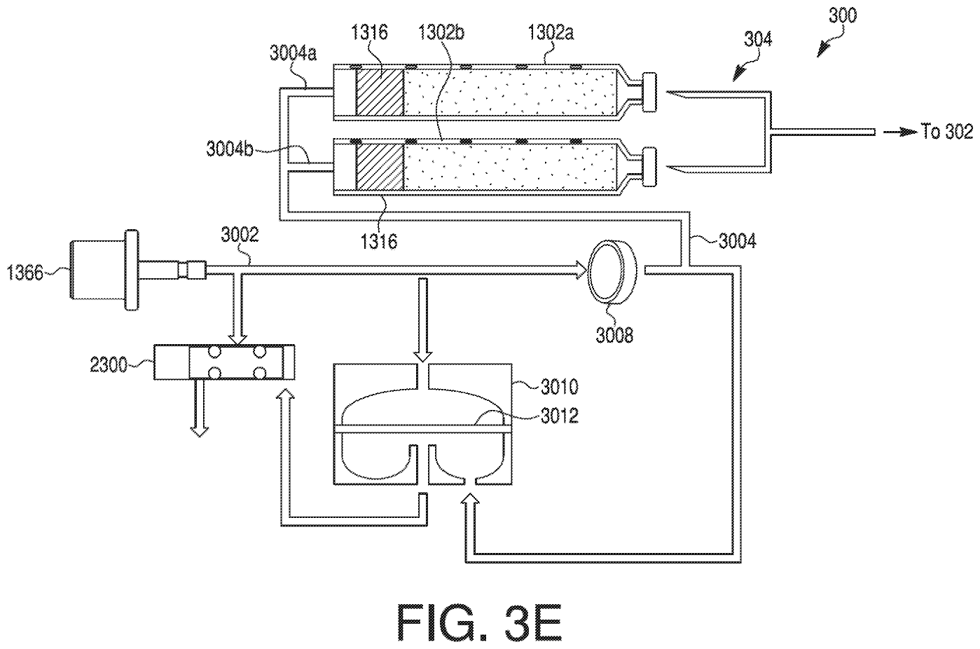

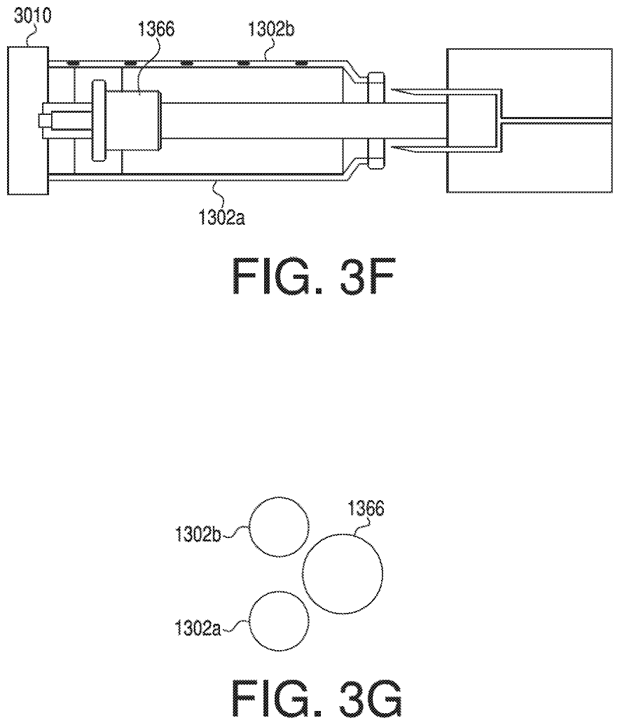

Referring to FIGS. 2 and 3A-3C, auto-injector 2 may include a primary container, chamber, syringe, cartridge, or container 1302 with a first end 1304 and a second end 1306. Container 1302 also may include a cavity 1308 having an opening at first end 1304 and extending toward second end 1306. Second end 1306 may include a seal 1314 configured to assist with closing and/or sealing of second end 1306, and allow for needle 308 (e.g., a staked needle shown in FIGS. 3A-3C) to be inserted into container 1302. Cavity 1308 may be closed at first end 1304 by a piston 1316.

The “nominal volume” (also called the “specified volume,” or “specified capacity”) of a container refers to the container's maximum capacity, as identified by the container's manufacturer or a safety standards organization. A manufacturer or a safety standards organization may specify a container's nominal volume to indicate that the container can be filled with that volume of fluid (either aseptically or not) and be closed, stoppered, sterilized, packaged, transported, and/or used while maintaining container closure integrity, and while maintaining the safety, sterility, and/or aseptic nature of the fluid contained inside. In determining the nominal volume of a container, a manufacturer or a safety standards organization may also take into account variability that occurs during normal filling, closing, stoppering, packaging, transportation, and administration procedures. As an example, a prefillable syringe may be either hand- or machine- filled with up to its nominal volume of fluid, and may then be either vent tube- or vacuum-stoppered, without the filling and stoppering machinery and tools touching and potentially contaminating the contents of the syringe. Alternatively, the stopping machinery and tools may be sterile or aseptic, and are able to contact the contents of the syringe and/or the syringe itself without resulting in any contamination.

Container 1302 may have about a 5.0 mL nominal volume in some examples, although any other suitable nominal volume (e.g., from about 0.5 mL to about 50.0 mL, or from about 2.0 mL to about 10.0 mL, or from about 3.0 mL to about 6.0 mL, or from about 1.0 mL to about 3.0 mL, or from about 2.0 mL to about 5.0 mL, or another suitable range) also may be utilized depending on the drug to be delivered. In other examples, container 1302 may have a nominal volume greater than or equal to about 0.5 mL, or greater than or equal to about 2.0 mL, or greater than or equal to about 3.0 mL, or greater than or equal to about 4.0 mL, or greater than or equal to about 5.0 mL. Container 1302 may contain and preserve a drug for injection into a user, and may help maintain sterility of the drug. In one embodiment, container 1302 may be configured to deliver a delivered quantity of medicament (e.g., from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL, greater than about 1.0 mL, greater than about 2.0 mL, greater than about 3.0 mL, greater than about 4.0 mL, greater than about 5.0 mL, greater than about 10.0 mL, greater than about 20.0 mL or another delivered quantity). The delivered quantity may be less than the nominal volume of container 1302. Furthermore, in order to deliver the delivered quantity of medicament to a user, container 1302 itself may be filled with a different quantity of medicament than the delivered quantity (i.e., a filled quantity). The filled quantity may be an amount of medicament greater than the delivered quantity to account for medicament that cannot be transferred from container 1302 to the user due to, e.g., dead space in container 1302 or fluid conduit 300. Thus, while container 1302 may have a nominal volume of 5 mL, the filled quantity and delivered quantity of medicament may be less than 5 mL.

In one embodiment, when container 1302 is used in a handheld auto-injector, the delivered quantity of medicament from container 1302 may be from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL. The delivered quantity of medicament may be related to the viscosity of the medicament and the hand-held nature of auto-injector 2. That is, in at least some embodiments, at certain viscosities, higher volumes of medicament may prohibit the ability of auto-injector 2 to complete an injection procedure in less than an acceptable amount of time, e.g., less than about 30 seconds. Thus, the delivered quantity of medicament from auto-injector 2 may be set such that an injection procedure, measured from 1) the point in time at which the auto-injector is placed onto a user's skin, to 2) the point in time at which the auto-injector is removed from the skin, is less than about 30 seconds or less than about another time period (e.g., less than about 25 seconds, less than about 20 seconds, less than about 15 seconds, or less than about 10 seconds). When the delivered quantity and viscosity of the medicament is too high, auto-injector 2 may not be able to function as a handheld auto-injector, since the time required to complete the injection procedure may be higher than commercially or clinically acceptable for handheld devices. Again, as stated above, in embodiments where container 1302 is used in a hand-held auto-injector, regardless of the nominal volume of container 1302, the delivered quantity of medicament from container 1302 may be set such that the injection procedure as defined above is completed in a relatively short period of time (so as to avoid the need for additional features to attach the auto-injector 2 to the user so that auto-injector 2 is a wearable auto-injector).

However, it is contemplated that various embodiments of the present disclosure relate to wearable auto-injectors that deliver relatively large quantities of medicament (e.g., greater than about 3.5 mL) and/or have relatively longer injection procedure times as opposed to handheld auto-injectors (e.g., longer than about 30 seconds, longer than about 1 minute, longer than about 2 minutes, longer than about 5 minutes, or longer than about 1 hour) to complete an injection procedure as measured from 1) the point in time at which the auto-injector is placed onto a user's skin, to 2) the point in time at which the auto-injector is removed from the skin).

Container 1302 may have about a 13 mm diameter neck, about a 45 mm length, and an internal diameter of about 19.05 mm. In another embodiment, container 1302 may be a standard 3 mL container having an 8 mm crimp top, a 9.7 mm inner diameter, and a 64 mm length. These values are merely exemplary, and other suitable dimensions may be utilized as appropriate. In some examples, container 1302 may be formed using conventional materials, and may be shorter than existing devices, which can help auto-injector 2 remain cost-effective and small. In some embodiments, container 1302 may be a shortened ISO 10 mL cartridge.

Auto-injectors of the present disclosure may be configured to deliver highly viscous liquid to a patient. For example, auto-injectors of the present disclosure may be configured to deliver liquid having a viscosity from about 0 cP to about 100 cP, from about 5 cP to about 45 cP, from about 10 cP to about 40 cP, from about 15 cP to about 35 cP, from about 20 cP to about 30 cP, or about 25 cP. Septum 1314 may include an uncoated bromobutyl material, or another suitable material. Piston 1316 may include a fluoropolymer coated bromobutyl material, and, in some embodiments, may include a conical nose to help reduce dead volume within container 1302. Piston 1316 may include one or more rubber materials such as, e.g., halobutyls (e.g., bromobutyl, chlorobutyl, florobutyl) and/or nitriles, among other materials.

Piston 1316 may be movable by a pressurized fluid expelled from a fluid source, such as, e.g., fluid source 1366 (FIGS. 3A-3C). Pressurized gas expelled from fluid source 1366 may translate piston 1316 and container 1302 in a direction toward second end 1306. The movement of piston 1316 toward second end 1306 causes piston 1316 to act against the contents within container 1302 (e.g., drugs, medications), which ultimately transfers force against second end 1306 of container 1302, causing container 1302 to move along longitudinal axis 40. In some embodiments, transverse auto-injectors may be oriented such that fluid source 1366 and piston 1316 are offset, or are otherwise not longitudinally aligned with one another.

Fluid source 1366 may include a non-latching can or a latching can. Fluid source 1366 may be configured to dispense liquid propellant for boiling outside of fluid source 1366 so as to provide a pressurized gas (vapor pressure) that acts on piston 1316. In some embodiments, once opened, the latching can may be latched open so that the entire contents of propellant is dispensed therefrom. Alternatively, in some embodiments, fluid source 1366 may be selectively controlled, including selectively activated and deactivated. For example, in an alternative embodiment, the flow of pressurized gas from fluid source 1366 may be stopped after flow is initiated.

The fluid from fluid source 1366 may be any suitable propellant for providing a vapor pressure to drive piston 1316. In certain embodiments, the propellant may be a liquefied gas that vaporizes to provide a vapor pressure. In certain embodiments, the propellant may be or contain a hydrofluoroalkane (“HFA”), for example HFA134a, HFA227, HFA422D, HFA507, or HFA410A. In certain embodiments, the propellant may be or contain a hydrofluoroolefin (“HFO”) such as HFO1234yf or HFO1234ze. In some embodiments, fluid source 1366 may be a high-pressure canister configured to hold a compressed gas.

To initiate movement of container 1302 along longitudinal axis 40, fluid source 1366 may be actuated so as to move to an open configuration in which propellant may exit the fluid source 1366 as a pressurized gas. In some embodiments, the actuation is irreversible such that the flow of pressurized gas from fluid source 1366 is not able to be stopped.

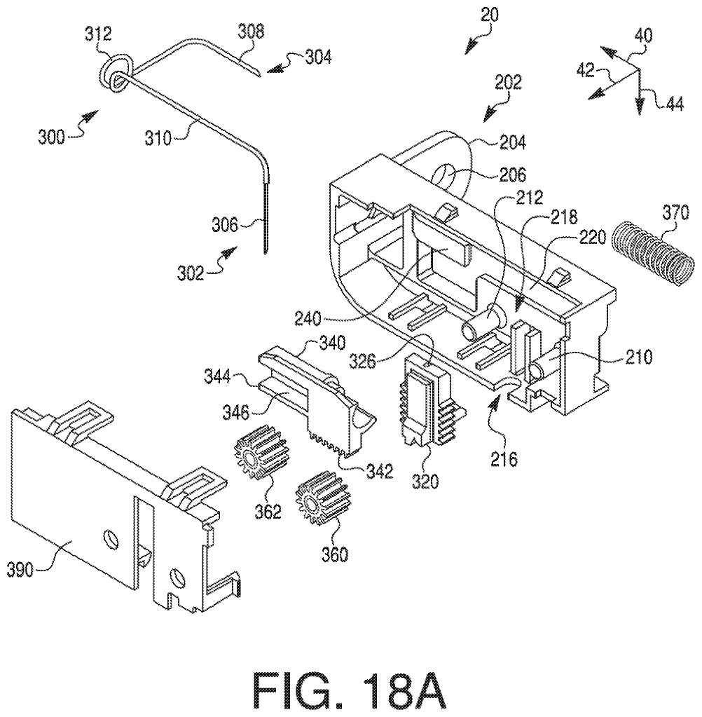

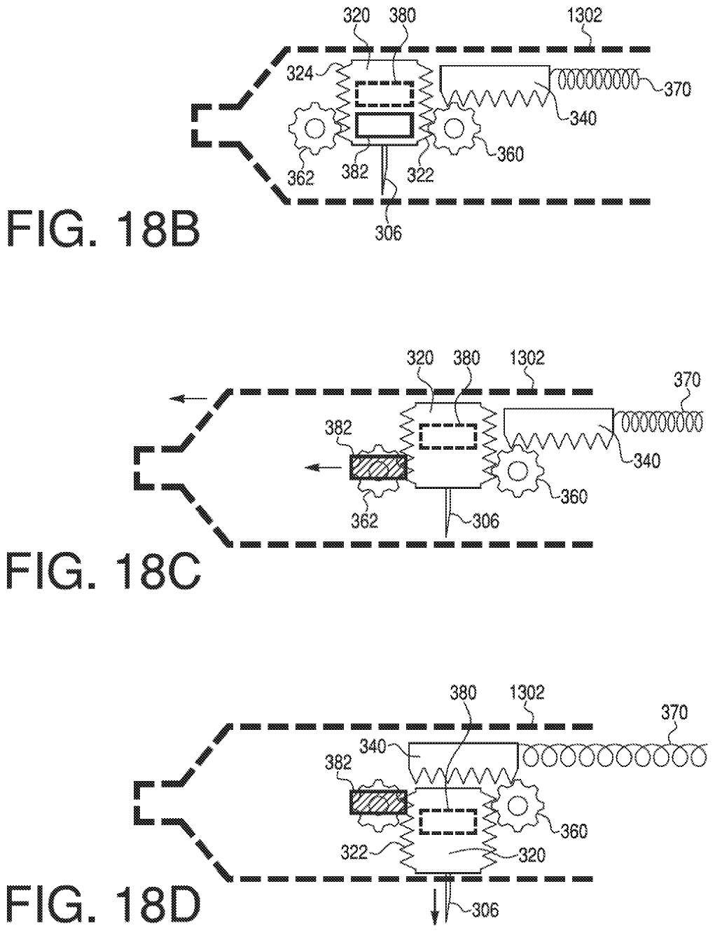

In the pre-activated state of auto-injector 2 shown in FIG. 3A, needle 308 may be spaced apart from the second end 1306 of container 1302. To move auto-injector 2 from the pre-activated state of FIG. 3A, fluid source 1366 may be activated as set forth above to move container 1302 along longitudinal axis 40 toward needle 308. Because the needle 308 is not yet in fluid communication with container 1302, activation of fluid source 1366 applies a pressure against the fluid contained in container 1302, which is then applied to container 1302 itself. This pressure causes container 1302 to move toward the needle 308, ultimately forcing needle 308 through the septum 1314 such that the needle 308 is in fluid communication with the contents of container 1302. This movement also may correspond to the movement of an impediment 382 relative to a protrusion 380 (FIGS. 18B-18D), which enables protrusion 380 to clear impediment 182 to inject a needle 306. In other words, pressurized gas from fluid source 1366 also may drive the movement of the impediment 382 relative to protrusion 380, to initiate the injection of a needle 306 into the user (described in further detail below). Once needle 308 is in fluid communication with container 1302, further movement of piston 1316 toward second end 1306 urges fluid through needle 308 and a remainder of fluid conduit 300 (shown in FIG. 18A).

FIGS. 3A-3C depict a drive system 3000 for providing the drive force to deliver fluid from container 1302 to a patient. Drive system 3000 includes fluid source 1366, a high pressure (first) line 3002, a low pressure (second line) 3004, and a third line 3006, a flow restrictor 3008, and a valve 3010. Valve 3010 includes a diaphragm 3012, a high pressure (first) inlet 3014, a low pressure (second) inlet 3016, and a conduit 3018. Conduit 3018 is formed within a valve seat 3020 that extends into the interior of valve 3010. Within valve 3010, diaphragm 3012 defines a high pressure (first) cavity 3022 and a low pressure (second) cavity 3024.

When fluid source 1366 is actuated, pressurized gas may flow through high pressure line 3002 and flow restrictor 3008, and then to container 1302. Some pressurized gas from high pressure line 3002 may be diverted to high pressure cavity 3022 via high pressure inlet 3014. This causes diaphragm 3012 to move toward and seal conduit 3018 in valve seat 3020 (FIG. 3B). Downstream of pressure restrictor 3008, reduced-pressure gas is diverted to low pressure cavity 3024 via low pressure line 3004 and low pressure inlet 3016. The pressure difference between high pressure cavity 3022 and low pressure cavity 3024 provides the force required to seal conduit 3018 by diaphragm 3012. The low pressure line 3004 also directs the pressurized gas to initiate movement of container 1302 toward needle 308, and to subsequently urge piston 1316 along or parallel to axis 40 and expel medicament through container 1302 until piston 1316 reaches the end of container 1302 (and bottoms out).

When piston 1316 bottoms out at the end of the injection (FIG. 3C), the pressure across high pressure cavity 3022 and low pressure cavity 3024 equilibrates, causing diaphragm 3012 to lift off of valve seat 3020 and open conduit 3018. This allows the gas from low pressure line 3004 to vent out of the system through conduit 3018 and third line 3006.

The mechanism by which low pressure line 3004 drives movement of container 1302 and piston 1316 is described with further reference to FIG. 3D. Fluid source 1366 may be configured to contain enough pressurized fluid so that release of the pressurized gas may actuate both movement of the container 1302 and piston 1316, as described in greater detail below. In some cases, fluid source 1366 may contain excess pressurized gas, i.e., more fluid than is necessary to complete delivery of the contents of container 1302.

Auto-injector 2 may further include a rail 1370 having a cylindrical structure extending along the longitudinal axis of auto-injector 2. Rail 1370 may have an inner surface which may define a lumen. Rail 1370 may coaxially surround at least a portion of container 1302. For example, container 1302 may be positioned inside the lumen formed by rail 1370. Rail 1370 may be spaced from the container 1302 such that the container 1302 may slide along the length of the rail 1370.

Rail 1370 may include a base 1371, as well as a rim 1373. Base 1371 may include a conduit 1355 configured to receive pressurized gas from low pressure line 3004. The pressurized gas may be delivered from conduit 1355 to a dispensing chamber (cavity) 1375 formed by the inner surface of rail 1370, a sliding seal 1390, piston 1316, and an outer wall of container 1302.

Sliding seal 1390 may be disposed between the container 1302 and the rail 1370 to facilitate movement of the container 1302 by preventing pressurized gas from leaking past the sliding seal 1390. For example, sliding seal 1390 may be positioned along an inner surface of rail 1370 and an outer surface of container 1302 to facilitate movement of container 1302 along rail 1370. The container 1302, sliding seal 1390, and rail 1370 may be concentric.

In some embodiments, sliding seal 1390 may be fixed to a position at the outer surface of container 1302, while sliding seal 1390 is configured to slide along the inner surface of rail 1370 with container 1302. For example, the positioning between sliding seal 1390 and container 1302 may remain static even as container 1302 moves relative to rail 1370. The sliding seal 1390 and container 1302 may move, as a unit, from the base 1371 of rail 1370 towards the rim 1373 of rail 1370. In other words, sliding seal 1390 and container 1302 may translate simultaneously together along the rail 1370. In another embodiment, the relative position of rail 1370 and sliding seal 1390 may be static, while container 1302 translates towards needle 308. In yet another embodiment, sliding seal 1390 may move relative to both rail 1370 and container 1302. In some embodiments, the position of container 1302 may remain static relative to the housing 3, while fluid conduit 300 is moved through seal 1314 to put container 1302 and fluid conduit 300 into fluid communication.

In some cases, rail 1370 may include one or more stoppers (not shown) along its inner surface. The stoppers may abut sliding seal 1390 and stop the motion of sliding seal 1390 along the longitudinal axis. Alternately or in addition, one or more stoppers may be positioned at the outer surface of container 1302 to stabilize or stop the motion of container 1302. Due to the coupling between the sliding seal 1390 and container 1302, translation of the container 1302 along the longitudinal axis may stop once the sliding seal 1390 is prevented from moving along the longitudinal axis. It also is contemplated that no such stopper may be required, and that longitudinal movement of container 1302 will cease once seal 1314 is punctured by needle 308, since further movement of piston 1316 at that point will urge medicament through needle 308.

Prior to use of the auto-injector 2, dispensing chamber 1375 may be at a first volume. After actuation of fluid source 1366, pressurized fluid released from the fluid source 1366 may fill the dispensing chamber 1375. The dispensing chamber 1375 may expand as compressed pressurized gas pushes piston 1316, container 1302, and sliding seal 1390, urging that entire assembly along the longitudinal axis. As previously described, sliding seal 1390 and container 1302 may shift towards to rim 1373, along or parallel to the longitudinal axis of auto-injector 2, until container 1302 (e.g., seal 1314) contacts needle 308. This contact between seal 1314 and the needle 308 may cause needle 308 to puncture seal 1314 and place fluid conduit 300 into fluid communication with container 1302. Pressurized gas may apply pressure to piston 1316 and thus push piston 1316 through the body of container 1302. As piston 1316 moves through container 1302, the movement of piston 1316 may force medicament to flow through fluid conduit 300 to the patient via needle 306.

In one embodiment, in a pre-activated state, needle 308 may be disposed within seal 1314. In other words, prior to the release of any pressurized gas from fluid source 1366, the end of needle 308 may be disposed within seal 1314 but not in communication with container 1302. In such an embodiment, seal 1314 may include a solid plug which is devoid of any holes, cavities, or openings, and which may be formed of a first rubber material. The first rubber material may be permeable to a sterilizing gas, such as, e.g., ethylene oxide or vaporized hydrogen peroxide. The first rubber material may include one or more of isoprene, ethylene propylene diene monomer (M-class) rubber (EPDM), and styrene-butadiene, among others. The permeability of the first rubber material to a sterilizing gas may allow needle 308, which is disposed within the plug, to be sterilized before use. The plug may be molded about needle 308, so that needle 308 is impaled into the plug. Seal 1314 also may include a base that is impermeable to the sterilizing gas to prevent contamination and/or alteration of a drug contained within container 1302. The base may include impermeable rubbers such as, e.g., halobutyls (e.g., bromobutyl, chlorobutyl, florobutyl) and/or nitriles, among other materials.

In some embodiments, container 1302, rail 1370, and sliding seal 1390 may be configured such that container 1302 may be replaceable. For example, rail 1370 and sliding seal 1390 may include one or more openings through which container 1302 may be inserted.

FIGS. 3E-G show a system similar to those described herein, except having more than one, e.g., a plurality of containers 1302 (e.g., containers 1302a and 1302b), enclosing medicament for delivery to a patient. Each of the containers 1302 in this embodiment may be substantially similar to any of the containers described herein. Furthermore, the low pressure line 3004 may include two branches 3004a and 3004b, and each of the two branches 3004a and 3004b may be diverted to one of the containers 1302. In particular, each of the branches 3004a and 3004b may be used to move one of the containers 1302 along its longitudinal axis to put the container 1302 in to fluid communication with a respective fluid conduit, and subsequently, to drive a piston 1316 through the respective container 1302. As discussed above and further herein, the system may also include fluid source 1366, high pressure line 3002, flow restrictor 3008, valve 3010 with diaphragm 3012, and a venting system 2300 fluidly connected by a number of fluid lines or conduits. Additional details regarding venting system 2300 are provided herein. In this embodiment, the piercing of and flow of fluid through the two containers is substantially simultaneous.

In this embodiment, fluid conduit 300 may be modified to include a branch at second end 304. Indeed, the branch at second end 304 may include a plurality of needles, each of the plurality of needles being configured to move into fluid communication with exactly one of the containers 1302. Thus, in the embodiment shown, where the system includes two containers 1302, fluid conduit 300 includes two substantially parallel needles at second end 304. The plurality of needles may flow into a common channel of fluid conduit 300, and the medicament may be delivered out of a single channel or lumen at first end 302. While two containers 1302 and two needles at second end 304 are shown in the figures, it is contemplated that any other suitable number of containers and needles may be utilized, including three, four, five or more.

As shown in FIGS. 3F and 3G, within the auto-injector, the plurality of containers 1302, valve 3010, and/or canister or fluid source 1366 may be arranged in a substantially parallel orientation relative to one another. For example, FIG. 3F is a side view of fluid source 1366, valve 3010, and containers 1302a and 1302b, and FIG. 3G is an end view of fluid source 1366 and containers 1302a and 1302b. However, it is also contemplated that in some embodiments, one of more of the plurality of containers 1302 and/or of the canister 1366 may extend along offset axes. Furthermore, it is contemplated that one or more, or a plurality of, canisters 1366 may be utilized such that each container 1302 and fluid conduit 300 is associated with a dedicated canister 1366.

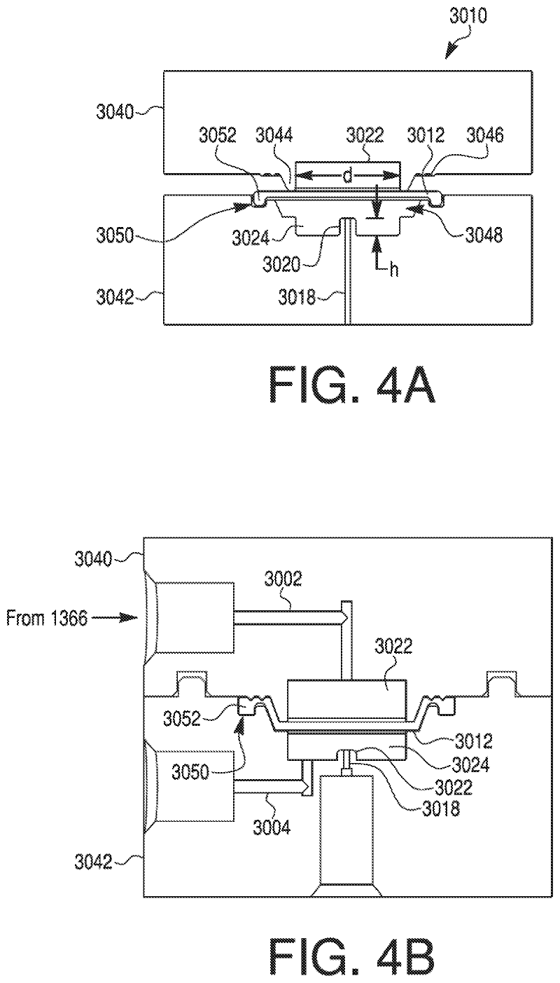

FIGS. 4A and 4B illustrate further detail relating to valve 3010. Valve 3010 may be designed to operate at a specific pressure, based on a balancing of one or more parameters including diaphragm thickness, diaphragm durometer, valve seat height h, and/or the diameter d of high pressure cavity 3022. During pressure equalization between the high pressure cavity 3022 and low pressure cavity 3024, the low pressure in conduit 3018 may create a retention force that may prevent diaphragm 3012 from returning to the neutral stage shown in FIG. 4A. This may be avoided by reducing the diameter of conduit 3018 and/or increasing the return force of the diaphragm 3012 by adjusting one or more of pre-tension, diaphragm thickness, diaphragm diameter, the seat height. For example, a flat, stamped diaphragm may shift in relation to the rest of the valve due to forces acting on it during deflection and may lose its return force.

Valve 3010 may include a first body portion 3040 and a second body portion 3042. First body portion 3040 may include high pressure cavity 3022, and a tenting boss 3044 surrounding high pressure cavity 3022 that stretches diaphragm 3012 (in a manner similar to a drum head), when first body portion 3040 and second body portion 3042 are mated to one another. First body portion 3040 also may include a clamping rib 3046 that encircles tenting boss 3044, and anchors diaphragm 3012 by a grip or clamp. Second body portion 3042 may include a recess 3048 configured to receive tenting boss 3044. Recess 3048 may have a corresponding shape to tenting boss 3044 such that when first body portion 3040 and second body portion 3042 are mated with one another, the outer surfaces of tenting boss 3044 are flush against the inner surfaces of recess 3048 (when diaphragm 3012 is not inserted between first body portion 3040 and second body portion 3042). Second body portion 3040 also may include a sealing groove 3050 configured to receive a sealing rib 3052 of diaphragm 3012. Sealing rib 3052 may be located on the outer periphery of diaphragm 3012 to provide increased material thickness, thereby improving the seal formed by diaphragm 3012.

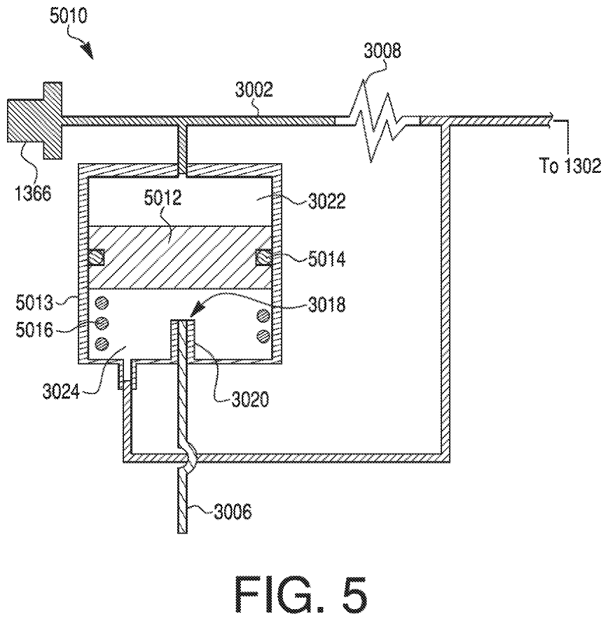

An alternative valve 5010 is shown in FIG. 5. Valve 5010 may be substantially similar to valve 3010 shown in FIGS. 3A-3C, except that valve 5010 may include a piston 5012 instead of a diaphragm 3012. Piston 5012 may include a seal 5014 disposed in a circumferential groove in the outer surface of piston 5012. Seal 5014 may help fluidically separate high pressure cavity 3022 from low pressure cavity 3024. Piston 5012 also may be connected to a spring 5016 coupled to the end of piston 5012 facing low pressure cavity 3024. Spring 5016 also may be coupled to a surface of valve 5010 defining the low pressure cavity 3024, and may be disposed entirely within low pressure cavity 3024. The resting position of spring 5016 is shown in FIG. 5. In the resting position, piston 5012 is spaced apart from valve seat 3020 and conduit 3018 is open. However, when fluid source 1366 is actuated, the greater pressure in high pressure cavity 3022 may act against piston 5012, compressing spring 5016 until piston 5012 abuts valve seat 3020 and closes conduit 3018. When piston 5012 reaches the end of injection (and bottoms out), the pressures in high pressure cavity 3022 and low pressure cavity 3024 will equilibrate, allowing spring to 5016 to expand to its resting position, opening conduit 3018. Alternatively, spring 5016 may extend from the end of piston 5012 facing high pressure cavity 3022, and extend through high pressure cavity 3022 to an opposite end of high pressure cavity 3022, connecting to the end of piston 5012 facing high pressure cavity 3022 and a surface defining the opposite end of high pressure cavity 3022. In this alternative embodiment, when high pressure cavity 3024 is filled with pressurized gas from fluid source 1366, spring 5016 may expand from its resting position to allow piston 5012 to seal conduit 3018.

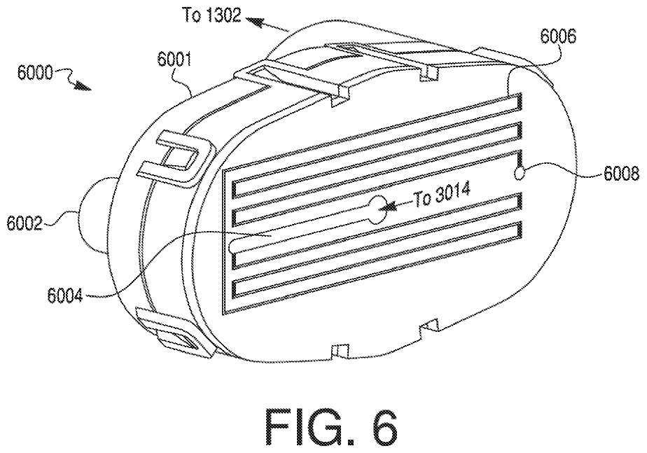

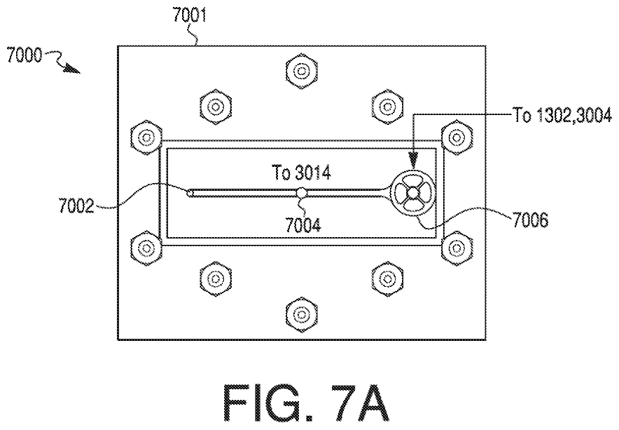

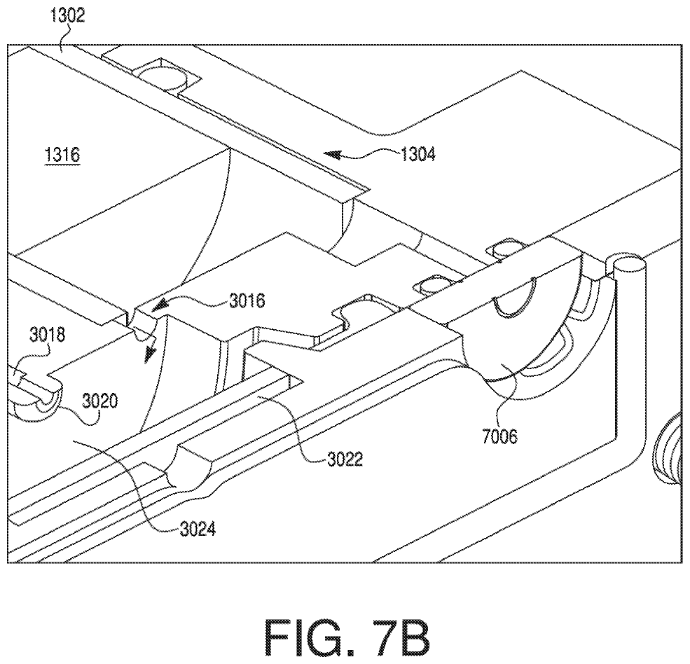

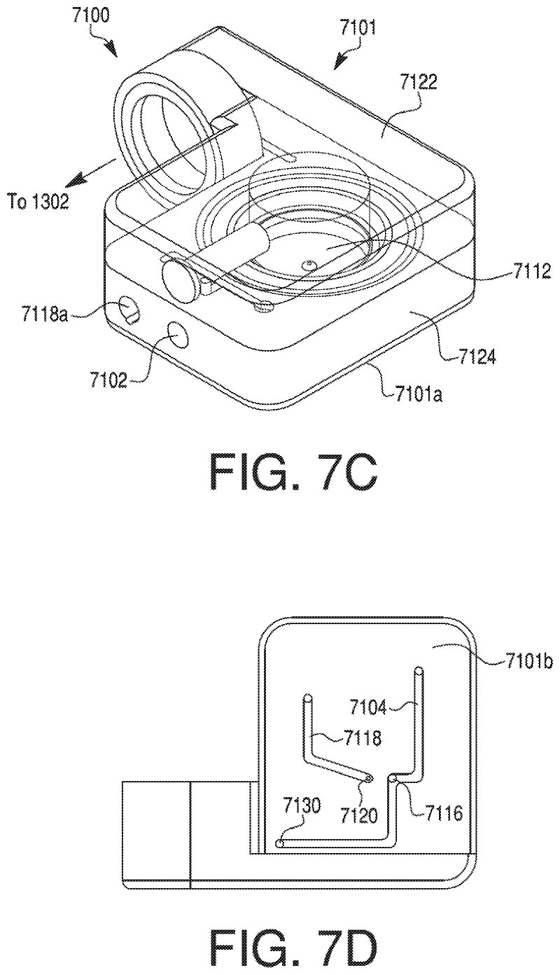

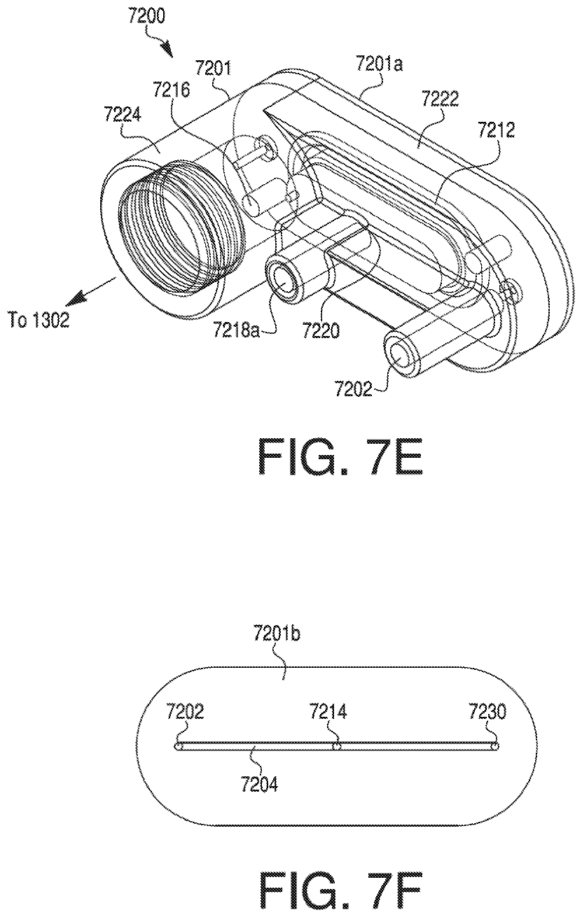

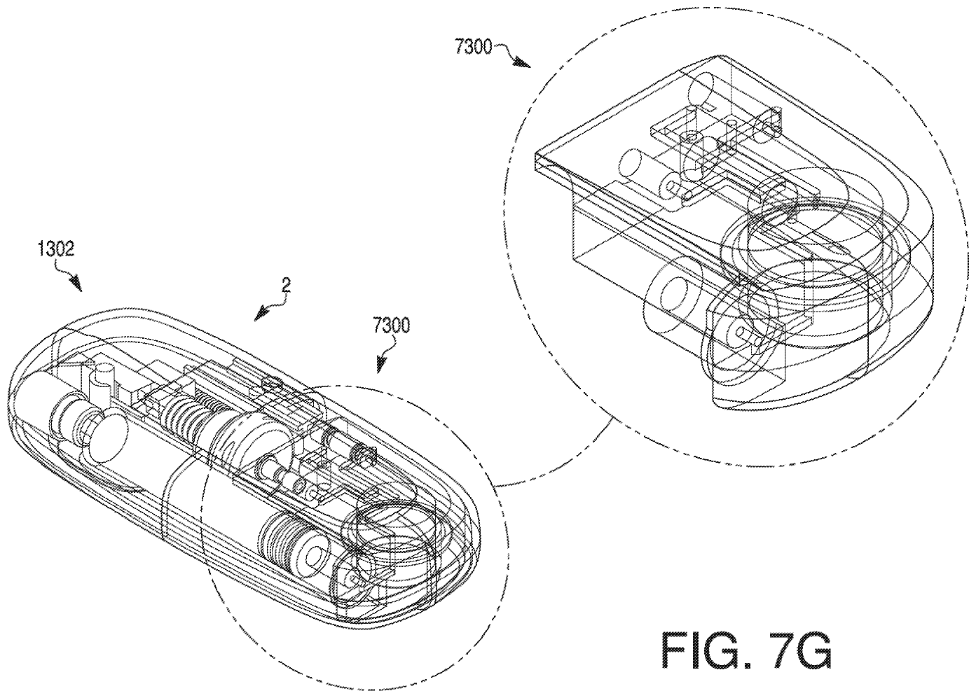

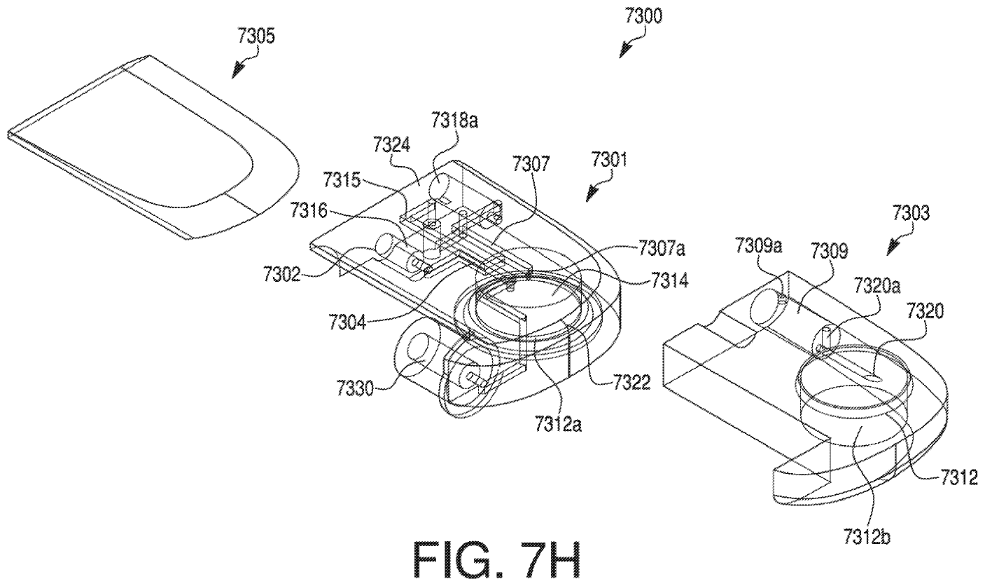

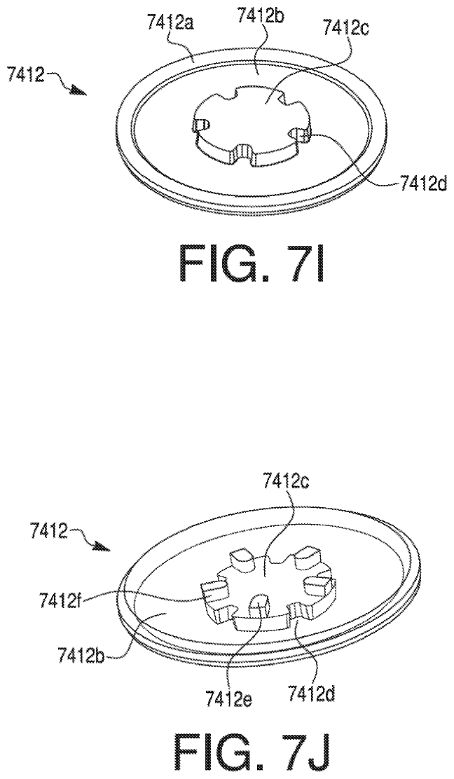

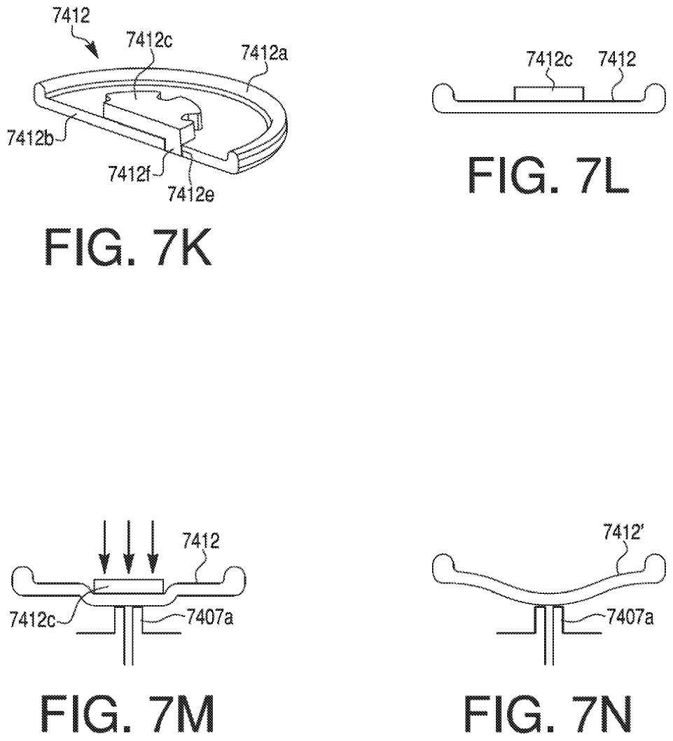

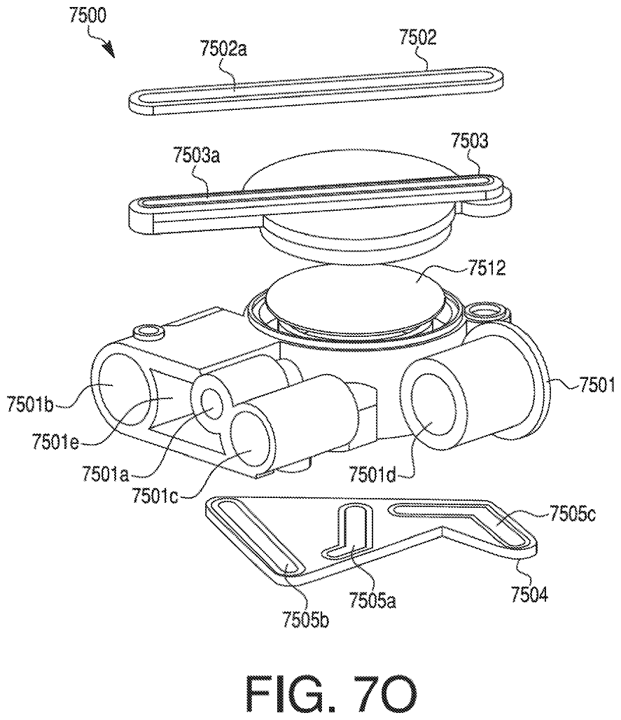

Exemplary flow restriction systems are shown in FIGS. 6, 7A, and 7B. A restriction system 6000 is shown in FIG. 6, and may be implemented herein anywhere that flow restrictor 3008 is shown. Flow restriction system 6000 may include a housing 6001 having an inlet 6002 that is connected to the output of fluid source 1366. Pressurized gas may be directed from inlet 6002 through conduit 6004 to high pressure line 3002 (referring to FIG. 3A). Pressurized gas from inlet 6002 also may be simultaneously diverted through conduit 6006 (the flow restrictor) and ultimately diverted to low pressure line 3004 and to container 1302 (referring again to FIG. 3A). The serpentine or tortuous path of conduit 6006 may result in a pressure drop of the pressurized gas flowing therethrough. This reduced-pressure gas is then diverted to low pressure line 3004 and container 1302 as described with reference to FIGS. 3A-3C.

A flow restriction system 7000 is shown in FIGS. 7A and 7B, and may be implemented anywhere that pressure restrictor 3008 is shown. Flow restriction system 7000 may be a cartridge 7001 having an inlet 7002 that is connected to the output of fluid source 1366. Pressurized gas may be directed from inlet 7002 through conduit 7004 to high pressure line 3002 (referring to FIG. 3A). Pressurized gas from inlet 7002 also may be simultaneously diverted through a flow restrictor (i.e., pressure reducer) 7006, which may be a frit comprising a porous material (e.g., microporous or macroporous), such as, for example, plastics (particularly sintered plastics), ceramics, or other suitable materials. The average pore size of the porous material may be from about 0.5 to about 15 microns, from about 1 micron to about 10 microns, from about 3 microns to about 6 microns, or about 5 microns, in diameter. The porous material causes a pressure drop to be experienced in the pressurized gas flowing through it, and the pressure-reduced gas is then diverted to low pressure line 3004 and container 1302 as described with reference to FIGS. 3A-3C. In particular, and as shown in greater detail in FIG. 7B, pressurized gas may flow through flow restrictor 7006 into container 1302 to drive piston 1316. Low pressure inlet 3024 may receive a portion of the reduced-pressure flow. It should be noted that low pressure line 3004 is omitted from FIG. 7B, but it is contemplated that a low pressure line 3004 may direct the reduced-pressure flow from flow restrictor 7006 to low pressure inlet 3016. However, as shown, low pressure inlet 3024 is an opening in a housing disposed adjacent to 1) the first end 1304 of container 1302, and 2) an outlet of flow restrictor 7006. Flow restriction system 7000 may be less prone to clogging and may be easier to manufacture than alternative flow restrictors.

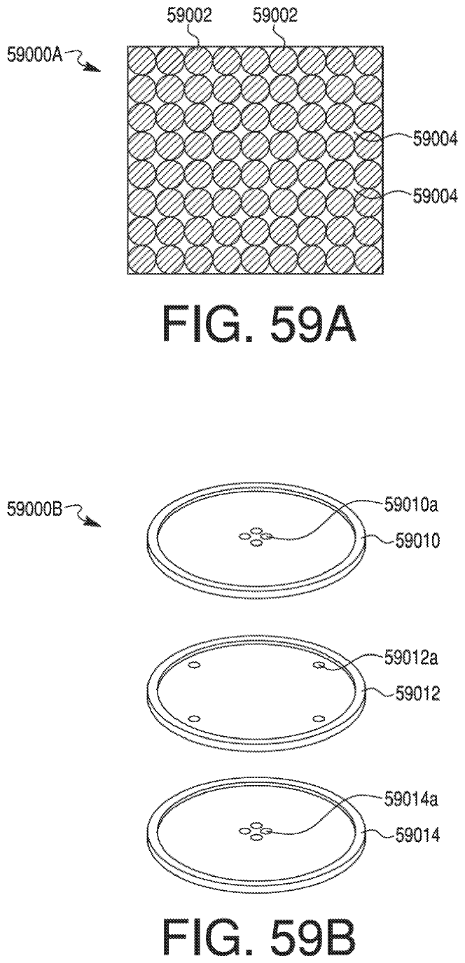

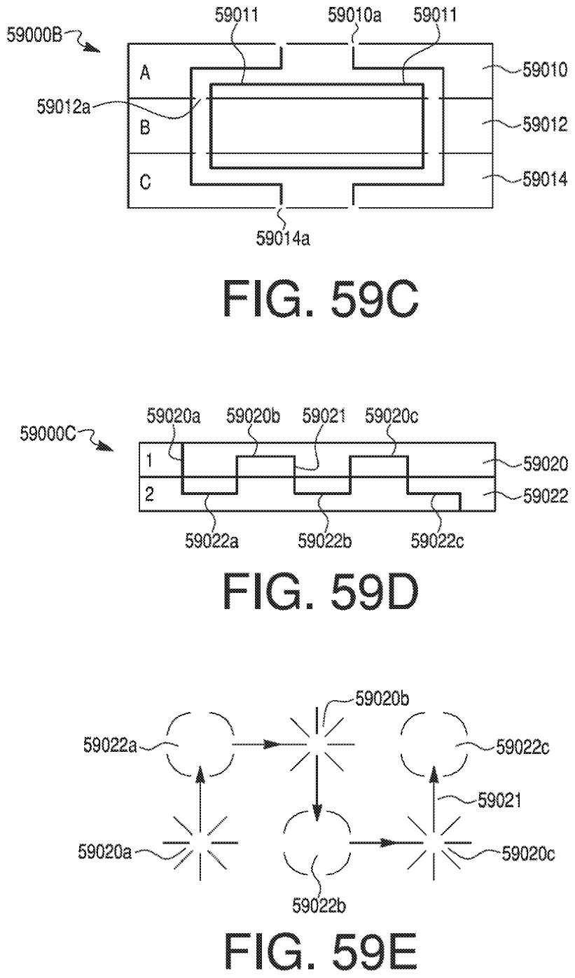

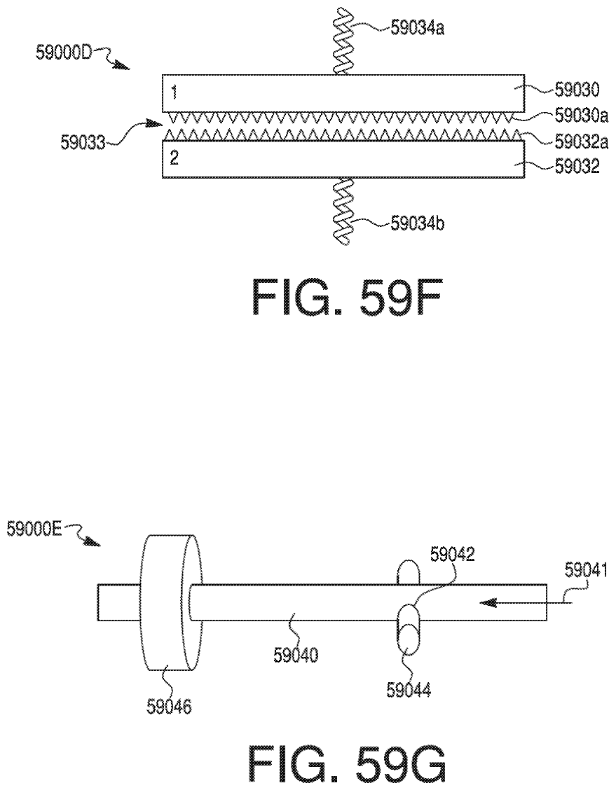

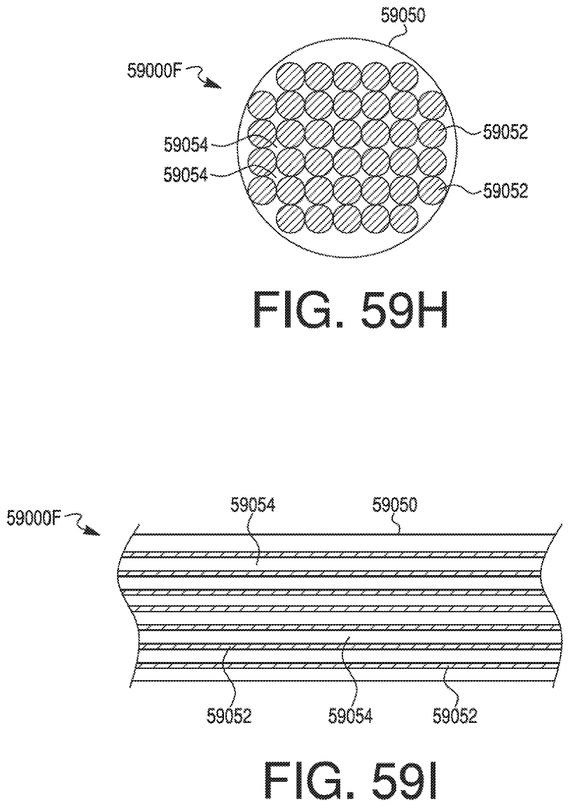

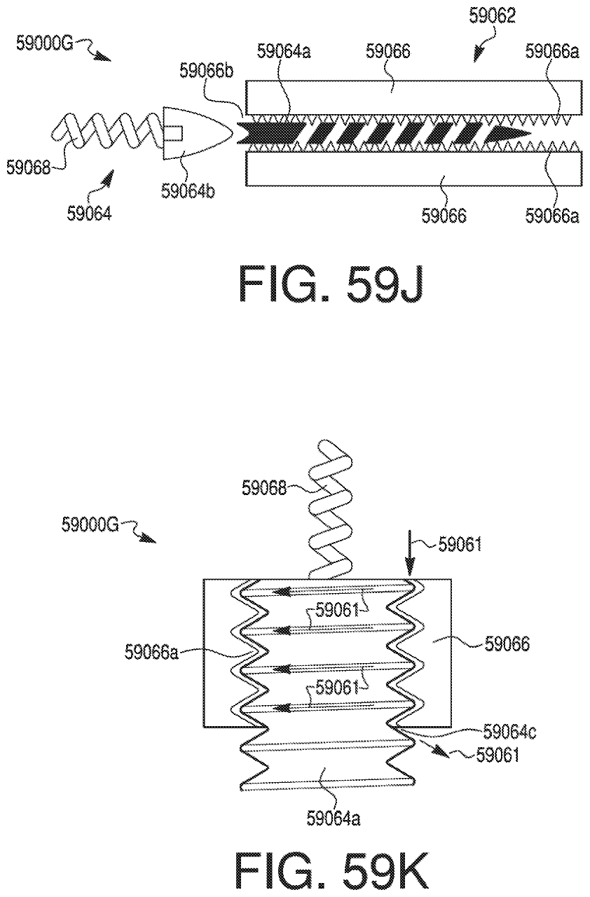

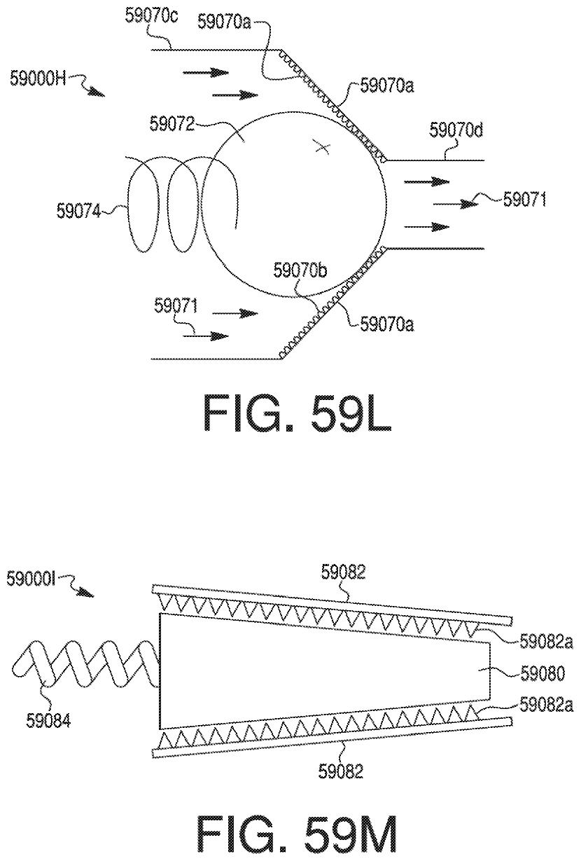

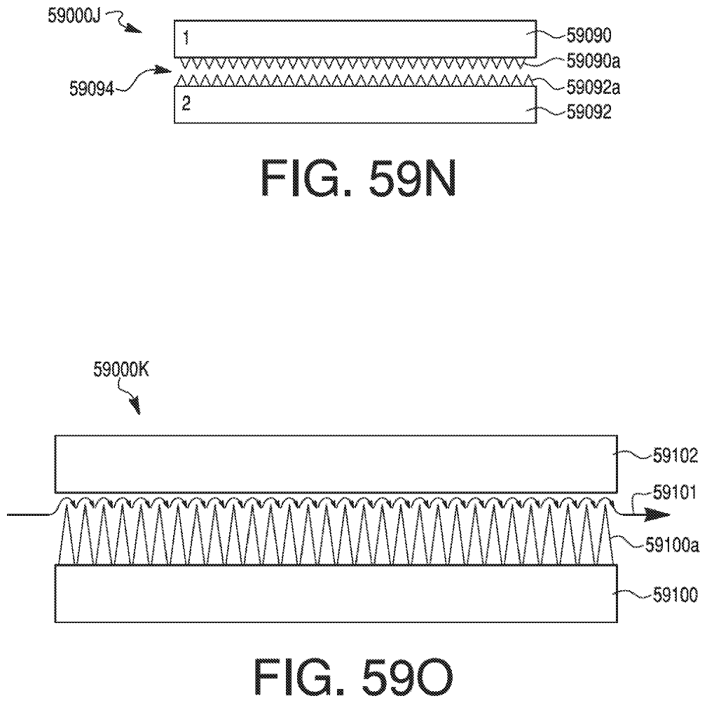

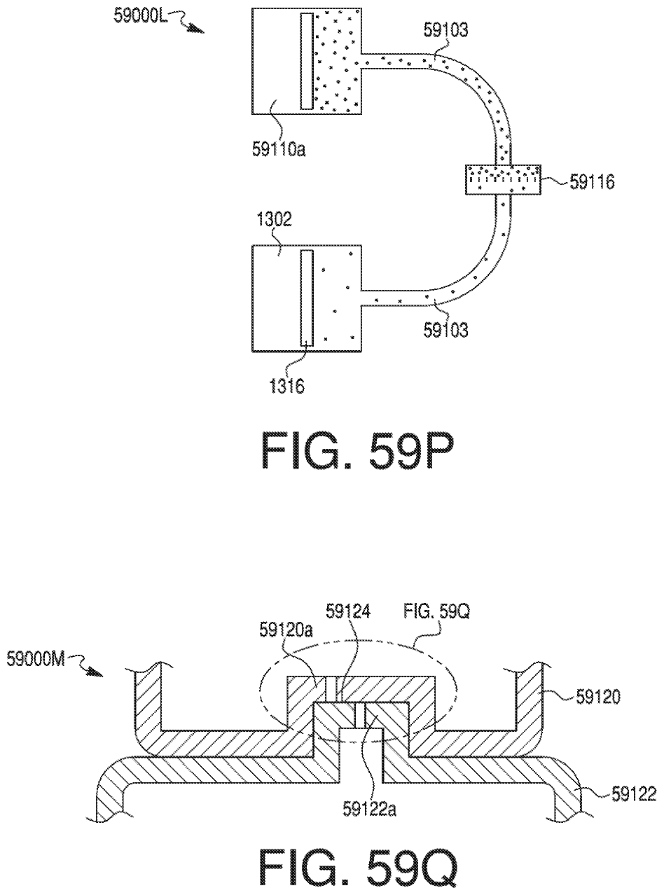

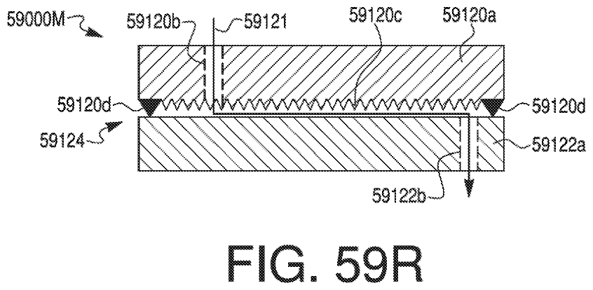

As mentioned above, pressurized gas from inlet 7002 may be diverted through flow restrictor (i.e., a pressure reducer) 7006, and flow restrictor 7006 may be a frit comprising a porous material, such as, for example, plastics (particularly sintered plastics), metals (e.g., stainless steel), ceramics, or other suitable materials. FIGS. 59A-59R illustrate various alternative flow restrictors that may be incorporated in flow restriction system 7000, as shown in FIGS. 7A and 7B.

FIG. 59A illustrates a cross-sectional view of one exemplary flow restrictor 59000A. Flow restrictor 59000A may be formed of or packed with a granular material. For example, flow restrictor 59000A may include a plurality of granules 59002 (e.g., particles of sand or other appropriate materials), with a number of gaps 59004 between adjacent granules 59002. Although not shown, granules 59002 may be packed in a tube, pipe, or other appropriate enclosed or partially-enclosed structure. Gaps 59004 between granules 59002 may create a tortuous path for gas passing through flow restrictor 59000A, and thus help to create a pressure drop on opposing sides of flow restrictor 59000A. Granules 59002 may be compressed at various pressures. In this aspect, the higher pressure of the compression, the more tightly granules 59002 are packed together, reducing the size of gaps 59004. Accordingly, the more tightly granules 59002 are packed together, the greater the pressure drop on opposing sides of flow restrictor 59000A. Granules 59002 may also be different sizes and/or shapes, which may help to control the pressure drop on opposing sides of flow restrictor 59000A. In this aspect, flow restrictor 59000A may create a pressure drop between opposing sides of flow restrictor 59000A.

FIGS. 59B and 59C illustrate an exploded view and a cross-sectional view of another exemplary flow restrictor 59000B. As shown, flow restrictor 59000B may include a plurality of plates, for example, plates 59010, 59012, and 59014, stacked in series. Plate 59010 includes one or more holes or openings 59010a, for example, in a central portion of plate 59010. Plate 59012 includes one or more holes or openings 59012a, for example, in an outer or peripheral portion of plate 59012, and plate 59014 includes one or more holes or openings 59014a, for example, in a central portion of plate 59010. Plates 59010 and 59014 may include the same general design or different designs. Openings in adjacent plates may be offset and/or unaligned with one another in the direction of gas flow, although it is contemplated that in at least some embodiments, certain adjacent plates may have the same or similar opening patterns. For example, a first plate (e.g., plate 59010) includes central openings (e.g., openings 59010a), and a second plate (e.g., plate 59012) includes outer openings (e.g., openings 59012a). Accordingly, the openings through adjacent plates are not aligned, regardless of the rotational orientation of the plates. In some embodiments, however, it is contemplated that at least some adjacent openings may be longitudinally aligned or otherwise aligned along an anticipated flowpath of the gas.

As shown in FIG. 59C, plates 59010, 59012, and 59014 may be stacked to form flow restrictor 59000B and may form one or more tortuous paths 59011 for gas to flow through flow restrictor 59000B. Gas flow is forced to pass through offset holes 59010a, 59012a, and 59014a in order to pass through flow restrictor 59000B. In these aspects, flow restrictor 59000B may be used to help create a pressure drop on opposing sides of flow restrictor 59000B, and may do so while also providing a clog resistance. Moreover, the pressure drop between opposing sides of flow restrictor 59000B may help to hold plates 59010, 59012, and 59014 together.

As shown in FIG. 59B, each plate 59010, 59012, 59014 may include four openings in the corresponding portion of each plate 59010, 59012, 59014. Alternatively, although not shown, each plate 59010, 59012, 59014 may include fewer than four openings, or a greater number of openings. Although not shown, flow restrictor 59000B may include two plates, or may include four or more plates. In these aspects, openings through adjacent plates may be offset, as discussed above, in order to create a pressure drop on opposing sides of flow restrictor 59000B. In one example, flow restrictor 59000B may include four or more plates of two designs, with the stack of plates including plates of one design being offset from one another by a plate of the other design. In one aspect, a larger number of plates may help to create a larger pressure drop between opposing sides of flow restrictor 59000B. Moreover, although plates 59010, 59012, and 59014 are shown as cylindrical, this disclosure is not so limited, as plates 59010, 59012, and 59014 may be different shapes and/or designs. Additionally, openings 59010a, 59012a, and 59014a may be formed by etching or any other appropriate procedure. In at least some embodiments, plates 59010, 59012, and 59014 may include etched channels. The etched channels may force gas flow to traverse a path from the center of the plate(s), out to the periphery of the plate(s), and back again to the center of the plate(s). In at least some embodiments, the rotational orientation of the multiple plates does not need to be controlled such that any rotational orientation will result in a functional pressure restrictor. The presence of multiple holes on each plate may help ensure that auto-injector 2 still functions properly in case one or more holes becomes clogged.

FIGS. 59D and 59E illustrate a cross-sectional view and a schematic illustration of another exemplary flow restrictor 59000C. As shown, flow restrictor 59000C may include a number of plates, for example, first and second plates 59020 and 59022. Plates 59020 and 59022 may be formed of any appropriate metallic or etchable material and may each include etched patterns (e.g., different etched patterns), with the etched patterns forming a tortuous flow path 59021 for gas flow. For example, as shown in FIGS. 59D and 59E, path 59021 may traverse through an etched pattern that includes etchings 59020a, 59020b, and 59020c in first plate 59020 and etchings 59022a, 59022b, and 59022c in second plate 59022. In this manner, plates 59020 and 59022 may form a tortuous flow path 59021 for gas flow to form a pressure drop on opposing sides of flow restrictor 59000C.

Flow restrictor 59000C may include fewer components (e.g., fewer plates) than flow restrictor 59000B, but each component (e.g., plates 59020 and 59022) may include more surface area and material (e.g., metal, etchable, or otherwise). In both aspects, however, the respective plates may be used to form a pressure drop on opposing sides of respective flow restrictors.

FIG. 59F illustrates a cross-sectional view of another exemplary flow restrictor 59000D. As shown, flow restrictor 59000D includes first and second plates 59030 and 59032, which face each other and form a gap or channel 59033 for gas flow (not shown) between plates 59030 and 59032. First and second plates 59030 and 59032 may each include surface finishes and/or textures, which may affect the roughness value and/or lay or fit of the surfaces of plates 59030 and 59032 against each other. In at least some embodiments, the surface finish may be formed by molding, stamping, machining, knurling, forging, sand blasting, shot blasting, chemical etching, or another appropriate method. For example, first plate 59030 may include a first surface finish 59030a, and second plate 59032 may include a second surface finish 59032a. First surface finish 59030a and second surface finish 59030b may be the same or similar surface finishes, or may be different surface finishes. In this aspect, channel 59033 between plates 59030 and 59032 may help to create a tortuous and/or impeded path for the gas flow and thus a pressure drop on opposing sides of flow restrictor 59000C.

Moreover, one or more springs (e.g., springs 59034a and 59034b) may bias one or more of plates 59030 and 59032 toward the other of plates 59030 and 59032. Spring(s) 59034a and 59034b may add pressure (e.g., push plates 59030 and 59032 toward each other), which may help to create a tortuous and/or impeded path for the gas flow and thus, may help create a pressure drop on opposing sides of flow restrictor 59000C. For example, spring(s) 59034a and 59034b may help to control a contact pressure between plates 59030 and 59032, which may help to provide a repeatable pressure drop and/or gas flow. Additionally, spring(s) 59034a and 59034b may compress one or more of plates 59030 and 59032 at all times to have a constant pressure on channel 59033 and a resulting tortuous and/or impeded path for the gas flow, which may also depend on surface finishes 59030a and 59032a. In another aspect, spring(s) 59034a and 59034b may compress one or more of plates 59030 and 59032 in order to fully close off flow of gas through flow restrictor 59000C in a first (pre-activated) state, and once a patient needle mechanism is activated, as discussed herein, the one or more springs may be relaxed or the compression on one or more of plates 59030 and 59032 may be reduced, such that channel 59033 opens and remains open for the remainder of the injection, with surface finishes 59030a and 59032a helping to form a tortuous and/or impeded path and a resulting pressure drop across flow restrictor 59000D. After completion of the injection, and for example withdrawal of the patient needle from the patient, a restriction on the springs 59034a and 59034b may be removed, allowing for expansion of the springs and closing of the flow path.

FIG. 59G illustrates a perspective view of another exemplary flow restrictor 59000E. As shown, flow restrictor 59000E includes a hollow channel, needle, or tube 59040. Tube 59040 may extend longitudinally, and may include one or more lateral openings 59042 extending through a side portion of tube 59040, for example, bored through two sides of tube 59040. Flow restrictor 59000E may also include a solid cylinder or rod 59044 (or other solid obstruction), which may be positioned within opening 59042 and through a portion of tube 59040. In this aspect, rod 59044 may help to restrict gas flow 59041 through tube 59040 by creating a restriction to gas flow.

Tube 59040 may be coupled to or staked to a disk 59046, and disk 59046 may help to separate high pressure and low pressure regions to create a pressure drop on opposing sides of flow restrictor 59000E. For example, disk 59046 may help divide the high and low pressure regions by allowing only air/gas/fluid to flow through a narrow channel (e.g., through tube 59040). Disk 59046 is shown as a cylindrical disk, but this disclosure is not so limited, as disk 59046 may take any shape and/or size to help divide the high and low pressure regions. In this aspect, tube 59040 may include a cross-sectional area that is smaller than the cross-sectional area of disk 59046. Accordingly, the smaller cross-sectional area of tube 59040 may help to restrict gas flow 59041, and thus help to create a pressure drop on opposing sides of flow restrictor 59000E. Accordingly, both the smaller cross-sectional area of tube 59040 and the obstruction created by rod 59044 through a portion of tube 59040 may help to create a pressure drop on opposing sides of flow restrictor 59000E.

FIGS. 59H and 59I illustrate cross-sectional views of another exemplary flow restrictor 59000F. FIG. 59H is a lateral cross-sectional view of flow restrictor 59000F, and FIG. 59I is a longitudinal cross-sectional view of a portion of flow restrictor 59000F. As shown, flow restrictor 59000F includes an outer pipe, needle, or tube 59050 and a plurality of wires or filaments 59052 within tube 59050. The plurality of filaments 59052 form a number of gaps or passages 59054 between adjacent filaments 59052. Passages 59054 between filaments 59052 may create a tortuous and/or impeded path for fluid passing through flow restrictor 59000F, and thus help to create a pressure drop on opposing sides of flow restrictor 59000F. Tube 59050 may be compressed, which may more tightly pack filaments 59052 within tube 59050, and thus reduce the size of passages 59054. Accordingly, the more tightly filaments 59052 are packed together, the greater the pressure drop on opposing sides of flow restrictor 59000F.

Although FIG. 59I illustrates filaments 59052 and passages 59054 being substantially straight through tube 59050, this disclosure is not so limited. For example, filaments 59052 may be coiled (e.g., in a spiral configuration) and/or otherwise manipulated to reduce the size of passages 59054 and affect the pressure drop on opposing sides of flow restrictor 59000F. Alternatively or additionally, filaments 59052 may be drawn or mechanically worked after assembly within tube 59050, for example, to reduce the size of passages 59054 and affect the pressure drop on opposing sides of flow restrictor 59000F.

FIGS. 59J and 59K illustrate cross-sectional views of another exemplary flow restrictor 59000G. FIG. 59J is a lateral cross-sectional view of flow restrictor 59000G, and FIG. 59K is a lateral cross-sectional view of a portion of flow restrictor 59000G. As shown, flow restrictor 59000G includes a housing 59062 and a screw structure 59064. Housing 59062 may be substantially cylindrical and include walls 59066. Walls 59066 include threading 59066a and form an opening 59066b. Screw structure 59064 includes a screw 59064a that may be threaded along threading 59066a to insert screw 59064a within opening 59066b. Screw structure 59064 also includes a screw head 59064b, which may include angled or tapered surfaces, for example, to abut and/or at least partially block opening 59066b. Additionally, screw structure 59064 may include a spring 59068.

As shown in FIG. 59K, with screw 59064a threaded into opening 59066b, flow restrictor 59000G may form a tortuous path 59061 for gas flow, for example, through small openings between screw 59064a and threading 59066a on walls 59066. For example, opening 59066b may be a standard threaded through-hole, and screw 59064a may be a standard machine screw. The small clearance between screw 59064a and threading 59066a may form a single helical passage 59061 for gas flow (FIG. 59K). The tightness of screw 59064a may be set to a desired tightness and/or insertion distance in order to control the desired pressure drop across flow restrictor 59000G. Moreover, the pitch and/or thread of screw 59064a and/or threading 59066a may affect the ability for gas flow to pass through flow restrictor 59000G. It is noted that screw head 59064b is not shown in FIG. 59K for clarity. Nevertheless, spring 59068 may help to compress screw 59064a within opening 59066b and/or help secure or tighten the connection between screw structure 59064 and housing 59062. In these aspects, a pressure drop may be formed and/or controlled between opposing sides of flow restrictor 5900G. The spring may help control contact pressure and increase repeatability of flow characteristics. The arrangement of FIGS. 59J and 59K may be similar to a needle valve.

FIG. 59L illustrates a cross-sectional view of another exemplary flow restrictor 59000H. As shown, flow restrictor 59000H includes a housing 59070, a ball bearing 59072, and a spring 59074 to create a tortuous path for gas flow 59071. Housing 59070 may include angled sides 59070a, which may at least partially abut a portion of ball bearing 59072. For example, angled sides 59070a may form a substantially cone-like shape, with circular longitudinal cross-sections. In this aspect, housing 59070 may include a wide portion 59070c, for example, to receive gas at a higher pressure, and a narrow portion 59070d, for example, to discharge gas at a lower pressure. Moreover, angled sides 59070a may include rough or textured surfaces 59070b.

Ball bearing 59072 may be substantially spherical. Ball bearing 59072 may include one or more textured surfaces, for example, to affect the contact with textured surface 59070b. For example, the textured surface may be formed by molding, stamping, machining, knurling, forging, sand blasting, shot blasting, chemical etching, or another appropriate method. Additionally, spring 59074 may securely couple ball bearing 59072 to another portion of a housing (not shown). Accordingly, both the force of the spring and input gas pressure (e.g., from wide portion 59070c) may push ball bearing 59072 against textured surfaces 59070b, which may form a partial seal and restrict gas flow into narrow portion 59070d. In some aspects, a higher input gas pressure (e.g., in wide portion 59070c) may more strongly push ball bearing 59072 against textured surface 59070b. Ball bearing 59072 may thus restrict gas flow 59071 from flowing to narrow portion 59070d at a higher strength, thus creating a larger pressure drop between sides of flow restrictor 59000H. In these aspects, a pressure drop may be formed and/or controlled between opposing sides of flow restrictor 59000H.

FIG. 59M illustrates a cross-sectional view of another exemplary flow restrictor 59000I. This embodiment also may include textured surfaces formed by molding, stamping, machining, knurling, forging, sand blasting, shot blasting, chemical etching, or another appropriate method. As shown, flow restrictor 59000I includes a plug 59080, a housing 59082, and a spring 59084. Plug 59080 may be partially conical (e.g., a conical frustum), for example, including a substantially tapered structure. As shown in FIG. 59M, plug 59080 may include a wider portion at a high pressure region (left side) and a narrower portion at a low pressure region (right side). Housing 59082 may include a shape that is at least partially complimentary to plug 59080. Additionally, in some aspects, housing 59082 includes a rough, threaded, or textured surface 59082a. Accordingly, plug 59080 may be at least partially received within housing 59082. Additionally, spring 59084 may push against the wide portion of plug 59080, for example, to apply pressure on plug 59080 and help to secure plug 59080 within housing 59082. In these aspects, gas flow (not shown) may flow through a labyrinth, impeded, and/or tortuous path formed between plug 59080 and housing 59082 (e.g., by textured surface 59082a). Additionally, the insertion distance of plug 59080 into housing 59082, the compression force of spring 59084, and/or other features may be adjusted to affect the gas flow path, and thus control the pressure drop. In these aspects, a pressure drop may be formed and/or controlled between opposing sides of flow restrictor 59000I.

FIG. 59N illustrates a cross-sectional view of another exemplary flow restrictor 59000J. As shown, flow restrictor 59000J includes a first side 59090 and a second side 59092. For example, if flow restrictor 59000J is substantially cylindrical, a longitudinal cross-section may form first side 59090 and second side 59092. Alternatively, flow restrictor 59000J may be rectangular, and first side 59090 and second side 59092 may be formed by opposing sides of flow restrictor 59000J. In these aspects, first side 59090 and second side 59092 may extend substantially parallel to each other, and may form a gap or channel 59094, for example, to receive a gas flow (not shown). First side 59090 includes a first coating 59090a, and second side 59092 includes a second coating 59092a, for example, to form a chromatography column. In some aspects, first coating 59090a and second coating 59092a may have a characteristic. The coating may be selected to have an opposite polarity of the gas or fluid that will subsequently flow through the channel. For example, coatings 59090a and 59092a may be hydrophobic, hydrophilic, have a polarity, etc. In one example, the fluid flowing through flow restrictor 59000J may be hydrophilic, and coatings 59090a and 59092a may be hydrophobic. In another example, the fluid flowing through flow restrictor 59000J may be hydrophobic, and coatings 59090a and 59092a may be hydrophilic. In these aspects, a pressure drop may be formed and/or controlled between opposing sides of flow restrictor 59000H. It is noted that the aspects discussed herein with respect to the coatings, for example, with respect to FIG. 59N, may be incorporated into any of the flow restrictors discussed herein.

FIG. 59O illustrates a partial cross-sectional view of another exemplary labyrinth seal flow restrictor 59000K. As shown, flow restrictor 59000K includes a shaft 59100 and a housing 59102. A gas flow 59101 or fluid path may travel in a channel (not labeled) between shaft 59100 and housing 59102. It is noted that FIG. 59O illustrates a portion, for example, a top half, of flow restrictor 59000K. As shown, shaft 59100 may include a plurality of projections 59100a. Accordingly, projections 59100a may create a tortuous path for gas flow 59101. For example, gas flow 59101 must traverse through the channel between projections 59100a and housing 59102, which may help to create a pressure drop between opposing sides of flow restrictor 59000K. For example, labyrinth seal flow restrictor 59000K may force the gas to expand after passing across each tooth (there being a small gap between housing 59102 and the tip of each tooth), and thus help to create the pressure drop between opposing sides of flow restrictor 59000K. The type and/or size of projections 59100a and other aspects of flow restrictor 59000K may be adjusted to control and/or adjust the pressure drop between opposing sides of flow restrictor 59000K. In these aspects, a pressure drop may be formed and/or controlled between opposing sides of flow restrictor 59000K.

FIG. 59P illustrates a schematic view of another exemplary flow restrictor 59000L. As shown, flow restrictor 59000L is configured to discharge a pressurized gas 59103 from a gas canister 59110a. Additionally, a frit 59116, a slit, small opening, or other flow restriction device discussed herein is positioned in the flowpath to create a pressure drop. As shown, material or gas 59103 may be present in a higher density before reaching frit 59116, and material 59103 may be present in a lower density after passing through frit 59116. After passing through frit 59116, the lower pressure fluid may extend through a low pressure line to be used in any suitable manner as described elsewhere in this specification, including to drive piston 1316 through container 1302. The embodiment of FIG. 59P may be substantially structurally similar to other frits and/or porous microfilters as discussed herein. However, it is contemplated that lower grade or lower specification structural components may be utilized in conjunction with a higher viscosity fluid or refrigerant (as opposed to, e.g., R32 refrigerant). For example, material or gas 59103 may be a gas at a higher density (i.e., higher pressure, higher atomic weight, etc.), or material or gas 59103 may be a liquid (e.g., water, oil, glycerin, or any other liquid that is bio-compatible and has a higher viscosity and/or density than the gas on the sides of flow restrictor 59000L.

It is also noted that, if material 59103 is viscous enough, a frit may not be necessary, as the material alone or the material along with a narrow slit may help to create the desired pressure drop between opposing sides of flow restrictor 59000L.

FIGS. 59Q and 59R illustrate cross-sectional views of another exemplary flow restrictor 59000M. FIG. 59Q is a cross-sectional view of flow restrictor 59000M, and FIG. 59R is an enlarged view of a portion of FIG. 59Q. As shown, flow restrictor 59000M includes a first housing 59120 and a second housing 59122. First housing 59120 and second housing 59122 may be formed of a plastic material, for example, via injection molding, a metal machined material, or another material. First housing 59120 and second housing 59122 may be in substantially abutting contact at an interface 59124 (e.g., in an interference or other suitable fit). First housing 59120 may include a first indented portion 59120a, and second housing 59122 may include a second indented portion 59122a. As shown in FIG. 59Q, second indented portion 59122a may be received within first indented portion 59120a, for example, to form an at least partially sealed portion between the periphery of second indented portion 59122a and the inner portion of first indented portion 59120a.

As shown in greater detail in FIG. 59R, first indented portion 59120a includes a first channel 59120b. Additionally, second indented portion 59122a includes a second channel 59122b. First channel 59120b and second channel 59122b may be offset from each other in the fluid flow direction, but nevertheless fluidly connected by an opening between first indented portion 59120a and second indented portion 59122a, for example, at interface 59124. Accordingly, a gas flow 59121 or fluid may flow through first channel 59120b, through the opening, and then through second channel 59122b. Additionally, one or more of first indented portion 59120a and/or second indented portion 59122a may include a surface texture. For example, as shown in FIG. 59R, first indented portion 59120a may include a textured surface 59120c facing the opening and second indented portion 59122a. While not shown in the figure, it is also contemplated that second indented portion 59122a also may include a similar or complementary textured surface. In at least some embodiments, the textured surfaces may be formed by molding, stamping, machining, knurling, forging, sand blasting, shot blasting, chemical etching, or another appropriate method.

Additionally, first indented portion 59120a and second indented portion 59122a may be welded or otherwise secured together via connections 59120d, or the connection may be achieved by one or more seals. In this manner, gas flow 59121 may traverse first channel 59120b, the opening between first indented portion 59120a and second indented portion 59122a, including textured surface 59120c, and second channel 59122b. Connections 59120d may help restrict gas flow 59121 from escaping from flow restrictor 59000M any other way except for as detailed above.

The tortuous path through first channel 59120b, the opening between first indented portion 59120a and second indented portion 59122a, including textured surface 59120c, and second channel 59120b may help to form a pressure drop between opposing sides of flow restrictor 59000M. The structure of flow restrictor 59000M may allow for a reduction in pressure without a frit or other additional materials, and instead rely on the existing structures of an auto-injector. Additionally, the size of first opening 59120b, the size of opening between first indented portion 59120a and second indented portion 59122a, the texture of textured surface 59120c, and the size of second opening 59122b may be adjusted to affect the path of gas flow 59121. In these aspects, a pressure drop may be formed and/or controlled between opposing sides of flow restrictor 59000M.

An implementation of valve 3010 is shown in FIGS. 7C and 7D as valve 7100. Valve 7100 may be compatible with a container 1302 whose longitudinal axis is perpendicular to the surface of the skin of a patient (instead of parallel to the surface of the skin as shown, for example, in FIG. 2). Valve 7100 may include a housing 7101 having an inlet 7102 that is connected to the output of fluid source 1366. Pressurized gas may be directed from inlet 7102 to high pressure line 3002 (referring to FIG. 3A but not shown in FIGS. 7C-D) and high pressure cavity 7122 shown in FIG. 7C. The high pressure gas in high pressure cavity 7122 may urge a diaphragm 7112 toward valve vent 7120 to seal valve vent 7120. Pressurized gas from inlet 7002 also may be simultaneously diverted through a flow restrictor (not shown), and then diverted to low pressure line 7104 and container 1302 (via a primary container inlet 7130). The flow restrictor used in this embodiment may be any suitable flow restrictor including the frit and/or serpentine conduits described herein. The flow restrictor may be disposed within inlet 7130, or upstream or downstream of inlet 7130. Pressurized gas may flow from the flow restrictor to low pressure line 7104 and primary container inlet 7130, into container 1302 to drive piston 1316. A low pressure portion 7124 of housing 7101 includes a low pressure cavity that receives a portion of the reduced-pressure flow via low pressure inlet 7116. A plate cover 7101a may be laser welded, ultrasonically welded, or otherwise coupled to a bottom surface 7101b (FIG. 7D) of housing 7101. Bottom surface 7101b may contain low pressure line 7104, low pressure cavity inlet 7116, and primary container inlet 7130, each of which may be etched within bottom surface 7101b. Furthermore, bottom surface 7101b also may include valve vent 7120 in communication with the low pressure cavity in low pressure portion 7124 and with exhaust line 7118. As described above with respect to FIGS. 3A and 3C, when pressure equilibrates between high pressure cavity 7122 and the low pressure cavity, diaphragm 7112 may lift off from and unseal valve vent 7120, allowing gas/fluid from the low pressure cavity to travel through valve vent 7120 and exhaust line 7118, through a vent port 7118a (FIG. 7C). A rod (not shown, but substantially similar to rod 8002 described below) may be disposed within vent port 7118a. In valve 7100, it is contemplated that one or more, or all, of low pressure line 7104, low pressure cavity inlet 7116, primary container inlet 7130, valve vent 7120, and exhaust line 7118, are co-planar.

Another implementation of valve 3010 is shown in FIGS. 7E and 7F as valve 7200. Valve 7200 may be compatible with a container 1302 whose longitudinal axis is perpendicular to the surface of the skin of a patient. Valve 7200 may include a housing 7201 having an inlet 7202 that is connected to the output of fluid source 1366. Pressurized gas/fluid may be directed from inlet 7202 to high pressure line 7204, high pressure inlet 7214 (FIG. 7F), and a high pressure cavity disposed within portion 7222 of housing 7201 (FIG. 7E). The high pressure gas/fluid in the high pressure cavity 7204 may urge a diaphragm 7212 toward valve vent 7220 to seal valve vent 7220. Diaphragm 7212 may have an oval or raceway shape. Pressurized gas/fluid from inlet 7002 also may be simultaneously diverted through a flow restrictor (not shown), and then diverted to a low pressure line (such as low pressure line 3004 of FIGS. 3A-3C and container 1302 (via an inlet 7230 shown in FIG. 7F). In particular, pressurized gas may flow through inlet 7230, into container 1302 to drive piston 1316. In some embodiments, a frit or other flow restrictor may be disposed within inlet 7230. It is also contemplated that the flow restrictor is either upstream or downstream of inlet 7230. A low pressure cavity in portion 7224 of housing 7201 may receive a portion of the reduced-pressure flow via low pressure inlet 7216. A plate cover 7201a may be laser welded, ultrasonically welded, or otherwise coupled to a bottom surface 7201b (FIG. 7F) of housing 7201. Bottom surface 7201b may contain high pressure line 7202, high pressure cavity inlet 7214, and primary container inlet 7230, each of which may be etched within bottom surface 7201b. As described above with respect to FIGS. 3A and 3C, when pressure equilibrates between the high pressure cavity and the low pressure cavity, diaphragm 7212 may lift off from and unseal valve vent 7220, allowing gas from the low pressure cavity to travel through valve vent 7220 and through a vent port 7218a (FIG. 7E). A rod (not shown, but substantially similar to rod 8002 described below) may be disposed within vent port 7218a. In valve 7200, it is contemplated that one or more, or all, of high pressure line 7202, high pressure cavity inlet 7214, and inlet 7230, are co-planar.

FIGS. 7G and 7H illustrate a perspective view and an exploded view, respectively, of an auto-injector 2 with a valve 7300. In particular, another implementation of valve 3010 is shown in FIG. 7G as valve 7300. The features and elements of valve 7300 may function similarly to the features and elements of previously described valves, for example, valve 7200, as described above.

Valve 7300 may be compatible with container 1302. As shown in FIG. 7H, valve 7300 may include a first housing 7301, a second housing 7303, and a base plate 7305. Second housing 7303 may be coupled to a bottom of first housing 7301, and base plate 7305 may be coupled to a bottom of second housing 7303 to form valve 7300. First housing 7301 may include an inlet 7302 (e.g., a canister inlet), which may be connected to the output of fluid source 1366 (FIG. 5). Pressurized gas/fluid may be directed from inlet 7302 to a high pressure line 7304 (in first housing 7301), high pressure inlet 7320 (in second housing 7303 via connection 7320a also in second housing 7303), and a high pressure cavity 7312b located in second housing 7303. High pressure line 7304 may include a plurality of channels, which may be arranged in a circuitous, tortuous, or serpentine configuration, for example, traversing various directions. In one aspect, channels of the high pressure line may include approximately two to ten turns, for example, four turns. The high pressure gas/fluid in high pressure cavity 7312b may urge a diaphragm 7312 toward a valve seat 7307a to seal valve vent 7307. Diaphragm 7312 may have a generally circular shape, and may be substantially similar to the diaphragms discussed elsewhere in this disclosure. Pressurized gas/fluid from inlet 7302 also may be simultaneously diverted through a flow restrictor (not shown), and then diverted to a low pressure line (such as low pressure line 3004 of FIGS. 3A-3C) and a container (e.g., 1302) via conduit 7309a disposed within a PNM flow channel 7309. In particular, pressurized gas may flow from high pressure line 7304, through connection 7320a, and then into PNM flow channel 7309. The pressurized gas may then flow from PNM flow channel 7309 through conduit 7309a to a channel 7315, then to a container inlet 7330, and into container 1302 to drive container 1302 onto fluid conduit 300, and subsequently drive piston 1316. In some embodiments, a frit or other flow restrictor may be disposed within inlet 7330 or otherwise somewhere between conduit 7309a and inlet 7330. Exemplary frits and flow restrictors have been described elsewhere in this disclosure, and the details of the frit that follow in the paragraph may be used with any of those other embodiments. For example, the frit may be formed of a stainless steel, a sintered plastic, or other appropriate material. The frit may be formed of materials that include a pore size of approximately 0.5 microns or larger. The frit may include a length of up to approximately 8 to 12 mm, for example, approximately 10 mm, and a diameter of approximately 1 to 5 mm, for example, approximately 3 mm. It is also contemplated that the flow restrictor may be either upstream or downstream of inlet 7330. A low pressure cavity 7312a in portion 7324 of first housing 7301 may receive a portion of the reduced-pressure flow via low pressure inlet 7316.

Second housing 7303 may be laser welded, ultrasonically welded, or otherwise coupled to bottom surfaces of first housing 7301, and base plate 7305 may be similarly coupled to bottom surfaces of second housing 7303. These components of valve 7300 may be welded by two laser weldings, for example, simultaneously or quasi-simultaneously. Additionally, components of valve 7300 may be welded together around channels, for example, approximately 1-2 mm from channels, and the welding may include a weld thickness of approximately 1 mm.

Various features of first housing 7301, second housing 7303, and base plate 7305 may be etched within portions of first housing 7301 (or molded or machined), second housing 7303, and base plate 7305. As described above with respect to FIGS. 3A and 3C, when pressure equilibrates between the high pressure cavity and the low pressure cavity, diaphragm 7312 may lift off from and unseal valve seat 7307a, allowing gas from the low pressure cavity 7312a to travel through valve vent 7307 and through a vent port 7318a. A rod (not shown, but substantially similar to rod 8002 described below) may be disposed within vent port 7318a.

In one aspect, diaphragm 7312 may be formed of various materials, thicknesses, etc. In yet another aspect, diaphragm 7312 may be formed via one or more molding processes, which may provide a large range of performance characteristics, for example, with respect to temperature. For example, higher temperatures may create a greater pressure within the system of valve 7300 and/or canister, thus causing changes in the pressure differential across diaphragm 7312, which may also affect the movement of diaphragm 7312 and/or venting of valve 7300. In particular, higher temperature may prevent or inhibit separation/lift of diaphragm 7312 from vent seat 7307a. Furthermore, diaphragm 7312 may be formed of a composite material, for example, with a rigid central section (e.g., formed via a two-shot molding process), which may also affect the movement, for example, with an easier lift off and/or separation from valve seat 7307a because diaphragm 7312 includes an increased rigidity where diaphragm 7312 contacts valve seat 7307a. Additionally, in one or more aspects, the position and/or location of valve seat 7307a may be modified, for example, to affect/improve the lift off and/or separation of diaphragm 7312 from valve seat 7307a under different pressures and/or temperatures. For example, valve seat 7307a may be offset from the center of diaphragm 7312, which may improve the lift off and/or separation of diaphragm 7312 from valve seat 7307a.

The following features may be optimized in any of the valves described herein to arrive at a desired combination for functionality at different temperature and/or pressures. The off-center or offset valve seat may help increase the lift off pressure (the pressure required to unseat the diaphragm—low pressure cavity pressure) as this is moved away from the center of the valve or cavity. The diaphragm is stiffer near the wall of the valve and thus has less flex. This may be achieved, in part, by moving the point of valve seat/diaphragm contact further away from the more flexed center portion of the diaphragm. The seating pressure (delta pressure) may be increased to allow the diaphragm to seat In some examples, about 0% to about 50% of the diameter may be offset from the center of the diaphragm.

The height of the valve seat also may be increased, enabling the valve seat to be closer to the diaphragm, and resulting in a decreased distance that the diaphragm must travel to seal the valve seat. This in turn also may decrease the seating pressure (delta pressure) required to seat the diaphragm onto the valve seat. However, this also may decrease lift off pressure (low pressure cavity), which is required to lift the diaphragm off of the valve seat. In some examples, the valve seat may be raised from about 0.5 mm to about 3 mm, from about 1 mm to about 2 mm, or about 1.5 mm.

The diameter of the valve seat/vent port/vent opening may also be optimized. As the diameter decreases, the area of the diaphragm being pulled by the opening decreases, improving lift off pressure because there is less force pulling on the diaphragm, and therefore less force required in the bottom cavity to push off. The vent hole may be open to the atmosphere, which is lower than the pressure in the same cavity, and as diameter decreases, the effective area of the pressure drop also decreases (i.e., less atmosphere contacting the low pressure area). The opening diameter may be from about 0.1 mm to about 1 mm, the lower range being limited by manufacturability. In other embodiments, the opening diameter may be about 0.5 mm.

The effective diameter of the diaphragm and/or cavity may be optimized. Increasing the diameter may lower the effective stiffness of the diaphragm, e.g., less rigid and more flexible/elastic. This may be beneficial for seating pressure, but may create an issue with lift off pressure. For example, the cavity may be from about 10 mm to about 20 mm, from about 12 mm to about 18 mm, from about 14 mm to about 16 mm, about 15 mm. In some embodiments, the diameter of the cavity may be about 12.7 mm. In some embodiments, the diameter of the cavity may be from about 0.25 inches to about 1.0 inches.

A composite diaphragm, such as the diaphragm discussed below with respect to FIGS. 7I-7K may include a more rigid portion of the diaphragm contacting the valve seat. This may increase the lift off pressure (low pressure cavity) by preventing localized deformation the vent port/valve seat, preventing seating until a higher pressure by preventing an otherwise flexible portion of the diaphragm from being pulled into the vent hole. The diameter of disc 7412c described below relative to the diameter of the diaphragm may be from about 0% to 90%, from about 50% to about 75%, or about 60%. The disc may be formed of a rigid plastic, while the remainder of the diaphragm may include a material from about 10 to about 90 Shore A durometer, or from about 30 to about 60 Shore A durometer, or from about 40 to about 50 Shore A durometer.

FIGS. 7I-7K illustrate different views of an exemplary diaphragm 7412, which may be incorporated in valve 7300 or any other valve as discussed herein. FIG. 7I is a perspective view of a first side of diaphragm 7412, and FIG. 7J is a perspective view of a second side of diaphragm 7412, with a portion of diaphragm 7412 shown as being partially transparent. FIG. 7K is a cross-sectional view of a portion of diaphragm 7412. Diaphragm 7412 may be generally circular. Diaphragm 7412 may include an outer rim or gland 7412a that extends around the periphery of diaphragm 7412. As shown in FIG. 7I, gland 7412a may extend away from the body of diaphragm in one direction, although it is contemplated that gland 7412a may extend away from the body in multiple opposing directions. Gland 7412a may include an increased thickness relative to inner portion 7412b of diaphragm 7412. Gland 7412a may also include a round face, for example, along an entire face of gland 7412a (e.g., the surface extending perpendicularly from the radial direction of diaphragm 7412). Additionally, diaphragm 7412 may include a disc 7412c positioned on and/or coupled to inner portion 7412b, for example, in a radially centered position on diaphragm 7412. Disc 7412c may be generally cylindrical, and may include a thickness (e.g., extending away from inner portion 7412b) that is approximately the same as the thickness of gland 7412a relative to inner portion 7412b), although it is contemplated that gland 7412a and disc 7412c may have different thicknesses. The thickness of any portion of disc 7412c, including up to an entirety of disc 7412c, may be about 1 mm, about 2 mm, from about 0.5 mm to about 10 mm, from about 1 mm to about 9 mm, from about 3 mm to about 8 mm, from about 4 mm to about 6 mm, or about 5 mm. In some embodiments, the thickness of disc 7412c may be at least 1 mm to assist with manufacturability. As shown, disc 7412c may include one or more indentations or recesses 7412d, for example, curved indentations extending radially inward from the outer circumferential face of disc 7412c. The indentations or recesses 7412d may be spaced from one another about the circumference of disc 7412c. Nevertheless, this disclosure is not so limited, and disc 7412c may be any shape and/or size.

Disc 7412c may be coupled to inner portion 7412b via an adhesive and/or in any other appropriate manner, such as, e.g., molding or other mechanical coupling. In one embodiment, the molding may be a two-shot mold process. As shown in FIGS. 7J and 7K, inner portion 7412b may include one or more holes or recesses 7412e, and disc 7412c may include one or more extensions 7412f, which may be positioned within recesses 7412e in order to couple disc 7412c to inner portion 7412b. Although recesses 7412e are shown in FIG. 7K as extending through an entirety of inner portion 7412b, this disclosure is not so limited. For example, instead, recesses 7412e may extend through only a portion (e.g., approximately 50%, 60%, 70%, 80% etc.) of inner portion 7412b. Correspondingly, extensions 7412f may be sized to be received within recesses 7412f and help couple disc 7412c to inner portion 7412b. In this manner, recesses 7412e and extensions 7412f may help to increase the mechanical bonding of inner portion 7412b and disc 7412c. An end of extensions 7412f may be flush with a face of inner portion 7412b, may protrude outwardly from the face, or may be disposed within the thickness of inner portion 7412b. The recesses may assist with moldability of the disc and attachment of the disc to the diaphragm.

Disc 7412c may be formed of a unitary, single, or composite material, or any other suitable material. Disc 7412c may be formed of a more rigid material than the remaining portions of diaphragm 7412. Disc 7412c may help to increase the stiffness of diaphragm 7412. For example, as shown in FIGS. 7L and 7M, diaphragm 7412 with disc 7412c may be able to receive a greater force and/or pressure, for example, such that diaphragm deflects and/or changes shape more uniformly, which may help during lift-off from a valve seat 7407a at higher pressures. As shown in FIG. 7N, a diaphragm 7412′ without a disc, may deform and/or deflect less uniformly, which may negatively affect, delay, or prohibit lift-off from valve seat 7407a.

Moreover, while one or more seals or vents may be formed within valve 7300 and container 1302, each seal or vent, for example, valve seat 7307a, may be formed in one or more additional or alternative locations. Additionally, one or more of lines, for example, channels may be re-routed and/or one or more connection ports may be moved, repositioned, reoriented, etc. in order to accommodate these features within different space constraints within different containers 1302.

Additionally, although valve 7300 is shown and discussed as being a three part valve (e.g., first housing 7301, second housing 7303, and base plate 7305), this disclosure is not so limited. For example, valve 7300 may be a four part valve. The four part valve may include an additional housing, for example, adjacent and/or coplanar with first housing 7301, and between second housing 7303 and base plate 7305. Alternatively or additionally, the four part valve may include an additional housing (e.g., similar to a portion of first housing 7301 or second housing 7303) or an additional base plate. The four part valve may help the coupling (e.g., welding), and for example, may help to avoid welding through bores, openings, or other portions of valve 7300. These components of valve 7300 may be welded by two laser welds, for example, simultaneously or quasi-simultaneously, for the outer components. Moreover, one or more inner layers or components (e.g., through-holes and high-pressure/low-pressure cavities) may be ultrasonically welded. Furthermore, the material of the valve may change based on compatibility with the gas or fluid moving through the valve. Furthermore, the type of weld used between various layers may be dependent upon the opacity of the layers.

As mentioned above, auto-injector 2 may include a four part valve, for example, a valve 7500, as shown in FIG. 7O. Similar to valve 7300, valve 7500 may be compatible with container 1302 and other systems herein showing a valve. As shown in FIG. 7O, valve 7500 may include a main housing 7501, a first auxiliary housing 7502, a second auxiliary housing 7503, and a base plate 7505. A bottom side of first auxiliary housing 7502 may be coupled to a top side of second auxiliary housing 7503, for example, via an ultrasonic welding. A bottom side of second auxiliary housing 7503 may be coupled to a top side of main housing 7501, for example, via a laser welding. Furthermore, second auxiliary housing 7503 and main housing 7501 may enclose a diaphragm 7512, as discussed above. A bottom side of main housing 7501 may be coupled to a top side of base plate 7505, for example, via a laser welding.

Main housing 7501 may include an inlet 7501a (e.g., a canister inlet), which may connected to the output of fluid source 1366 (FIG. 5), as discussed above. Main housing 7501 may also include a push rod cavity 7501b (similar to PNM flow channel 7309 described herein, used to route gas flow to the device patient needle mechanism, shuttles, and the like) and a dump valve cavity 7501c (used to vent the system after equilibration between the high and low pressure sides) Main housing 7501 may also include a container attachment portion 7501d for connecting to container 1302. Additionally, main housing 7501 may include one or more gaps or spaces, for example, opening 7501e, which may be cored out or otherwise void of material, which may aid in the formation (e.g., molding) of main housing 7501. First auxiliary housing 7502 may help to form a high pressure slide, and may include one or more channels 7502a (i.e., channels associated with high pressure line 3002). Second auxiliary housing 7503 may include one or more channels 7503a (also associated with high pressure line 3002), as discussed above. Base plate 7505 may include a number of channels 7505a-7505c, which may be channels associated with low pressure line 3004 as discussed above. The four part valve may enable push rod cavity 7501b and dump rod cavity 7501c to be larger than in other devices, enabling pressure to be distributed over the larger surface area of a larger rod/dump valve body, thereby potentially improving device performance, particularly at cold temperatures.

Accordingly, various components of valve 7500, including diaphragm 75012, may function similarly to valve 7300 and diaphragm 7312 in order to selectively block and/or lift off from a valve seat (not shown) in order to help control the flow of gas from between high pressure regions and low pressure regions.

Valve 7500 may help to provide for the fluid flow with a simple channel arrangement. The arrangement of the components of valve 7500 may also help to allow for simple welding to form valve 7500. As with valve 7300, the weldings may be one or more of ultrasonic and/or laser weldings. Moreover, valve 7500 may include a smaller overall size than other valves, which may help to provide for more available space with an auto-injector and/or a smaller auto-injector. Additionally, first auxiliary housing 7502 and second auxiliary housing 7503 may be coupled via an ultrasonic welding to form a high pressure subassembly. Main housing 7501 and bottom plate 7504 may be coupled via a laser welding to form a low pressure subassembly. The high pressure subassembly may be coupled to main housing 7501 via a laser welding, for example, to couple the high pressure subassembly to the low pressure subassembly. In this embodiment, the diaphragm may not include any tenting feature, outer rib, or diaphragm jog, although it is contemplated that the diaphragm may include such features in other embodiments used with the four-part valve. The removal of these features may help reduce the footprint or surface area of the diaphragm and valve, and thus help reduce the overall size of auto-injector 2.

The different parts of a valve may be welded using different techniques and/or a different order of operations based on various parameters. Material options for clear or black, e.g., -polystyrene, -ABS, or -polycarbonate (which may not compatible with ultrasonic). The material of the different valve parts may be selected based on the gas/fluid/liquid selected to drive the device, e.g., styrenes may not be compatible with particular gases, e.g., HFA. In one embodiment, the low pressure valve half 7501 is carbon black, or otherwise black in color. In one embodiment, the high pressure valve half 7503 and low pressure slide 7504 are clear.

In one embodiment, a first step may include welding the high pressure valve half 7503 and low pressure slide 7504 onto the low pressure valve half 7501 using laser welding. The order in which the high pressure valve half 7503 or the low pressure slide 7504 is welded to the low pressure valve half 7501 may be interchangeable. A second step may include ultrasonically welding the high pressure slide 7502 onto high pressure valve half 7503.

In another embodiment, an inverse approach may be utilized. That is, in a first step, the high pressure slide 7502 may be ultrasonically welded onto the high pressure valve half 7503. The combined feature may be welded to the low pressure valve half 7501, and the low pressure slide 7504 may be welded onto the low pressure valve half 7501—these two welds being interchangeable in order.

In some embodiments, ultrasonic welding may be performed first because particulate matter may be created and it may be desirable to remove the particulate matter before laser welding. Alternatively, ultrasonic welding can follow laser welding. In this alternative order of operation, it may be desirable to clear dust and other particulate matter from the parts without trapping the dust and particulate matter in the valve near the frit, or if there is minimal to no dust.

In another embodiment, the high pressure valve half 7503 and low pressure valve half 7501 may be carbon black, or otherwise black, opaque, or darker in color, and the high pressure slide 7502 and low pressure slide 7504 are clear. In this embodiment, the high pressure valve half 7503 and low pressure valve half 7501 may be ultrasonically welded, and the high pressure slide 7502 and low pressure slide 7504 may then be laser welded.

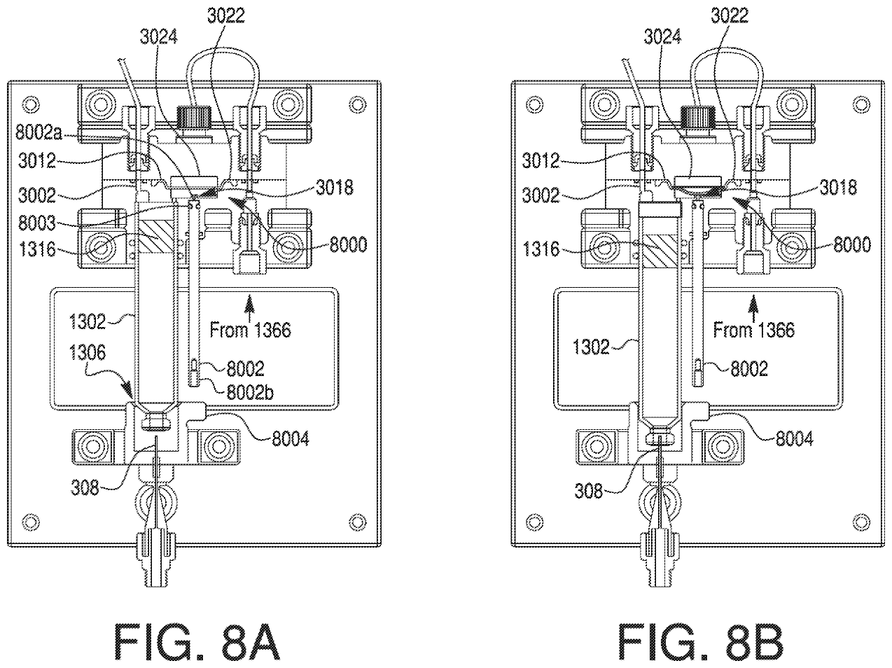

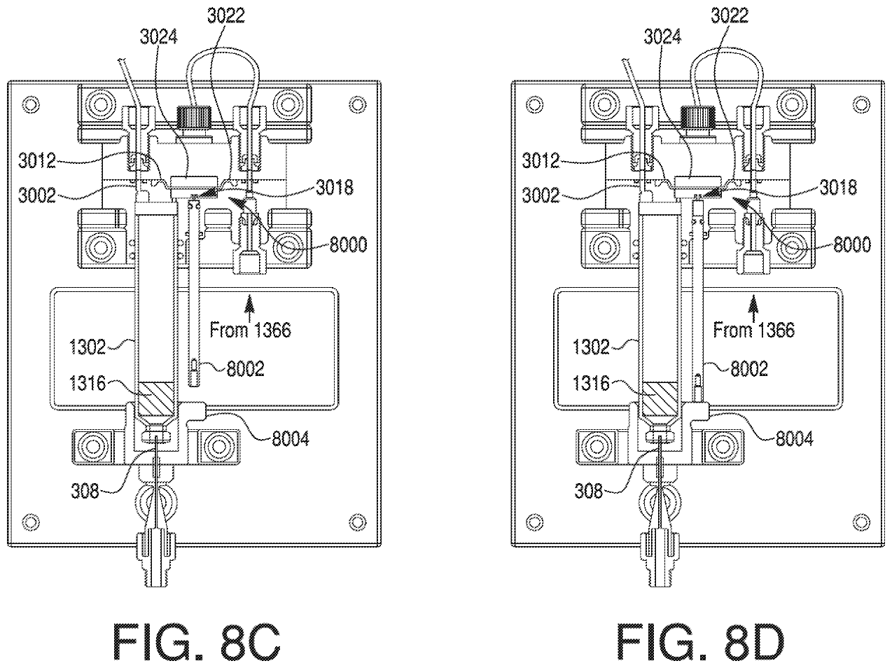

FIGS. 8A-8D show one embodiment of a venting system 8000 according to the disclosure. Venting system 8000 includes a rod or other actuatable member 8002 disposed in conduit 3018. Rod 8002 may extend from a first end 8002a toward a second end 8002b. Rod 8002 may include a seal 8003 at or adjacent to first end 8002a. Pressurized gas from conduit 3018 may contact first end 8002a and not second end 8002b. Rod 8002 is movable from a first position shown in FIGS. 8A-8C to a second position shown in FIG. 8D, where rod 8002 is shown contacting and activating a needle retraction mechanism 8004. Seal 8003 may help ensure that pressurized fluid travelling through conduit 3018 displaces rod 8002 (instead of merely travelling around rod 8002).

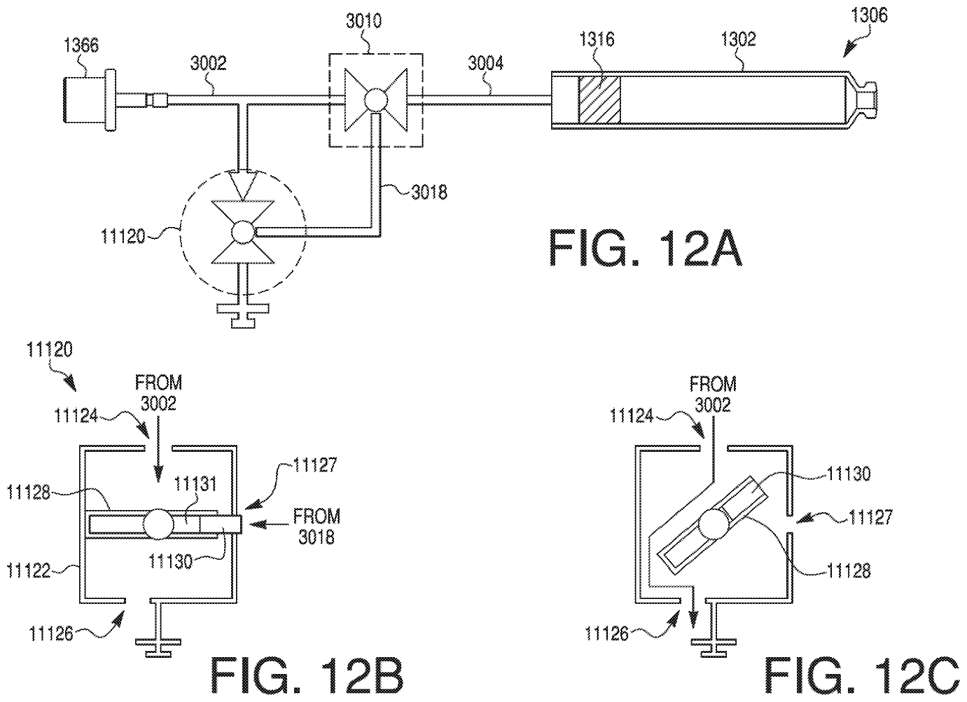

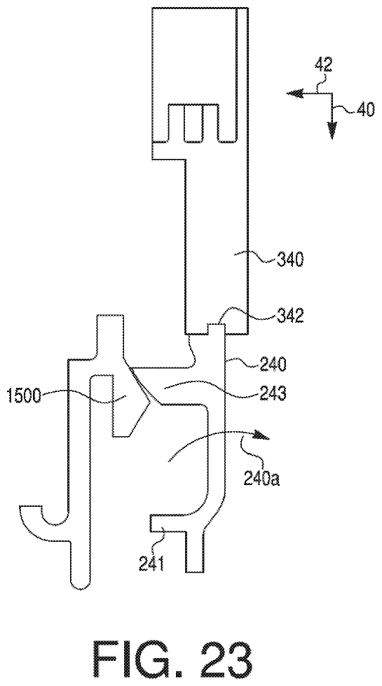

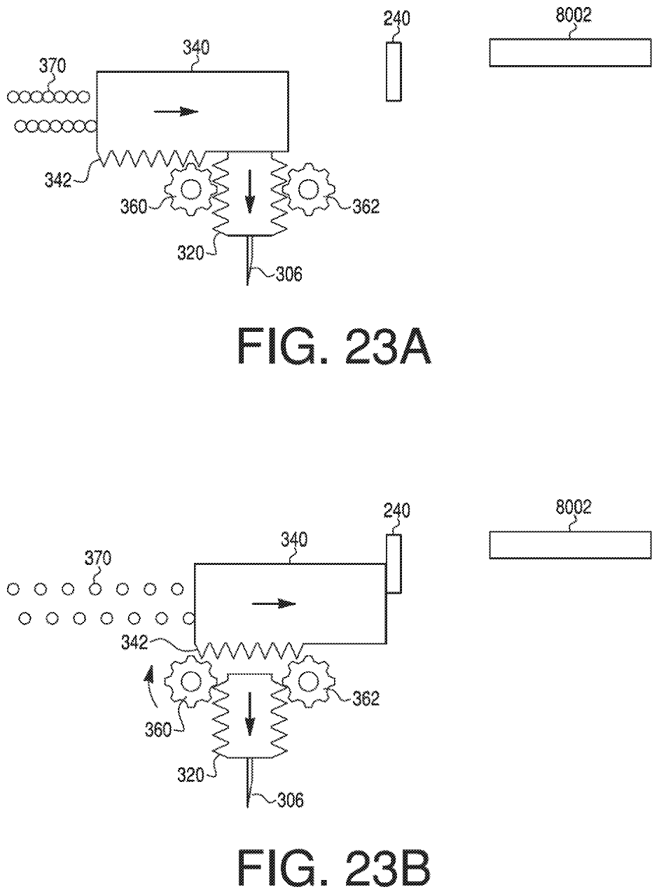

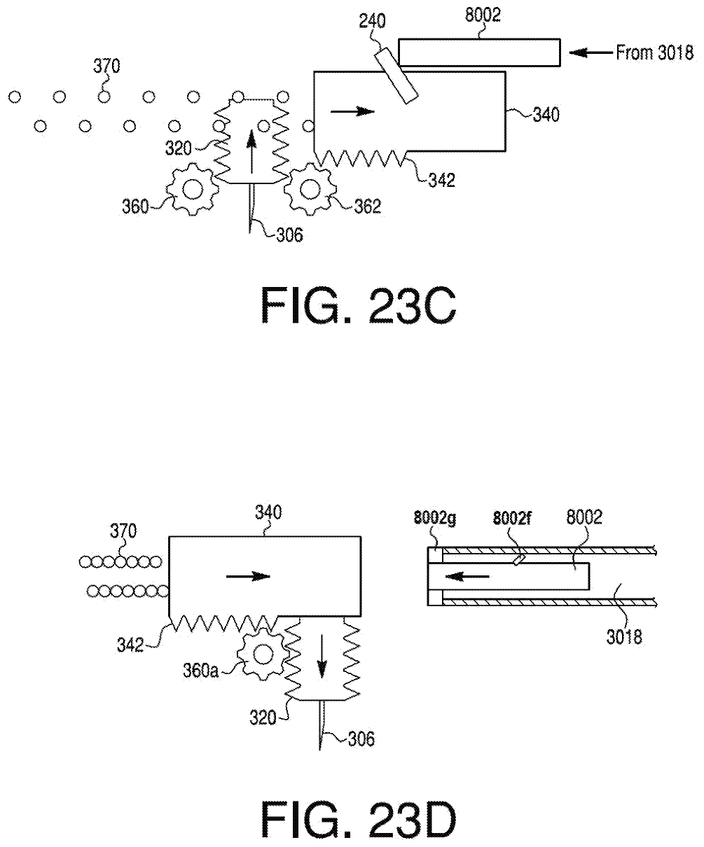

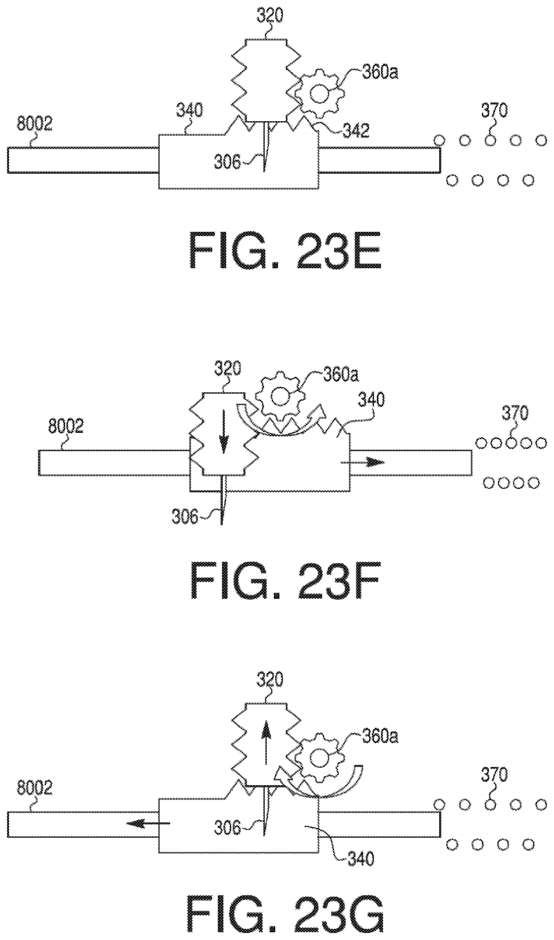

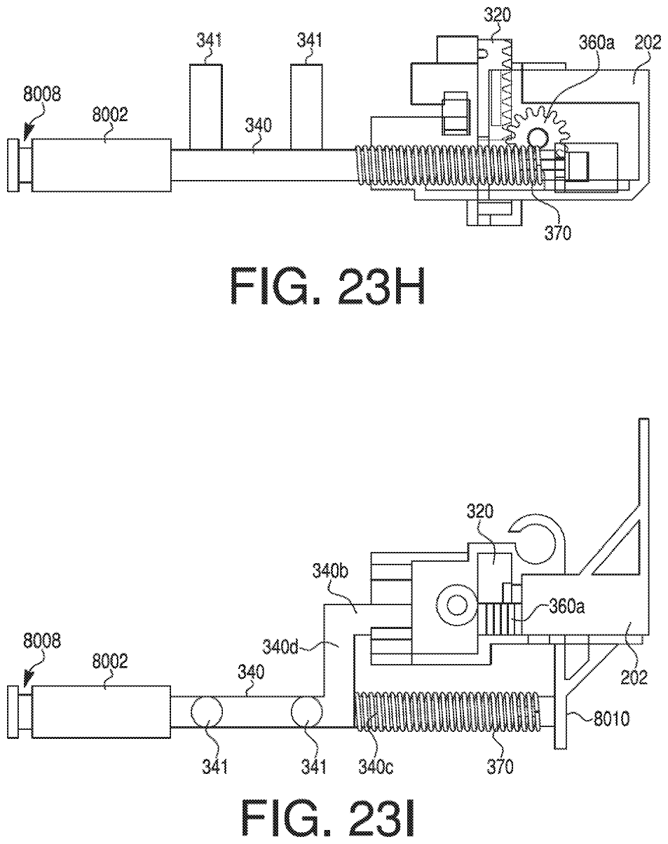

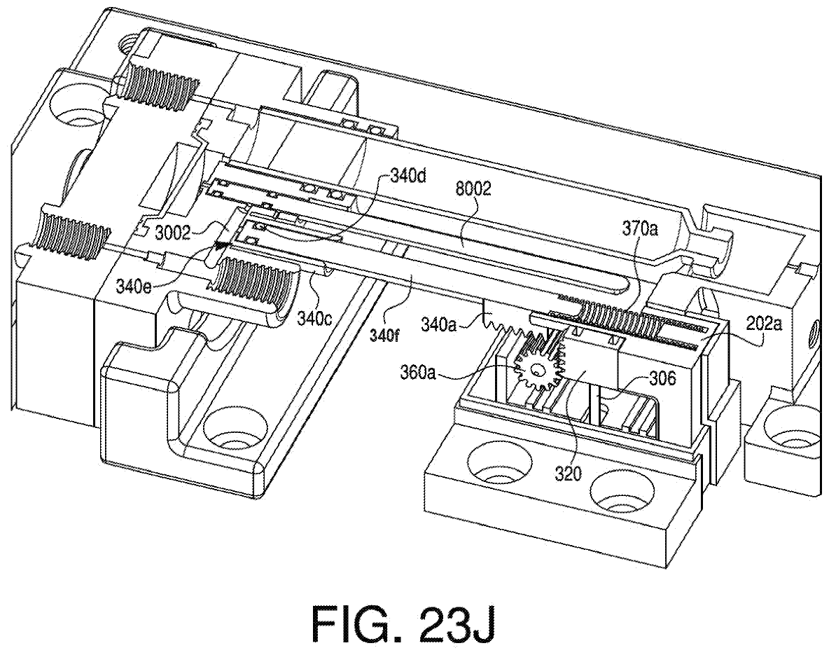

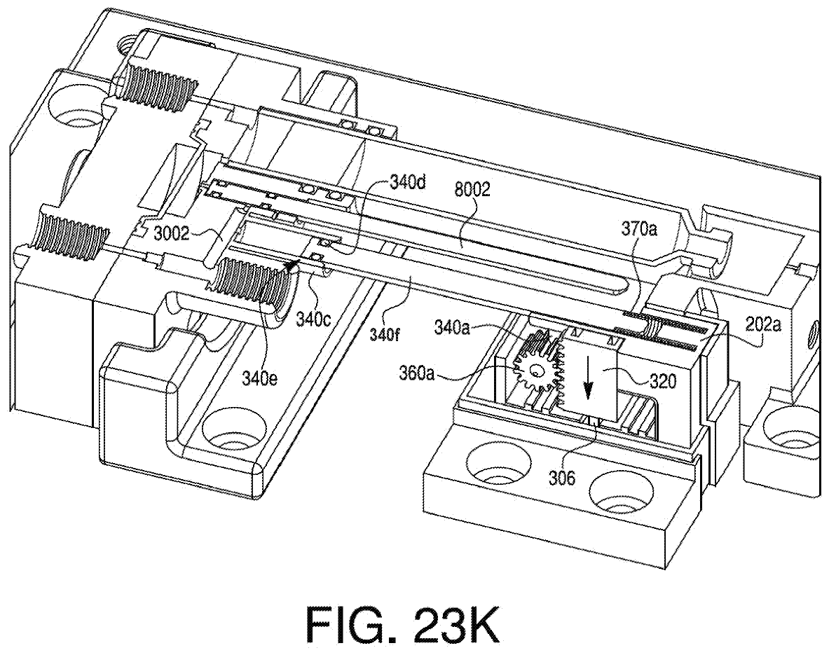

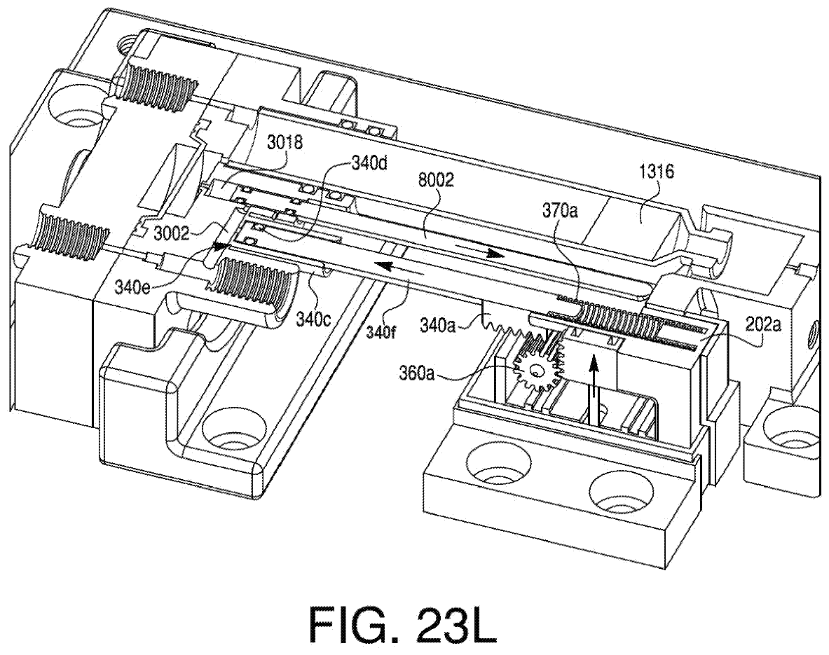

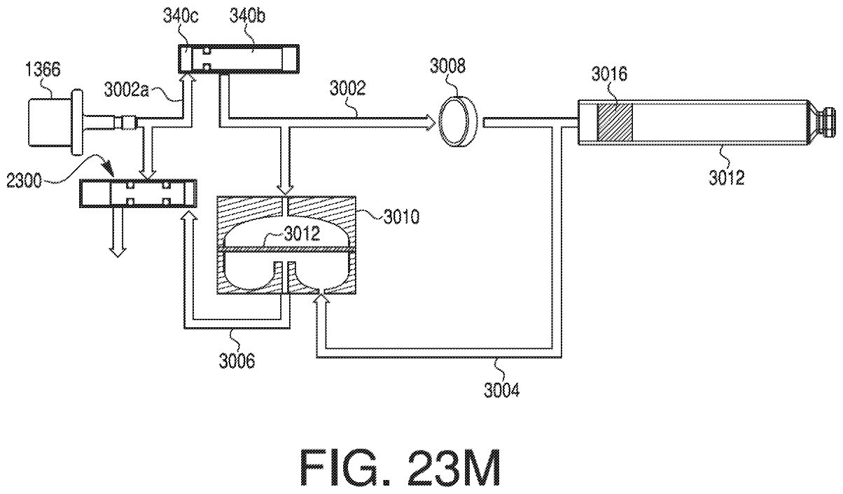

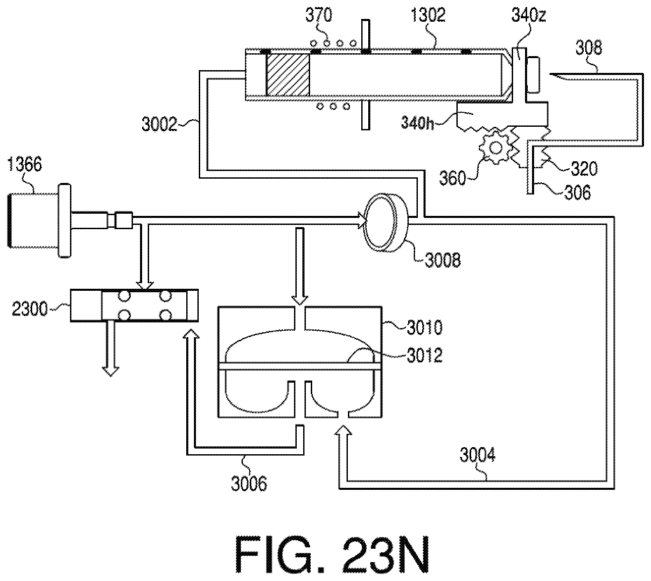

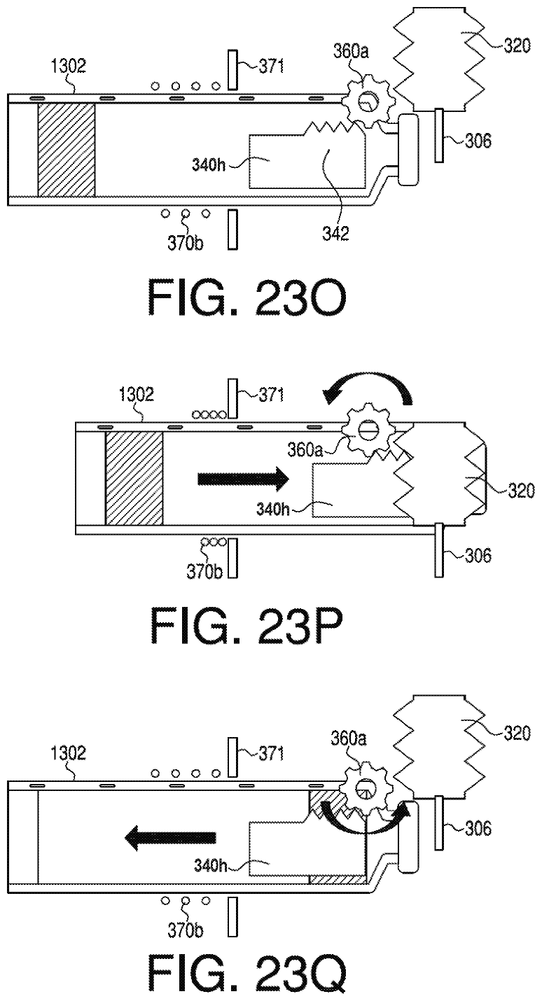

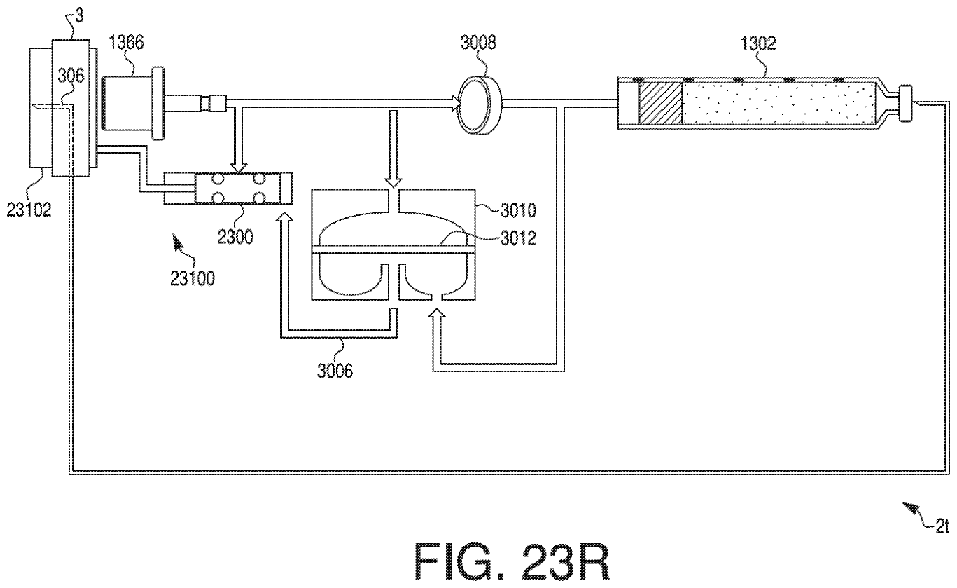

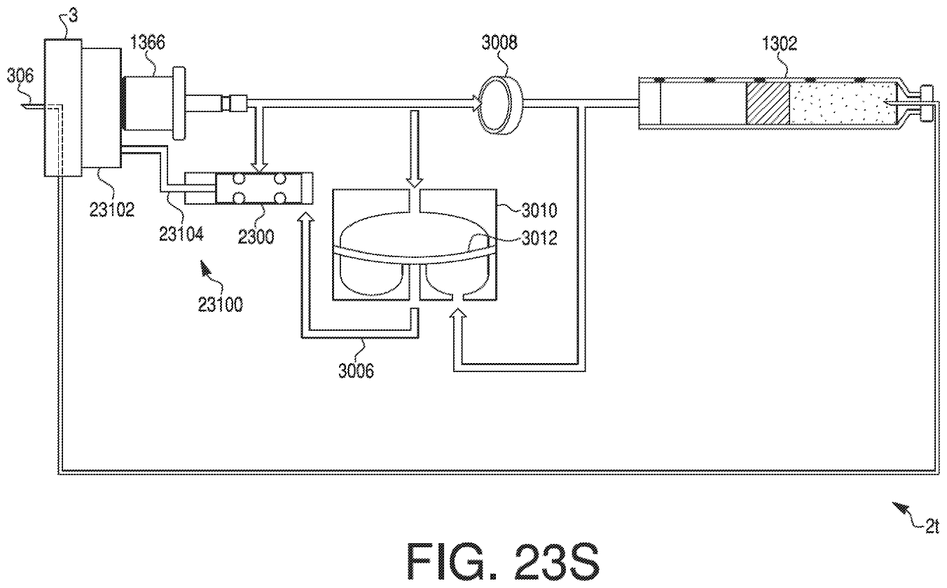

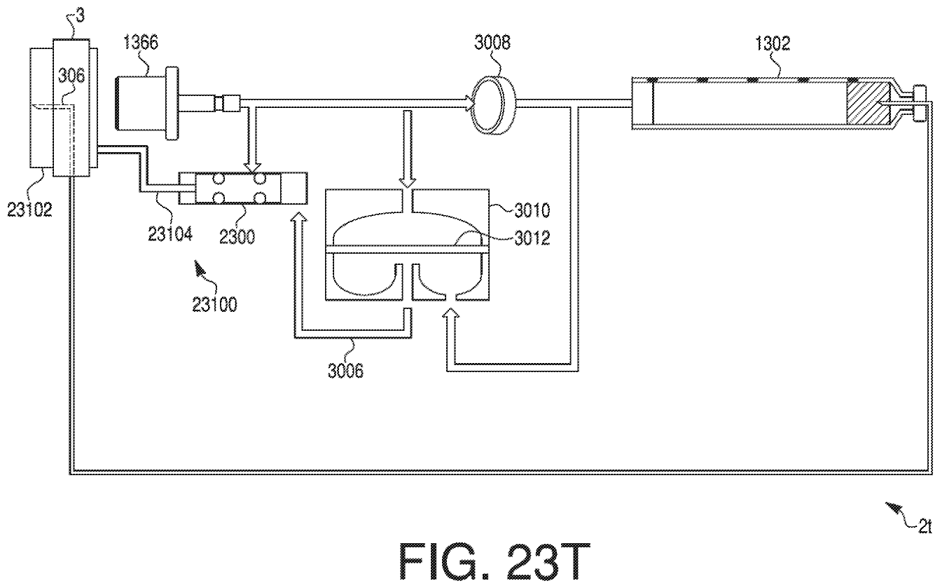

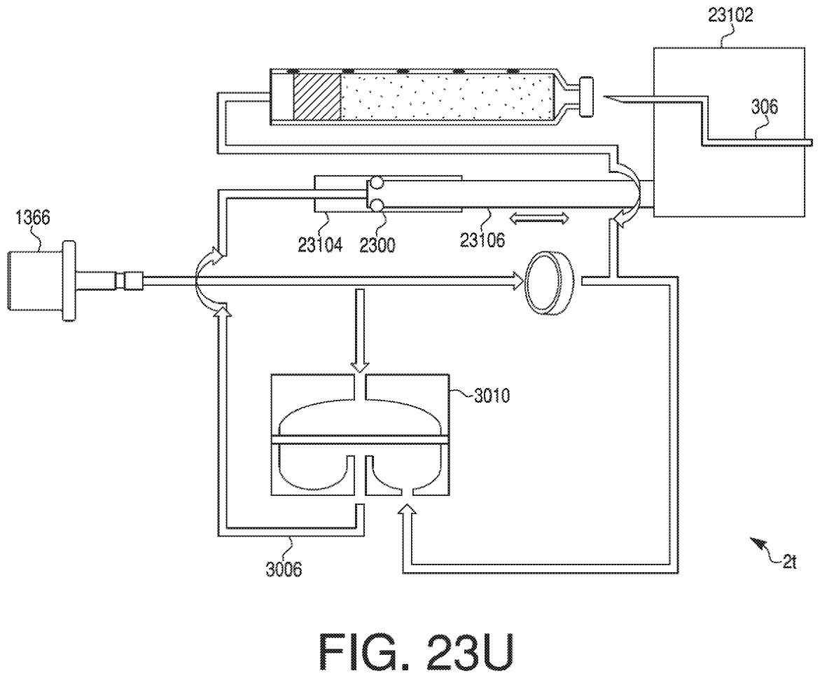

FIG. 8A depicts the system prior to the release of any pressurized gas from fluid source 1366. In FIG. 8A, diaphragm 3012 is in a neutral state, and the second end 1306 of container 1302 is spaced apart from needle 308. FIG. 8B depicts needle 308 in fluid communication with container 1302 after pressurized gas is released from fluid source 1366. In FIG. 8B, piston 1316 is being driven through container 1302, and diaphragm 3012 is pressed against conduit 3018. FIG. 8C shows completion of the injection. In FIG. 8C, piston 1316 has traveled through the entirety of container 1302 (piston 1316 has “bottomed-out”). As set forth above, at this stage, the pressures in high pressure cavity 3022 and low pressure cavity 3024 equilibrate, and diaphragm 3012 returns to its neutral state, opening conduit 3018. As fluid source 1366 may contain more pressurized gas than is needed to complete the injection, the excess pressurized gas may need to be vented out of auto-injector 2. The pressurized gas being diverted through conduit 3018 may drive second end 8002b of rod 8002 into contact with needle retraction mechanism 8004 (FIG. 8D). It is contemplated that the activation of needle retraction mechanism 8004 by rod 8002 may cause a needle (e.g., needle 306 depicted in FIGS. 12A-12C) to retract from a deployed configuration (inside of a patient) to a retracted configuration (inside of auto-injector 2). In one embodiment, needle retraction mechanism 8004 may include one or more of stop 240 and/or ramp 1500 set forth in additional detail below (FIG. 23). For example, rod 8002 may push ramp 1500 and/or stop 240 in order to initiate needle retraction. In such an embodiment, retraction or movement of container 1302 is not needed to initiate retraction of needle 306 from the patient. In some embodiments, once retraction of needle 306 is complete, the flow of pressurized fluid from fluid source 1366 may be stopped so that some amount of pressurized fluid remains in fluid source 1366. In other embodiments, fluid source 1366 may be vented by an alternative mechanism.

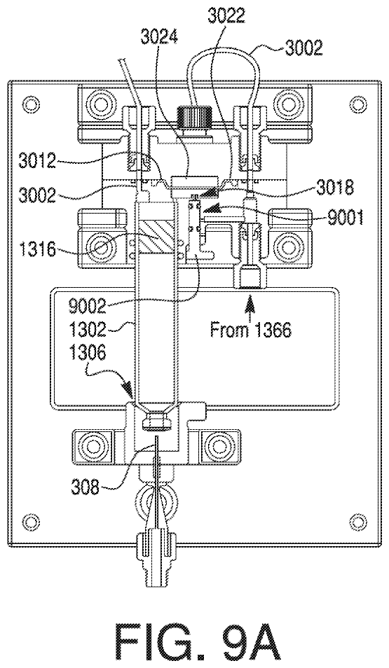

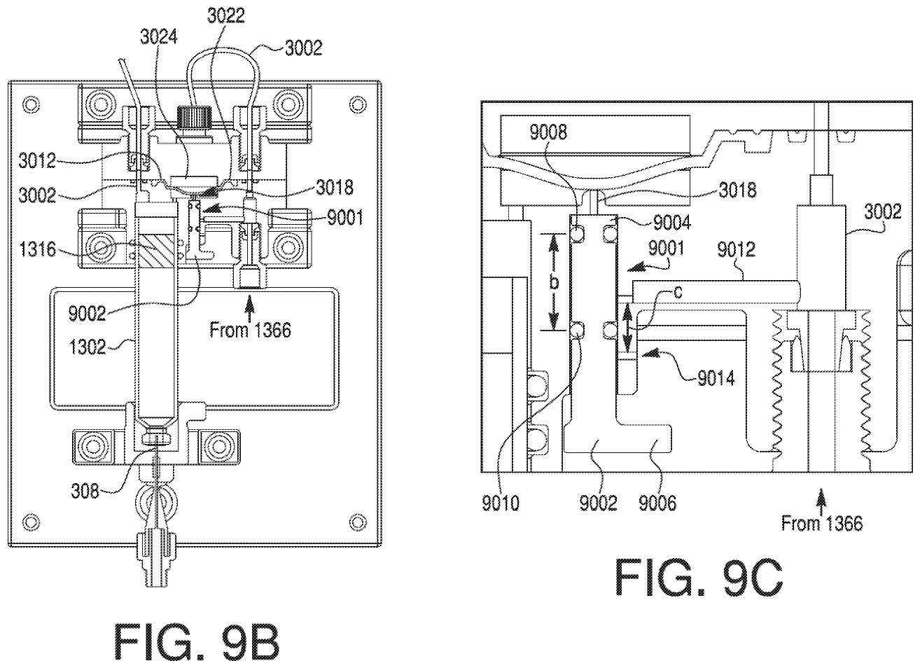

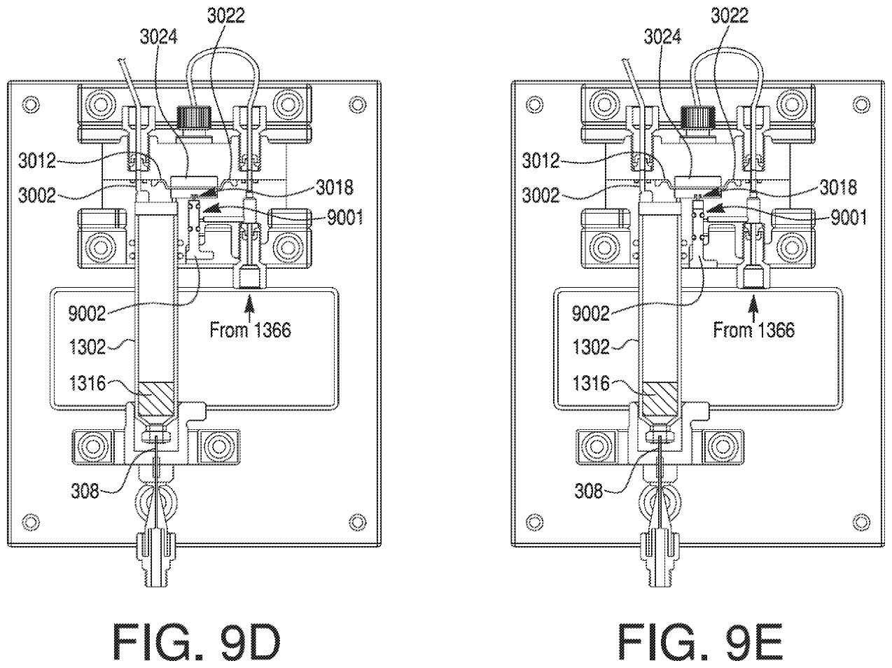

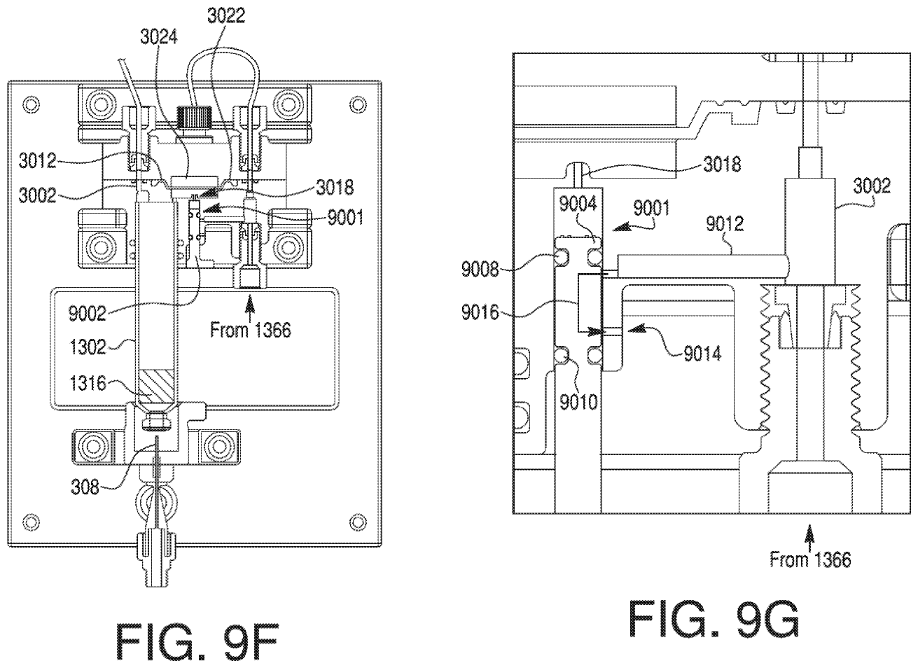

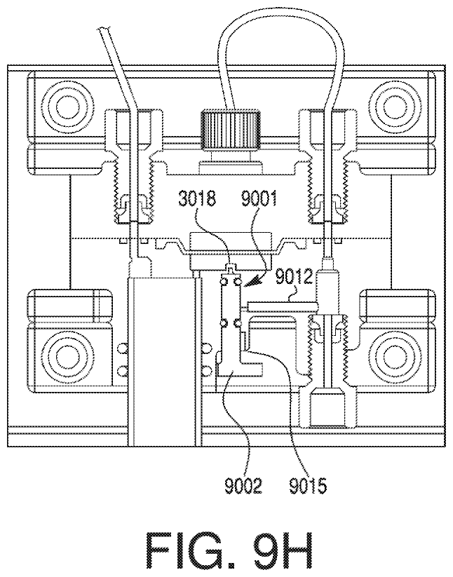

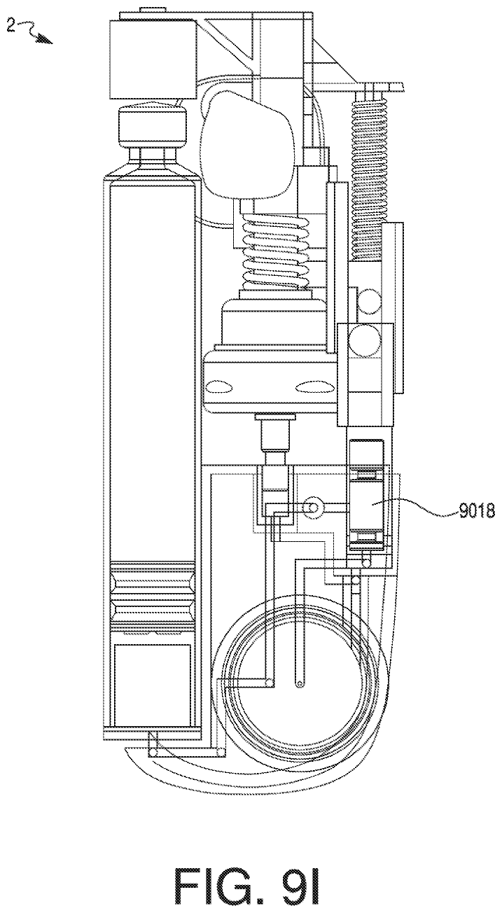

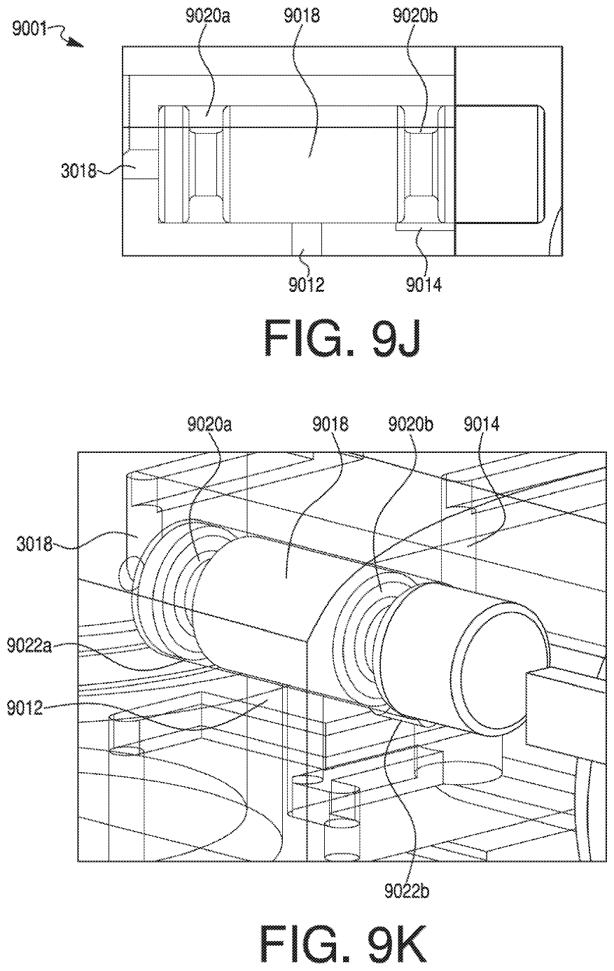

FIGS. 9A-9H illustrate a venting system 9001 according to another embodiment of the disclosure. Venting system 9001 may include a piston 9002 disposed within conduit 3018, forming a valve. Piston 9002 extends from a first end 9004 (best seen in FIGS. 9C and 9G) to a second end 9006. Piston 9002 may have a larger diameter at second end 9006 than at first end 9004. The larger diameter at second end 9006, may serve as a stop to limit movement of piston 9002. For example, an impediment (not shown) can be positioned to precisely limit the range of motion of piston 9002 during venting. Second end 9006 may be used to actuate a needle retraction mechanism as described in other embodiments of the disclosure (e.g., rod 8002). Piston 9002 may be substantially rod-shaped except for the larger diameter extension at second end 9006 described above. The rod portion of piston 9002 may have a slightly smaller diameter than conduit 3018 to enable the escape of gas through conduit 3018 along the outer surface of piston 9002. Piston 9002 may include a first seal 9008 disposed at or adjacent to first end 9004, and a second seal 9010 disposed between first end 9004 and second end 9006. In other words, second seal 9010 may be closer to second end 9006 (and further from first end 9004) than first seal 9008. First seal 9008 and second seal 9010 may be disposed in circumferentially-extending recesses of piston 9002 as shown in FIGS. 9A-9G, or may be disposed around an otherwise uniform outer surface of piston 9002. It is further contemplated that a diameter of piston 9002 between first seal 9008 and second seal 9010 may be smaller than adjacent portions of piston 9002 (to facilitate venting).

Venting system 9001 also may include a secondary channel/line 9012 that is diverted from the inlet receiving pressurized gas from fluid source 1366. Secondary channel 9012 may receive pressurized gas before (or after) pressurized gas flows into high pressure line 3002. Secondary channel 9012 may connect to conduit 3018 downstream of the inlet of conduit 3018. Conduit 3018 may include an outlet 9014, where pressurized gas is released into an interior cavity of auto-injector 2 and/or into the atmosphere. A distance b between seals 9008 and 9010 may be greater than a distance c between the outlet of secondary channel 9012 and outlet 9014 of conduit 3018. In an alternative embodiment shown in FIG. 9H, venting system 9001 may include an enlarged opening or slot 9015 at the end of conduit 3018, instead of outlet 9014. In particular, opening 9015 may be a portion at the end of conduit 3018 having a larger diameter than a remaining portion of conduit 3018. Opening 9015 may serve a similar or same function as outlet 9014 (i.e., to enable release of pressurized gas from fluid source 1366 into an interior cavity of auto-injector 2 and/or into the atmosphere.

FIG. 9A shows portions of auto-injector 2 before release of any pressurized gas from fluid source 1366. In FIG. 9A, diaphragm 3012 is in a neutral state, and the second end 1306 of container 1302 is spaced apart from needle 308. FIG. 9B depicts needle 308 in fluid communication with container 1302 after pressurized gas is released from fluid source 1366. In FIG. 9B, piston 1316 is being driven through container 1302, and diaphragm 3012 is pressed against conduit 3018. FIG. 9C is an enlargement of FIG. 9B, focusing on venting system 9001. During the injection, piston 9002 is disposed in a first position, where first end 9004 is adjacent to and/or in contact with valve seat 3020. In this position second seal 9010 is disposed between the outlet of secondary channel 9012 and outlet 9014 of conduit 3018. Thus, the flow of pressurized gas from secondary channel 9012 to outlet 9014 (and the atmosphere) is blocked by seal 9010.

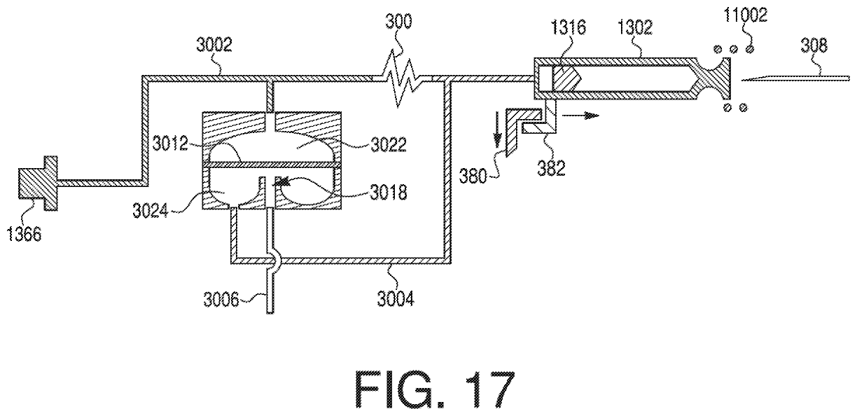

FIG. 9D shows completion of the injection. In FIG. 9D, piston 1316 has traveled through the entirety of container 1302 (piston 1316 has “bottomed-out”). As set forth above, at this stage, the pressures in high pressure cavity 3022 and low pressure cavity 3024 equilibrate, and diaphragm 3012 returns to its neutral state, opening conduit 3018. As fluid source 1366 may contain more pressurized gas than is needed to complete the injection, the excess pressurized gas may be vented out of auto-injector 2. The pressurized gas being diverted through conduit 3018 may drive piston 9002 through conduit 3018 and away from valve seat 3020, as shown in FIGS. 9E-9G. Piston 9002 may be driven away from valve seat 3020 until, e.g., second end 9006 abuts an impediment (not shown), and piston 9002 reaches a second position. While piston 9002 is in the second position shown in FIGS. 9E-9G, secondary channel 9012 may be in fluid communication with outlet 9014, enabling the venting of pressurized gas to the atmosphere. The pressurized gas may travel from secondary channel 9012, between the outer surface of piston 9002 and the inner surface of conduit 3018, and out of outlet 9014 into the atmosphere. This may occur along a flow path 9016 shown in FIG. 9G. FIG. 9F shows container 1302 in a retracted configuration. In this embodiment, a spring 11002 (described below with reference to FIG. 17) may be configured to cause container 1302 to retract. Venting system 9001 (which includes a dump valve) may facilitate relatively quick venting of fluid source 1366 (and subsequent retraction of needle 306). For example, if venting takes too long, retraction of needle 306 and completion of the injection procedure could be delayed by about 10 seconds, about 15 seconds, or even longer periods of time.

FIGS. 9I-9K illustrate portions of auto-injector 2 with additional features of venting system 9001, according to another embodiment of the disclosure. The embodiment show additional details of the dump valve rod and conduit 3018 described in FIGS. 9A-9H. As mentioned above, venting system 9001 may include a dump valve, for example, including a dump valve rod 9018 that extends through conduit 3018. As shown, dump valve rod 9018 and conduit 3018 each may be substantially cylindrical. Conduit 3018 may also include a radial indent (recessed area) 9022 that is in communication with outlet 9014. Indent 9022 may be an indentation on a radially-inward facing surface of conduit 3018, and indent 9022 may help to allow gas (e.g., flow path 9016 described with reference to FIG. 9G above) to release and/or vent from venting system 9001, for example, into the atmosphere. In particular, gas may travel from secondary channel 9012, through a gap between the inner surfaces of conduit 3018 and dump valve rod 9018, and through indent 9022 and outlet 9014. Dump valve rod 9018 may also include gaps 9022a and 9022b, which may receive and/or accommodate one or more seals. The embodiment shown in FIGS. 9I-9K has the same function as the embodiment disclosed in FIGS. 9A-9H, but is smaller and more discrete, allowing it to fit within smaller device housings. For example, indent 9022/outlet 9014 is a scalloped channel instead of a through-hole. This structure may simplify the molded part and thus may also be easier to manufacture.

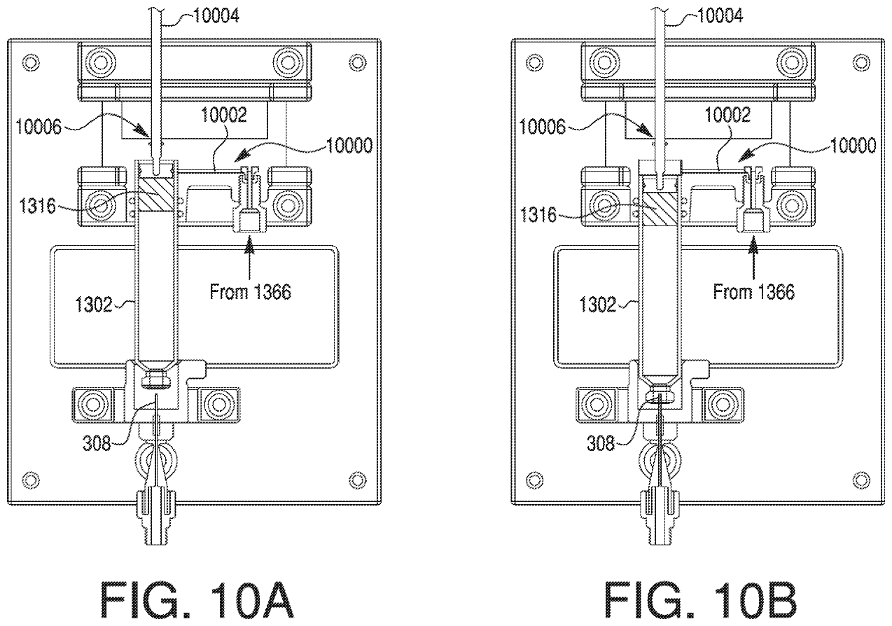

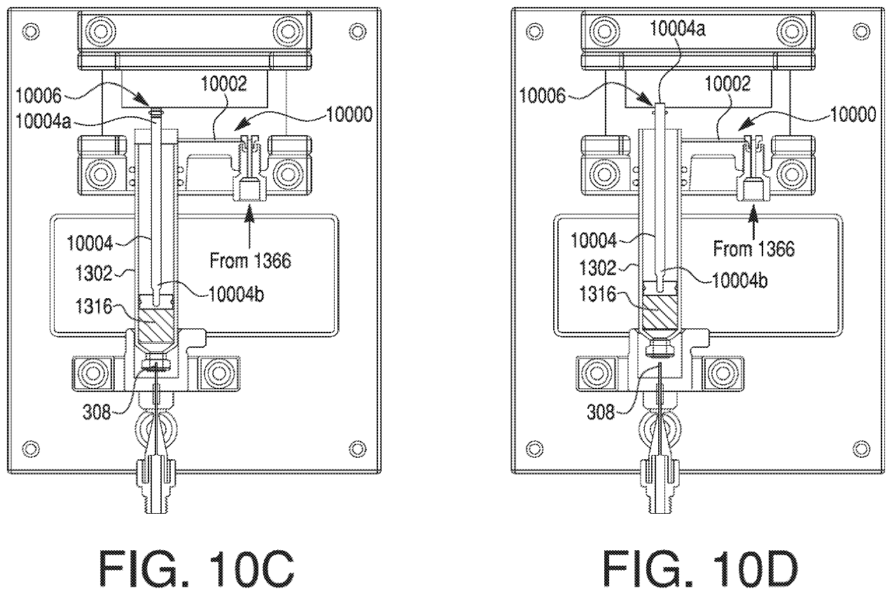

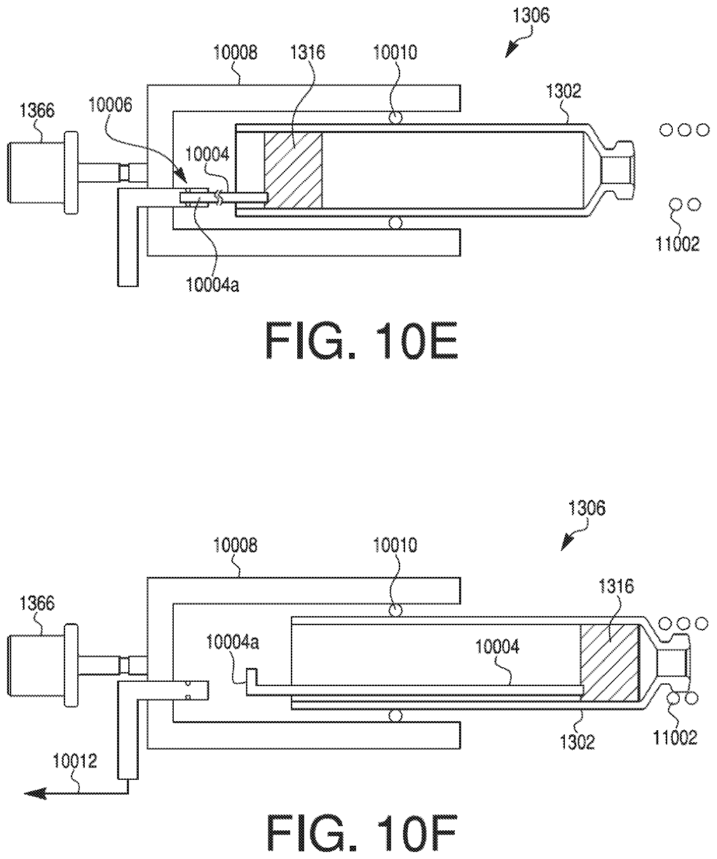

FIGS. 10A-10D show venting system 10000 according to the disclosure. Venting system 10000 is configured to be used without valve 3010 described above, whereas venting systems 8000 and 9001 may be used in conjunction with valve 3010. Venting system 10000 includes line 10002 configured to deliver pressurized gas from fluid source 1366 to container 1302 to initiate fluid communication between container 1302 and needle 308, and also to drive piston 1316 through container 1302. A rod 10004 may extend from a first end 10004a (see FIG. 10D) toward a second end 10004b, where rod 10004 is coupled to a rear (non-medicament-contacting) side of piston 1316. Rod 10004 also may extend through a conduit 10006, as shown in FIGS. 10A and 10B. While rod 10004 is disposed in vent 10006, conduit 10006 is sealed and pressurized gas from fluid source 1366 must act against piston 1316 to drive piston 1316 through container 1302 (see FIG. 10B). When piston 1316 reaches second end 1306 of container 1302 (as shown in FIG. 10C), rod 10004 may be pulled completely through conduit 10006, opening conduit 10006 and allowing pressurized gas from line 10002 to escape therethrough. The pressurized gas will continue to act on piston 1316 (against spring 11002 shown in FIG. 17) and vent simultaneously, until the spring force of the spring 11002 is greater than the force of the pressurized gas acting on piston 1316. At this point, the system is fully vented, and expansion of the spring will cause container 1302 to retract as shown in FIG. 10D (or retract in alternative embodiments). Spring 11002 may return container 1302 to its original, undeployed position, or to a different position than the original undeployed position (e.g., longitudinally offset from the original, undeployed position). The offset position could be closer or further from needle 308 than the original, undeployed position.

FIGS. 10E and 10F show additional views of venting system 10000. In particular, FIG. 10E shows venting system 10000 when first end 10004a of rod 10004 extends through conduit 10006, and before any medicament has been ejected from container 1302 by piston 1316. In FIG. 10F, venting system 10000 is shown after completion of the injection, where piston 1316 has travelled to second end 1306 of container 1302, pulling first end 10004a of rod 10004 out of conduit 10006. As seen in FIG. 10F, first end 10004a may transition from a first configuration shown in FIG. 10E, to a second configuration shown in FIG. 10F. In the first configuration, first end 10004a of rod 10004 may extend along a first axis, e.g., which may be the same axis that a remainder of rod 10004 extends along. In the second configuration shown in FIG. 10F, first end 10004a may extend along a second axis that is offset from the first axis. The offset second configuration shown in FIG. 10F may help prevent first end 10004a from inadvertently re-entering conduit 10006, and inadvertently inhibiting the venting process. In some embodiments, first end 10004a may be biased toward the offset second configuration. For example, rod 10004 may include a shape memory material, such as, e.g., nitinol, that is set into the offset second position. In such embodiments, proximal end 10004a may be urged into the first configuration (e.g., held in the first configuration by conduit 10006), and may revert to the offset second configuration when it is pulled out of conduit 10006. The offset configuration may be achieved, by, for example, tabs, curled plastic, or any other suitable structure. In this embodiment, a seal 10010 may be disposed around container 1302 against the inner surface of a chamber 10008. Furthermore, an outflow 10012 of conduit 10006 may be directed into the surrounding environment/atmosphere, or may be used to actuate other mechanisms described herein. For example, outflow 10012 may be directed to move rod 8002 described above to control needle retraction. The embodiment of FIGS. 10A-10F may remove any need for valve 3010 to sense an end of the injection, as conduit 10006 will automatically open at the end of the injection.

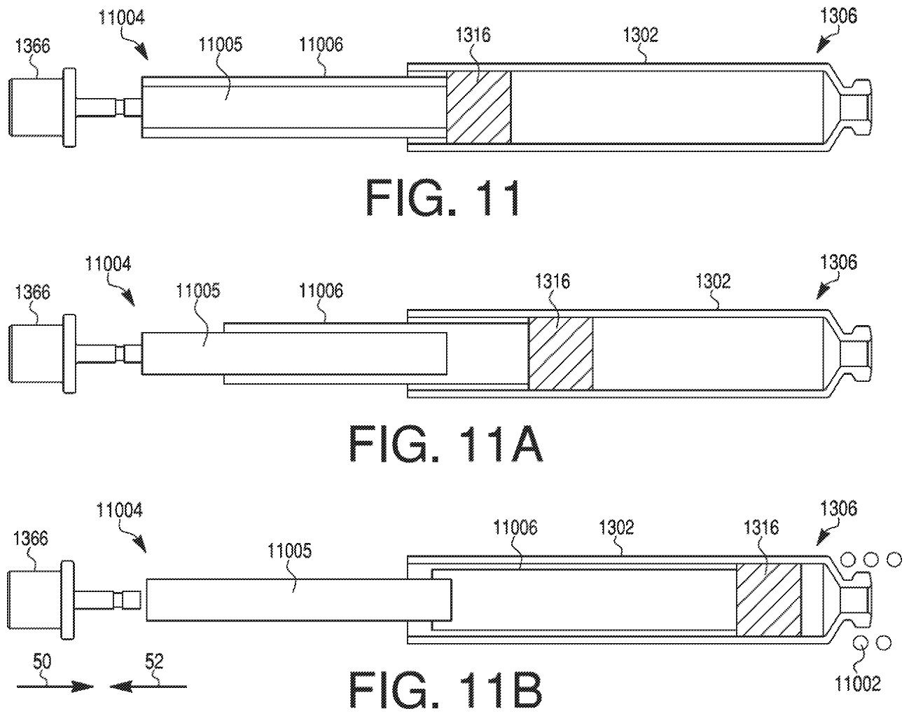

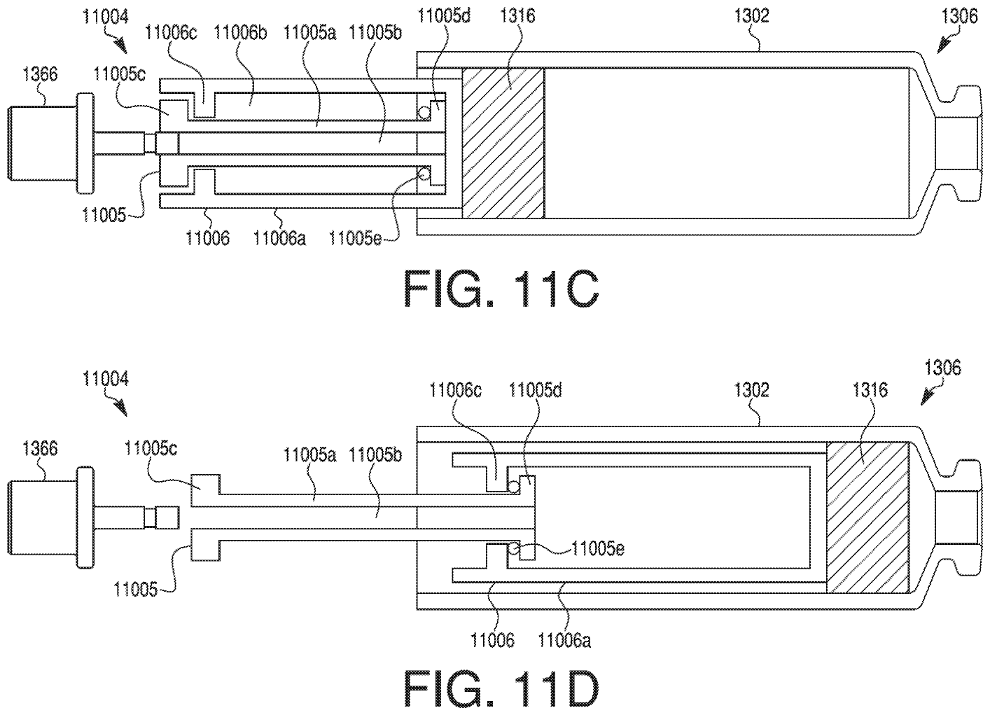

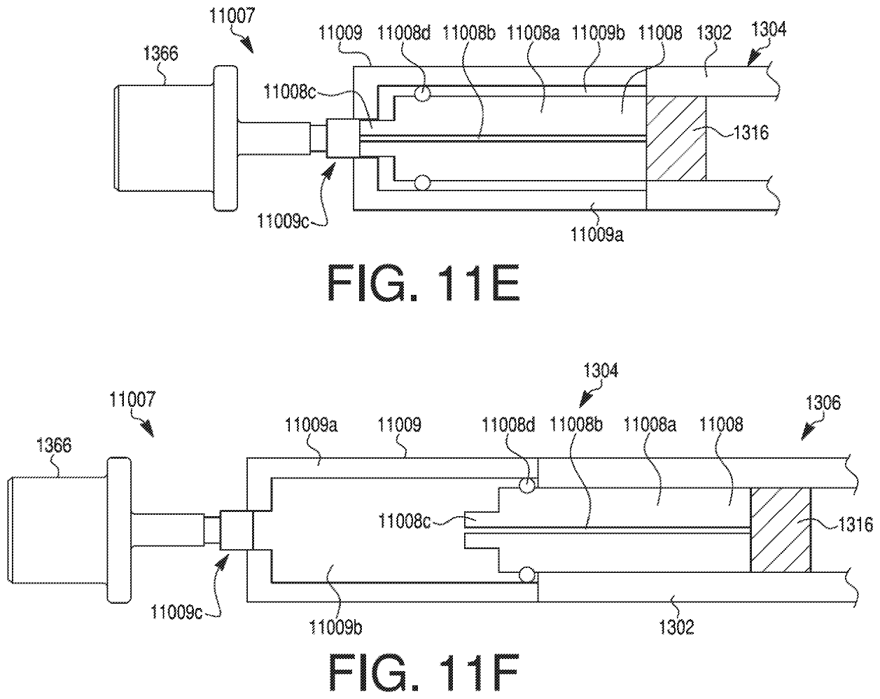

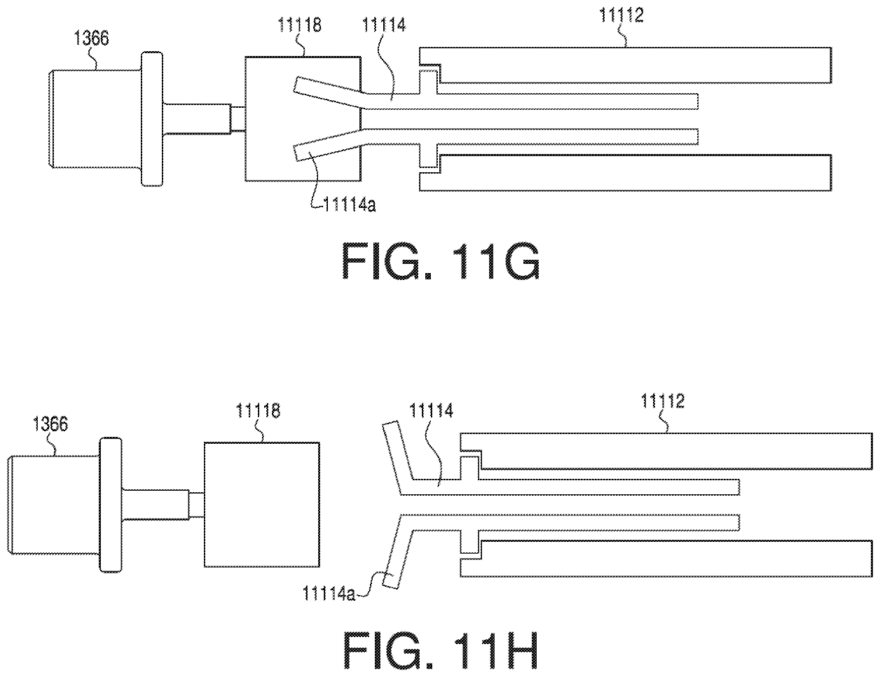

Various venting mechanisms will now be described with reference to FIGS. 11 and 11A-11H that may help expedite venting of fluid source 1366. A venting system 11004 is shown in FIGS. 11, 11A, and 11B, which may include a first straw 11005 and a second straw 11006. First straw 11005 may have a smaller diameter than second straw 11006 and may be contained within second straw 11006 in one or more configurations. For example, first straw 11005 and second straw 11006 may form a telescoping arrangement. The proximal end of first straw 11005 may be coupled to fluid source 1366, and the distal end of second straw 11006 may be coupled to piston 1316. FIG. 11 shows venting system 11004 before fluid source 1366 is activated. In this configuration, first straw 11005 may be completely nested within second straw 11006. It is further noted that in at least some embodiments, first straw 11005 and second straw 11006 may have the same length, although it is contemplated that first straw 11005 and second straw 11006 may have different lengths.

After fluid source 1366 is activated, pressurized fluid may travel through a lumen of first straw 11005 and drive piston 1316. The distal end of first straw 11005 is, in some embodiments, not directly coupled to piston 1316, and thus, the pressurized fluid may urge piston 1316 and second straw 11006 (directly coupled to piston 1316) in a direction toward second end 1306 of second container 1302 (see FIG. 11A). At the end of the injection (see FIG. 11B), when piston 1316 has reached second end 1306 of container 1302, the proximal end of second straw 11006 may catch on an impediment (not shown, described in further detail in other figures) of first straw 11005, preventing further relative movement between first straw 11005 and second straw 11006. At this point, the additional flow of pressurized fluid from fluid source 1366 forces the proximal end of first straw 11005 to disconnect from fluid source 1366, stopping the flow of fluid from fluid source 1366, or allowing fluid source 1366 to vent the remainder of its propellant and pressurized fluid into the environment. The disconnection of first straw 11005 from fluid source 1366 may remove the only force acting on container 1302 in the direction from first end 1304 toward second end 1306. The force acting in the direction, from first end 1304 toward second end 1306, may compress spring 11002 (shown in FIG. 11B) during injection. The absence of the force in that direction may allow spring 11002 to expand, urging container 1302 in a direction from second end 1306 toward first end 1304 (e.g., in an opposite direction). Alternatively, the spring 11002 could be configured to expand during injection, and the absence of force may allow spring 11002 to compress, urging container 1302 in a direction from second end 1306 toward first end 1304.

FIGS. 11C and 11D show further details of venting system 11004, where pressurized fluid from fluid source 1366 causes the outer second straw 11006 to move relative to inner first straw 11005. First straw 11005 may include an elongated body portion 11005a having a lumen 11005b extending therethrough. Fluid source 1366 may include an extension received by lumen 11005b so that pressurized fluid exiting fluid source 1366 flows directly into lumen 11005b. First straw 11005 also may include a proximal flange 11005c and a distal flange 11005d. A seal 11005e, such as, e.g., an O-ring or the like, may be coupled to a proximally-facing surface of distal flange 11005d. Second straw 11006 may include a body portion 11006a having a closed distal end and an open proximal end. Second straw 11006 may enclose a volume 11006b, and may include a flange 11006c adjacent to its proximal end. Before fluid source 1366 is activated, a distal-facing surface of proximal flange 11005c may abut and/or be proximate to a proximal facing surface of flange 11006c.

When fluid source 1366 is activated, the pressurized fluid may flow through lumen 11005b of first straw 11005, and act on the closed distal end of second straw 11006, urging straw 11006 and piston 1316 toward second end 1306 of container 1302. After the end of the injection, when piston 1316 has travelled through container 1302 to second end 1306 (shown in FIG. 11D), a distally-facing surface of flange 11006c may abut seal 11005e and/or the proximally-facing surface of distal flange 11005d. When piston 1316 bottoms out, it may pull second straw 11006, and first straw 11005 (all coupled together) away from fluid source 1366, severing the connecting between first straw 11005 and fluid source 1366. When the connection between first straw 11005 and fluid source 1366 is severed, the flow of pressurized fluid may be stopped, or any further pressurized fluid expelled from fluid source 1366 may vent into its surroundings, and/or into the atmosphere.

FIGS. 11E and 11F show an embodiment of a venting system 11007 similar to the venting system 11004 shown in FIGS. 11C and 11D, except that in venting system 11007, an inner first straw 11008 is driven by fluid source 1366 relative to an outer second straw 11009. Inner first straw 11008 includes an elongate body portion 11008a having a lumen 11008b extending therethrough. Body portion 11008a may include a narrowed proximal end 11008c, and the distal end of body portion may be coupled to a proximal surface of piston 1316. A seal 11008d, such as an O-ring, may extend around at least a part of body portion 11008a. Second straw 11009 may include a body portion 11009a enclosing a volume 11009b through which first straw 11008 travels. The proximal end of second straw 11009 may include an opening 11009c configured to receive a conduit of fluid source 1366. The distal end of second straw 11009 may be coupled to and closed by first end 1304 of container 1302.

After fluid source 1366 is activated, pressurized fluid may travel through lumen 11008b of first straw 11008 and drive piston 1316. The distal end of first straw 11005 may be directly coupled to piston 1316, and thus, the pressurized fluid may urge piston 1316 and first straw 11008 in a direction toward second end 1306 of second container 1302 (see FIG. 11F). At the end of the injection (see FIG. 11F), when piston 1316 has reached second end 1306 of container 1302, first straw 11008 cannot move any further distally, and the continuing release of pressurized gas from fluid source 1366 may push container 1302, first straw 11008, and second straw 11009 (all coupled together) away from fluid source 1366, severing the connecting between second straw 11009 and fluid source 1366 (not shown). When the connection between second straw 11009 and fluid source 1366 is severed, the flow of pressurized fluid may be stopped, or any further pressurized fluid from fluid source 1366 may vent into its surroundings, and ultimately, into the atmosphere.

FIGS. 11G and 11H show examples of features that can be used with either venting system 11004 or 11007 described above. In particular, these figures show a coupler 11118 attached to the outflow of fluid source 1366. Coupler 11118 may be attached to the proximal end 11114a of a first straw 11114 (which could be the proximal end of any of the straws set forth above). A second straw 11112 may be coupled to piston 1316 (not shown in FIGS. 11G and 11H) and may be driven by pressurized fluid from fluid source 1366. As described above, at the end of an injection, piston 1316 may bottom out and reach second end 1306 of container 1302 (not shown in FIGS. 11G and 11H), and the further expulsion of pressurized fluid from fluid source 1366 may cause each of first straw 11114, second straw 11112, and container 1302, to sever from coupler 11118 and/or fluid source 1366. While a coupler 11118 is shown in FIGS. 11G and 11H, it is contemplated, that in at least some embodiments, that first straw 11114 may be coupled directly to fluid source 1366 to receive the pressure gas from fluid source 1366 directly.

After proximal end 11114a of first straw 11114 is severed from coupler 11118 and/or fluid source 1366, proximal end 11114a may transition from a first configuration shown in FIG. 11G to a second configuration shown in FIG. 11H. In some embodiments, proximal end 11114a may be biased into the second configuration. While coupled to coupler 11118 and/or fluid source 1366, proximal end 1366 may be maintained into the first configuration by the geometry of coupler 11118 and/or fluid source 1366. For example, proximal end 11114a may be inserted into coupler 11118 and/or a conduit of fluid source 1366, that constrains proximal end 11114a in the first configuration, and upon its removal from coupler 11118 and/or fluid source 1366, proximal end 11114a may revert to the second configuration shown in FIG. 11H.

In one embodiment, proximal end 11114a may include a shape memory material, e.g., SMA, smart metal, memory metal, memory alloy, muscle wire, smart alloy, that is biased into the second configuration. In another embodiment, proximal end 11114a may include a frangible material that breaks off from a remainder of first straw 11114 after first straw 11114 detaches from coupler 11118 and/or fluid source 1366. In the second configuration, first straw 11114 may be substantially prevented or hindered from reattaching to coupler 11118 and/or fluid source 1366, allowing fluid source 1366 to vent any remaining propellant or pressurized gas into its surroundings, and ultimately, to the atmosphere, or to stop the flow of pressurized gas from fluid source 1366 altogether.

FIGS. 12A-12C show a valve (e.g., a butterfly valve) 11120 that can be used in conjunction with various embodiments disclosed herein, such as, e.g., the embodiment shown in FIGS. 3A-3C. In particular, valve 11120 may be coupled to high pressure line 3002 and conduit 3018 of valve 3010. Referring now to FIG. 12B, valve 11120 is shown in a closed configuration, where flow diverted from high pressure line 3002 is prevented from travelling through valve 11120. Valve 11120 may include a housing 11122 having a first inlet 11124 (configured to receive a flow from high pressure line 3002), an outlet 11126, and a second inlet 11127 that is configured, in some embodiments, to receive a flow from conduit 3018 of valve 3010. Valve 11120 may include a movable member 11128 configured to move within and relative to housing 11122.

In the closed configuration shown in FIG. 12B, movable member 11128 may substantially or entirely block the flow of pressurized gas from high pressure line 3002 through valve 11120. Movable member 11128 may be rotatable within housing 11122 about an axis, and may include a movable pin 11130. Movable pin 11130 may be disposed in and reciprocally movable within a lumen 11131 of movable member 11128. However, other suitable configuration also are contemplated. For example, movable pin 11130 may slide relative to a slot or recess of movable member 11128. In the closed configuration shown in FIG. 12B, fluid flow through second inlet 11127 is blocked by movable pin 11130, which is disposed through second inlet 11127. As shown in FIG. 12C, movable pin 11130 may slide within lumen 11131 of movable member 11128, releasing movable member 11128 from its first position shown in FIG. 12B, so that movable member 11128 rotates or moves to a second position shown in FIG. 12C. FIG. 12C shows valve 11120 in an open configuration, where pressurized gas from high pressure line 3002 may flow through valve 11120, venting the remaining pressurized gas from fluid source 1366 into the surrounding environment, and ultimately, into the atmosphere.

Before auto-injector 2 is initiated, valve 11120 may be in the closed configuration shown in FIG. 12B, and may remain in the closed configuration after activation of fluid source 1366 and during an injection. That is, valve 11120 may be in the closed configuration while piston 1316 is driven through container 1302 and until piston 1316 reaches second end 1306 (and bottoms out). At the end of the injection, diaphragm 3012 (shown in FIGS. 3A-3C) of valve 3010 may return to its neutral state, enabling flow through conduit 3018. The flow through conduit 3018 may act on movable pin 11130 (e.g., pushing movable pin 11130 into lumen 11131), allowing movable member 11128 to release from its locked first position. Once movable member 11128 is released from the locked first position shown in FIG. 12B, pressurized gas flowing through high pressure line 3002 may travel through valve 11120 to vent any remaining propellant stored in fluid source 1366.

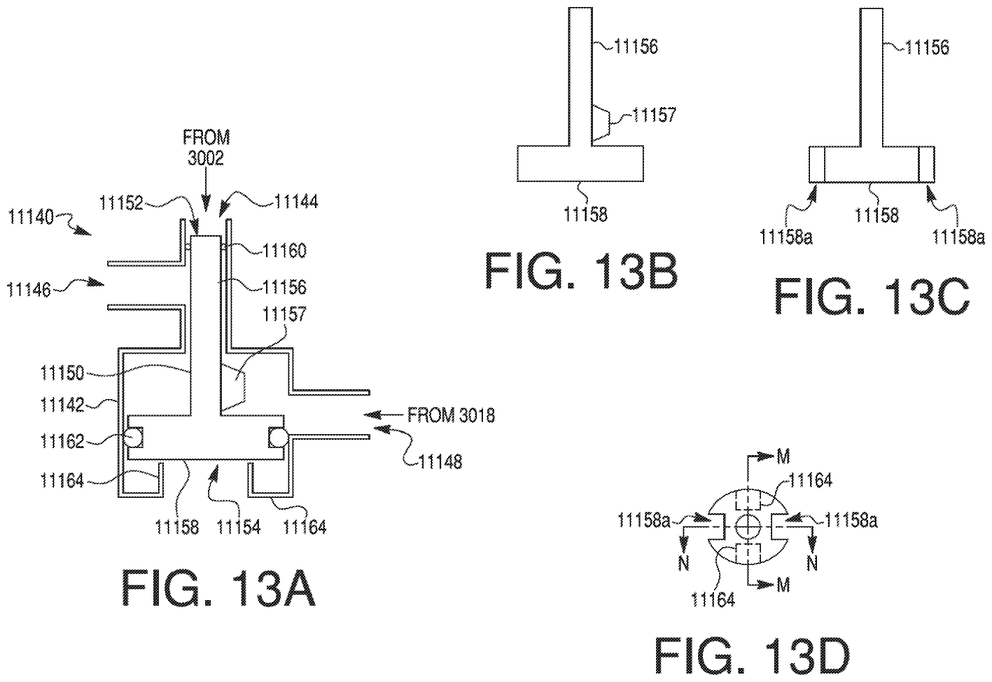

FIGS. 13A-13D show a valve 11140 that can be used in conjunction with various embodiments disclosed herein, such as, e.g., the embodiment shown in FIGS. 3A-3C. Furthermore, valve 11140 may be positioned within auto-injector 2 in a similar manner as valve 11120. For example, valve 11140 may be coupled to high pressure line 3002 and conduit 3018.

Referring now to FIG. 13A, valve 11140 is shown in a closed configuration, where flow diverted from high pressure line 3002 is prevented from travelling through valve 11140. Valve 11140 may include a housing 11142 having a first inlet 11144 (configured to receive a flow from high pressure line 3002), an outlet 11146, and a second inlet 11148 that is configured, in some embodiments, to receive a flow from conduit 3018 of valve 3010. Valve 11140 may include a piston 11150 configured to move within and relative to housing 11142. An elongate member, e.g., a shaft 11156 of piston 11150 may extend from a first end 11152 toward a second end 11154. A sail 11157 may be disposed on shaft 11156. Sail 11157 may be configured to catch a flow of pressurized gas through second inlet 11148, and cause piston 11150 to rotate about a longitudinal axis of shaft 11156. Sail 11157 may include a woven fabric in some embodiments. The fabric may include nylon, Dacron, aramid fibers, or other suitable fibers.

A flange 11158 may be disposed at second end 11154 and may be coupled to an end of shaft 11156. Referring to FIG. 13D, Flange 11158 may have a generally circular cross-section having one or more cavities 11158a extending radially inward from an outer circumference. In the embodiment shown in FIG. 13D, flange 11158 includes two opposing cavities 11158a that are separated from one another by about 180 degrees. However, it is contemplated that any other suitable number of cavities 11158a may be utilized. Furthermore, it also is contemplated that flange 11158 may have another suitable shape, such as, e.g., rectangular, square, or the like.

Referring back to FIG. 13A, housing 11142 may include one or more stops 11164 configured to abut against surfaces of flange 11158, to maintain piston 11150 in a closed configuration shown in FIG. 13A. When piston 11150 is in the closed configuration, valve 11140 may be closed such that pressurized gas from high pressure line 3002 is prevented from flowing through valve 11140. Piston 11150 may be rotated (e.g., about 90 degrees), so that cavities 11158a align with stops 11164. Once cavities 11158a are aligned with stops 11164, piston 11150 may be movable longitudinally along the longitudinal axis of shaft 11156, creating a flow path though valve 11140 (from first inlet 11144 to outlet 11146).

Before auto-injector 2 is initiated, valve 11140 may be in the closed configuration shown in FIG. 13A, and may remain in the closed configuration after activation of fluid source 1366 and during an injection. That is, valve 11140 may be in the closed configuration while piston 1316 is driven through container 1302 and until piston 1316 reaches second end 1306 (and bottoms out). At the end of the injection, diaphragm 3012 (shown in FIGS. 3A-3C) of valve 3010 may return to its neutral state, enabling flow through conduit 3018. The flow through conduit 3018 may act on sail 11157, rotating piston 11150 about the longitudinal axis of shaft 11156 and aligning cavities 11158a with stops 11164. Once cavities 11158a are aligned with stops 11164, pressurized gas from high pressure line 3002 may urge piston 11150 along the longitudinal axis of shaft 11156, to create a flow path through valve 11140, and allowing pressurized gas flowing through high pressure line 3002 to vent into the surrounding area, and/or, into the atmosphere via outlet 11146.

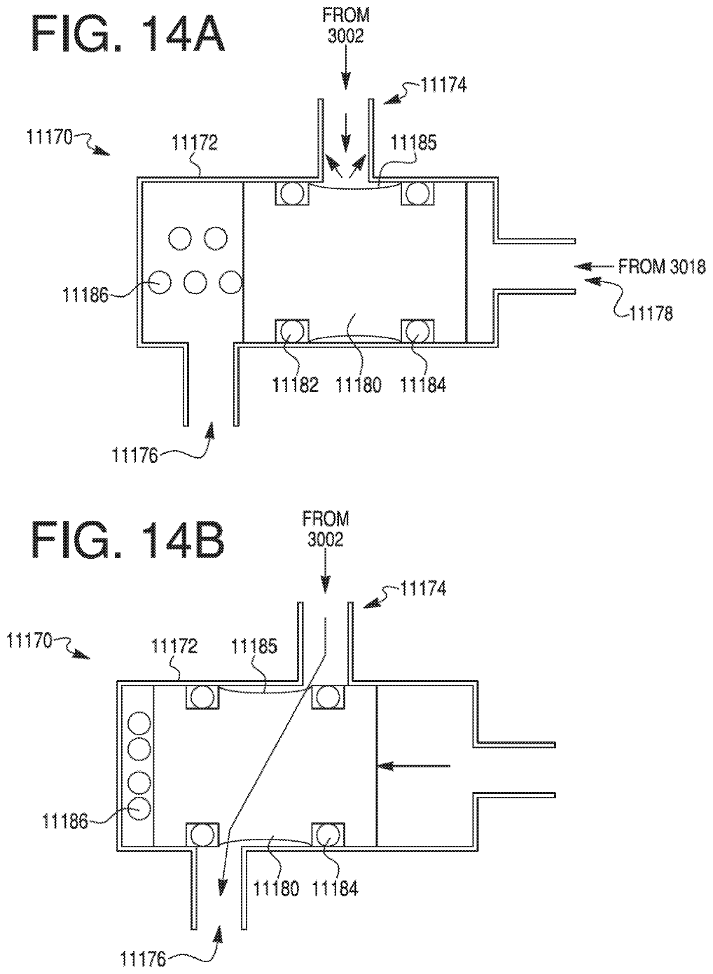

FIGS. 14A and 14B show a valve 11170 that can be used in conjunction with various embodiments disclosed herein, such as, e.g., the embodiment shown in FIGS. 3A-3C. In particular, valve 11170 may be coupled to high pressure line 3002 and conduit 3018 of valve 3010. Referring now to FIG. 14A, valve 11170 is shown in a closed configuration, where flow diverted from high pressure line 3002 is prevented from travelling through valve 11170. Valve 11170 may include a housing 11172 having a first inlet 11174 (configured to receive a flow from high pressure line 3002), an outlet 11176, and a second inlet 11178 that is configured, in some embodiments, to receive a flow from conduit 3018 of valve 3010. Valve 11170 may include a piston 11180 configured to move within and relative to housing 11172. A first seal 11182 and a second seal 11184 may be disposed around the outer circumference of piston 11180. In some embodiments, each of first seal 11182 and second seal 11184 may be disposed in circumferential recesses of piston 11180. However, it also is contemplated that first seal 11182 and second seal 11184 may be disposed around a continuous and uninterrupted outer surface of piston 11180. In some embodiments, an interior portion 11185, disposed between first seal 11182 and second seal 11184, may have a reduced diameter relative to a remaining portion of piston 11180, and also relative to the inner surfaces of housing 11172. Valve 11170 also may include a resilient member, e.g., a spring 11186 coupled to piston 11180. Spring 11186 may be coupled to an end of housing 11172 furthest away from second inlet 11178, and may be biased into an expanded configuration shown in FIG. 14A. In such an embodiment, a force acting on piston 11180 may compress spring 11186 and transition valve 11170 to an open configuration shown in FIG. 14B. In the open configuration shown in FIG. 14B, pressurized gas may flow from high pressure line 3002, through inlet 11174, through a space between housing 11172 and reduced diameter portion 11185 of piston 11180, and out of valve 11170 via outlet 11176. In an alternative embodiment, spring 11186 may be coupled to an end surface of housing 11172 adjacent to second inlet 11178, and may be biased toward a compressed state when valve 11170 is in the closed configuration. In the alternative embodiment, a force acting on piston 11180 may expand spring 11186 to move valve 11170 to the open configuration.

In the closed configuration shown in FIG. 14A, first seal 11182 may substantially or entirely block the flow of pressurized gas from high pressure line 3002 through valve 11170. Before auto-injector 2 is initiated, valve 11170 may be in the closed configuration shown in FIG. 14A, and may remain in the closed configuration after activation of fluid source 1366 and during an injection. That is, valve 11170 may be in the closed configuration while piston 1316 is driven through container 1302 and until piston 1316 reaches second end 1306 (and bottoms out). At the end of the injection, diaphragm 3012 (shown in FIGS. 3A-3C) of valve 3010 may return to its neutral state, enabling flow through conduit 3018. The flow through conduit 3018 may act on piston 11180 and compress spring 11186. Once valve 11170 is moved from the closed configuration shown in FIG. 14A to the open configuration shown in FIG. 14B, pressurized gas flowing through high pressure line 3002 may travel through valve 11170 to vent any remaining propellant stored in fluid source 1366.

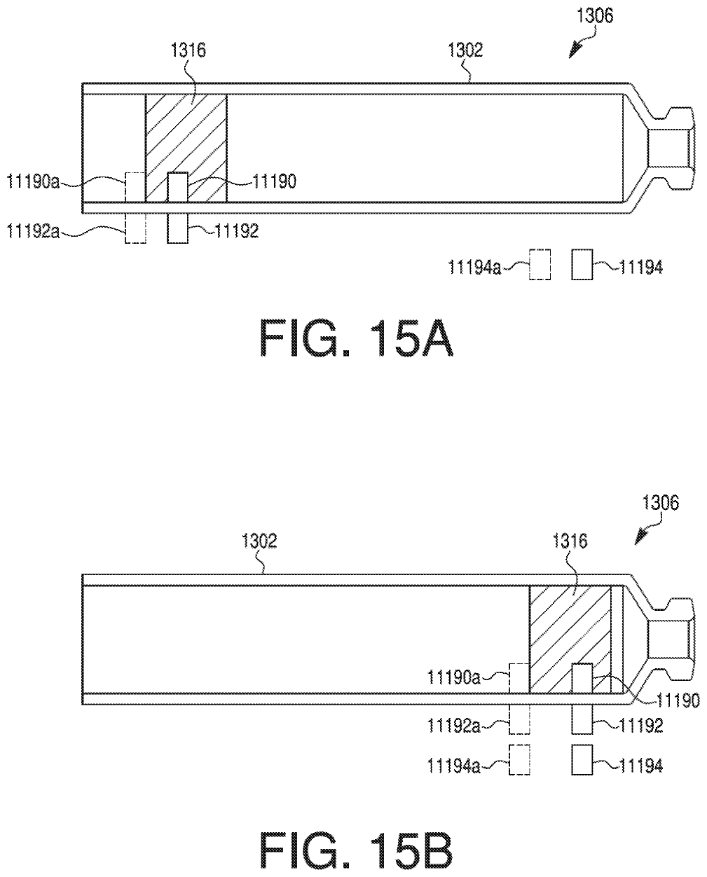

FIGS. 15A and 15B show an embodiment utilizing one or more magnets to initiate venting of fluid source 1366 (not shown in FIGS. 15A and 15B). In one embodiment, piston 1316 may contain or otherwise be coupled to a first magnet 11190. First magnet 11190 may be coupled to an outer side surface of piston 1316, embedded within piston 1316, or coupled to a rear and trailing surface of piston 1316 (this position being shown as 11190a). A second magnet 11192 (or 11192a) may be disposed outside of container 1302, and due to its attraction with first magnet 11190 (or 11190a), may travel along container 1302 when piston 1316 travels through container 1302.

At the end of an injection, piston 1316 may be disposed at second end 1306 of container 1302, and move second magnet 11192 (or 11192a) into contact or into alignment with an actuator 11194 (or 11194a). Actuator 11194 may itself be a magnetically actuated switch configured to initiate venting and/or retraction of needle 306 according to one of the embodiments described herein. In another embodiment, second magnet 11192 (or 11192a) may be coupled to an electrical contact that interacts with a corresponding electrical contact on actuator 11194 (or 11194a), to initiate venting and/or needle retraction as set forth above.

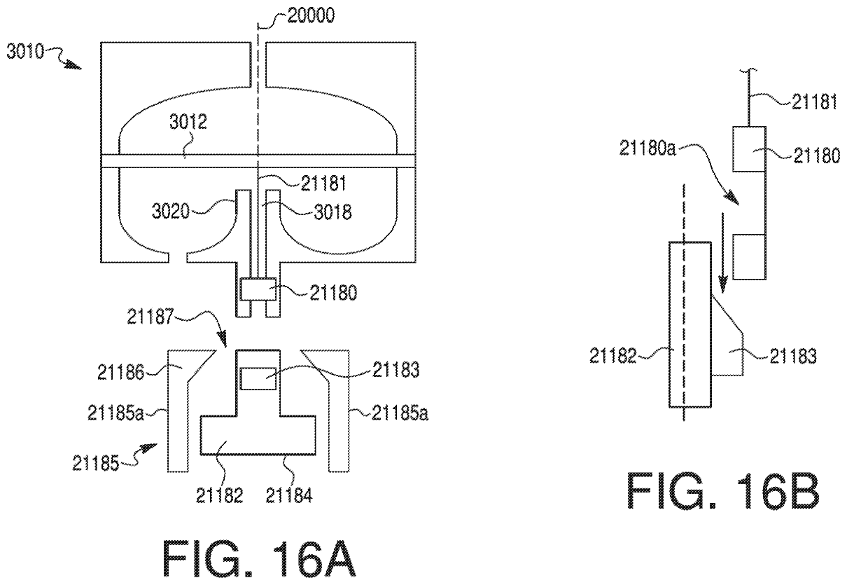

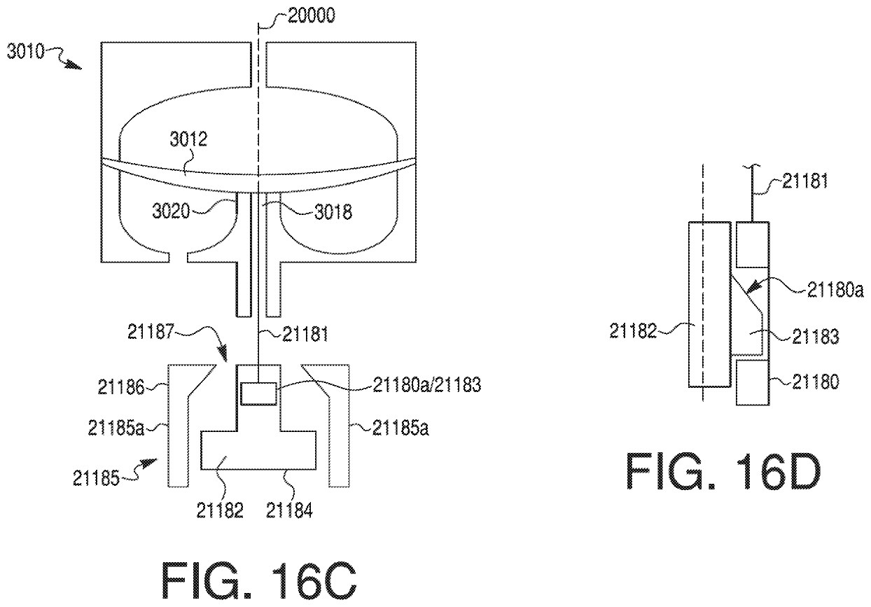

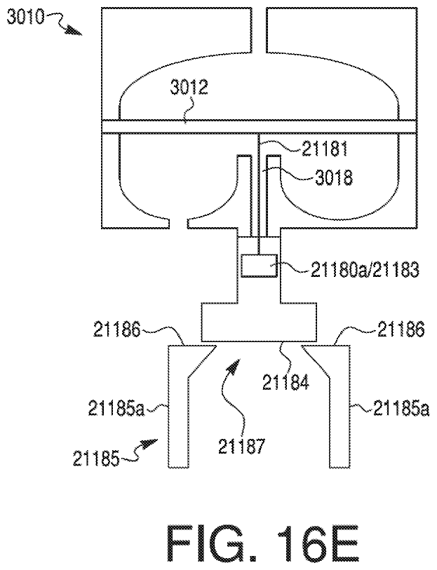

FIGS. 16A-16E illustrate valve 3010 including features for preventing diaphragm 3012 from re-sealing conduit 3018 when diaphragm 3012 returns to its neutral state at the end of an injection. Valve 3010 may include a first locking member 21180 coupled to diaphragm 3012 by a linkage 21181. First locking member 21180 may include a locking cavity 21180a configured to receive a correspondingly shaped locking element. As shown in FIG. 16A, before initiation of fluid source 1366, when valve 3010 is in its original configuration, first locking member 21180 may be disposed within conduit 3018 or may otherwise be coupled to conduit 3018. Valve 3010 also may include an assembly 21185 spaced apart from conduit 3018. Assembly 21185 may include a plurality of spaced apart arms 21185a, defining an opening 21187. In particular, each arm 21185a includes a stop 21186 having a ramped surface and a flat surface. The ramped surfaces of arms 21185a may help permit one-way travel of a second locking member 21182 through assembly 21185, as explained in further detail below. Second locking member 21182 may include a ramped locking member 21183 configured to mate with cavity 21180a of first locking member 21180. Second locking member 21182 also may include a flange 21184.

When valve 3010 is in the first position shown in FIG. 16A, activation of fluid source 1366 may cause diaphragm 3012 to move downward to seal conduit 3018. Because first locking member 21180 is coupled to diaphragm 3012 by linkage 21181, first locking member 21180 also is moved downward toward second locking member 21182 (see FIG. 16B), until ramped locking member 21183 is received by cavity 21180a, and first and second locking members 21180 and 21182 are coupled to one another (FIGS. 16C and 16D). Valve 3010 may stay in the configuration shown in FIGS. 16C and 16D during an injection while piston 1316 moves through container 1302. At the end of the injection, diaphragm 3012 may return to its neutral state shown in FIG. 16E, opening conduit 3018. First and second locking members 21180 and 21183, being coupled to one another at this point and linked to diaphragm 3012 by linkage 21181, may move with diaphragm 3012. In particular, the combined first and second locking members 21180 and 21183 may be moved such that flange 21184 slides against the ramped surfaces of arms 21185a, urging arms 21185a slightly radially outward and temporarily enlarging opening 21187, until first and second locking members 21180 and 21183 are pulled through opening 21187 (see FIG. 16E). In this third configuration, flange 21184 may be prevented from moving downward and/or away from conduit 3018 by stops 21186. This blockage also prevent diaphragm 3012 from moving downward and re-sealing conduit 3018.

Referring to FIGS. 17, 18A-D, and 19-23, a needle mechanism 20 includes a carrier 202. Needle mechanism 20 also may include a fluid conduit 300 that is mounted to carrier 202, and which may be deployed into a user, and retracted by a driver 320. A shuttle 340 (e.g., a shuttle actuator) may be configured to move driver 320 via a deployment gear 360, and a retraction gear 362. Shuttle 340 may be coupled to a resilient member (e.g., a spring 370). A cover 390 may be coupled to carrier 202 to enclose various components of needle mechanism 20. The use of one or more gears in the patient needle mechanism (to assist deployment and retraction of needle 308 along the transverse axis) may help reduce a profile or length of auto-injector 2 relative to auto-injectors where the patient needle and the medicament container are in-line with one another. For example, the length of auto-injectors according to the present disclosure may be reduced along longitudinal axis 40.

Referring to FIG. 18A, fluid conduit 300 may extend from a first end 302 to a second end 304. First end 302 may include a needle 306 that is configured to be injected into a user. Needle 306 may include a sharp and/or beveled tip, and may extend generally along or parallel to axis 44. Second end 304 may include needle 308 (described previously with respect to FIGS. 3A-3C) that is substantially similar to needle 306, but may be positioned within auto-injector 2 to penetrate container 1302 (described previously) to access drugs to be injected into the user. Fluid conduit 300 may include an intermediate section 310 including one portion extending along or parallel to axis 40, and a second portion extending along or parallel to axis 40. The first and second portions of intermediate section 310 may be joined in a coil 312 that facilitates flexion of fluid conduit 300 and movement of needle 306 along axis 44 during deployment into the user, and during retraction out of the user. While a coil 312 is shown, any other suitable shape, e.g., a serpentine, curved, or other shape that enables flexion of fluid conduit 300 is also contemplated. Coil 312, or similar structure, may act as a cantilever when needle 306 is deployed and/or retracted. Once needle 308 penetrates and establishes fluid communication with container 1302 (see, e.g., FIG. 3B), drugs may travel from container 1302, through needle 308, intermediate section 310, and needle 306 (pierced through the user's skin), and into the user. In some examples, fluid conduit 300 may include only metal or a metal alloy. In other examples, fluid conduit 300 may be include any other suitable material, such as, e.g., polymers or the like. Needle 308 and intermediate portion 310 may define a 22 or 23 Gauge, thin-walled needle, while needle 306 may be a 27 Gauge needle. In other words, fluid conduit 300 may have a varying needle gauge across its length, and in particular, needle 306 and needle 308 may have different needle gauges. Other needle sizes ranging from, e.g., 6 Gauge to 34 Gauge, also may be utilized as appropriate. Fluid conduit 300 may reduce the amount of material that contacts the drugs, reduce joints and assembly steps, and require less sterilization than conventional devices.

Carrier 202 may be formed of plastic (e.g., injection-molded plastic), a metal, metal alloy, or the like, and may include a flange 204 with an opening 206, and posts 210 and 212. Carrier 202 also may include an opening 216 through which a needle or other fluid conduit may be deployed. Opening 216 may be a slot that is recessed from an end surface of carrier 202, or, in an alternative embodiment, an entirety of the perimeter of opening 216 may be defined by material of carrier 202. Carrier 202 also includes a driver path 218. Driver path 218 may be a slot in carrier 202 that extends along or parallel to axis 44. Driver path 218 may be configured to receive a protrusion of driver 320, such as, e.g., protrusion 380 discussed in further detail below. Carrier 202 also may include a shuttle path 220, along which shuttle 340 may move, as described in further detail below.

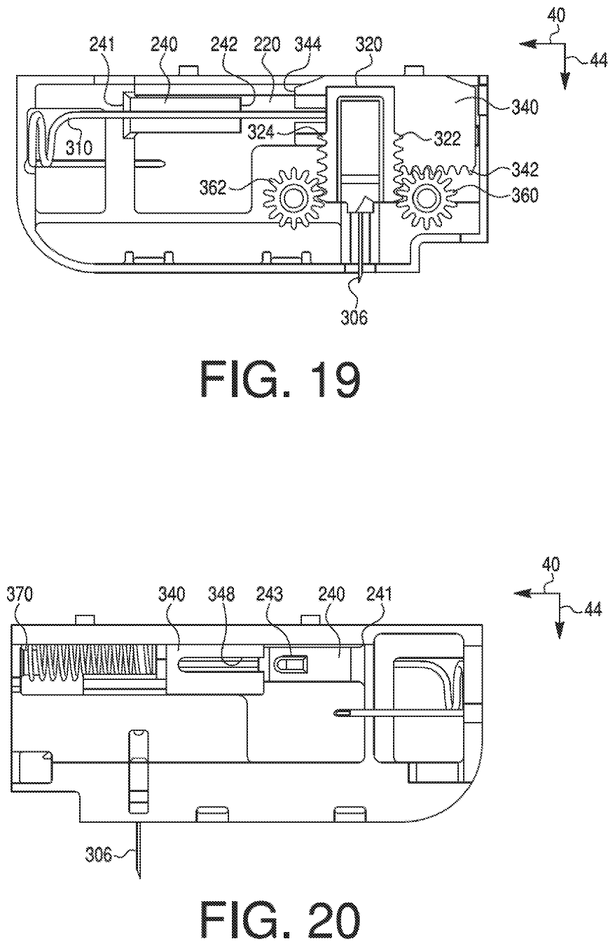

Carrier 202 also may include a stop 240 that is configured to engage shuttle 340. Stop 240 may be a cantilever having a fixed end 241 (FIG. 19) and a free end 242 (FIG. 19). Stop 240 may include an inclined ramp 243 (FIGS. 20 and 23) that, when engaged or pushed by a ramp 1500 (described with reference to FIG. 23), causes stop 240 to deflect about fixed end 241. In a first position, free end 242 may block or otherwise impede movement of shuttle 340, and in a second configuration, may permit movement of shuttle 340. The relationship between stop 240 and shuttle 340 will be discussed in further detail later in the application.

Driver 320 includes two racks 322 and 324 (shown in FIGS. 18A-18C and 19) parallel to one another and disposed on opposing sides of driver 320. Racks 322 and 324 may include teeth and may be configured to engage with and drive rotation of deployment gear 360 and retraction gear 362, respectively. Driver 320 may include a lumen 326 (or a track, recess, or other suitable structure) (FIG. 18A) that is configured to receive needle 306 of fluid conduit 300. Driver 320 also may include protrusion 380 (FIGS. 17 and 18B-18D) that is configured to slide within driver path 218 of carrier 202. Protrusion 380 may include a hook-like configuration that can “catch” on impediment 382, as described in further detail below.

With continuing reference to FIG. 18A-18D, shuttle 340 may include a rack 342 configured to engage with gears 360 and 362. Shuttle 340 also may include an end surface 344, and a recess 346 that extends along a length of shuttle 340 in the same direction as rack 342. A slot 348 (FIG. 20) may extend along the length of recess 346. Slot 348 may extend through the middle of recess 346 and may extend along an entirety or substantial entirety of recess 346.

Shuttle 340 may move along track 220 from a first, starting position (FIGS. 18B and 19), to a second, intermediate position (FIGS. 18D, 20, and 21), and from the second position to a third, final position (shown between the second and third positions in FIG. 22). As shuttle 340 moves along track 220, rack 342 may first engage deployment gear 360, and then retraction gear 362. Rack 342 engages at most one of deployment gear 360 and retraction gear 362 at any given time. In some examples, such as when rack 342 is disposed longitudinally between deployment gear 360 and retraction gear 362, rack 342 is not engaged with either of deployment gear 360 and retraction gear 362. Shuttle 340 may be configured to move only along one axis (e.g., axis 40) and only in one direction along the one axis. The force required to move shuttle 340 along track 220 may be provided by expansion of spring 370. Spring 370 may be compressed from a resting state, and the expansion of spring 370 may move shuttle 340 along track 220 through the series of positions/configurations set forth above. At various positions of shuttle 340, different features of auto-injector 2 may directly or indirectly block movement of shuttle 340. Alternatively, spring 370 may be expanded from a resting state, and the compression of spring 370 may move shuttle 340 along track 220 through the series of positions/configurations set forth above. In such an embodiment, shuttle 340 may be coupled to a different and opposite side of shuttle 340, and may be coupled to an opposing end of auto-injector 2.

The first position of shuttle 340, shown in FIGS. 18B and 19, may correspond to an unused, undeployed, and/or new state of auto-injector 2. In this first position, driver 320 may be in an undeployed state. Shuttle 340 is maintained in the first position by the positioning of an impediment 382 in the path of protrusion 380 (FIGS. 17 and 18B). Impediment 382, which may be a protrusion or other blocking component or device coupled to container 1302, may prevent movement of driver 320 by engaging and/or retaining protrusion 380. Therefore, because driver 320, deployment gear 360, and rack 342 are coupled to one another, the blockage of driver 320 also prevents movement of shuttle 340. Shuttle 340 may move from the first position to the second position by moving impediment 382 relative to carrier 202 (or vice versa). In one example, impediment 382 is moved when container 1302 is driven by pressurized gas from fluid source 1366 into fluid communication with needle 308 (FIG. 18C), while carrier 202 remains stationary.

When the path of driver 320 is free from impediment 382 (FIG. 18C), spring 370 may expand and move shuttle 340 along track 220. This linear movement of shuttle 340 may rotate deployment gear 360 counter-clockwise (or clockwise in other examples) via rack 342, and the rotation of deployment gear 360 may move driver 320 downward along axis 44, via rack 322 of driver 320. This downward movement of driver 320 may cause needle 306 to pierce through the skin of a user. In some examples, driver 320 may be configured to move, relative to carrier 202, along only axis 44.

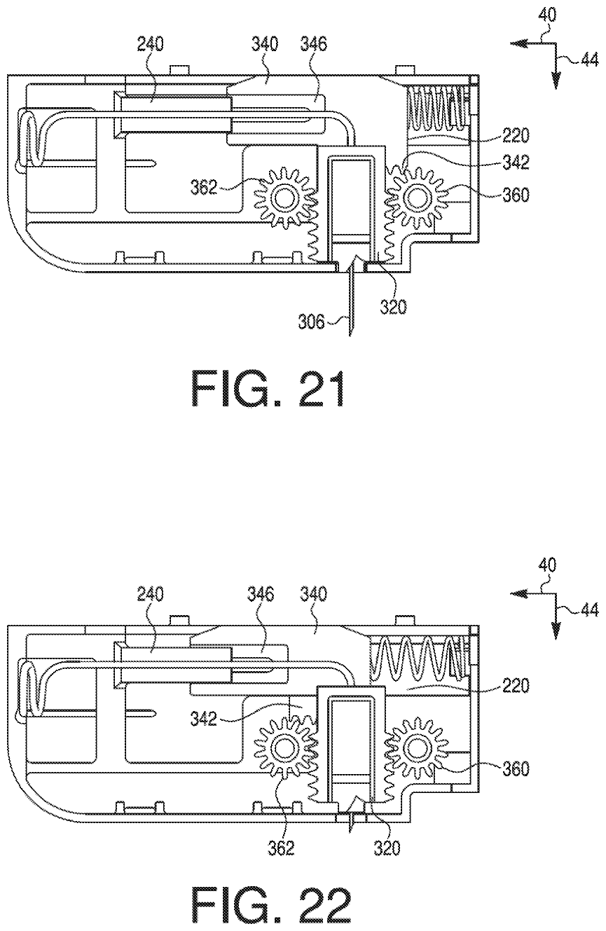

Shuttle 340 may be moved by the expansion of spring 370 until its end surface 344 abuts free end 242 of stop 240 such that shuttle 340 is maintained in the second position shown in FIGS. 20 and 21. At this point, free end 242 may prevent further expansion of spring 370 and further movement of shuttle 340 along track 220. In this second position, needle 306 may be deployed within a user, and fluid from container 1302 may be injected into the user via fluid conduit 300. Additionally, while shuttle 340 is in the second position, rack 342 may be engaged with deployment gear 360 to maintain needle 306 in the deployed configuration. Shuttle 340 may move from the second position to the third position by the flexion of stop 240 about its fixed end 241. Further details of this flexion are set forth below with respect to FIG. 23. The flexion of stop 240 may allow spring 370 to continue expanding, urging shuttle 340 further along track 220. In some examples, stop 240 may be received by and/or within recess 346 of shuttle 340, and ramp 243 may slide within slot 348, as shuttle 340 moves from the second position to the third position.

The movement of shuttle 340 from the second position to the third position may correspond to the retraction of needle 306 from the user into housing 3. In particular, rack 342 may engage with and rotate retraction gear 362 in the same direction (e.g., counter-clockwise or clockwise) as deployment gear 360 was rotated. The rotation of retraction gear 362 may urge driver 320 back to a retracted position via rack 324. Shuttle 340 may reach the third position, where driver 320 is fully-retracted, when its end surface 344 engages a wall of carrier 202, when free end 242 of stop 240 reaches an end of recess 346, and/or when spring 370 reaches a resting state.

In some embodiments, once driver 320 moves from the deployed state back to the retracted state, it may be prevented from moving out of the retracted state. As a result, needle 306 will be prevented from re-deployment into the user. In this configuration, auto-injector 2 may be a single-use device (e.g., discarded after completing one injection). In other embodiments, auto-injector 2 may be reset and reused. Furthermore, deployment gear 360 and retraction gear 362 may be the only rotating gears disposed within auto-injector 2, in some examples.

After drugs/medicament have been delivered to the user via needle 306, needle 306 may be automatically withdrawn from the user. For example, a spring can expand (or contract) and cause container 1302 to move in an opposite direction along axis 40 (as compared to during fluid delivery and insertion of needle 306). The movement of container 1302 in the opposing direction may cause ramp 1500 in FIG. 23 (which is attached to wall 1391) to push against ramp 243 of stop 240. This may cause stop 240 to deflect about its fixed end 241 in the direction of arrow 240a, and allow shuttle 340 to move from its second position to its third position to retract needle 306 as set forth above. in this way, withdrawal and insertion of the needle into a patient can both be accomplished with a single spring within the device.

FIGS. 23A-23C illustrate another embodiment for the injection and retraction of needle 306 (or other patient needle) as described herein. FIGS. 23A and 23B, in particular, show the same steps and structure for the insertion of needle 306 into the patient as set forth above in FIGS. 18B-18D and 19-21. As alluded to above with respect to FIGS. 12A-12C and FIG. 23, retraction of needle 306 may be assisted by rod 8002 and the force of gas/fluid from vent 3018. That is, after injection is completed, and the pressure between a high pressure cavity and a low pressure cavity equilibrates (for example, as described above with respect to valve 3010), gas/fluid from fluid source 1366 may vent through vent 3018, to translate rod 8002. Rod 8002 may directly contact and move stop 240 out of a path of shuttle 340 (as shown in FIG. 23C), or, as described above with respect to FIG. 23, may act against a ramp 1500 that directly contacts stop 240.

FIG. 23D shows an alternative embodiment for needle insertion and retraction using one rotating gear 360a instead of gears 360 and 362 set forth above. Needle insertion is initiated in a substantially similar manner as set forth above with respect to FIGS. 18B-18D and 19-21, where expansion of spring 370 moves shuttle 340 linearly. The linear movement of shuttle 340 causes gear 360a to rotate as a result of being driven by rack gear 342. The rotation of gear 360a in a first direction causes driver 320 and needle 306 to deploy in the downward direction (toward the skin surface). In this embodiment, the retraction of needle 306 is carried out by causing shuttle 340 to revert to its initial position. In particular, pressurized gas/fluid from vent 3018 may push rod 8002 into contact with shuttle 340. The action of rod 8002 against shuttle 340 may compress spring 370 and cause shuttle 340 to move back to its initial position. Shuttle 340 may move back to its initial position along the same path (in reverse) that shuttle 340 travelled to deploy needle 306. The reversed path of shuttle 340 may cause gear 360a to rotate in a second direction opposite to the first direction, causing driver 320 and needle 306 to retract out of the patient and into auto-injector 2. A lockout feature 8002f may be coupled to rod 8002 and may be configured to prevent rod 8002 from retracting. In this embodiment, the retraction of rod 8002 back into vent 3018 may cause an inadvertent redeployment of needle 306. To help prevent such redeployment, lockout feature 8002f may be activated at some point during the retraction of needle 306. In one embodiment, lockout feature 8002f may be an elastic or otherwise flexible member extending from a circumferential side surface of rod 8002, and that is biased to an expanded configuration. Before retraction is initiated, lockout feature 8002f may be constrained by the inner surfaces of vent 3018 through which rod 8002 is disposed. Once rod 8002 is pushed past a certain point, for example, when lockout feature 8002f exits the vent 3018, lockout feature 8002f may be unconstrained and urge itself radially outward toward its resting expanded configuration. Once in the resting and expanded configuration, lockout feature 8002f may be unable to re-enter the vent 3018, and a periphery of the channel, such as, e.g., periphery 8002g may act as a stop acting against lockout feature 8002f. In yet another embodiment, lockout feature 8002f may be a magnet configured to be secured against a magnet at the periphery 8002g of vent 3018, or against a magnet disposed within or along the vent 3018. For example, the inner surface of a portion of the vent 3018 may include a magnet.

FIGS. 23E-23G show another alternative embodiment for needle insertion and retraction using rotating gear 360a and a different arrangement of the elements of the system illustrated in FIG. 23D. As shown in these Figures and as discussed herein, shuttle 340 may be above or below spur gear 360 relative to the skin. As shown in FIG. 23E, shuttle 340 may be positioned below gear 360a (closer to the tissue-contacting surface/injection site), and push rod 8002 and spring 370 may be substantially parallel to at least a portion of shuttle 340. Push rod 8002 may be in contact with a portion of spring 370 as discussed above. Additionally, shuttle 340 may be coupled to and/or may be integrally combined with push rod 8002. Needle insertion may be initiated from initial pressure from gas from a gas canister, as discussed herein. The linear movement of shuttle 340 in a first linear direction causes gear 360a to rotate as a result of being driven by rack gear 342. As shown in FIG. 23F, the rotation of gear 360a in a first rotational direction causes driver 320 and needle 306 to deploy in the downward direction (toward the skin surface). The linear movement of shuttle 340, and thus also push rod 8002, also causes spring 370 to compress (or expand in alternative embodiments). Then, as shown in FIG. 23G, when the force of gas acting on push rod 8002 is less than the force of spring 370, spring 370 may expand (or compress in alternative embodiments) and bias push rod 8002, and thus shuttle 340, in a second linear direction opposite to the first linear direction. The linear movement of shuttle 340 in the second linear direction causes gear 360a to rotate in a second rotational direction opposite to the first rotational direction. The rotation of gear 360a in the second rotational direction causes driver 320 and needle 306 to retract in the upward direction (away from the skin surface).

FIGS. 23H and 23I are different views of a patient needle mechanism that may perform the steps shown and discussed above with respect to FIGS. 23E-23G. As shown, the needle mechanism includes push rod 8002, a modified shuttle 340, driver 320, spur gear 360, spring 370, and, although not shown, a needle. Push rod 8002 may include a seal gap 8008, for example, to receive a seal, as discussed herein. As shown in FIG. 23I, shuttle 340 may include two parallel portions 340b and 340c. Additionally, shuttle 340 may include one or more prongs 341, for example, two prongs 341. Prongs 341 may extend vertically from shuttle 340, for example, perpendicularly to portions 340b and 340c. Prongs 341 may connect to an indicator (not shown, described in further detail below) to allow for translation of the indicator in order to indicate, for example, to a user, the progress of the needle mechanism, as discussed herein.

Portions 340b and 340c may be connected via a portion 340d, which may be perpendicular to portions 340b and 340c (and also perpendicular to prongs 341). As shown, portion 340d is in the same plane as portions 340b and 340c, and perpendicular to prongs 341. Portion 340b may include rack 342 (not shown in FIG. 23H or 23I, which may contact and/or engage with spur gear 360a, and thus control the movement of spur gear 360, driver 320, and the patient needle (not shown), as discussed above. Portion 340c may extend parallel to a section of portion 340b, and may interact with spring 370. For example, portion 340c be surrounded by a portion of spring 370. In another example, although not shown, portion 340c may be fixedly coupled to or attached to a portion of spring 370. In either aspect, spring 370 may surround and/or otherwise be coupled to a spring cover 8010, which is stationary relative to carrier 202. Spring cover 8010 may extend from a carrier, for example, carrier 202 and/or may be formed by a portion of a cover of carrier 202 or otherwise formed internal to auto-injector 2. Accordingly, spring 370 may bias portion 340c, and thus bias the entirety of shuttle 340 and push rod 8002. Carrier 202 may include a button translator in this embodiment, and also may support at least a portion of a sterile connector shown in FIG. 9I

In the aspects discussed with respect to FIGS. 23H and 23I, the biasing force of spring 370 is in line with push rod 8002, which may help reduce creep and/or bending of the shuttle and/or associated components. Portion 340b of shuttle 340 is offset and parallel to portion 340c, which may allow for the needle to be in a central position, for example, under an actuation button. Furthermore, although not shown in FIG. 23I, the shuttle teeth are underneath the spur gear 360a, for example, relative to the skin. Additionally, spur gear 360a may be to the right of needle driver 320 (and thus needle driver 320 is to the left of spur gear 360a), as shown in FIG. 23H. One or more of these aspects may help to accommodate the needle drive assembly within one or size, space, or arrangement constraints within auto-injector 2. For example, as shuttle 340 is activated (e.g., moves to the right in FIGS. 23H and 23I based on the actuation force on push rod 8002), shuttle 340 causes spur gear 360a to rotate counterclockwise to insert the needle, and as shuttle 340 is retracted (e.g., moves to the left in FIGS. 23H and 23I based on the biasing force of spring 370), shuttle 340 causes spur gear 360a to rotate clockwise to retract the needle. Of course, any one or more of the directions or orientations could be adjusted based on a particular application.

Push rod 8002 and shuttle 340, including portions 340b, 340c, and 340, may be formed of one, two, three, or more pieces or components. In one aspect, push rod 8002 may be formed of a single piece, and shuttle 340 may be formed of a single piece. In this aspect, push rod 8002 may be contained in a valve sub-assembly, and shuttle 340 may be contained in a patient needle mechanism sub-assembly. These sub-assemblies may help to increase in the ease of assembly and/or manufacture.

Although not shown, one or more additional features as discussed above, for example, lockout feature 8002f, may be incorporated in the embodiment shown in FIGS. 23E-23I. The arrangement of elements shown in FIGS. 23E-23I may help to provide a smaller and/or more discrete needle deployment mechanism, which may be easier and/or more economical to fit within an enclosure, for example, within auto-injector 2.

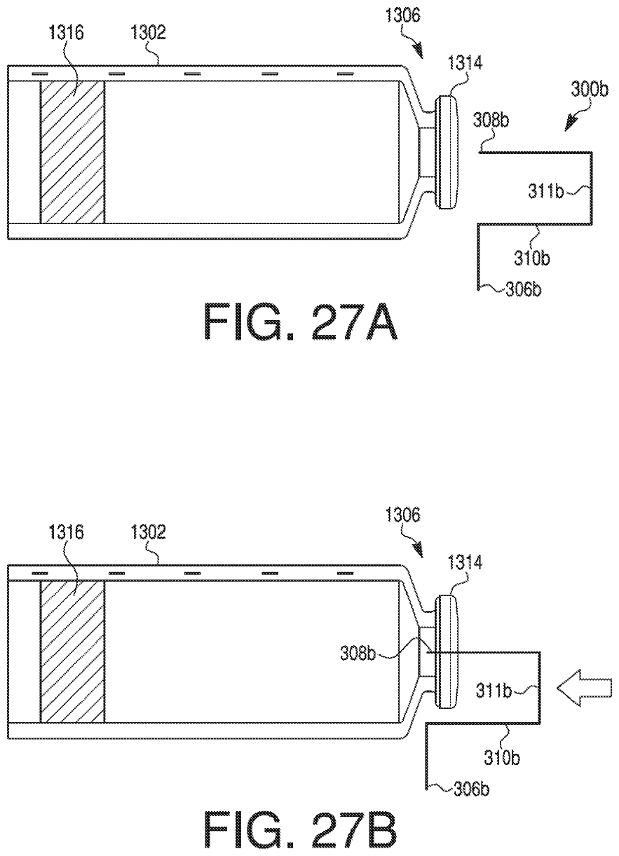

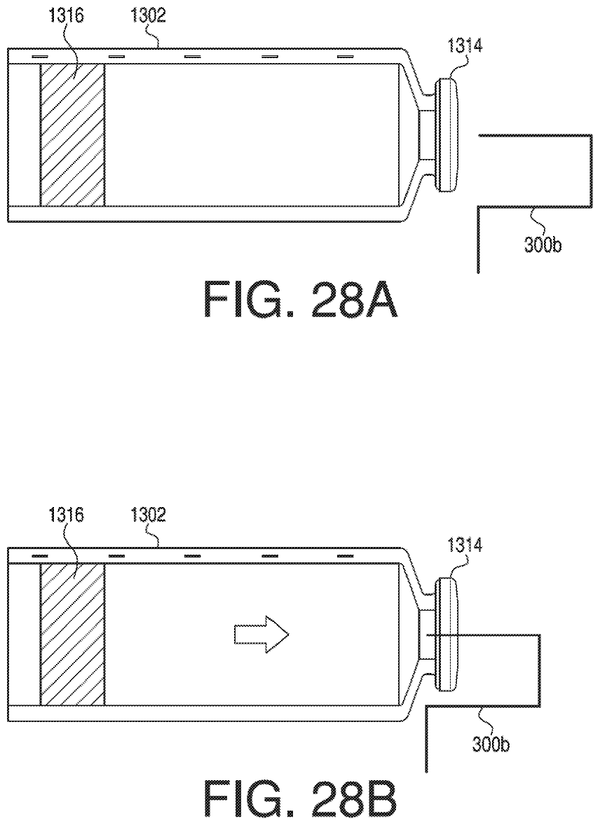

FIGS. 23J-L show yet another alternative embodiment for needle insertion and retraction. In particular, the embodiment shown in these figures may utilize a portion of high-pressure flow from fluid source 1366 (via high pressure line 3002) to drive needle insertion. A carrier 202a may include spur gear 360a and driver 320 as described above. Rotation of gear 360a in a first direction causes driver 320 to deploy, and rotation of gear 360a in a second direction (opposite of the first direction) causes driver 320 to retract. Gear 360a may be rotated by a shuttle 340a. Shuttle 340a may be similar to shuttle 340 described above, except that shuttle 340a may include a rod 340f, which may be disposed in a high pressure channel 340c configured to receive high pressure gas/fluid from high pressure line 3002. While rod 340f is shown in FIGS. 23J-K as integral with shuttle 340a, it is contemplated that rod 340f and shuttle 340a may not be integral with one another, and instead may be separate components that are brought into and out of contact with one another. When rod 340f and shuttle 340a are separate components, their orientation relative to one another may be constrained by other portions of auto-injector 2, such as, for example, one or more channels formed in carrier 202a. Rod 340f may include a seal 340d at or adjacent to a first end 340e (the end disposed furthest from shuttle 340a. Seal 340d may help ensure that pressurized fluid travelling through high pressure channel 340c displaces rod 340f (instead of merely travelling around rod 340f). Rod 340f may extend from a remainder of shuttle 340 and may be any suitable length, including less than, equal to, or longer than the length of the remainder of shuttle 340a. For example, rod 340f may be about 0.5×, about 0.6×, about 0.7×, about 0.8×, about 0.9×, about 1×, about 2×, about 3×, or about 4× the length of the remaining portion of shuttle 340a. Of course, any other suitable values are also contemplated. Carrier 202a also may include an elastic member or spring 370a that is expanded in a resting configuration shown in FIG. 23J. Spring 370a may be coupled to an end of shuttle 340a opposite of rod 340f, and the spring force of spring 370a may maintain gear 360a in an initial configuration (and thus needle driver 320 and needle 306 in a retracted/undeployed configuration). Upon release of pressurized gas/fluid from fluid source 1366 (e.g., described with reference to FIGS. 3A-3C), the flow of gas/fluid through high pressure line 3002 and channel 340c may push rod 340f and shuttle 340a against spring 370a, compressing spring 370a. As shuttle 340a moves linearly to compress spring 370a, rack gear 342 disposed on shuttle 340a causes gear 360a to rotate and deploy driver 320 into a deployed/injection configuration (FIG. 23K). FIG. 23L shows completion of the injection and retraction of driver 320 and needle 306. In FIG. 23L, piston 1316 has traveled through the entirety of container 1302 (piston 1316 has “bottomed-out”). As set forth above, at this stage, the pressures in high pressure cavity 3022 and low pressure cavity 3024 equilibrate (described above with respect to valve 3010), resulting in venting of gas/fluid through vent 3018. After equilibration, the pressure in high pressure cavity 3022, high pressure line 3002, and channel 340c may be less than the spring force of spring 370a, enabling spring 370a to expand towards its resting and expanded configuration. The expansion of spring 370a then moves shuttle 340a back to its initial position. During this movement of shuttle 340a back to its initial position, rack 342 causes gear 360a to rotate in the second direction, thereby retracting driver 320 and needle 306 into auto-injector 2. Container 1302 is shown as stationary in FIGS. 23J-K, for example, as would be the case in an embodiment where needle 308 is moved through a stationary container 1302 (as described below with reference to FIGS. 27A and 27B) to establish fluid communication between fluid conduit 300 and container 1302. However, it is contemplated that container 1302 may translate in the direction from first end 1302 toward second end 1304, onto a stationary needle 308, in order to establish fluid communication between container 1302 and fluid conduit 300 (as described with below with reference to FIGS. 28A and 28B). FIG. 23M shows a drive system 3000a for providing the drive force to deliver fluid from container 1302 to a patient. Drive system 3000a may be substantially similar to drive system 3000 set forth above with respect to FIGS. 3A-3C, and may further be configured such that a patient needle mechanism (including, e.g., rod 340f) must be actuated by pressurized gas from fluid source 1366, before any pressurized gas from fluid source 1366 reaches high pressure line 3002 (which is used to establish fluid communication between container 1302 and fluid conduit 300). Thus, pressurized gas may exit fluid source 1366 via conduit 3002a, and then enter high pressure channel 340c to push against rod 340f. As set forth above, the pressurized gas acting on rod 340f ultimately causes deployment of needle 306 into the user. Only after rod 340f has travelled a sufficient distance (such as, e.g., a distance sufficient to partially or fully drive needle 306 into the user) through high pressure channel 340c, will pressurized gas flow from conduit 3002a to high pressure line 3002. After travelling the sufficient distance, the pressurized gas may flow through drive system 3000a in a substantially similar manner as set forth above with respect to drive system 3000 (FIGS. 3A-C). This arrangement, and in particular, requiring the patient needle mechanism to deploy before pressurized gas is allowed to travel through drive system 3000a, may help prevent inadvertent and premature movement of container 1302 and needle 308 (FIG. 18A) toward one another. In other words, this arrangement may help prevent the premature establishment of fluid communication between container 1302 and fluid conduit 300, which may result in operational failure of auto-injector 2 (e.g., by leaking of medicament within auto-injector 2). Drive system 3000a also may include a venting system 2300a (which may be similar to any of the venting systems described herein, including, but not limited to venting system 9100, or the like). For example, venting system 2300a may include a dump valve.

It is further contemplated that fluid conduit 300 may be the only fluid conduit of auto-injector 2 configured to be in fluid communication with container 1302. Thus, drugs/medicament from container 1302 may be deployed only through fluid conduit 300 and into the user during normal operation of auto-injector 2. Additionally, needle 306 may be the only needle of auto-injector 2 configured to be deployed into a patient. In this way, a single (only one) piece of metal or plastic can be used to carry the fluid from container 1302 to a patient.

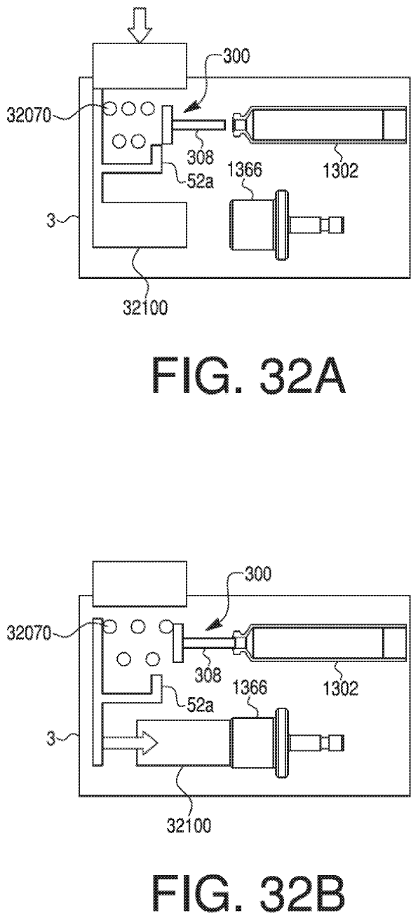

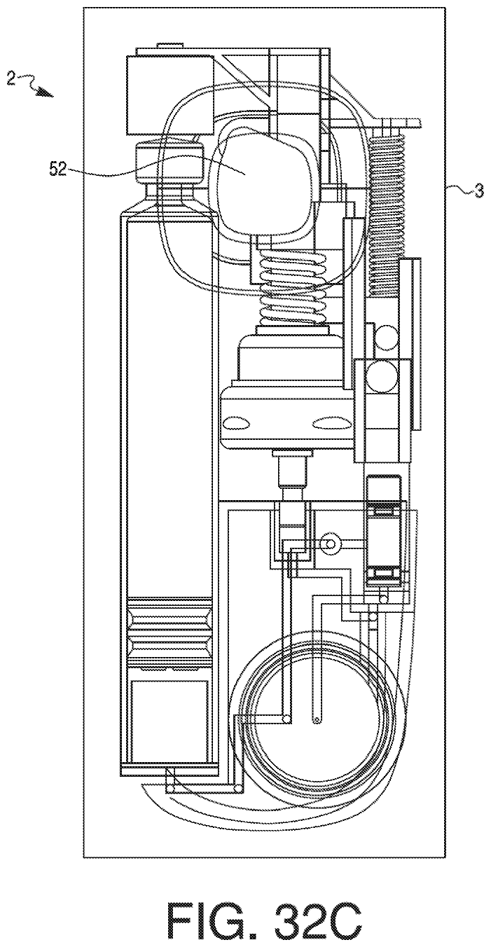

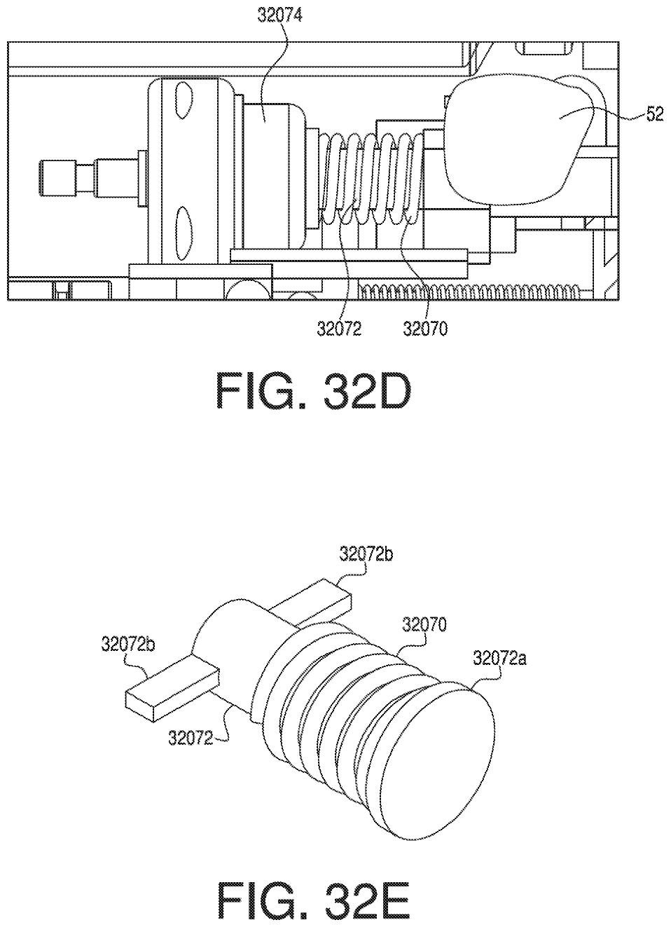

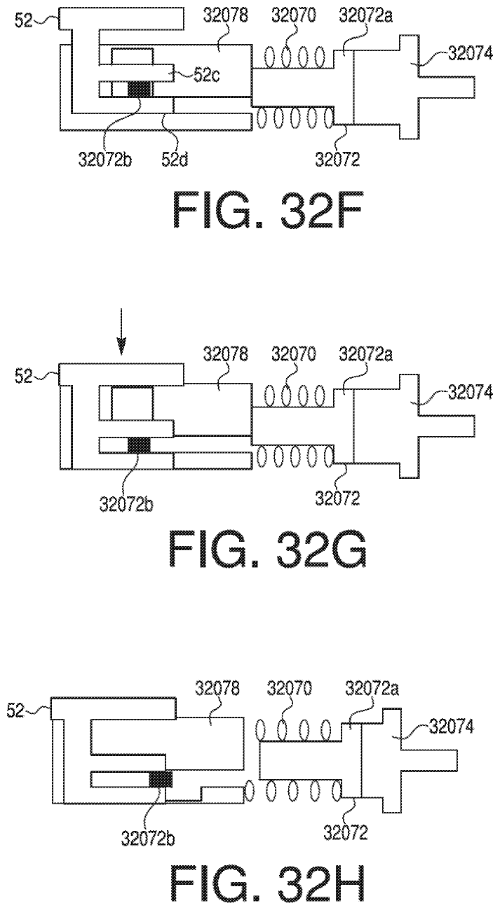

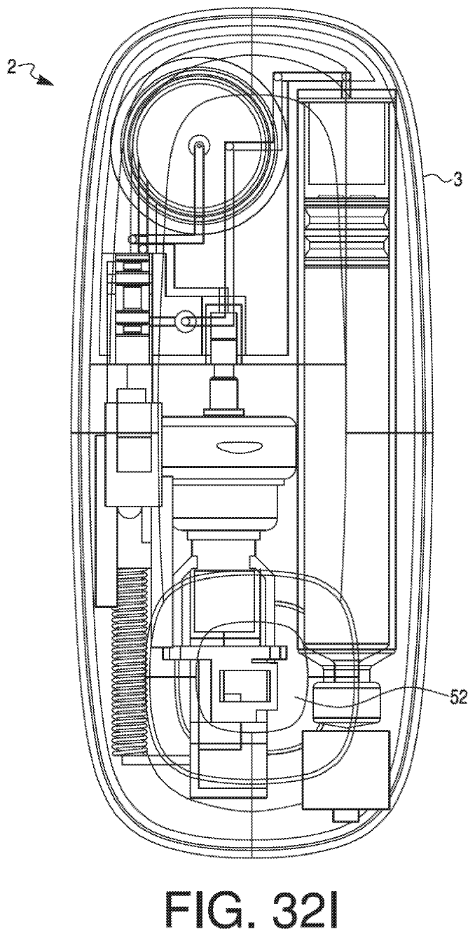

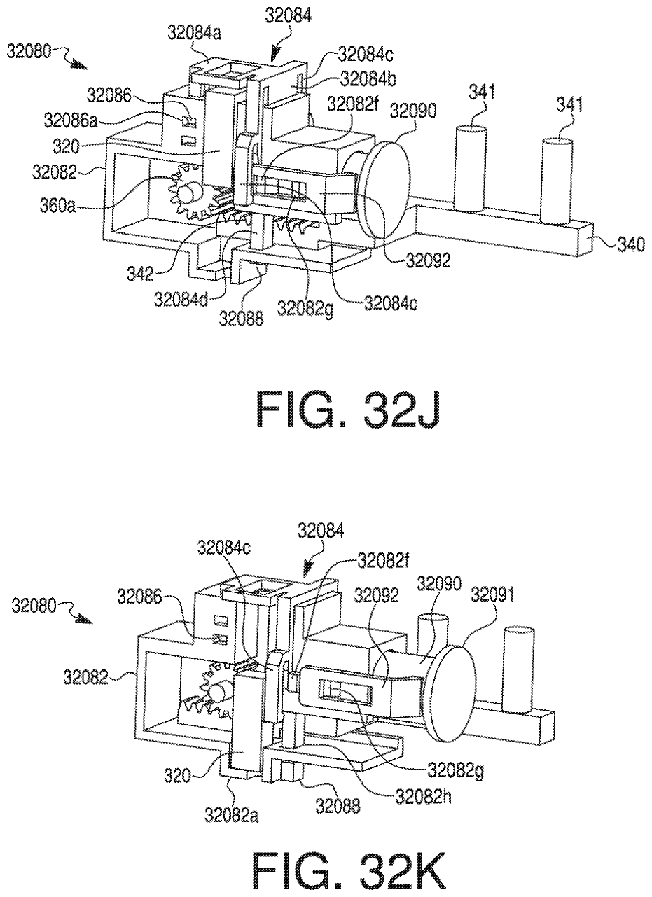

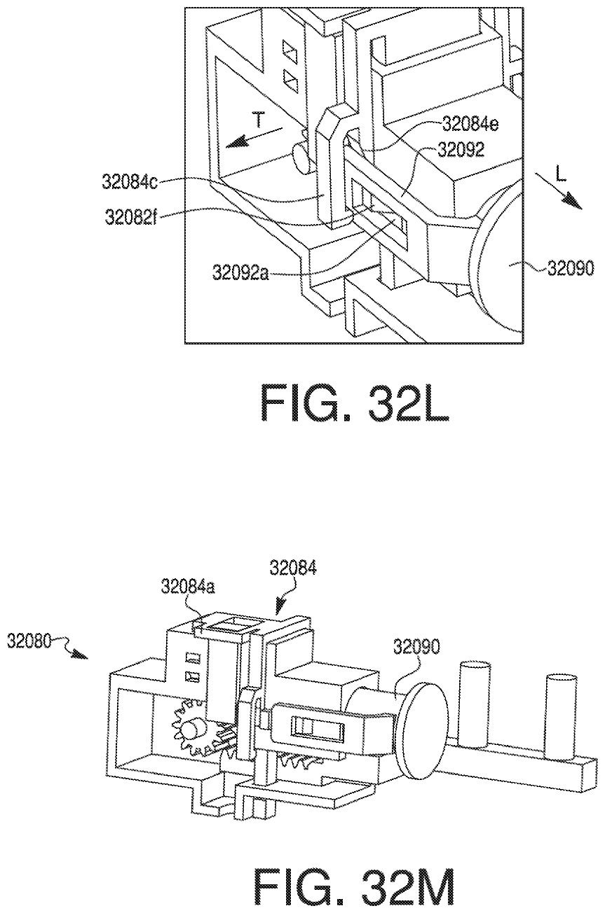

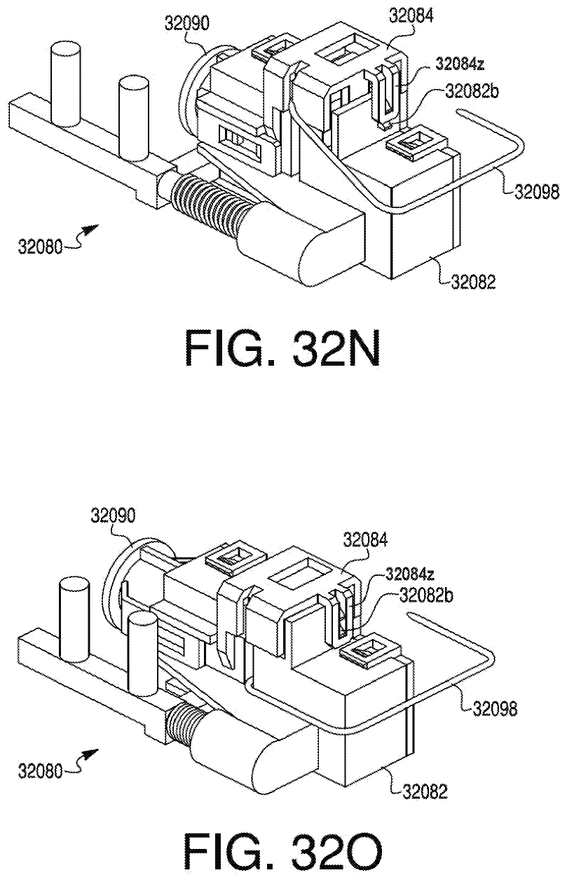

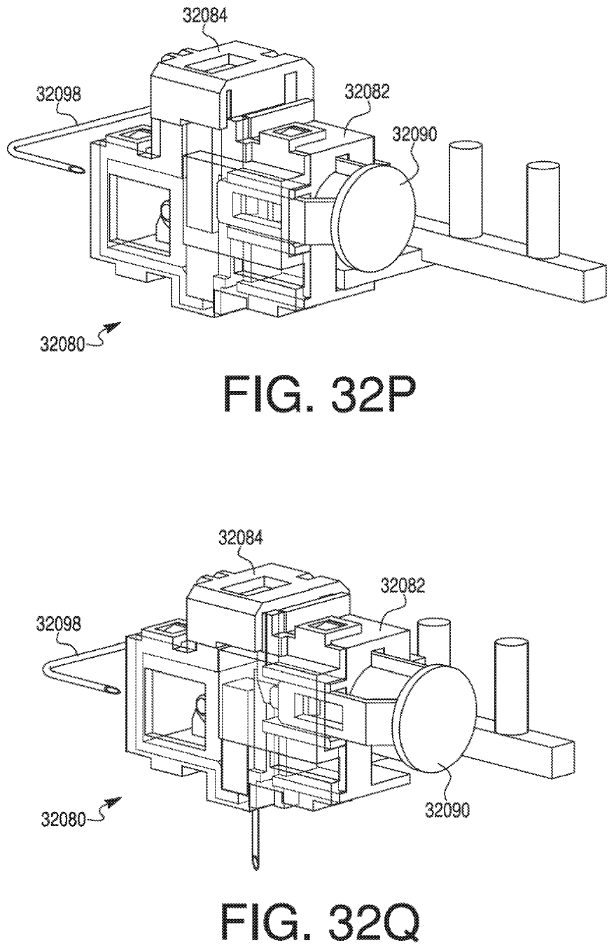

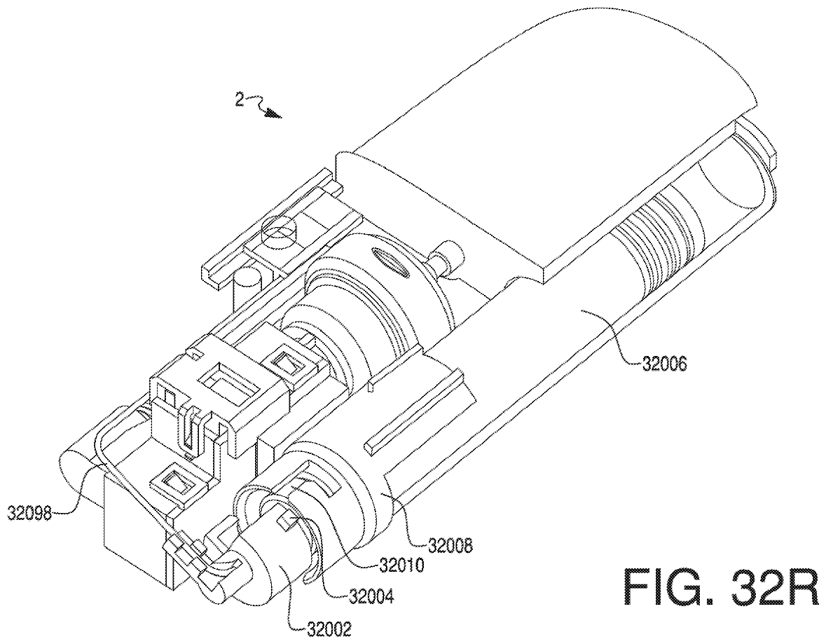

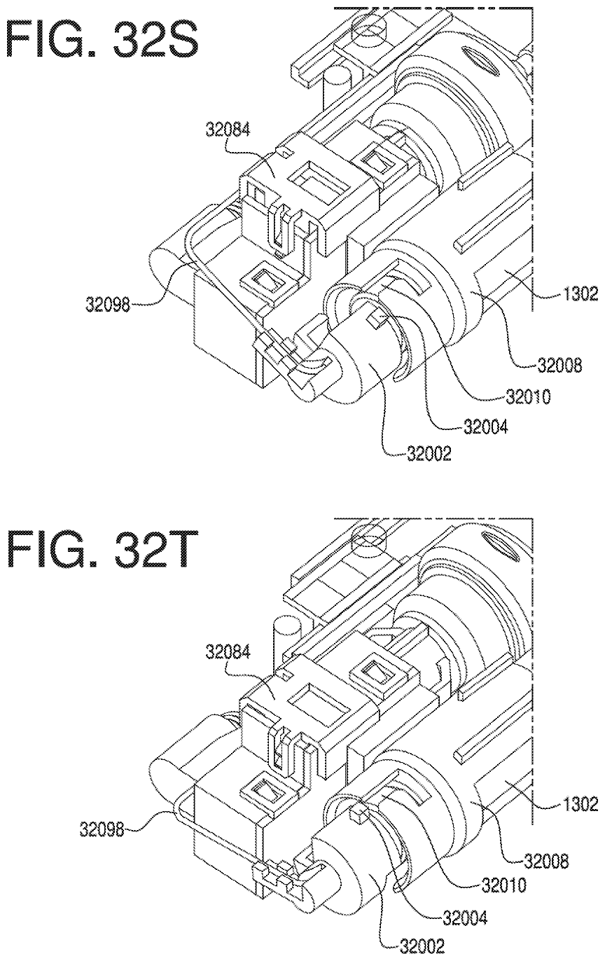

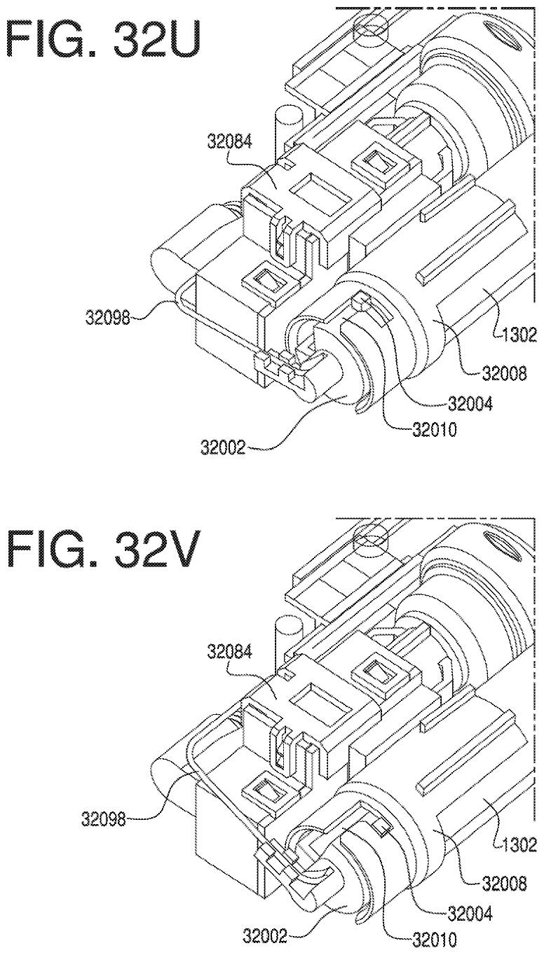

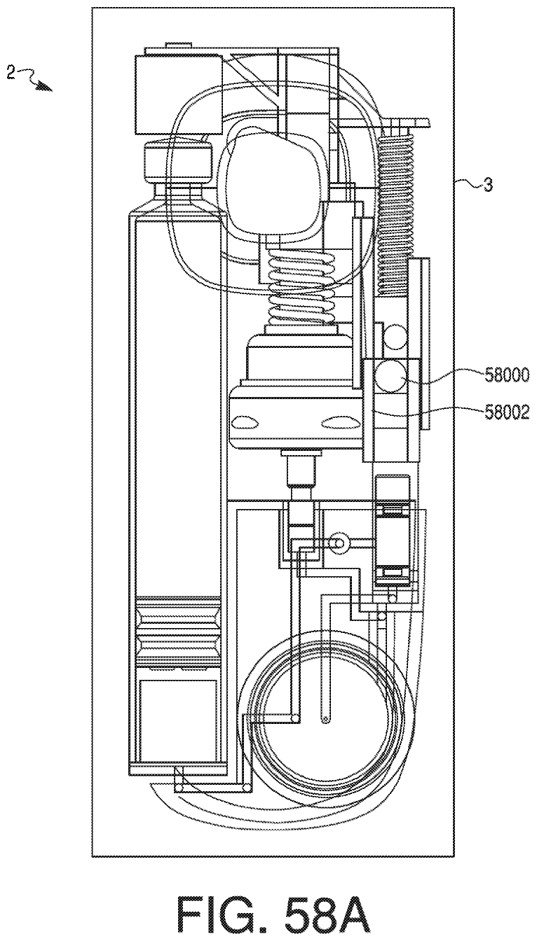

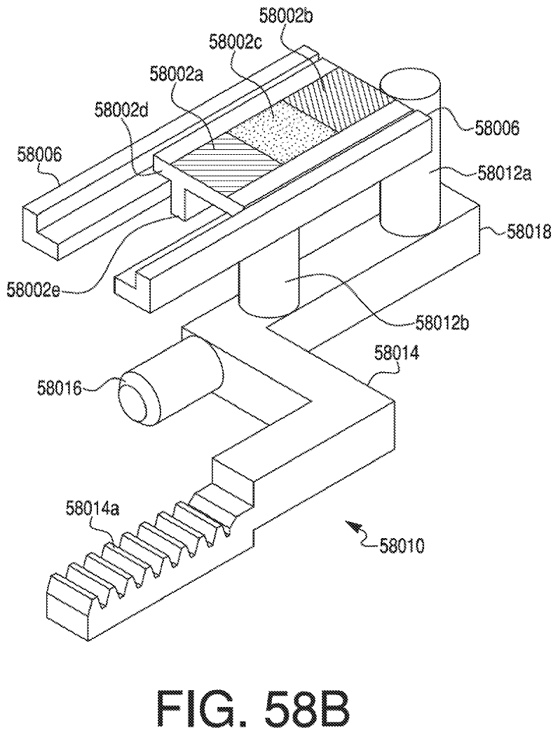

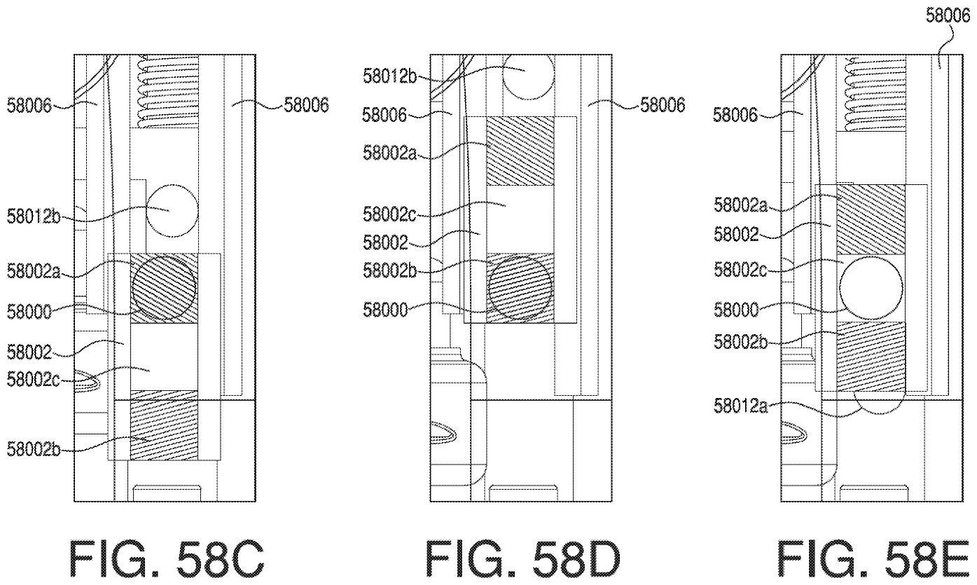

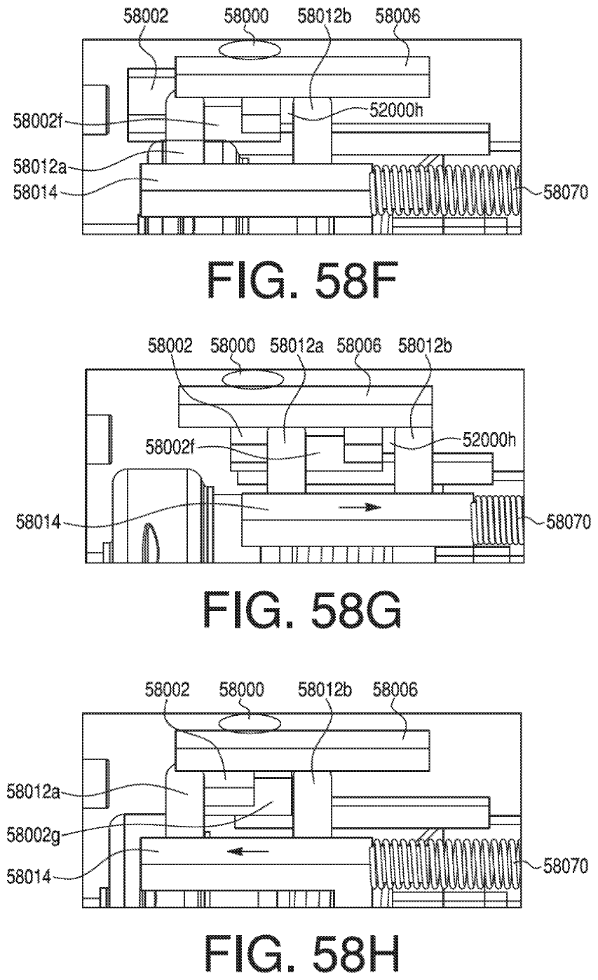

FIGS. 23N-Q show yet another alternative embodiment for needle insertion and retraction. In particular, in this alternative embodiment, the shuttles disclosed herein may be directly coupled to container 1302. For example, as shown in FIG. 23N a shuttle 340h may be coupled to container 1302, via, e.g., a collar 340z, extending from the body of shuttle 340h, that wraps around a neck of container 1302. Any other suitable connection also is contemplated. Additionally, in one or more embodiments, collar 340z may correspond to or otherwise may be coupled to sleeve 32008 described herein with respect to FIGS. 32R-V. Thus, a combined shuttle (of the patient needle mechanism) and sterile connector are contemplated. Furthermore, collar 340z may wrap around or may otherwise be coupled to another portion of container 1302, such as, for example, around the body of container 1302. In some embodiments, it is contemplated that shuttle 340h may be coupled to a standard container or cartridge, while in other embodiments, a custom container 1302 may be utilized, including, for example a container 1302 having one or more protrusions, recesses, or other features configured to interact with and secure to shuttle 340h. Shuttle 340h may include any of the features described herein with respect to any of the other shuttles, including rack gears, multiple offset and/or parallel extensions, and rods or pegs for interfacing with the indicator system described herein in FIGS. 58A-58H.

A spring 370b may be coupled to container 1302 and/or shuttle 340h, and may be configured to bias the container 1302/shuttle 340h into the position shown in FIG. 23O, and to help provide the force needed to return shuttle 340h toward its initial position (or to a third position at or near the initial position)—i.e., to help provide the force needed to retract needle driver 320 (e.g., via gear 360a) and withdraw the patient end of needle 306 from the patient. Spring 370b may be configured to compress as container 1302/shuttle 340h move from the initial (first) position to a deployed (second) position. One end of spring 370b may be coupled to container 1302 and/or shuttle 340h, while an opposite end of spring 370b may be coupled to an otherwise fixed or stationary portion of auto-injector 2, such as, e.g., housing 3 or carrier 202, to form a spring stop 371.

As shown in FIGS. 23O-Q, shuttle 340h may be positioned below gear 360a (closer to the tissue-contacting surface/injection site). However, it is also contemplated that shuttle 340h may be disposed above gear 360a (farther from the tissue-contacting/injection site). Needle insertion may be initiated from initial pressure from gas from a gas canister/fluid source 1366, as discussed herein. The linear movement of shuttle 340h in a first linear direction causes gear 360a to rotate as a result of being driven by rack gear 342 as discussed herein with respect to e.g., FIG. 23E and other figures. As shown in FIG. 23P, the rotation of gear 360a in a first rotational direction causes driver 320 and needle 306 to deploy in the downward direction (toward the skin surface). The initial linear movement also causes spring 370b to compress. Then, as shown in FIG. 23Q, when the force of gas acting on the container 1302/shuttle 340h is less than the force of spring 370b, spring 370b may expand and bias container 1302/shuttle 340h, in a second linear direction opposite to the first linear direction. The linear movement of shuttle 340h in the second linear direction causes gear 360a to rotate in a second rotational direction opposite to the first rotational direction. The rotation of gear 360a in the second rotational direction causes driver 320 and needle 306 to retract in the upward direction (away from the skin surface).

FIGS. 23R-U are schematic views that show the system flow within auto-injector 2t (described in further detail below with respect to FIGS. 48A-C and 48H-I), which may be substantially similar to the system flow shown in, for example, FIGS. 3A and 23M. As shown, auto-injector 2t may include a retraction system 23100 similar to venting system 2300A described herein. As shown, retraction system 23100 includes a shroud 23102, which may be movable relative to needle 306 and a portion of housing 3. Additionally, shroud 23102 may be proximate to gas canister or fluid source 1366 and venting system 2300, which may include a dump valve, as discussed herein. As discussed above, auto-injector 2t may also include container 1302, flow restrictor 3008, valve 3010 with diaphragm 3012, vent line 3006, and other components coupled via a number of conduits.

As shown in FIG. 23S, retraction of shroud 23102 relative to housing 3 initiates fluid source 1366. For example, as shown in FIGS. 48H and 48I, an initiation rod 48012 may be coupled to shroud 23102, and, when shroud 23102 is retracted, initiation rod 48012 activates fluid source 1366 in a manner similar as to other gas canister or fluid source activation mechanisms described herein. Then, gas flows through the system and valve 3010 as described herein, urging medicament through the fluid conduit and patient needle 306 that is extended from shroud 23102 and is inserted into the patient, as shown in FIG. 23S.

There is a further conduit or connection, for example, conduit 23104, which connects shroud 23102 and venting system 2300. While in the high pressure state, where diaphragm 3012 is sealing vent line 3006, gas is prevented from flowing through conduit 23104 by the dump valve in retraction system 23100. When the pressure equilibrates, and diaphragm 3012 lifts off of the valve seat, vent line 3006 urges the dump valve in retraction system 23100 into a configuration which allows gas from fluid source 1366 to flow through conduit 23104. The force of gas flowing through conduit 23104 may then urge shroud 23102 to extend such that needle 306 is in the retracted state, as shown in FIGS. 23T and 48C.

FIG. 23U illustrates an alternative schematic for auto-injector 2t. As shown, shroud 23102 may be coupled to venting system 2300 via a physical connection. For example, venting system 2300 may include or be coupled to a piston or push rod 23106 disposed within conduit 23104, which may be moveable to control the position of shroud 23102 relative to the housing of auto-injector 2t and needle 306 as discussed herein. In this aspect, the flow of fluid from fluid source 1366, valve 3010, vent line 3006, and venting system 2300 may control the position of push rod 23106, and thus control the position of shroud 23102.

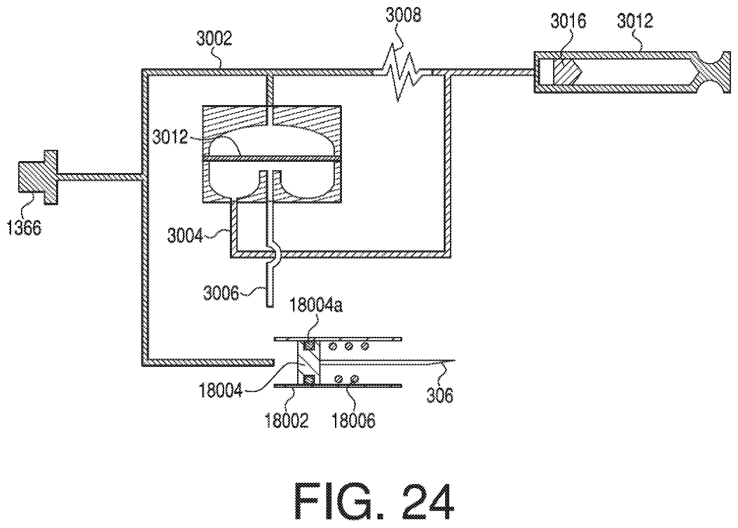

FIG. 24 shows an alternative mechanism for driving needle 306 into a user/patient. In this embodiment, pressurized gas may be diverted from high pressure line 3002 toward a housing 18002. A piston 18004 including a seal 18004a may be coupled to needle 306 inside housing 18002. A spring, or other resilient member 18006, may be coupled to piston 18004 and may bias piston 18004 into a retracted state (contained within housing 18002, for example). When fluid source 1366 is actuated, pressurized gas may act upon piston 18004, compressing spring 18006, and extending needle 306 out of housing 18002 and into the user/patient. Needle 306 may retract when the spring force of spring 18006 is greater than the force of the pressurized gas acting upon piston 18004 (e.g., after fluid source 1366 expels most of its propellant).

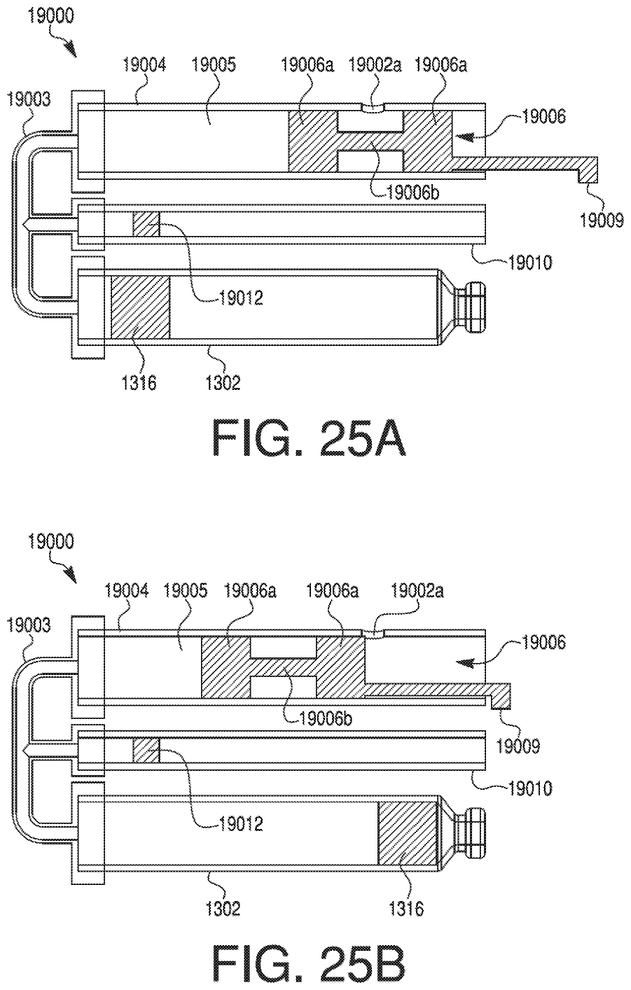

FIGS. 25A and 25B depict an alternative arrangement of an auto-injector 19000. Here, auto-injector 19000 still includes container 1302, piston 1316, and fluid source 1366. FIGS. 25A and 25B also depict a fluid connection 19003, a secondary cylinder 19004, hydraulic fluid 19005, a dumbbell piston 19006, an activation lever 19009, and an actuation cylinder 19010. Secondary container 19002 may include a port 19002a extending through a circumferential side surface of secondary container 19002.

Piston 1316 seals the medicament contained in container 1302 from the hydraulic fluid 19005, and serves as an interface to expel the medicament through container 1302 (e.g., from left to right as shown in FIGS. 25A and 25B). Fluid connection 19003 allows for movement of hydraulic fluid 19005 from the secondary container 19002 to container 1302 to move piston 1316. Fluid connection 19003 also allows for diversion of hydraulic fluid 19005 to the actuation cylinder 19010, which includes a piston 19012 that may be configured to actuate additional components of the device (e.g., actuating or retracting a needle mechanism, firing a sterile connector, etc.). Dumbbell piston 19006 in the secondary container 19002 includes a propulsion interface that pressurized gas from fluid source 1366 acts upon, and serves as an interface between fluid source 1366 and the hydraulic fluid 19005. Furthermore, dumbbell piston 19006 includes two heads 19006a coupled together by a shaft 19006b. Heads 19006a may have a substantially similar diameter. Furthermore, any of the configurations of pistons described in U.S. Publication No. 2016/0243309, incorporated herein by reference, may be utilized instead of dumbbell piston 19006. Furthermore, dumbbell piston 19006 may be used anywhere herein as an alternative to piston 1316.

When acted upon by pressurized gas from fluid source 1366, dumbbell piston 19006 exerts a force on the hydraulic fluid 19005. The space between the ends of dumbbell piston 19006 may be collapsible such that events may be triggered by activation lever 19009 prior to the dumbbell piston 19006 moving hydraulic fluid 19005 through fluid connection 19003. Activation lever 19009 may be configured to trigger a variety of events upon movement of the lever by pressure against the propulsion interface of dumbbell piston 19006. For example, the activation lever 19009 may actuate needle 306, retract needle 306, or move container 1302 (or another suitable container).

As shown in FIG. 25A, the trailing piston head 19006a may initially be disposed upstream of port 19004a. For example, port 19004a may be disposed longitudinally between piston heads 19006a as shown in FIG. 25A. Alternatively, port 19004a may be disposed downstream of an entirety of piston 19006. Trailing piston head 19006a eventually may be pushed past (downstream) of port 19002a (FIG. 25B), at which point pressurized gas from fluid source 1366 no longer pushes piston 19006 through secondary container 19002, but vents through port 19002a. The vented pressurized gas may flow into the interior of auto-injector 2 and/or into the atmosphere.

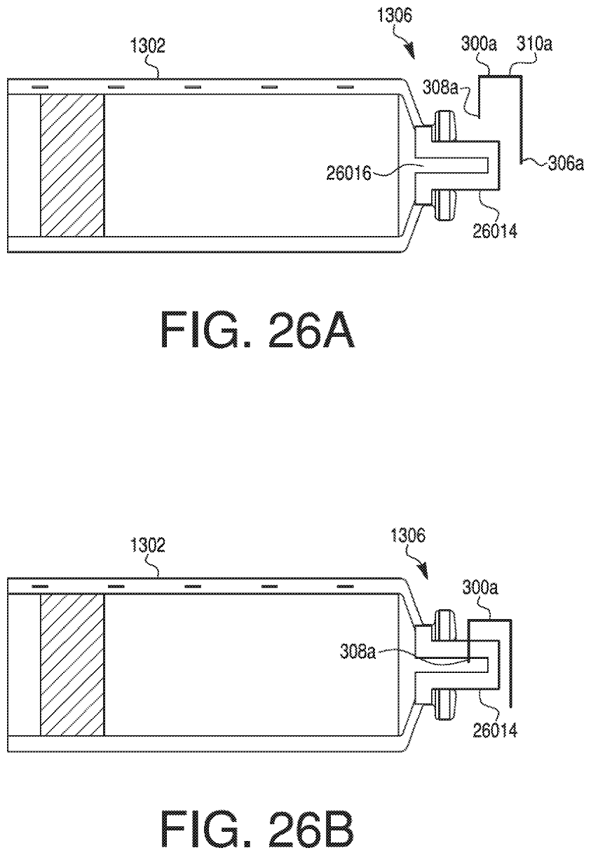

FIGS. 26A and 26B show container 1302 having a seal 26014 instead of a seal 1314 at second end 1306. Seal 26014 may be a plug, for example, including the same materials as seal 1314. However, seal 26014 also may include an interior cavity 26016 that is in fluid communication with the contents of container 1302. Cavity 26016 may protrude away from second end 1306 of container 1302 and away from the interior of container 1302. Seal 26014 may be pierced by one end of a fluid conduit 300a to establish fluid communication between container 1302 and the fluid conduit 300a. Fluid conduit 300a may include a needle 306a, an intermediate section 310a, and a needle 308a. Needle 306a may be similar to needle 306 described above, and may be configured to be inserted into a patient. Needle 308a may extend substantially parallel to needle 306a, and needle 308a may be configured to pierce seal 26014 along a path that is substantially perpendicular to the longitudinal axis of container 1302. When needle 308a pierces seal 26014, it may enter cavity 26016 to bring fluid conduit 300a and container 1302 into fluid communication with one another. That is, once needle 308a is within cavity 26016, medicament may be able to flow from container 1302 into cavity 26016 and needle 308a. Then, medicament may travel through the remainder of conduit 300a into a user/patient. Both needle 306a and needle 308a may extend substantially perpendicularly to the longitudinal axis of container 1302. Intermediate section 310a may fluidically couple needle 308a with needle 306a, and may extend substantially perpendicular to both needle 306a and needle 308a. Thus, intermediate section 310a may extend substantially parallel to the longitudinal axis of container 1302, and adjacent linear sections of fluid conduit 300a may be perpendicular to one another. The configuration shown in FIGS. 26A and 26B may enable fluid conduit 300a to have fewer bends and turns, thereby potentially improving flow through the conduit (i.e., by reducing the number of bends in the fluid conduit, thereby lowering restriction to fluid flow). Fluid conduit 300a may be moved either by an expanding spring, or by a button coupled directly to fluid conduit 300a, whereby the depression of the button causes fluid conduit 300a to move and needle 308a to pierce seal 26014. Or, fluid conduit 300a may be driven by a flow of pressurized fluid/gas from fluid source 1366. Furthermore, with the embodiment shown in FIG. 26A, regardless of the driving force, it is contemplated that the same force may be used to simultaneously pierce seal 26014 with needle 308a, and to eject needle 306a from the auto-injector and into the user/patient.

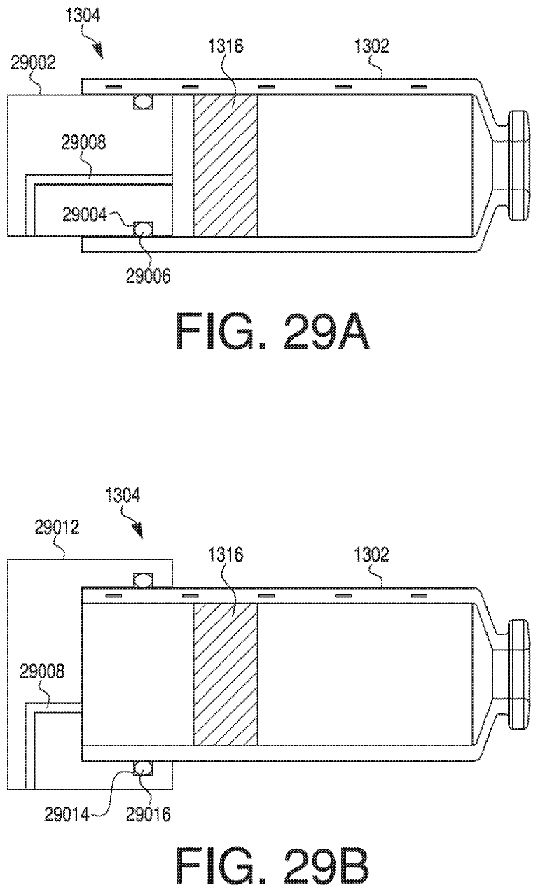

FIGS. 27A and 27B depict an embodiment where a fluid conduit 300b may move relative to a stationary container 1302 to move into fluid communication with container 1302. Fluid conduit 300b may include a needle 306b substantially similar to needles 306 and 306a described above. Needle 308b may extend substantially perpendicular to needle 306b, and needle 308b may be configured to pierce seal 1314 along a path that is substantially parallel to the longitudinal axis of container 1302. Intermediate sections 310b and 311b may fluidically couple needle 306b and needle 308b to one another. After fluid conduit 300b pierces seal 1314, medicament may be able to flow from container 1302 into needle 308b, intermediate section 311b, intermediate section 310b, and then into needle 306b. Intermediate section 310b may be substantially parallel to the longitudinal axis of container 1302, while intermediate section 311b may be substantially perpendicular to the longitudinal axis of container 1302. Similar to fluid conduit 300a, adjacent linear sections of fluid conduit 300b may be perpendicular to one another. The embodiment shown in FIGS. 27A and 27B may have sub-optimal speed (with conduit 300a moving faster than an optimal speed) and coring (where portions of the seal are removed from the seal by needle 308, and where some of the removed portions travel through and plug the fluid conduit) relative to other embodiments, but may be able to accompany an interior seal (described below with respect to FIG. 29A) since container 1302 is stationary. The use of a seal interior to container 1302 may help reduce the overall size of auto-injector 2. In other words, the use of an interior seal can reduce the envelope size of a housing for the container 1302 and an associated valve (e.g., valve 3010), because a smaller valve height and width can be used compared to when container 1302 is configured to move relative to a stationary fluid conduit 300b during the piercing step (as described below).

The embodiment shown in FIGS. 28A and 28B is similar to the embodiment of FIGS. 27A and 27B, except that container 1302 moves toward a stationary fluid conduit 300b to bring container 1302 into fluid communication with fluid conduit 300b. This particular embodiment may require a seal that wraps around an exterior of container 1302 (described below with respect to FIG. 29B), which generally, is larger than an interior seal having sealing rings inside of container 1302. Furthermore, the embodiment of FIGS. 28A and 28B may experience some needle alignment issues due to the relatively small target area that fluid conduit 300b presents to container 1302, and since container 1302 may wobble. However, this embodiment may be easier to control than the embodiment of FIGS. 27A and 27B because in this embodiment, the pressurized gas acts on container 1302, which is heavier than fluid conduit 300, and thus moves slower than fluid conduit 300, when acted on by an equivalent amount of pressurized gas.

FIGS. 29A and 29B show different mechanisms for sealing a volume around first end 1304 of container 1302. In the embodiments shown in FIGS. 29A and 29B, the sealed volume is configured to receive gas or fluid from fluid source 1366, to move container 1302 toward fluid conduit 300 to establish fluid communication between container 1302 and fluid conduit 300, and to drive piston 1316 through container 1302. In the embodiment shown in FIG. 29A, a seal housing 29002 includes a circumferential groove 29004 in a radially outer surface of seal housing 29002. A seal 29006 is disposed within groove 29004. At least a portion of seal housing 29002, and the substantial entireties of groove 29004 and seal 29006 are inserted into container 1302 at first end 1304. In some embodiments, seal housing 29002 and seal 29006 are maintained within container 1302 by a press or friction fit. Seal housing 29002 also may include a conduit 29008 through which pressurized gas/fluid from fluid source 1366 travels into container 1302 for pushing piston 1316 through container 1302. While only one seal 29006 and groove 29004 are shown, it is contemplated that additional seals and grooves may be utilized. In some embodiments, there may be a relatively small space behind piston 1316 within housing 3, particularly when the dose of medicament within container 1302 is relatively high (requiring piston 1316 to be relatively close to first end 1304 of container 1302). The embodiment shown in FIG. 29A may work well with a piercing mechanism where container 1302 remains stationary, and a fluid conduit (e.g., fluid conduit 300) is moved toward container 1302. Furthermore, by sealing inside of container 1302 (with rings of seal 29006 contacting the radially interior surface of container 1302), the embodiment of FIG. 29A is smaller than other embodiments (e.g., where the seal contacts the external and radially outer surfaces of container 1302), and may help enable the use of container 1302 in smaller auto-injector housings/envelopes. Seal housing 29002 may be fixed relative to housing 3 of auto-injector 2.

Although not shown, container 1302 may be any appropriate size and/or shape, for example, in order to accommodate container 1302 within housing 3 of auto-injector 2. For example, container 1302 may be sized and/or shaped to include a 3 mL fluid cartridge, and container 1302 may include a length that extends approximately 6 to 10 mm, for example, approximately 8 mm, beyond the fluid cartridge. The size and/or shape of container 1302 may allow for additional space (e.g., within container 1302) to accommodate one or more seals behind the piston, to allow for the fluid cartridge to slide toward and/or onto the needle, etc. Additionally, as discussed herein, container 1302 may include one or more seals, for example, dynamic seals on an interior or inside portion of container 1302.

In the embodiment shown in FIG. 29B, a seal housing 29012 includes a circumferential groove 29014 in a radially inner surface of seal housing 29012. A seal 29016 is disposed within groove 29014, and at least a portion of seal housing 29012, groove 29004, and seal 29006 are positioned exterior to container 1302 at first end 1304. In some embodiments, seal housing 29012 and seal 29016 are maintained around container 1302 by a press or friction fit. Seal housing 29012 also may include a conduit 29018 through which pressurized gas/fluid from fluid source 1366 travels into container 1302 for pushing piston 1316 through container 1302. While only one seal 29016 and groove 29014 are shown, it is contemplated that additional seals and grooves may be utilized. The embodiment shown in FIG. 29B may be well suited for use with an activation mechanism where container 1302 moves toward a stationary fluid conduit. In particular, seal 29016 may be positioned along the exterior of container 1302 to allow for the movement of container 1302 relative to seal 29016 without risking disengagement of seal 29016. For example, seal 29016 may be positioned closer to second end 1306 of container 1302 (enabling container 1302 to travel a greater distance) without affecting the dosing capacity of container 1302. Thus, seal housing 29012 may be able to accommodate larger doses within container 1302, or larger containers 1302 within a given auto-injector 2, than seal housing 29002. However, the embodiment shown in FIG. 29B may occupy a larger volume than the embodiment shown in FIG. 29A. Seal housing 29012 may be fixed relative to housing 3 of auto-injector 2.

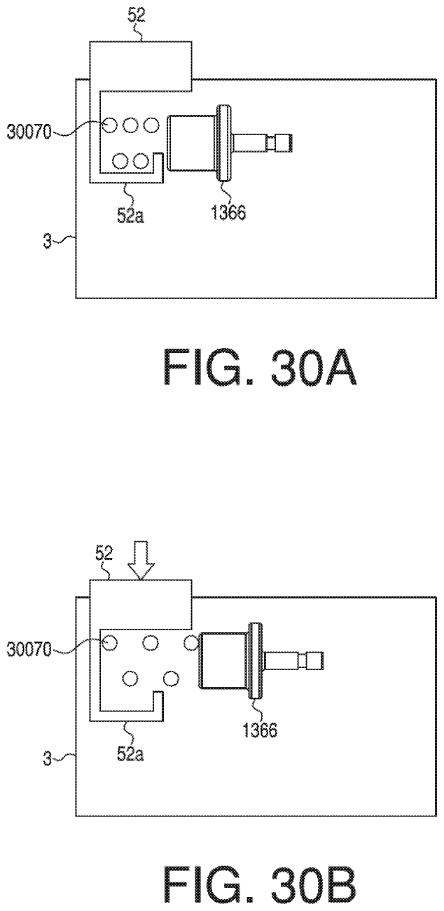

FIGS. 30A and 30B show a mechanism for activating fluid source 1366 that includes, e.g., button 52 movable relative to housing 3 of auto-injector 2. In this embodiment, button 52 may include a stop 52a configured to maintain a spring 30070 in a collapsed configuration (FIG. 30A). While spring 30070 is in the collapsed configuration, fluid source 1366 may be deactivated (i.e., not dispensing any fluid or gas). For example, spring 30070 may maintain a valve stem into a closed configuration when spring 30070 is collapsed. Upon depression of button 52 (or relative movement between button 52 and housing 3), stop 52a may clear out of the path of spring 30070, enabling spring 30070 to expand (FIG. 30B). This expansion may move the valve stem into an open configuration to activate the flow of fluid/gas from fluid source 1366. In other embodiments, the valve stem may remain fixed within auto-injector 2, and spring 30070 may be coupled to a portion of fluid source 1366 that moves relative to the stationary valve stem, to activate/deactivate fluid source 1366.

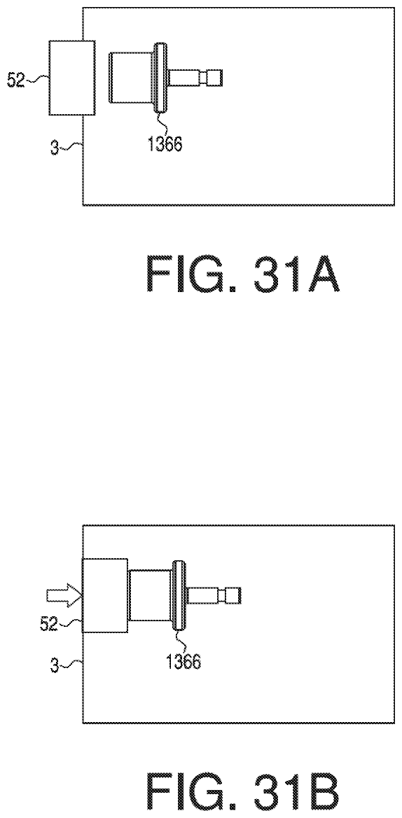

FIGS. 31A and 31B show a mechanism for activating fluid source 1366, where depression of button 52 directly activates fluid source 1366. For example, pushing button 52 relative to housing 3 may cause button 52 to directly contact a portion of fluid source 1366. For example, button 52 may contact and move a valve stem of fluid source 1366 into an open configuration, to enable the flow of fluid/gas from fluid source 1366. Or, the valve stem may remain fixed within auto-injector 2, and button 52 may be coupled to a portion of fluid source 1366 that moves relative to the stationary valve stem, to activate/deactivate fluid source 1366.

FIGS. 32A and 32B show yet another mechanism for activating fluid source 1366, that includes, e.g., button 52 movable relative to housing 3 of auto-injector 2. In this embodiment, button 52 may include a stop 52a configured to maintain a spring 32070 in a collapsed configuration (FIG. 32A). Spring 32070 may be coupled to a fluid conduit (e.g., fluid conduit 300 described above), and may drive needle 308 or another similar needle into fluid communication with container 1302. Upon depression of button 52 (or relative movement between button 52 and housing 3), stop 52a may clear out of the path of spring 32070, enabling spring 32070 to expand (FIG. 30B). The expansion of spring 32070 also may directly or indirectly drive a patient needle mechanism as set forth above, such that a needle (e.g., needle 306) exits the auto-injector and enters the patient. The patient needle mechanism is shown generically in FIGS. 32A and 32B as patient needle mechanism 32100. Patient needle mechanism 32100 may represent any portion of the patient needle mechanism disclosed herein, including, e.g., the various shuttles, rods, racks, drivers, fluid conduits, carriers, or other movable structure used to deploy a needle into the patient. Any one of the features may be configured to contact and activate the canister, for example, by moving a valve stem from a closed configuration to an open configuration, or by moving another portion of the canister relative to a stationary valve stem.

FIGS. 32C-32H illustrate additional aspects of another mechanism for activating the fluid source, for example, via button 52. As shown in FIG. 32C, button 52 may be positioned on or flush with an outer face of housing 3 of auto-injector 2. As shown in greater detail in FIGS. 32D and 32E, button 52 may be coupled to spring 32070, which may surround a spring carrier 32072, and spring 32070 may be connected to a gas canister 32074. Spring carrier 32072 may be substantially cylindrical, with a widened circular end 32072a on one end and carrier posts 32072b extending laterally outward from the cylindrical portion of spring carrier 32072, in opposing directions, on the other end of spring carrier 32072.

FIG. 32F illustrates an unused or inactive state of button 52 (before activation). As shown in FIG. 32F, carrier post 32072b is blocked by patient needle mechanism carrier and button block 32078 (which may be substantially similar to carrier 202 or other carriers described herein), preventing spring 32070 from releasing. FIG. 32G illustrates button 52 being activated (e.g., pressed down by a user). In this aspect, activation of button 52 also pushes carrier post 32072b down (or rotates spring carrier 32072, which rotates carrier post 32072b). FIG. 32H illustrates the fully activated position. As shown in FIG. 32H, carrier post 32072b is clear of the blocking portion of patient needle mechanism carrier and button block 32078, allowing spring 32070 to expand, and the expansion of spring 32070 may push spring carrier 32072 into a portion of gas canister 32074. In one aspect, pushing spring carrier 32073 into a portion of gas canister 32074 provides enough force to trigger gas release from canister 32074. For example, spring 32070 may provide approximately 20 to 40 N of force, for example, approximately 30 N of force, on spring carrier 32072.

The above activation system may include exactly three components, providing a simple construction of the system. For example, the above activation system may help to increase in the ease of assembly and/or manufacture.

FIGS. 32I-32M illustrate additional aspects of another mechanism for activating the fluid source, for example, via button 52, which may be positioned on or within an outer face of housing 3 of auto-injector 2, as discussed above. As shown in greater detail in FIGS. 32J-32M, button 52 may actuate an activation mechanism 32080.

FIG. 32J illustrates activation mechanism 32080 in an unused or inactive state. As shown, activation mechanism 32080 includes a carrier 32082 (which could include one or more features of other patient needle carriers disclosed herein) and an actuator 32084. Actuator 32084 may be coupled to and controlled (e.g., moved) by button 52. Actuator 32084 may include a substantially horizontal portion 32084a, for example, that extends parallel to an outer face of button 52. Actuator 32084 may also include a substantially vertical portion 32084b. Vertical portion 32084b may include two arms 32084c. Moreover, movement of actuator 32084 may be at least partially restricted or blocked by a peel tab (not shown) at a peel tab interface 32088 disposed on or adjacent the bottom or tissue-engaging side of auto-injector 2. The peel tab may be disposed on at least a portion of the tissue engaging surface of auto-injector 2, through which the patient needle extends. Although only one is shown in the figures, actuator 32084 may include two snap tabs 32086, for example, positioned on both sides of vertical portion 32084b. Snap tabs 32086 may include a downward and radially-inward oriented taper, and an upward facing shoulder, which may allow it to move downward when button 52 is initially depressed. During this downward movement toward the skin surface and bottom of the auto-injector 2, snap tab 32086 may be received into a recess 32086a. Then, when the user removes her finger from button 52, actuator 32084 moves in the upward direction away from the skin surface, but is eventually locked into place via the interaction of snap tab 32086 with the surfaces surrounding recess 32086a. Or, snap tab 32086 may lock into recess 32086a upon entry into recess 32086a. Thus, with actuator 32084 vertically fixed within the auto-injector 2, subsequent depression of button 52 by a user is prevented (or has no effect). In some embodiments, after assembly of auto-injector 2, snap tab 32086 may be disposed in a first recess 32086a, which helps secure the button assembly together until activation by a user. Then, upon depression of button 52 by the user, snap tab 32086 may be locked into an adjacent recess 32086a that is closer to the skin surface (or otherwise closer to the bottom of auto-injector 2).

Peel tab interface 32088 may be disposed on or adjacent to the bottom of auto-injector 2. For example, a vertical portion 32084b of actuator 32084 may include a leg 32084d that extends to peel tab interface 32088. Although not shown, peel tab interface 32088 may include an opening 32082h in carrier 32082 and a peel tab. With the peel tab in place, leg 32084d, and thus actuator 32084, is blocked from moving through the opening 32082h in carrier 32082 and thus blocked from any downward movement. Thus, the peel tab, before being removed from auto-injector 2, may prevent inadvertent activation of auto-injector 2 by, e.g., pressing button 52 or dropping of auto-injector 2.

As shown, activation mechanism 32080 includes a canister activator 32090. Canister activator 32090 may include a cylindrical portion and a widened end portion or flange 32091. Moreover, canister activator 32090 may include one or more (e.g., 2) snap arms 32092. Snap arm 32092 may interact with a portion of actuator 32084, for example, with arm 32084c. For example, movement of actuator 32084 downward may help to transition canister activator 32090 from a locked and retracted position, as shown in FIG. 32J, to an unlocked and extended position, as shown in FIG. 32K. Furthermore, although not shown, a spring may be positioned internal to canister activator 32090, which may help transition canister activator 32090 to the extended position in FIG. 32K. The position of the spring inside canister activator 32090 may help keep the spring aligned.

FIG. 32L illustrates additional details of the interaction between snap boss 32092, carrier 32082, and a portion of actuator 32084, for example, with arm 32084c. As shown, arm 32084c may include a ramp portion 32084e. Also, carrier 32082 may include a first peg or boss 32082f. In the initial configuration, for example, as shown in FIG. 32J, peg 32082f may be received within an opening 32092a in snap arm 32092. Peg 32082f may act as a stop that prevents expansion of the spring within activator 32090 by abutting against an internal surface of snap arm 32092 that surrounds opening 32092a. However, as actuator 32084 is urged downward when button 52 is depressed, for example, as shown in FIG. 32K, ramp portion 32084e may push, guide, or otherwise help to move a portion of snap boss 32092 outward in the direction T that is substantially perpendicular to a direction L along which canister activator 32090 travels. In moving snap arm 32092 in the direction T, snap arm 32092 is moved away from peg 32082f, such that peg 32082f no longer inhibits the travel of canister activator 32090 in the direction L. This may allow the spring disposed within canister activator 32090 to expand, which causes canister activator 32090 to move along the direction L away from carrier 32082, thereby activating the gas canister.

As mentioned above, FIG. 32K illustrates activation mechanism 32080 in an activated state and with needle driver 320 in the deployed position (with the patient needle inserted into the patient). In FIG. 32K, the peel tab has been removed (as compared to FIG. 32J), allowing for leg 32084d to extend through opening 32082h in carrier 32082. The path of canister activator 32090 further along direction L is blocked by a second peg or boss 32082g. Thus, after activator of auto-injector 2 by initially pressing button 52, canister activator 32090 is now fixed in the position shown in FIG. 32K. In FIG. 32K, actuator 32084 is locked into carrier 32082 (by engagement of snap tab 32086 and opening 32086a) so that the user cannot depress button 52 after the initial depression and release. FIG. 32M illustrates activation mechanism 32080 when needle driver 320 is in its retracted position and the patient needle is withdrawn from the patient. As discussed above with respect to FIG. 32K, the locking arrangement of snap tabs 32086 and recesses 32086a prevent further depression of button 52 by a user.

One or more aspects of activation mechanism 32080 may help to facilitate the transition of button 52, and thus, the activation of activation mechanism 32080. For example, the use of two snap tabs 32086 on opposing sides of actuator 32084 may help to balance the user's downward force, which may also help translate button 52. Moreover, the positions and/or arrangement of various elements of activation mechanism 32080 may help in the manufacture of activation mechanism 32080. For example, the position of snap arm 32092 outside of canister activator 32090, and the position of the activator spring inside of activator 32090, may allow for the components to be molded or otherwise manufactured easily, quickly, economically, etc. The presence of two snap tabs 32086 on opposing sides of actuator 32084 may help to form the lock out position via one or more recesses 32086a, as discussed with respect to FIG. 32M, which may help disable button 52 from being depressed (after initial depression of button 52 and activation of auto-injector 2). Moreover, the use of two snap tabs 32086 may help to apply an equal and/or balanced force on actuator 32084 which is partially centered under button 52. This may help to prevent bending and/or deformation of actuator 32084. The peel tab may also help to prevent accidental activation (e.g., caused by vibrations, drops, impacts, or other forces on activation mechanism 32080), by blocking the downward path of actuator 32084 and button 52. In these embodiments, stronger components or wings, such as, e.g., snap arms 32092 may help reduce creep within the button assembly. Furthermore, snap tabs 32086 may help prevent accidental activation of button 52 from drops by, e.g., friction.

FIGS. 32N-32V illustrate additional features that may be incorporated in auto-injector 2. FIGS. 32N and 32P are perspective views of a portion of activation mechanism 32080 in an unused or inactive state, with canister activator 32090 retracted relative to carrier 32082. Activation mechanism 32080 in this embodiment has a different snap tab 32084z that extends from actuator 32084. In particular, as shown in FIG. 32N-Q, snap tab 32084z may include a window that can receive and interact with a snap peg or boss 32082b on carrier 32082. The snap peg or boss 32082e may be a ramp with a downward-facing shoulder that enables actuator 32084 to move in the downward (skin-facing) direction, while also preventing actuator 32084 from moving upward after its initial depression. Thus, similar to the embodiment discussed above with respect to FIGS. 32I-M, button 52 is not able to be depressed again, after initial depression by a user (or any subsequent depression would not have any effect on the device).

FIGS. 32R-V illustrate a mechanism for preventing the early fluid communication between needle 308 and container 1302 (e.g., from an accidental drop). The mechanism shown in FIGS. 32R-V can be used with any other embodiment disclosed herein. As shown, fluid conduit 32098 (which may be substantially similar to other fluid conduits discussed herein, including, e.g., fluid conduit 300) may be coupled to a connector 32002. Connector 32002 may be rotatable and may include a connector boss 32004. Connector boss 32004 may be an outward protrusion extending radially outward from an outer surface of connector 32002. Connector 32002 may be configured interact with a sleeve disposed around container 1302. Sleeve 32008 may be coupled to and disposed around container 1302. Connector 32002 may be movable relative to sleeve 32008 in some configurations. Sleeve 32008 may snap or click onto container 1302, and thus, may be stationary relative to container 1302. As shown in FIG. 32R, sleeve 32008 may include a slot 32010, which may be configured to receive connector boss 32004. For example, slot 32010 may include a longitudinal portion extending longitudinally through a portion of sleeve 32008 and a lateral portion extending laterally/circumferentially through a portion of sleeve 32008. In this aspect, and as discussed below, the lateral/circumferential portion of slot 32010 may receive connector boss 32004 to form a substantially locked configuration between connector 32002 and sleeve 32008. The substantially locked configuration between connector 32002 and sleeve 32008 may secure connector 32002 after completion of the injection, and the presence of the lateral circumferential portion of slot 32010 may enable retraction of needle driver 320. Connector boss 32004 may help to prevent accidental or unintentional connection between connector 32002 and container 1302, for example, in case a user accidentally drops auto-injector 2. In particular, connector boss 32004 may act as a stop preventing relative movement between connector 32002 and container 1302 until the patient needle has been deployed by downward movement of needle driver 320.

FIG. 32S illustrates an enlarged view of the interaction of connector 32002 and sleeve 32008 in an initial or unused state. As shown, connector boss 32004 may include a width approximately equal to or slightly less than a width of slot 32010. Furthermore, in the initial or unused state, actuator 32084 may be extended, and connector boss 32004 is unaligned with slot 32010. In this aspect, connector boss 32004 helps to block or inhibit sleeve 32008 and container 1302 from moving toward connector 32002 (or inhibit connector 32002 from moving toward container 1302 in other embodiments), which would cause the fluid conduit to pierce container 1302 and cause medicament to discharge through the patient end of the needle. Thus, in the initial configuration, connector boss 32004 may prevent the relative movement between connector 32002 and sleeve 32008/container 1302.

FIG. 32T illustrates the interaction of connector 32002 and cartridge sleeve 32008 in an inserted state, for example, when a needle is inserted into the patient by the downward movement of needle driver 320. The downward movement of needle driver 320 urging the patient end of fluid conduit 300 out of housing 3 and into the patient (not shown in FIG. 32R), causes a center portion of fluid conduit 32098 (and connector 32002/connector boss 32004) to rotate in a first direction. The rotation of connector 32002/connector boss 32004 in the first direction may put connector boss 32004 into longitudinal alignment with slot 32010 so that connector 32002 and sleeve 32008/container 1302 may move relative to one another, e.g., by the force of pressurized gas from a gas canister as described elsewhere herein.

FIG. 32U illustrates the interaction of connector 32002 and sleeve 32008 after those components have moved toward one another to establish fluid communication between fluid conduit 32098 and container 1302. As shown, container 1302 and sleeve 32008 may be advanced toward connector 32002 by the force of fluid from the gas canister, with connector boss 32004 being received within slot 32010.

FIG. 32V illustrates the interaction of connector 32002 and cartridge sleeve 32008 in a retracted state, for example, when the patient needle is retracted from the patient as needle driver 320 moves upwardly and away from the skin surface. As shown, as the needle is retracted, fluid conduit 32098 and connector 32002 may rotate in a second direction that is opposite of the first direction. For example, when the first direction is clockwise, the second direction may be counter-clockwise. In other embodiments, when the first direction is counter-clockwise, the second direction is clockwise. The lateral/circumferential portion of slot 32010 may ensure the ability of needle driver 320 to move upwardly, and thus, may ensure the ability to withdraw the patient needle after delivery of medicament from container 1302. That is, without the lateral/circumferential portion of slot 32010, fluid conduit and connector 32002 would be prevented from rotating in the second direction.

As mentioned, the aspects above may help to ensure that fluid is not unintentionally delivered from container 1302 to fluid conduit 32098 until the patient needle has been deployed into the patient. In particular, connector boss 32004 may help prevent fluid conduit 32098 and container 1302 from prematurely establishing fluid communication with one another, causing fluid conduit to prematurely discharge medicament from the patient needle before the patient needle is deployed into the patient. Moreover, rotating connector 32002 to engage with container 1302 (via sleeve 32008) may help to reduce a risk of breakage or failure of fluid conduit 32098 by, e.g., crimping, bending, or the like. Furthermore, it is contemplated that connector boss 32004 and slot 32010 may be alternative structures so long as they are complementary to one another. For example, connector boss 32004 could be a slit, recess, or opening, and slot 32010 could be a protrusion extending radially outward from sleeve 32008 (but arranged with the same geometry and path as slot 32010 is shown in the figures).

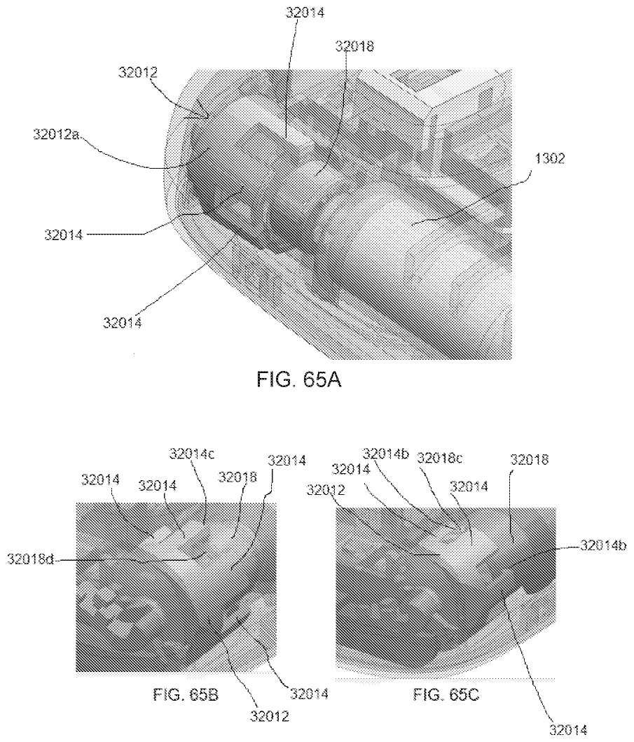

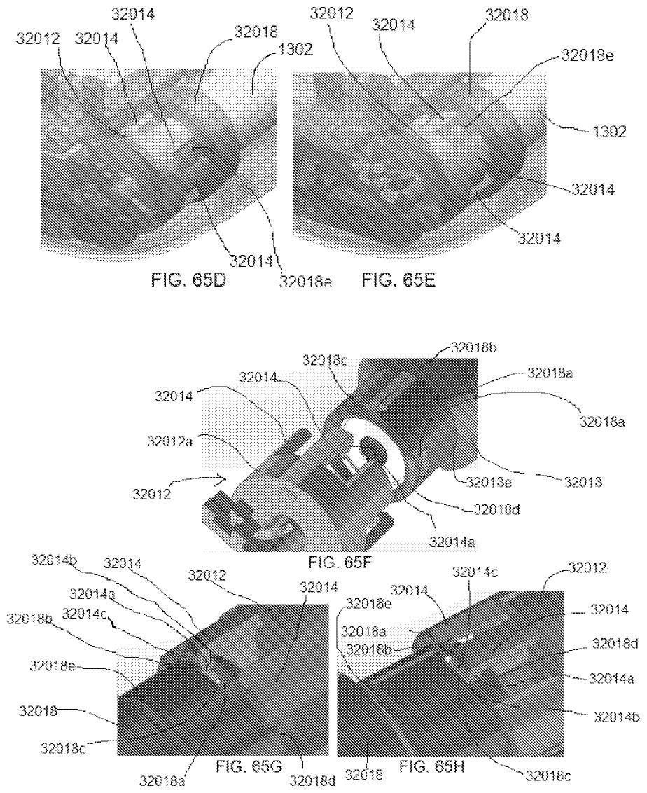

FIGS. 65A-H illustrate another mechanism for preventing the early fluid communication between the needle (not shown) and container 1302 (e.g., from an accidental drop). The mechanism shown in FIGS. 65A-H can be used with any other embodiment disclosed herein. Although not shown, the fluid conduit may be coupled to a connector 32012, as discussed above with respect to FIGS. 32R-V. Connector 32012 may be rotatable and may include at least one connector prong 32014. For example, connector 32012 may include two, three, four, or more connector prongs 32014 circumferentially spaced from one another and arranged and extending from a base 32012a of connector 32012. Connector prong(s) 32014 may be longitudinal extensions that extend from base 32012a of connector 32012 toward container 1302, and may each include an inward protrusion 32014a extending radially inward from an inner surface of connector prong 32014, for example, from an end portion of connector prong 32014. Additionally, each connector prong 32014 may include a slanted or ramped portion 32014b, for example, a reduced thickness portion at an end of connector prong 32014. Each connector prong 32014 may also include a flat end portion 32014c. Connector 32012 may be configured to interact with a sleeve 32018 disposed around or extending from container 1302. Sleeve 32018 may be coupled to and/or disposed around a portion of container 1302, and thus may be stationary relative to container 1302. Connector 32012 may be movable relative to sleeve 32018 in some configurations, as discussed above, for example, with respect to FIGS. 32R-V.

As shown in FIGS. 65B-E, connector 32012 is selectively rotatable and longitudinally movable relative to sleeve 32018. Although not shown, the rotation may be conveyed from the fluid conduit 300 and driver 320, as discussed above with respect to FIGS. 32R-V. Moreover, FIGS. 65F-H illustrate portions of connector 32012 and sleeve 32018 in various stages of assembly and activation. As shown, sleeve 32018 may include one or more grooves 32018a, which may extend through a circumferential outer portion of sleeve 32018. Each groove 32018a may extend through a circumferential thickness of sleeve 32018, or each groove 32018a may be a circumferential indentation in an outer portion of sleeve 32018. Moreover, groove 32018a includes a flat portion 32018b (e.g., perpendicular to the circumference of groove 32018a), and a slanted or ramped portion 32018c, for example, circumferentially arranged in groove 32018a. Sleeve 32018 may include any number of grooves 32018a, for example, a number of grooves 32018a corresponding to the number of connector prongs 32014. Sleeve 32018 may also include a boss portion 32018d, for example, at an end of sleeve 32018 opposite to container 1302. Furthermore, sleeve 32018 may include a collar portion 32018e, for example, at an end opposite boss portion 32018d, and proximate to container 1302. The collar portion 32018e may be secured to, e.g., a neck of container 1302 by a snap, interference, or screw fit.

For example, FIG. 65B illustrates an enlarged view of the interaction of connector 32012 and sleeve 32018 in an initial or unused state. As shown, connector prongs 32014 may be snapped on boss portion 32018d of sleeve 32018. In this configuration, connector 32012 may be rotatable relative to sleeve 32018, but may be at least partially restricted from longitudinal movement relative to sleeve 32018 and container 1302. In this aspect, boss portion 32018d of sleeve 32018 may help to prevent accidental or unintentional fluid connection between fluid conduit 300 (secured to connector 32012) and container 1302, for example, in case a user accidentally drops the auto-injector. In particular, boss portion 32018d may act as a stop to help prevent relative longitudinal movement between connector 32012 and sleeve 32018 (and container 1302) until the patient needle has been deployed by downward movement of the needle driver 320 (not shown). Although not shown in FIG. 65B, flat portion 32018b may interact with flat end portion 32014c to help prevent relative longitudinal movement between connector 32012 and sleeve 32018 (and container 1302).

FIG. 65C illustrates the interaction of connector 32012 and sleeve 32018 in a patient needle inserted state, for example, when a needle is inserted into the patient by the downward movement of the needle driver (not shown). The downward movement of the needle driver causes a center portion of fluid conduit 300 (not shown) and connector 32012 and connector prong(s) 32014 to rotate in a first direction. The rotation of connector 32012/connector prong(s) 32014 in the first direction may put connector prong(s) 32014 into longitudinal alignment with groove 32018a. Additionally, the rotation of connector 32012/connector prong(s) 32014 may put slanted portion 32014b of connector prong(s) 32014 into longitudinal alignment with slanted portion 32018c of sleeve 32018, so that connector 32012 and sleeve 32018/container 1302 may move relative to one another, e.g., by the force of pressurized gas from a gas canister as described elsewhere herein such that slanted portions 32014b and 32018c may help urge connector prong(s) 32014 out of groove(s) 32018a. In particular, the opposing ramps of slanted portions 32014b and 32018c may push connector prongs 32014 radially outward so that connector prongs 32014 can clear the outer surface of sleeve 32018, enabling longitudinal movement of sleeve 32018 relative to connector 32012.

FIG. 65D illustrates the interaction of connector 32012 and sleeve 32018 after those components have moved toward one another to establish fluid communication between the fluid conduit 300 (not shown) and container 1302. As shown, container 1302 and sleeve 32018 may be advanced toward connector 32012 by the force of fluid from the gas canister (not shown), with connector prong(s) 32014 being pushed out of the groove(s) (not shown). Additionally, connector prong(s) 32014 may lock onto or otherwise be received around collar portion 32018e of sleeve 32018. In this orientation, connector 32012 and sleeve 32018 may rotate relative to one another, but connector prong(s) 32014 may help to prevent longitudinal movement of connector 32012 and sleeve 32018 relative to one another, for example, in the reverse direction.

FIG. 65E illustrates the interaction of connector 32012 and cartridge sleeve 32018 in a patient needle retracted state, for example, when the patient needle is retracted from the patient as the needle driver 320 (not shown) moves upwardly and away from the skin surface withdrawing the patient end of the needle from the patient. As shown, as the needle is retracted, the fluid conduit 300 (not shown) and connector 32012 may rotate in a second direction that is opposite of the first direction. For example, when the first direction is clockwise, the second direction may be counter-clockwise. In other embodiments, when the first direction is counter-clockwise, the second direction is clockwise. The configuration of connector prong(s) 32014 and collar portion 32018e (rotatable relative to one another in FIG. 65D) may ensure the ability of the needle driver 320 to move upwardly, and thus, may ensure the ability to withdraw the patient needle after delivery of medicament from container 1302. That is, without connector prong(s) 32014 and collar portion 32018e being rotatable relative to one another, the fluid conduit and connector 32012 would be prevented from rotating in the second direction.

Moreover, as mentioned above, FIGS. 65F-H illustrate portions of connector 32012 and sleeve 32018 in various stages of assembly and activation. For example, FIG. 65F illustrates a pre-assembled configuration of connector 32012 and sleeve 32018. FIG. 65G illustrates an assembled configuration of connector 32012 and sleeve 32018. As shown, connector 32012 includes connector prongs 32014, each of which include inward protrusion 32014a. Additionally, in the assembled configuration of FIG. 65G, which is similar to the initial state shown in FIG. 65B, connector prongs 32014 may be locked on to sleeve 32018 (e.g., on boss portion 32018d), and longitudinal movement may be restricted by, for example, flat portion 32014c of connector prong 32014 and flat portion 32018b of groove 32018a, which may help to prevent relative movement of connector 32012 and sleeve 32018 (and thus container 1302) until insertion of the patient needle via the patient needle mechanism, as discussed herein. As shown in FIG. 65H, which is an enlarged view of a portion of the configuration shown in FIG. 65C, slanted portion 32014b of connector prong 32014 and slanted portion 32018c of groove 32018a are aligned, and connector 32012 and sleeve 32018 are in an unlocked configuration. Accordingly, connector 32012 and sleeve 32018/container 1302 may move relative to one another, e.g., by the force of pressurized gas from a gas canister as described elsewhere herein such that slanted portions 32014b and 32018c may help urge connector prong(s) 32014 radially outward and out of groove(s) 32018a.

As mentioned, the aspects above may help to ensure that fluid is not unintentionally delivered from container 1302 to the fluid conduit until the patient needle has been deployed into the patient. In particular, connector prongs 32014 and sleeve 32018 may help prevent the fluid conduit and container 1302 from prematurely establishing fluid communication with one another, causing fluid conduit to prematurely discharge medicament from the patient needle before the patient needle is deployed into the patient. Moreover, rotating connector 32012 to engage with container 1302 (via sleeve 32018) may help to reduce a risk of breakage or failure of the fluid conduit by, e.g., crimping, bending, or the like. Furthermore, it is contemplated that connector prongs 32014 and grooves 32018a may be alternative structures so long as they are complementary to one another. Moreover, the above embodiments may help to lock connector 32012 to sleeve 32018 before connector 32012, sleeve 32018, container 1302, etc. are assembled into the final assembly, for example, to form a locked arrangement after partial assembly between connector 32012 and sleeve 32018 before final assembly. Additionally, although not shown, the above embodiments may help to improve the alignment of cartridge needle with container 1302.

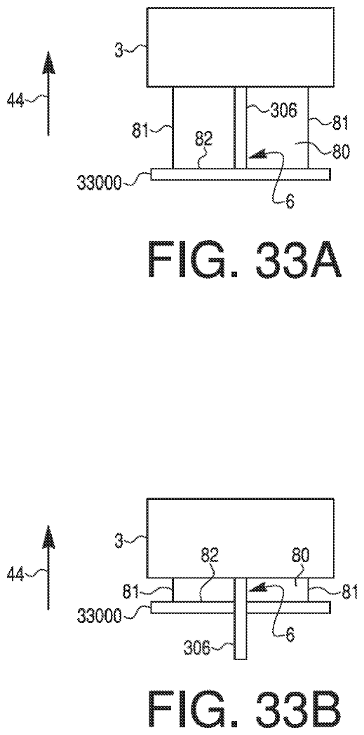

FIGS. 33A and 33B show a configuration of auto-injector 2 where a retractable shroud 80 extends from housing 3 and is movable relative to housing 3. Shroud 80 may retract along the transverse axis 44, into housing 3 by application of a force to housing 3 from a user. Shroud 80 may have sidewalls 81 and a tissue-engaging (e.g., bottom) surface 82. The sidewalls 81 may retract into housing 3 (see FIG. 33B) upon application of the force from the user.

Housing 3 and shroud 80 may be biased toward the initial state shown in FIG. 33A by one or more coils, elastic materials, pneumatic mechanisms, etc. The tissue-engaging surface 82 of shroud 80 may include an opening 6 through which needle 306 (or another patient needle) may be deployed. Retraction of shroud 80 (i.e., the movement of housing 3 and shroud 80 toward one another) may cause needle 306 to extend out of shroud 80, where it can be inserted through the user/patient skin 33000 and into the user/patient. After completion of an injection, fluid vented from a valve disclosed herein (e.g., valve 3010) may be diverted to urge tissue engaging surface 82 toward the skin 33000 to cover needle 306. For example, fluid/gas from fluid source 1366 that is vented through, e.g., vent 3018, may be diverted toward the skin along the transverse axis 44. The vented fluid/gas may push against shroud 80 along transverse axis 44, causing shroud 80 to move away from housing 3, and revert back to the configuration shown in FIG. 33A. Alternatively, vented gas/fluid may directly or indirectly trigger a spring or other mechanism to push shroud 80 away from housing 3 so that needle 306 is retracted and covered. In some examples, needle 306 may already be retracted by another mechanism when the vented air is used to revert shroud 80 to the configuration shown in FIG. 33A. Furthermore, it is contemplated that retraction of shroud 80 itself may trigger activation of fluid source 1366, for example by causing relative movement between a valve stem and another portion of fluid source 1366.

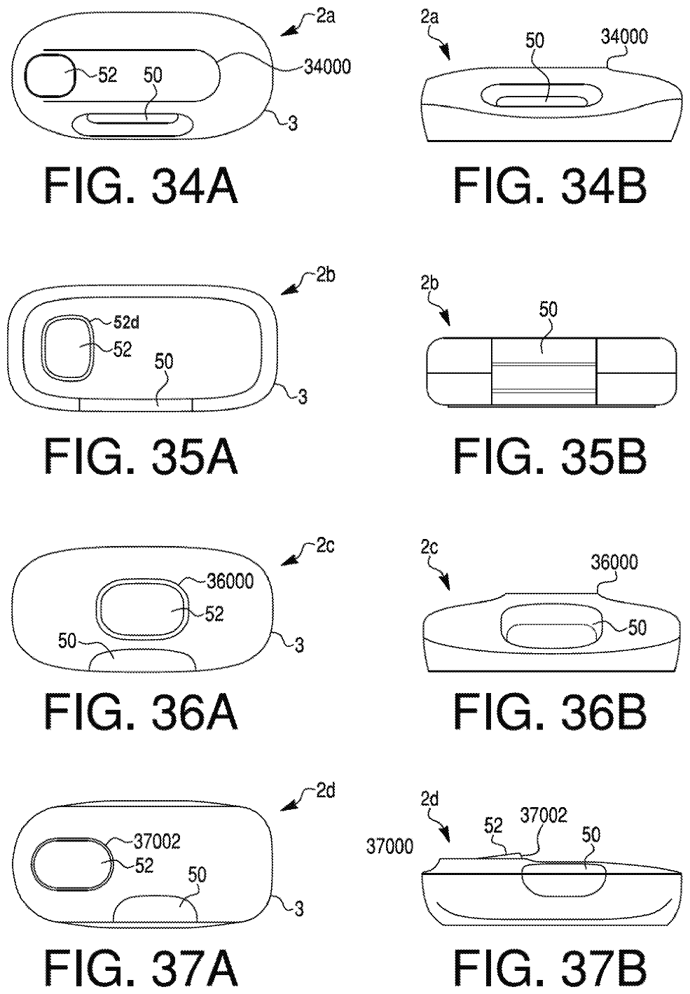

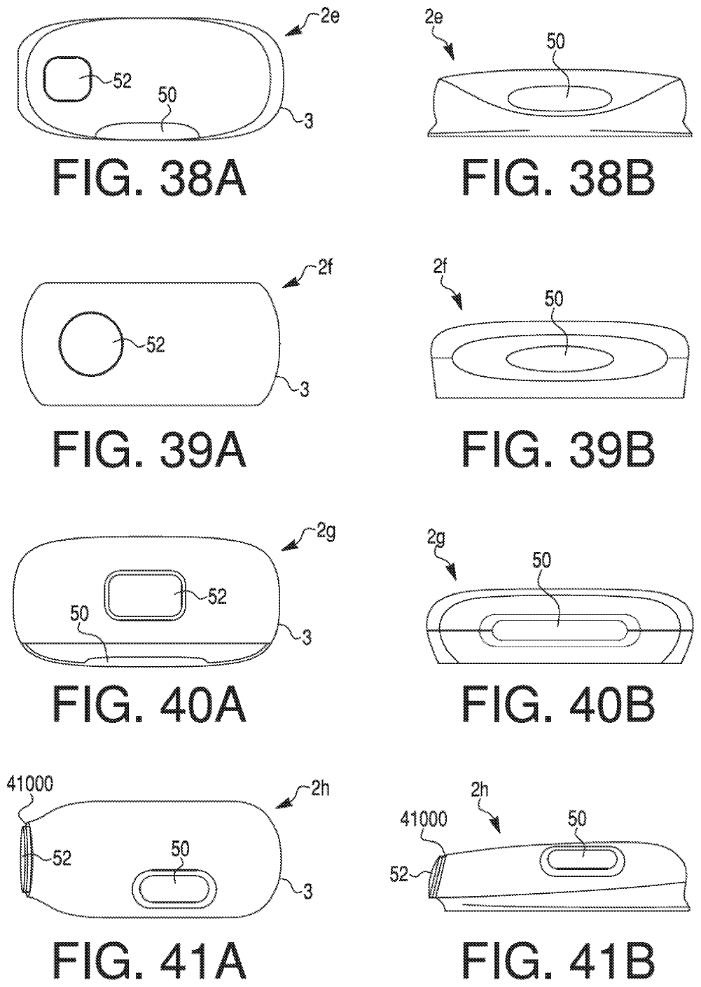

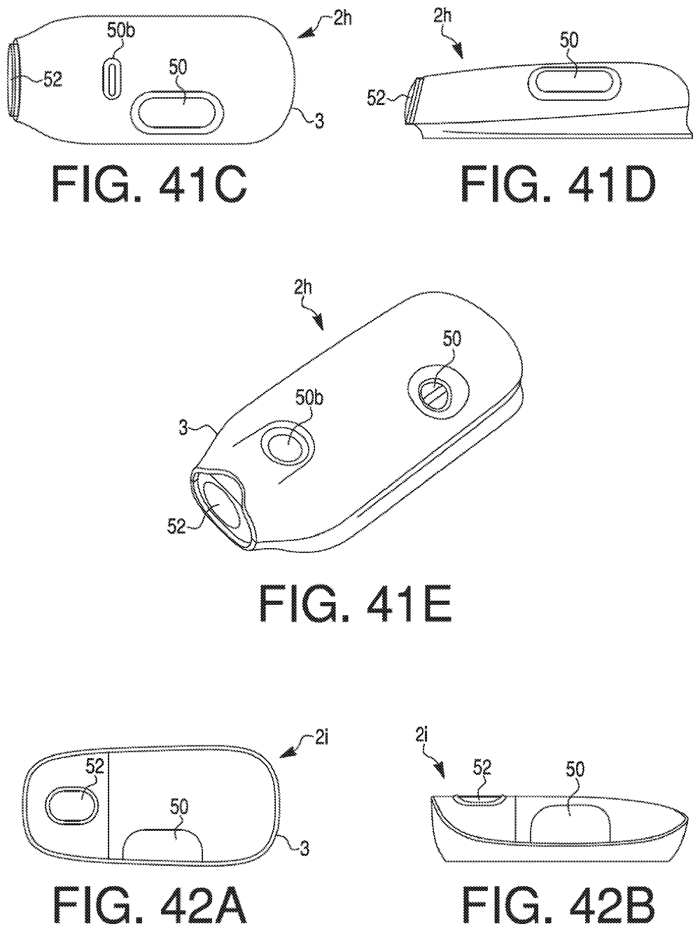

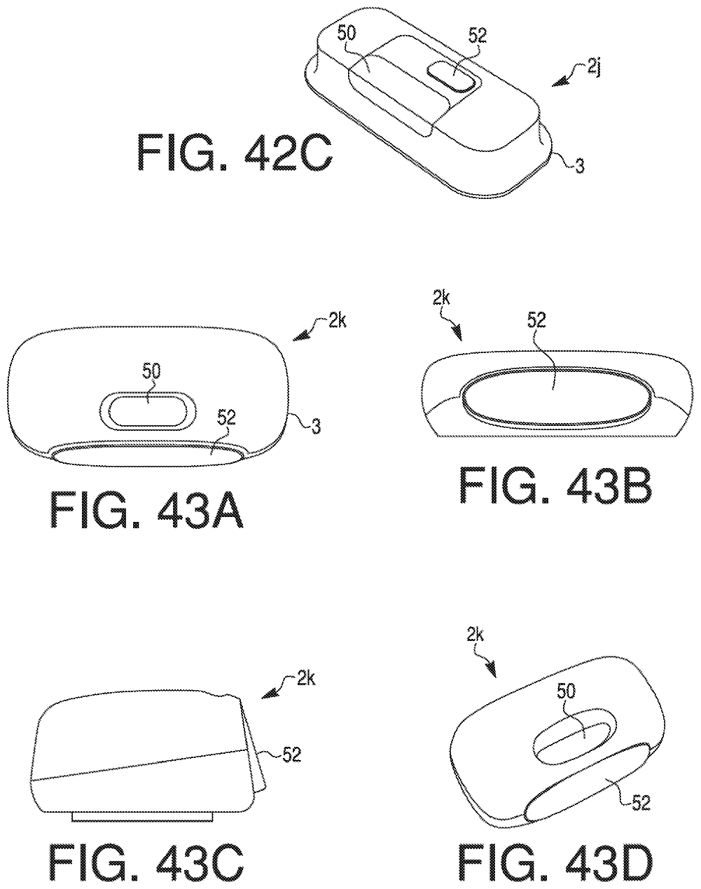

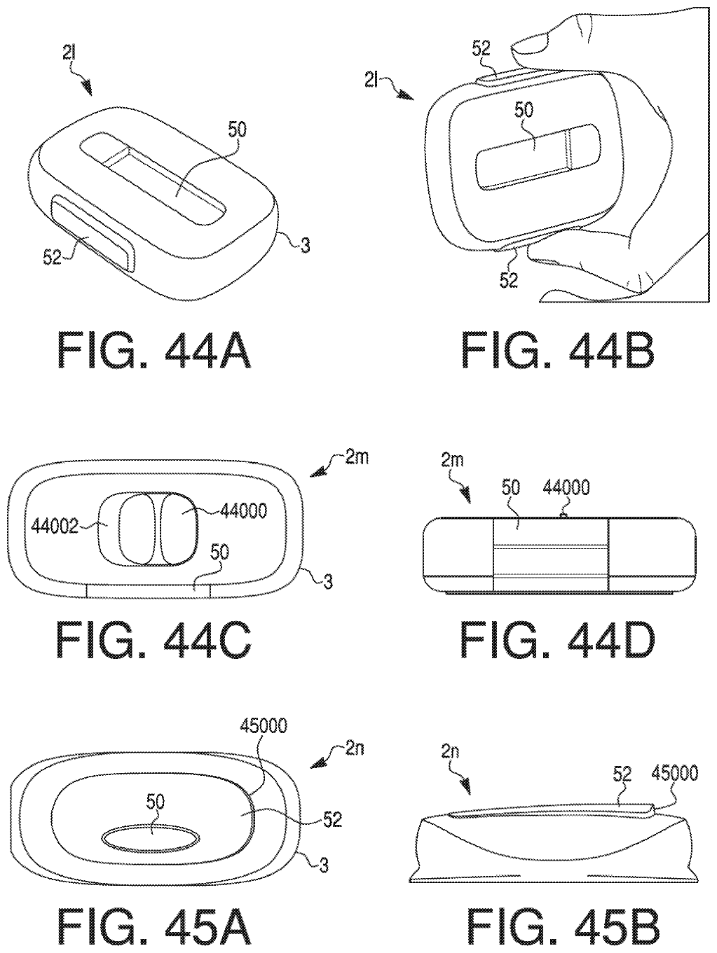

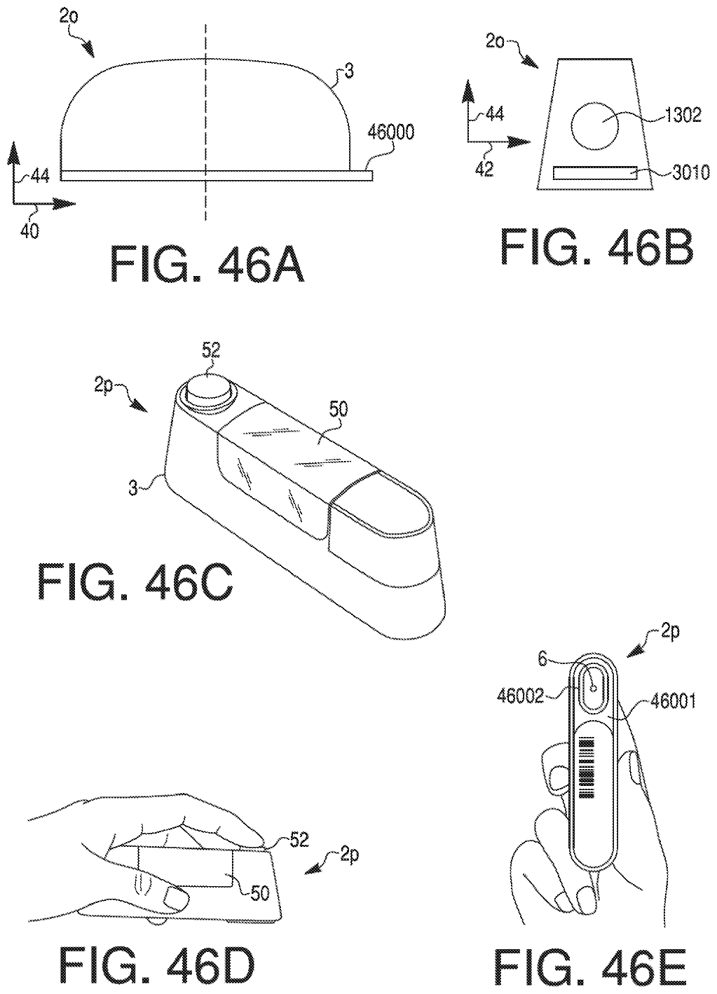

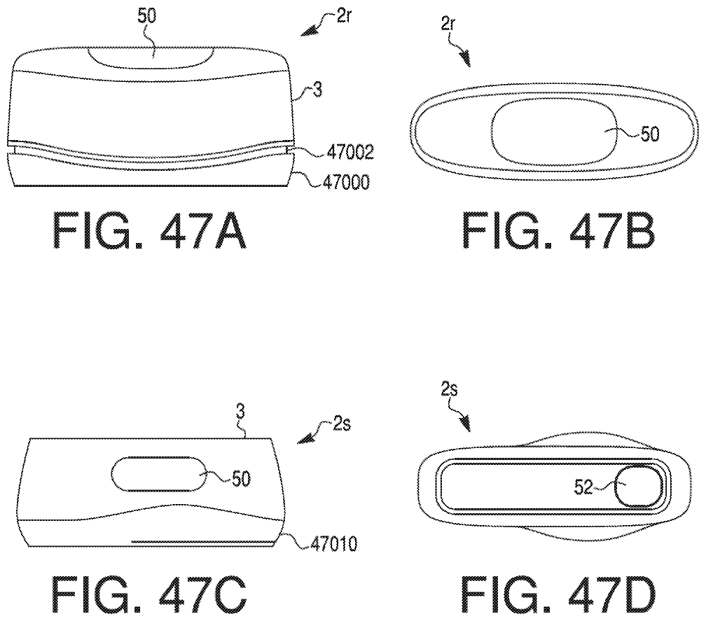

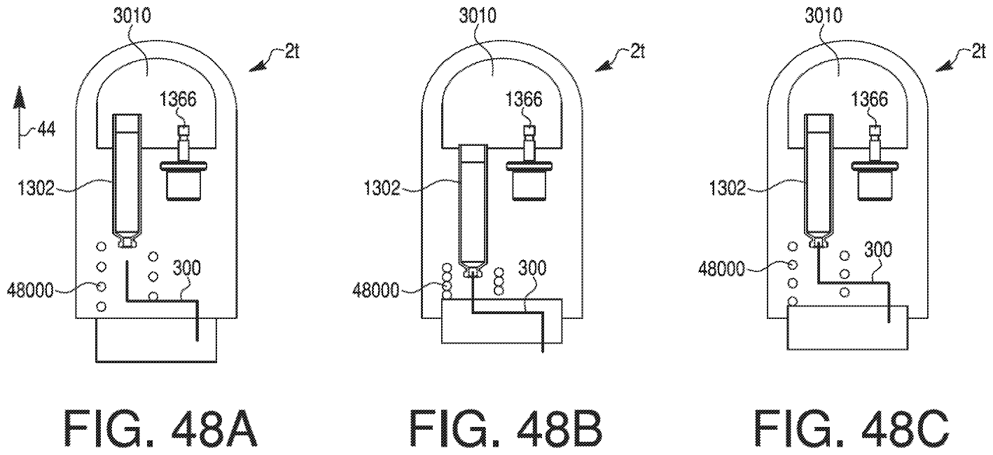

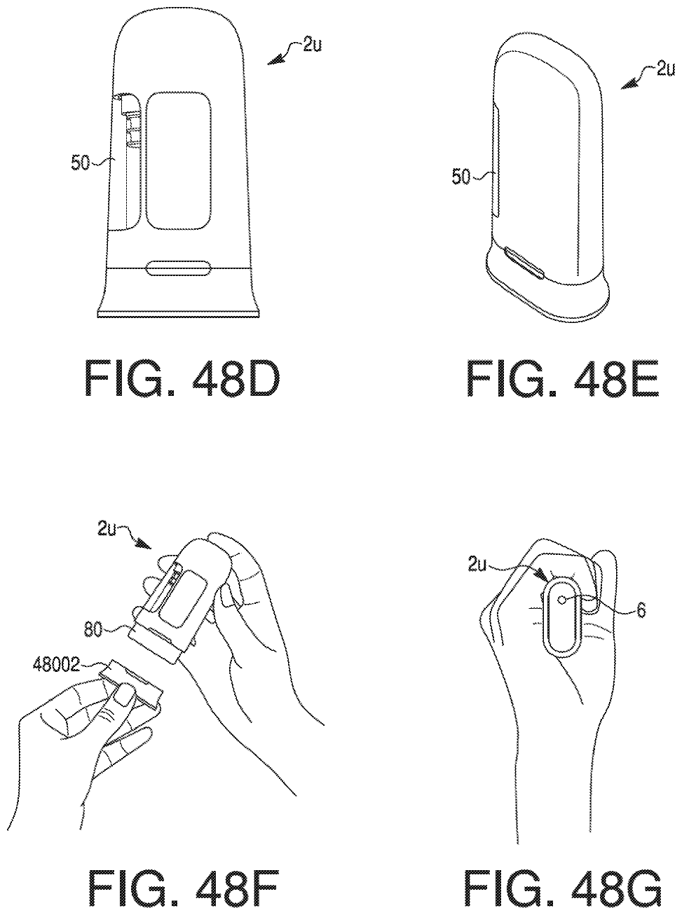

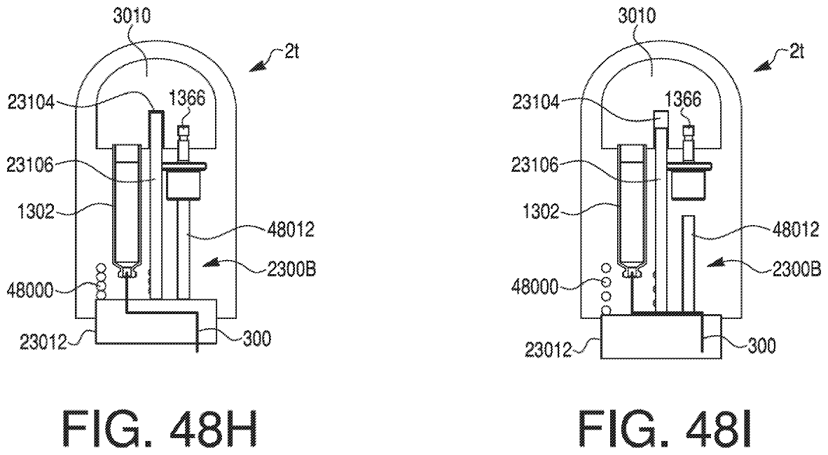

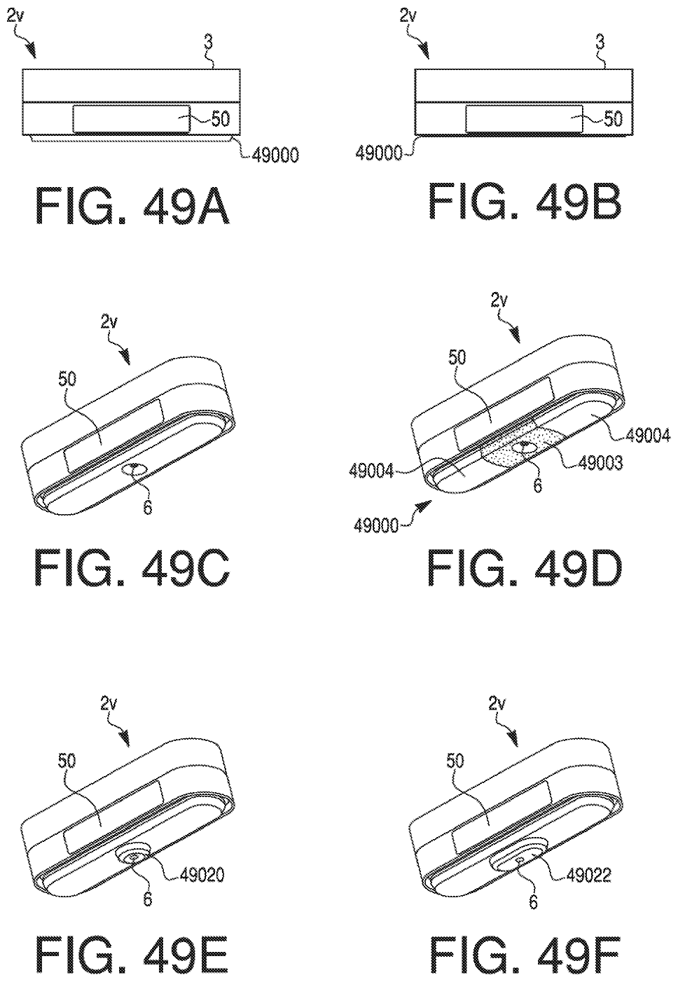

FIGS. 34A-B, 35A-B, 36A-B, 37A-B, 38A-B, 39A-B, 40A-B, 41A-E, 42A-C, 43A-D, 44A-D, and 45A-B illustrate various exemplary transverse auto-injectors of the present disclosure that may have a longer dimension along its longitudinal axis (parallel to the skin surface) than along its transverse axis (perpendicular to the skin surface). In that regard, these embodiments are similar to the auto-injectors 2 shown in FIGS. 1 and 1A described above. Furthermore, the auto-injectors shown by these figures may have a larger dimension along a lateral axis (parallel to the skin surface but perpendicular to the longitudinal axis) than along the transverse axis. Thus, these embodiments may have a “flattened” appearance against the skin surface.

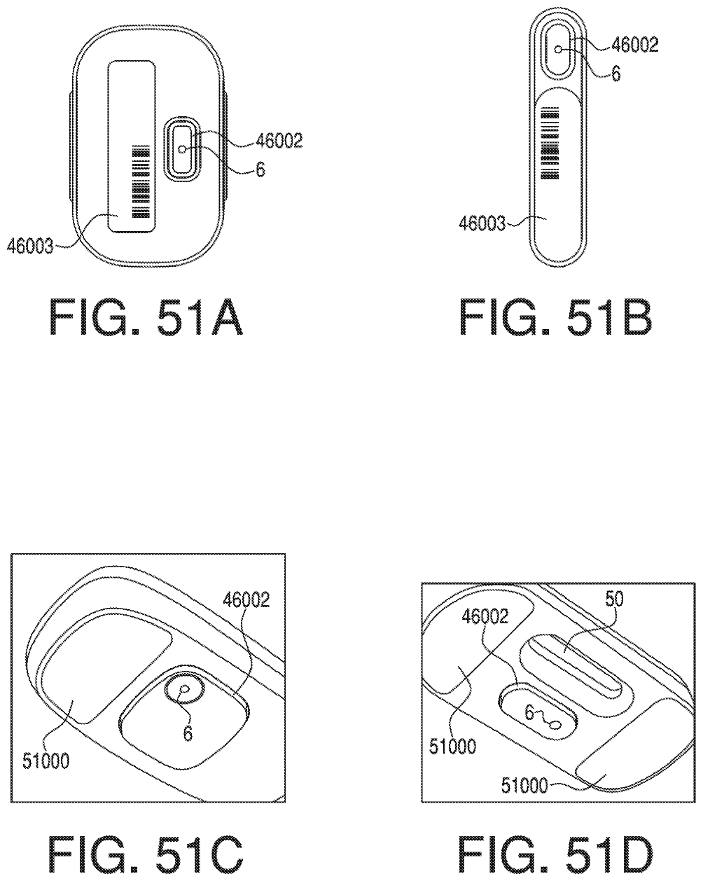

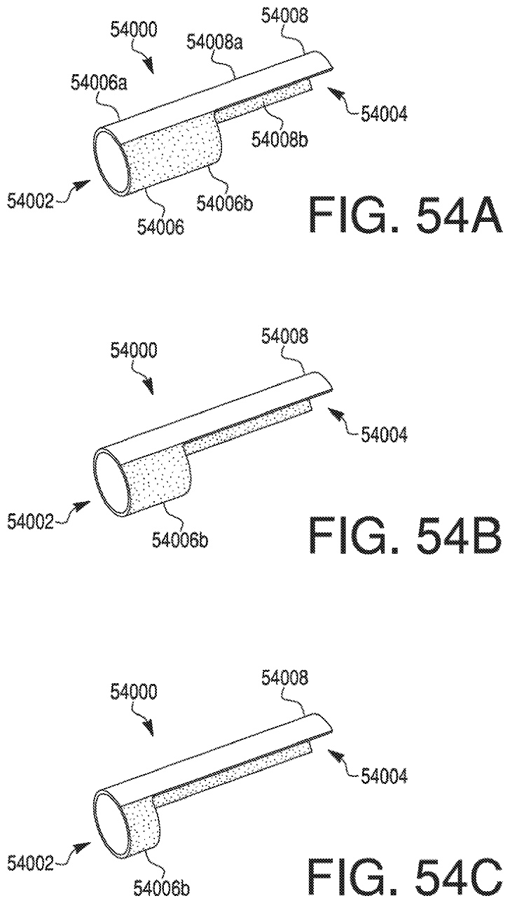

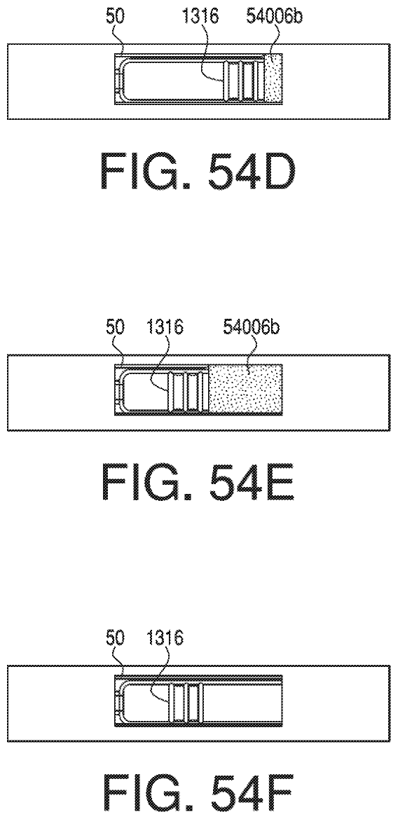

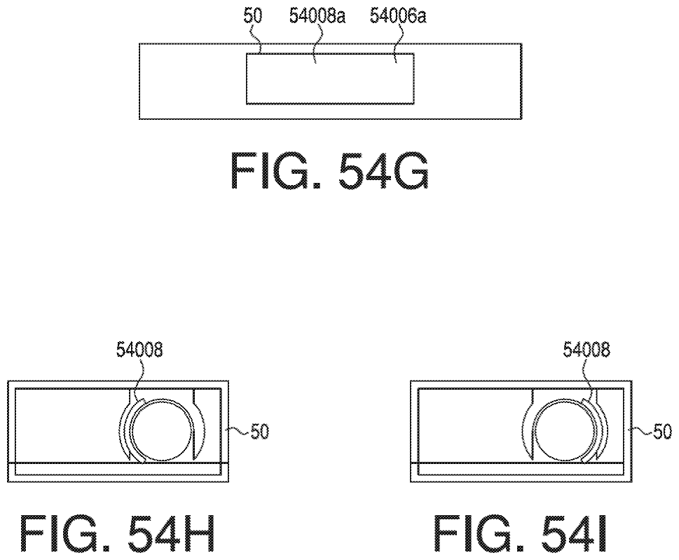

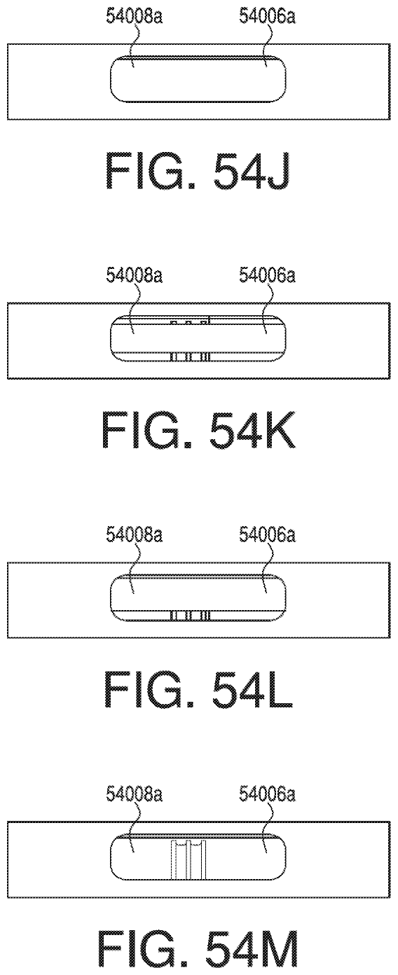

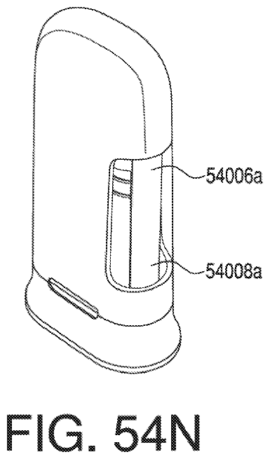

As will be illustrated in further detail below, the placement of window 50 and button 52 in the transverse auto-injectors of the present disclosure is not particularly limited. For example, windows 50 and/or buttons 52 may be positioned along top or side surfaces of housing 3, and/or may encompass the intersections of top and side surfaces, or the intersection of longitudinally-extending and laterally-extending side surfaces of housing 3. In yet other embodiments, one or more windows 50 and/or buttons 52 may be placed along a bottom, skin-contacting surface of housing 3. For example, a window 50 on a bottom surface (see FIG. 51D) may enable the interior of auto-injector 2 to be visualized when another window 50 of auto-injector 2 becomes obstructed during use of auto-injector 2 by a movable flag or the like (described in further detail below with respect to, e.g., FIGS. 54G-54I). Windows 50 and/or buttons 52 may be positioned in central and/or offset positions on a respective surface. For example, windows 50 and/or buttons 52 may be placed at a radial center of a top surface or a side surface of auto-injector 2, or may be offset longitudinally, transversely, and/or laterally from the radial center of a given surface. Windows 50 and/or buttons 52 may be recessed or raised relative to adjacent surfaces of auto-injector 2, or may be flush with the adjacent surfaces. Further details regarding the particular shape, material, appearance, size, and placement of windows 50 and buttons 52 is described in further detail below.

Button 52 may be a finger push button. In some examples, the button itself may be coupled to the needle (e.g., needle 306) being deployed into the patient, such that upon depression of the button, the needle is deployed through the user's skin. In other examples, button 52 may indirectly cause needle deployment and/or activation of fluid source 1366. For example, button 52 may trigger a spring or other force used to drive the patient needle mechanism. These examples are discussed in further detail below. Other examples of actuating mechanisms that can be used in lieu of button 52 are sliders, triggers, dials, flip lids, paddles, pull cords, or the like.

Window 50 may enable a user to clearly view container 1302 and/or piston 1316. The window 50 may be configured to help visualize different doses used with a same platform device. Window 50 may wrap around various surface of the auto-injector. Window 50 may be sized or modified to help reduce confusion when a relatively large container 1302 is used for a smaller dose (explained in further detail below). Window 52 also may be disposed on the tissue contacting surface itself, in some embodiments.

For example, in the auto-injector 2a shown in FIGS. 34A-B, housing 3 includes a platform 34000 raised relative to a remainder of the top surface of housing 3. The raised platform 34000 extends along a majority of the longitudinal axis of housing 3, and button 52 is positioned at a longitudinal end of the raised platform 34000. The top surface of button 52 may be flush with the top surface of the raised platform 34000 such that, in at least some embodiments, when the auto-injector 2a of this embodiment is viewed directly from the side, button 52 is not visible. Other configurations where button 52 is raised or recessed relative to the raised platform 34000 also are contemplated. Window 50 in this embodiment extends along a majority of the longitudinal axis of auto-injector 2a, and is visible when the auto-injector 2a is viewed from directly above and when viewed directly from the side. Window 52 is positioned within a longitudinally-extending recess in housing 3, although it also is contemplated that window 52 may be flush or raised relative to the surface of housing 3.

In the embodiment shown in FIGS. 35A-B, button 52 is positioned at a longitudinal end of a recessed top surface of auto-injector 2b. A periphery 52d of button 52 has a different visual appearance than the surrounding portions of the top surface of auto-injector 2b, and a different visual appearance the button 52. For example, periphery 52d may be a different color (i.e., the top surface and button 52 may be white, while periphery 52d is black). Alternatively, periphery 52d may include a different material such as, e.g., a clear plastic, while the top surface and button 52 are formed from an opaque plastic. In this embodiment, window 50 may extend longitudinally along a side surface of auto-injector 2b, and may be at least partially visible when auto-injector 2b is viewed directly from above and/or from the side.

In the embodiment shown in FIGS. 36A-B, button 52 may be positioned on a raised platform 36000 of auto-injector 2c in a manner similar to the embodiment of FIGS. 34A-B. However, unlike in the embodiment of FIGS. 34A-B, in the embodiment of FIGS. 36A-B, the raised platform 36000 may occupy a smaller surface area of the top surface. As shown, button 52 may occupy a substantial entirety of the raised platform 36000. Furthermore, button 52 may positioned at the radial center of the top surface. In this embodiment, window 50 may be flush with the outer surface of housing 3. Window 50 in this embodiment extends along the longitudinal axis of auto-injector 2c, and is visible when the auto-injector 2c is viewed from directly above and when viewed directly from the side.

Auto-injector 2d of FIGS. 37A-B includes a button 52 on a top surface of housing 3, and positioned within a substantial entirety of a raised platform 37000 that is at a longitudinal end of the top surface. In this embodiment, button 52 is a rocker button movable between two different positions. The sides of the rocker button 52 may be marked or colored in order to help a user determine a state of auto-injector 2d. For example, as shown in FIG. 37B, when rocker button 52 is in a first position, an exposed side 37002 of rocker button 52 may be visible to the user, and may be colored green, for example. The green color may indicate to the user that the auto-injector 2d has not been activated, and otherwise contains a dose ready for delivery to the user. After the user presses button 52, the first exposed (green) side 37002 may no longer be visible, and instead a second exposed side portion (not shown) is visible to the user. The second exposed side may have a different color or appearance than the first exposed side 37002, and is not visible while auto-injector 2d is in the first configuration. For example, the second exposed side may be the same color as a remainder of housing 3 (e.g., white), or may be another color (e.g., red, blue, etc.). Window 50 in this embodiment may be similar to any of the previously described windows, and may be visible when auto-injector 2 is viewed directly from the top or directly from the side.

In the embodiment shown in FIGS. 38A-B, button 52 is positioned at a longitudinal end of a flat or slightly rounded top surface of auto-injector 2e. Button 52 may be flush with the adjacent surfaces of housing 3, or may be slightly recessed. When this embodiment is viewed directly from the side, button 52 may not be visible. Furthermore, in this embodiment, window 50 may extend longitudinally along a side surface of auto-injector 2e, and may be at least partially visible when auto-injector 2e is viewed directly from above and/or from the side.

The embodiment shown in FIGS. 39A-B is similar to the embodiment shown in FIGS. 38A-B, with button 52 positioned at a longitudinal end of a flat or slightly rounded top surface of auto-injector 2f. As shown in FIG. 39A, button 52 is flush or recessed with the adjacent surfaces of housing 3. When this embodiment is viewed directly from the side, button 52 may not be visible. Furthermore, in this embodiment, window 50 may extend longitudinally along a recessed side surface of auto-injector 2f, and is visible only when auto-injector 2f is viewed directly from the side. In the depicted embodiment, window 50 is not visible when auto-injector 2f is viewed directly from above.

The embodiment shown in FIGS. 40A-B is similar to the embodiment shown in FIGS. 39A-B, except that button 52 is positioned at a radial center of a flat or slightly rounded top surface of auto-injector 2g. Furthermore, while a recess containing window 50 may be visible when auto-injector 2g is viewed directly from above, the window 50 itself may not be visible from that vantage point.

In the embodiment of FIGS. 41A-B, button 52 is positioned along a laterally-extending side surface of auto-injector 2h. As depicted, button 52 encompasses a substantial entirety of one laterally-extending side surface, although it is contemplated that button 52 may encompass a smaller portion of that surface. Button 52 may be raised relative to adjacent surfaces of auto-injector 2h, and, in a pre-activated or undeployed configuration, may have exposed side surfaces 41000 visible to the user. The sides 41000 of button 52 may be marked or colored in order to help a user determine a state of auto-injector 2h, as described above with respect to FIGS. 37A-B. For example, as shown in FIGS. 41A-B, when button 52 is in the pre-activated or undeployed configuration, an exposed side 41000 of button 52 may be visible to the user, and may be colored green, for example. The green color may indicate to the user that the auto-injector 2h has not been activated, and otherwise contains a dose ready for delivery to the user. After the user presses button 52, the exposed (green) side 41000 may no longer be visible, indicating that the device has been activated. Furthermore, after completion of an injection, visual inspection of button 52 will not reveal any of the previously exposed colored or marked surfaces, indicating to the viewed that the auto-injector 2h has been used. In some embodiments, button 52 may be prevented from returning to its initial position (with exposed colored or marked surfaces 41000) after being depressed, by a lock or other mechanism. Such a locking mechanism may help ensure the reliability of a visual inspection of auto-injector 2h. FIGS. 41C-E show embodiments similar to those shown in FIGS. 41A-B, but with an additional status window 50b positioned on the top surface. The status window can include any suitable information regarding the state of auto-injector 2h. In one embodiment, the status window may display the same color or appearance as the exposed side 41000 of button 52, when the auto-injector 2h is in the pre-activated or undeployed state. After depression of button 52, window 50b may display a different color or appearance to indicate that auto-injector 2h has been activated. In one embodiment, window 50b may display a same color or appearance as a remainder of button 52 or of housing 3 to indicate that auto-injector 2 has been used. Additional details on the types of images and marks that may be displayed in window 50b are discussed below.

The embodiment shown in FIGS. 42A-B is similar to the embodiment shown in FIGS. 39A-B, except that button 52 may be visible when auto-injector 2i is viewed directly from the side, due to a curvature of the top surface of auto-injector 2i. Additionally, window 50 may be visible when auto-injector 2i is view either directly from above or directly from the side.

FIG. 42C shows auto-injector 2j with a button 52 disposed on the top surface of the auto-injector 2j, and with a window 50 extending along both the top surface and an adjacent longitudinally-extending side surface. In auto-injector 2j, window 50 and button 52 may be adjacent to one another on the top surface of housing 3.

In the embodiment shown in FIGS. 43A-D, button 52 may be positioned on a longitudinally-extending side surface of auto-injector 2k. Button 52 may be a rocker button movable between two positions. At least a portion, or an entirety, of button 52 may have a different color or otherwise a different physical appearance than housing 3. Button 52 may be visible when auto-injector 2k is viewed directly from above or directly from the side. In this embodiment, window 50 may be positioned in a recess of the top surface of auto-injector 2k such that window 50 is visible when the auto-injector 2k is viewed from directly above, but not when viewed directly from the side.

The auto-injector 2l shown in FIGS. 44A-B includes two longitudinally-extending buttons 52—one on each longitudinally-extending side surface of the auto-injector 2l. A user may be required to depress both of the two buttons 52 in order to initiate deployment of a needle, and dispensation of medicament. For example, one of the buttons 52 may be coupled to a locking mechanism blocking some portion of the patient needle mechanism, while another portion of the locking mechanism may be configured to activate fluid source 1366. In some embodiments, the two buttons 52 may be required to be pressed simultaneously or in a particular sequence in order to initiate needle deployment. A longitudinally-extending window 50 may be disposed on the top surface of the auto-injector.

FIGS. 44C-D show an auto-injector 2m with a slider 44000 positioned in a recessed top surface. Slider 44000 may be movable from a first position to a second position. Auto-injector 2 may be pre-activated or undeployed when slider 44000 is in the first position, and movement of slider 44000 to the second position may initiate needle deployment and medicament dispensation. In the first position, a first color, mark, or appearance on an indicator panel 44002 may be displayed by slider 44000 (e.g., underneath the sliding component itself). For example, a green or other color may be visible to the user to indicate pre-activated or undeployed status of the auto-injector. Once slider 44000 is moved to the second position, a second color, mark, or appearance (different than the first color, mark, or appearance) may be displayed by slider 44000 on a second indicator panel, to provide a visual indication that the auto-injector 2m has been previously used. In the second position, the first indicator panel 44002 is covered by the sliding component of slider 44000 and is not visible. The window 50 of this embodiment may be substantially similar to the window 50 described above with respect to FIGS. 35A-B.

FIGS. 45A-B show an auto-injector 2n with a button 52 on a top surface of the auto-injector 2 that may be a snap-click button. In a pre-activated or undeployed configuration of auto-injector 2, button 52 may have exposed side surfaces 45000 having a color, mark, or appearance visible to the user to indicate pre-activated or undeployed status of the auto-injector 2n. Once button 52 is pressed and moved to the second position, the first color, mark, or appearance on exposed side surface 45000 is no longer visible to the user from any exterior viewing angle, thus indicating that auto-injector 2n has been used. After being pressed, button 52 may snap or click into a second position. Button 52 may encompass a majority or even substantial entirety of the top surface of the auto-injector 2. Furthermore, window 50 may be disposed on button 52 itself.

FIGS. 46A-B show a transverse auto-injector 2o having a greater dimension along a transverse axis 44 (perpendicular to the skin surface) than along a lateral axis 42 parallel to the skin surface. Transverse auto-injector 2o still may have a longest dimension along a longitudinal axis 40 that is parallel to the skin surface, and in such embodiments a container 1302 within transverse auto-injector 2o may be oriented substantially parallel to the skin surface and to a longitudinal axis of the transverse auto-injector 2o. In order to accommodate all of the functionality required, the valve (e.g., valve 3010) described herein may be placed closer to the skin-contacting surface of auto-injector 2o. Container 1302 may extend along the longitudinal axis 44 of auto-injector 2o, and may be positioned above the valve 3010. Auto-injector 2o may include a removable seal 46000 positioned on a portion or an entirety of the skin-contacting surface of auto-injector 2o. In some embodiments, seal 46000 may be permeable to a sterilant (such as, e.g., ethylene oxide or vaporized hydrogen peroxide) and placed on auto-injector 2o before sterilization. Seal 46000 may include Tyvek, or another suitable material. It is contemplated that any of the auto-injectors disclosed herein may include a removable seal (like seal 46000) covering a portion or an entirety of a bottom, skin-contacting surface of the respective auto-injector.

FIGS. 46C-E show an embodiment of an auto-injector 2p having a button 52 disposed on a top surface of the auto-injector at a longitudinal end of the top surface. Window 50 may extend longitudinally along the top surface adjacent to button 52. Window 50 also may extend to each longitudinally-extending side surface of auto-injector 2p. FIG. 46E shows a bottom, tissue-engaging surface 46001 of auto-injector 2p. The tissue-engaging surface 46001 may include a label 46003 comprising various identifying information. More details regarding the label with be discussed below. Auto-injector 2p also may include a contact detection switch 46002 at a longitudinal end of the tissue-engaging surface 46001. Depression of the contact switch 46002 may be required for needle deployment. In some cases, depression of the contact switch 46002 may move a mechanical impediment out of the path one of or more structures within auto-injector 2p, such as, out of the path of a shuttle, needle driver, gear, or other movable portion of the patient needle mechanism. For example, depression of the contact switch may move an impediment out of the path of one or more portions of the patient needle mechanism. The contact switch 46002 may have a hollow interior (may be ring-shaped) so that needle 306 may pass through opening 6 of the tissue-contacting surface 46001 and through the hollow interior of the switch 46002.

FIGS. 47A-47B shown an auto-injector 2r utilizing a shroud 47000 for needle deployment and device activation. The shroud 47000 may extend from the housing 3 of the auto-injector 2r and operate in the same manner as described above with respect to FIGS. 33A-B. The auto-injector 2r of FIGS. 47A-47B may include a window 50 that extends longitudinally along the top surface of the auto-injector 2r, but that, because of a downward curvature of the top surface, may be visible from both the top and side of the auto-injector 2r. Furthermore, while auto-injector 2r is in a pre-activated and undeployed state, an exposed portion 47002 of shroud 47000 may be visible to the user when auto-injector 2r is viewed from the side. The exposed portion 47002 may have a different color (e.g., green), mark, or appearance, than a remainder of the auto-injector 2r (which may be white, for example). The previously-exposed portion 47002 and color may not be visible once the auto-injector 2r has been activated (with shroud 47000 retracted). Retraction of shroud 47000 may directly or indirectly insert needle 306 (referring to, e.g., FIG. 18A). For example, needle 306 may be coupled to housing 3 such that relative movement of shroud 47000 and housing 3 causes needle 306 to be inserted into the user (direct insertion). In other examples, retraction of shroud 47000 may initiate another mechanism, such as, e.g., a fluid source, a spring, or other mechanism to drive needle insertion (indirect insertion).

FIGS. 47C-47D show an auto-injector 2s that, like auto-injector 2o, has a greater dimension along a transverse axis (perpendicular to the skin surface) than along a lateral axis parallel to the skin surface. Button 52 may be disposed in a recessed top surface of housing 3, and may not be visible when auto-injector 2s is viewed directly from the side. Window 50 may extend along a longitudinally-extending side surface of housing 3, and may not be visible when auto-injector 2 is viewed from directly above. A bottom portion 47010 may comprise a grippy or tacky coating, such as, e.g., rubber, in order to facilitate grip of auto-injector 2s by a user, and also to help prevent slipping of auto-injector 2s on the skin. The grip may cover a majority or entirety of a bottom, tissue-engaging surface of auto-injector 2s, and also may extend upwardly from the tissue-engaging surface along the lateral and longitudinal side surfaces of auto-injector 2s.

FIGS. 48A-C are schematic illustrations of a “vertical” auto-injector 2t having a longest dimension along the transverse axis that is perpendicular to the skin surface. Auto-injector 2t may include the same or similar components as any of the previously-described auto-injectors. For example, fluid from fluid source 1366 may move container 1302 relative to a stationary housing 3 and fluid conduit 300, to put container 1302 into fluid communication with fluid conduit 300. A spring 48000 may be coupled to second end 1306 of container 1302, and may be in an expanded state before the auto-injector 2t is activated (FIG. 48A). As container 1302 is moved onto fluid conduit 300, spring 48000 may be compressed (FIG. 48B). Needle 306 of fluid conduit 300 also may be deployed using any of the mechanisms described herein (see FIG. 48B). After completion of the injection, fluid/gas from fluid source 1366 may be vented instead of being routed to container 1302. At this point, with the pressure of fluid from fluid source 1366 no longer acting against the spring 48000, spring 48000 may expand and urge both container 1302 and fluid conduit 300 away from the skin surface (i.e., retraction of needle 306). Fluid source 1366 could be activated by a button or any of the activation mechanisms described herein. It is also contemplated that auto-injector 2t may include a shroud, and that activation of fluid source 1366 and deployment of needle 306 into the user is caused by applying a pressure to the auto-injector 2t against the skin to retract the shroud. FIGS. 48D-F show a vertical auto-injector 2u having a window 50 extending along a transverse axis of the auto-injector. Auto-injector 2u also may include a removable cap 48002 (see FIGS. 48D-E), which, when removed, exposes a shroud 80 containing a needle opening 6.

FIGS. 48H and 48I illustrate additional features of the system flow within auto-injector 2t, which may be substantially similar to the system flow shown in FIG. 3A. This embodiment also may include vent or push system 2300 used divert gas that otherwise would be vented out of the auto-injector, to be used in assisting pushing a shroud 23102 away from the remainder of auto-injector 2t, after delivery of a medicament dose.

As discussed above, retraction of shroud 23102 may initiate gas canister 1366. For example, shroud 23102 may be coupled to an initiation rod 48012. When shroud 23102 is retracted, initiation rod 48012 activates gas canister 1366 in a manner similar as to other gas canister activation mechanisms described herein. Then, gas flows through the system and the valve, urging medicament through the fluid conduit and patient needle 300 that is now inserted through the patient as shown in FIG. 48H.

There is a further conduit or connection 23104 between shroud 48010 and the gas can/vent line. While in the high pressure state, where diaphragm 3012 is sealing the valve seat 3020, gas is prevented from flowing through conduit 23104. When the pressure equilibrates in the system and valve, and the diaphragm lifts off of the valve seat 3020, gas flowing through the vent conduit 3018 urges the dump valve of push system 2300 into a configuration which allows gas from the canister 1366 to flow through conduit 23104. The force of gas flowing through conduit 23104 then urges and/or pushes shroud 48010, via push rod 23106, to a position where needle 300 is in a retracted state, as shown in FIG. 48C and FIG. 48I. In particular, with reference to FIGS. 48H and 48I, piston or push rod 23106 may be coupled to shroud 48010. Push rod 48014 may be received in conduit 23104 of auto-injector 2t, and upon discharge of vent pressure within conduit 23104, push rod 23106 may urge shroud 23102 to the configuration shown in FIGS. 48C and 48I. Moving the shroud 23102 to the configuration shown in FIGS. 48C and 48I may serve as an indication to the user that the injection is complete and also may serve as a preventative measure against accidentally injury caused by the patient end of the needle (i.e., sharps mitigation or prevention).

FIGS. 49A-F illustrate various examples of auto-injectors 2v having a shroud. In some examples, such as in FIGS. 49A-D, the shroud 49000 may comprise a substantial entirety of the skin-contacting surface of the auto-injector 2v. In the embodiment of FIG. 49D, shroud 49000 may include sections having different colors to help a user identify an approximate location of needle opening 6. In FIG. 49D, the needle opening 6 may be disposed at the radial and longitudinal center of the tissue-contacting surface of the shroud. A central portion 49003 of the shroud may have a different color, marking, or appearance, than adjacent portions 49004 of the shroud, in order to help a user visualize the approximate location of needle deployment without the needle opening 6 being in the user's direct line of sight. In another embodiment, central portion 49003 may be movable relative to adjacent portions 49004, and may retract within the auto-injector 2v to deploy a patient needle. FIGS. 49E-F illustrate embodiments where the movable piece encompasses only a portion of the tissue-contacting surface of the auto-injector 2v. For example, shroud 49000 may include a circular protrusion 49020 (FIG. 49E) or ovular protrusion 49022 (FIG. 49F) that retracts into the auto-injector 2v when placed against the skin with pressure applied to the auto-injector 2v. It is also contemplated that any other shaped protrusion may be utilized. The protrusions 49020 or 49022 shown in FIGS. 49E-F may have a different color, mark, or appearance than a remaining portion of the tissue-contacting surface of the auto-injector 2v. The embodiments of FIGS. 49A-F may help mitigate a fear of needles of a user, since the user can be confident of a relatively short needle length when visually inspecting the respective auto-injectors.

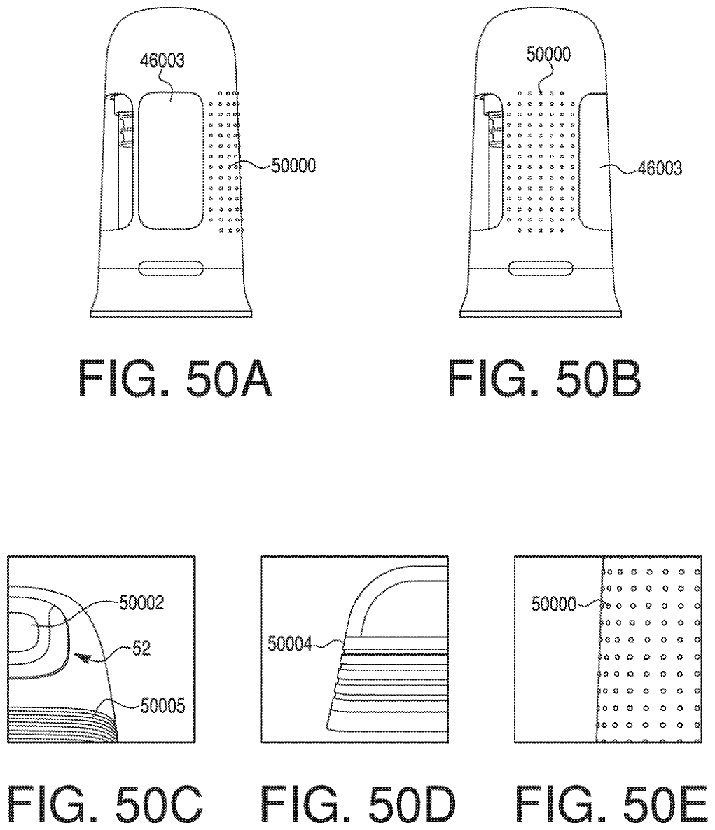

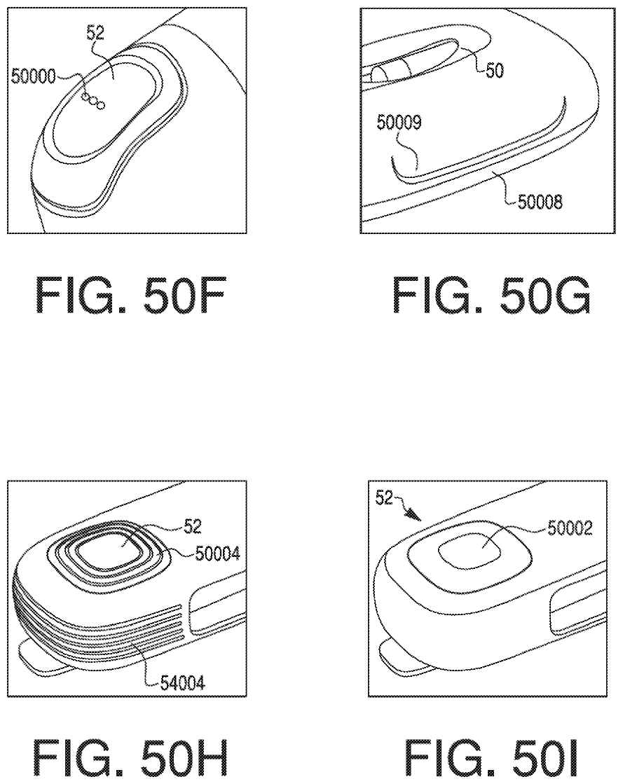

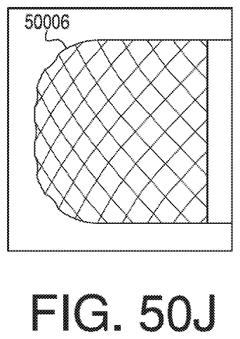

Various surfaces of the auto-injectors disclosed herein may be modified to assist users during operation of the auto-injectors. For example, on buttons 52, one or more bumps 50000 (FIG. 50F), divots 50002 (FIGS. 50C, 50I), or ribs 50004 (FIG. 50H) may be used to provide a clear indication to the user that button 52 is the button used to activate the auto-injector, and also to provide clarity to the user that the user is handling the top surface of the auto-injector. The surface features also help guide a user's fingers to the button itself, and assist with grip on the button. Furthermore, at least the divots may provide a more comfortable user experience when pressing button 52. Various surface modifications also may be applied to other portions of the outer surface of the auto-injectors described herein. For example, surfaces of housing 3 may include one or more bumps 50000 (FIGS. 50A, 50B, and 50E), raised ribs 50005 (FIG. 50C), recessed ribs 50004 (FIGS. 50D and 50H), tacky or rubber surfaces 50008 (FIG. 50G), recesses 50009 (FIG. 50G), and/or knurling 50006 (FIG. 50J). The surface modifications may be positioned around the various auto-injectors where it is intended for a user to hold/grip the auto-injector. The surface modifications may be placed along one or more of the top surface, laterally-extending side surface, or longitudinally-extending side surfaces of the disclosed auto-injectors.

FIGS. 51A-51D show various needle positions relative to the tissue-contacting surfaces of the disclosed auto-injectors. For example, needle openings 6 may be centered (e.g., along one or more of the lateral or longitudinal axes of the auto-injector), or offset from one or more of the lateral or longitudinal axes. In some embodiments, the needle opening 6 may extend through a movable shroud of the auto-injector (FIGS. 51C and 51D) and may be centered relative to the movable shroud, or offset from one or more axes of the shroud (as shown in FIGS. 51C-D). As illustrated in FIGS. 51A-B, the needle opening may be disposed within the hollow interior of a ring-shaped contact switch, such that needle 306 must pass through the interior of the contact switch during deployment into the patient. In other embodiments, the contact switch 46002 may be a solid button through which the needle opening 6 extends (FIGS. 51C-D). In yet other embodiments, the needle opening 6 may be offset from the contact switch 46002. In various embodiments, the contact switch 46002 may include a grippy or rubber material, and/or surface textures (such as ribbing), to facilitate contact with skin and to prevent slipping.

In some embodiments, the skin contacting surface of the disclosed auto-injectors may include one or more grippy or tacky surfaces to assist with securing the auto-injector to the skin during use. For example, referring to FIGS. 51C-D, one or more grips 51000, e.g., rubber grips, may be positioned on the skin-contacting surface of an auto-injector.

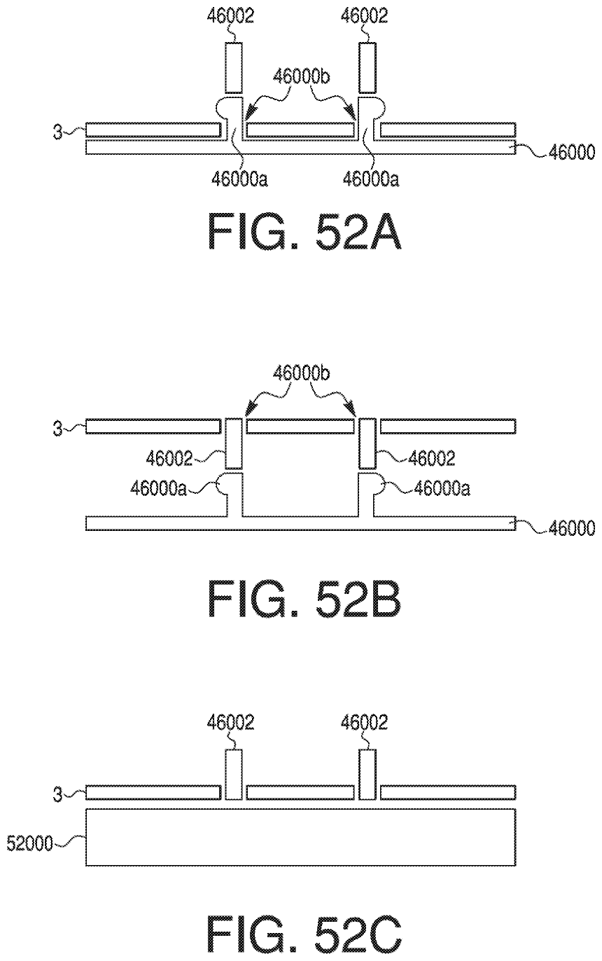

Referring to FIGS. 52A-52C, various auto-injectors of the present disclosure may include a pull tab or seal 46000, as previously discussed with reference to FIGS. 46A-B. Seal 46000 may include one or more protrusions 46000a configured to extend into one or more openings 46000b of housing 3. While protrusions 46000a are disposed in openings 46000b, auto-injector 2 may be sterilized by exposure to a sterilant that is permeable through seal 46000 (e.g., EtO or VHP). Opening 46000b may be a same opening that the contact switch 46002, (described above with respect to FIGS. 46C-E), extends out of housing 3. Contact switch 46002 may be biased to extend outside of housing 3 via opening 46000b, but is maintained entirely within housing 3 while protrusions 46000a extend through the openings 46000b. While contact switch 46002 is held within housing 3 and while protrusion 46000a is disposed through opening 46000b, the auto-injector is not capable of deploying needle 306 or initiating injection. That is, in some embodiments, removal of seal 46000 is a necessary step that must occur before needle deployment. Thus, depression of button 52, for example, while protrusion 46000a is extended through opening 46000b, will not deploy needle 306 or otherwise start any injection. For example, an impediment may be coupled to contact switch 46002, and the impediment may block a path of one or more portions of the patient needle mechanism such as, e.g., a needle driver, shuttle, gear, or the like. As seal 46000 is removed from housing 3, contact switch 46002 may extend through opening 46000b and out of housing 3 (FIG. 52B). Once contact switch 46002 is extended outside of housing 3, it may operate as described above with respect to FIGS. 46C-E, such that upon contact with the skin (FIG. 52C), depression of contact switch 46002 readies the auto-injector for activation. For example, while contact switch 46002 is pressed, and only when pressed, will activation of button 52 initiate deployment of needle 306. Furthermore the presence of seal 46000 on an auto-injector may serve as a clear visual indicator that the auto-injector has not been used, or has not been tampered with.

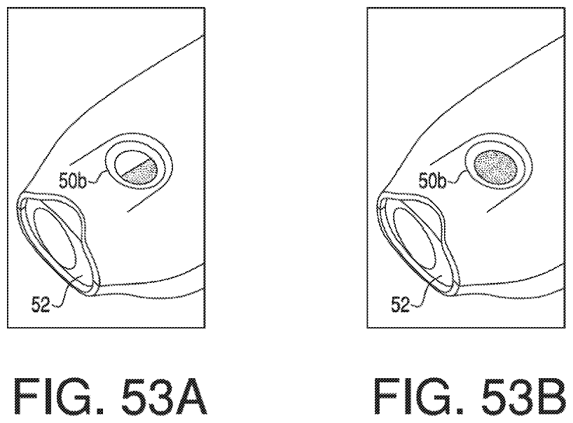

FIGS. 53A-B show further examples of a status indicator 50b configured to help a user or observer visually determine a state of the device. For example, the indicator 50b may display a first indication, e.g., a first color, mark, or appearance, when the device is in a pre-activated and undeployed condition. The indicator 50b may display a second color, mark, or appearance, after completion of the injection and retraction of the needle 306. For example, the second color may be “Green” or the indicator may display a textual or symbol reference, such as, e.g., “END” or a checkmark to indicate completion of injection. The indicator 50b also may include one or more other colors, marks, or appearances to indicate other statuses. For example, one color may be displayed when seal 46000 is attached to an auto-injector, and another color may be displayed after removal of seal 46000 from an auto-injector. Yet another different color may be displayed when contact switch 46002 has been pressed but before injection has started. It also is contemplated that the indicator 50b can show real-time progress of an injection. For example, in a transition from a first color to a second color, the indicator 50b may gradually decrease the area of the indicator window occupied by the first color, while gradually increasing the area of the indicator window occupied by the second color. This transition may continue until the end of the injection, at which point the indicator window shows only the second color, and none of the first color. The change in indicator status may be triggered by depression of button 52 as set forth above. The change in indicator status also may be triggered by gas from the valve. For example, a portion of the gas from fluid source 1366 may be diverted to move an indicator from a first position to a second position. In one embodiment, the movement of push rod 8002 (driven by vented gas) may be used to urge the indicator from the first position to the second position. The indicator 50b may be calibrated relative to the anticipated time of the injection in order to show the gradual progress as set forth above. Or, the diverted gas may simply trigger the conversion of a binary indicator from a first state (indicating pre-activation) to a second state (indicating completion).

FIGS. 54A-54C show various status flag indicators 54000 that may be used in conjunction with the disclosed auto-injectors to help an observer visually determine the state of a given auto-injector. The flags 54000 may be partially tubular structures extending from a first end 54002 toward a second end 54004. The first end 54002 of the structure may include a substantially tubular portion 54006 that extends around an entirety of a circumference of the flag 54000. The second end 54004 of the structure may include a partially tubular portion 54008 that extends around only a portion of the circumference of the flag 54000. It is contemplated that the partially tubular portion 54008 may extend around an arc length of about 180 degrees around a radial center of the flag 54000. The radially outer surfaces 54008a of the partially tubular member 54008 may be a first color, and the radially outer surfaces 54006a of the substantially tubular member 54006, extending around the same arc as the partially tubular member 54008, may also be the first color. When visible from a window 50, the first color of surfaces 54006a and 54008a may indicate that the injection is complete (or in progress). The inner surfaces 54008b of the partially tubular member 54008 may be a second color that is different than the first color. Furthermore, the outer surfaces 54006b of the substantially tubular member 54006, that do not share the same arc as the partially tubular member 54008, may also be the second color. The second color may help provide a contrast against which the contents of container 1302 can be viewed and inspected. The inner surfaces 54008b of the partially tubular member 54008 and the outer surface 54006b of the substantially tubular member 54006 may be visible from a window 50 of the auto-injector at the same time. The indicator may be opaque, translucent, or frosted.

Before activation of the auto-injector, only the second color of outer surface 54006b or of outer surface 54008b may be visible through the window 50. As medicament is delivered through container 1302, the flag 54000 may rotate about the container 1302 to gradually reveal the first color through window 50 as the injection progresses, until the injection is complete. Upon completion of the injection, it is contemplated that only the first color will be visible to the user through a window 50 (e.g., only the outer surfaces 54008a of the partially tubular member 54009, and the outer surfaces 54006a of the substantially tubular member 54006 may be visible. It is contemplated that rotation of the indicator may be gradual, so as to provide a real-time indication of the progress of the injection. In other embodiments, flag 54000 may act as a binary indicator, and may not rotate until after injection is completed. When used as a binary indicator, rotation may be driven by gas vented from fluid source 1366, via, e.g., vent 3018. FIGS. 54F-I illustrate an example of a binary indicator. For example, while injection is in progress (FIGS. 54F and 54H), the flag 54000 is in its initial position. However, once injection is complete (FIGS. 54G and 54I), the flag 54000 is rotated to occupy the entire viewing area of window 50. FIGS. 54H and 54I show the position of partially tubular member 54008 relative to window 50 before activation (FIG. 54H) and at completion of the injection (FIG. 54I).

The length of the substantially tubular portion 54006 may be adjusted to accommodate different doses set for container 1302. For example, the same model and type of auto-injector 2 and container 1302 may be used to deliver different doses of medicament. For smaller doses, a same type container 1302 (e.g., with the same specifications) may still be used, but may be filled with medicament to a lesser capacity. Thus, there may be a volume of unused space behind piston 1316 moving toward first end 1304 of container 1302. This unused and empty space, along with the positioning of piston 1316 toward the middle of container 1302, before injection, may lead to user confusion. For example, at the initiation of injection, a user may be confused when visualizing piston 1316 in the center of container 1302 and window 50. For example, the user may be led to believe that the device was activated, was improperly filled, or may contain some other defect. The length of the substantially tubular portion 54006 of the flag 54000 may help reduce user confusion. Or, certain portions of window 50 or of container 1302 may be frosted or painted to cover or otherwise indicate the unused space in container 1302. Containers 1302 with larger doses may have relatively little unused space, and may be used with a flag 54000 having a relatively short substantially tubular portion 54006 (e.g., FIGS. 54C and 54D). Containers 1302 with smaller doses may have more unused space, and may be used with an indicator having a relatively longer substantially tubular portion 54006 (which blocks the user's view of the unused space before injection is initiated—see FIGS. 54A and 54E).

The flag 54000 may partially or completely occupy the viewing window 50. For example, window 50 is completely occupied by the indicator in FIGS. 54J and 54M, but only partially occupies the viewing window in FIGS. 54K, 54L, and 54N. In FIG. 54M, flag 54000 may be slightly transparent to enable a portion of piston 1316 to be visible through the flag 54000.

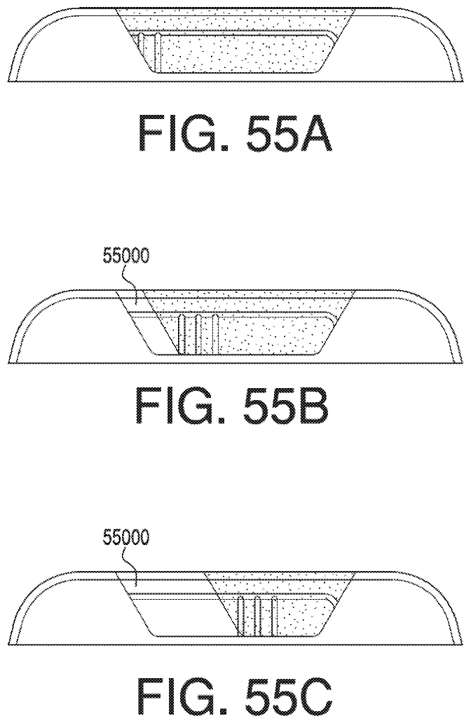

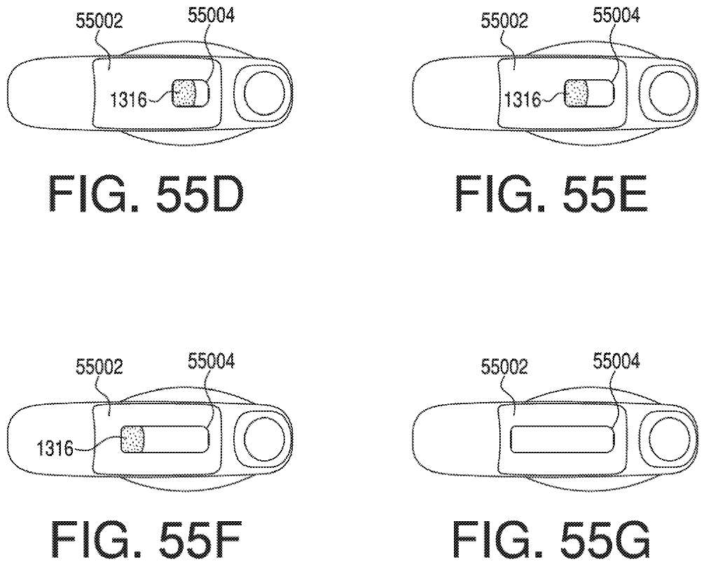

Window 50 also can be tinted or covered for different doses in container 1302. For example, referring to FIGS. 55A-55C, different levels of tint 55000 may be used to distinguish auto-injectors configured for different doses. In particular, for a first dose, e.g., a maximum dose, shown in FIG. 55A, window 50 may not contain any tint. For a smaller doses than the maximum dose shown in FIG. 55A, window 50 may be tinted so as to cover the unused space at the first end 1304 of the container 1302. Alternatively, instead of a tint, a cover piece 55002 may be used to cover the unused space for different doses. For example, cover piece 55002 may be configured to cover longer lengths of window 50 for smaller doses, while exposing more of window 50 for larger doses contained in container 1302. FIG. 55G shows a relatively large dose and FIG. 55D shows a relatively small dose in container 1302. In FIG. 55G, substantially all of window 50 is visible, and indeed, piston 1316 may not be visible at all. Alternatively, in FIG. 55D, cover piece 55002 covers a larger proportion of window 50 (than in FIG. 55G). FIGS. 55E-F show intermediate doses between those shown in FIGS. 55D and 55G. In alternative embodiments, a cover piece may be placed directly around container 1302 itself (within the auto-injector) as opposed to over an outer surface of the auto-injector as shown.

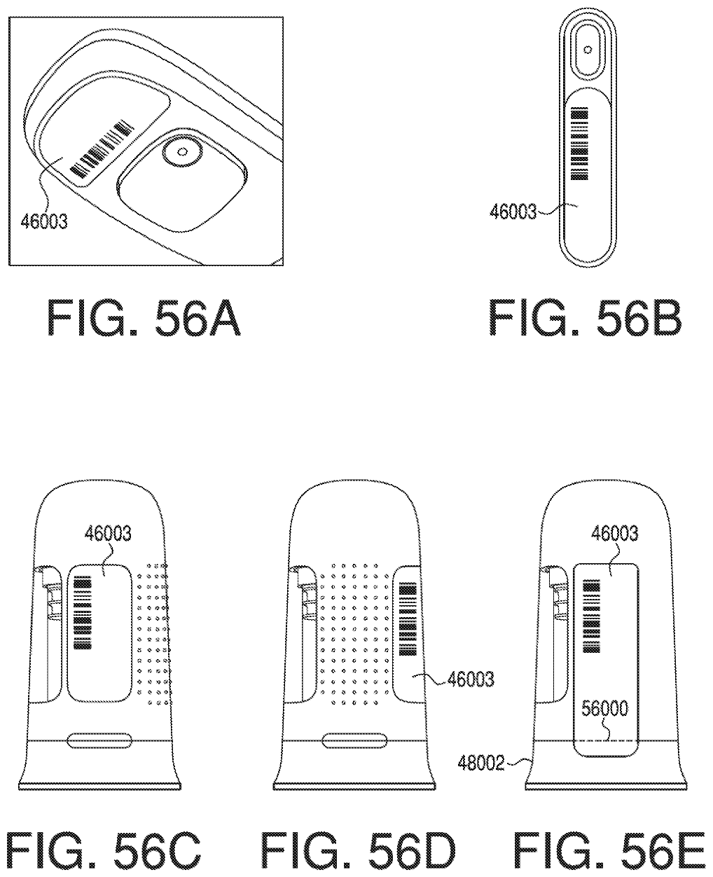

FIGS. 56A-E show various locations for labels 46003 on the outer surface of an auto-injector. For example, a label 46003 may be positioned on a bottom, skin-contacting surface of the auto-injector (FIGS. 56A-B). Or, labels 46003 may be placed on a side surface of the auto-injector (FIGS. 56C-E). In some embodiments, the label 46003 may be positioned on both an outer surface of housing 3, and onto a removable cap. A perforation 56000 may be disposed on the label at the intersection of the cap 48002 and housing 3. Perforation 56000 may serve as yet an additional indicator to the user that the device has not been tampered with. Upon removal of the cap 48002 from housing 3, the perforation 56000 is broken. In other embodiments, labels 46003 or identifying information may be placed on the top surface of the auto-injector.

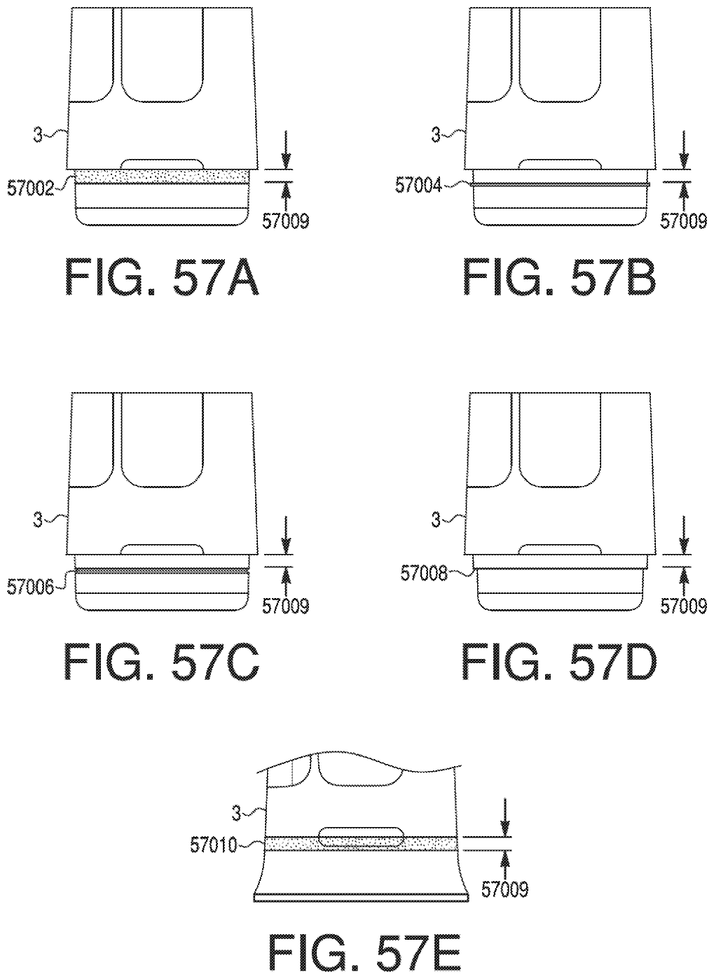

FIGS. 57A-D show various features for visually indicating an approximate length 57009 of needle 306 that will be inserted into the patient. For example, colored band 57002 (FIG. 57A), a protruding rib 57004 (FIG. 57B), a recess 57006 (FIG. 57C), or an offset step 57008 (FIG. 57D) may be incorporated into a shroud 80 to indicate to the approximate length 57009 of needle 306 that will penetrate the skin. In particular, the injection length of needle 306 may correspond to or may be substantially equal to the distance from the features described in FIGS. 57A-57D to the end of housing 3 from which shroud 80 extends. FIG. 57E shows an embodiment with a removable cap, where a colored band 57010 is disposed around a circumference of the cap. The width of the colored band may provide a visual cue to the user representative of the penetration length 57009 of needle 306. This feature may be particularly effective with vertically-oriented auto-injectors, which commonly invoke a greater sense of anxiety in patients, as patients associate the longer transverse height dimension with a longer needle.

FIGS. 58A-H illustrate additional features that may be incorporated into auto-injector 2. As shown in FIG. 58A, auto-injector 2 may include a status window 58000, which may be positioned on an outer face of housing 3 of auto-injector 2 similar to any of the windows described herein. Status window 58000 is shown as circular, but could be any suitable shape such as, e.g., ovular, rectangular, square, irregular or the like. As shown in greater detail in FIGS. 58B-58H, a status indicator 58002 may be moveable relative to status window 58000 in order to display different states, stages, portions, etc., of an injection. Additionally, status indicator 58002 may include one or more of the features discussed herein, for example, as discussed with respect to FIGS. 53A-53B. Still further, the position of status window 58000 is not limited, and in some embodiments, status window 58000 may be positioned closer to button 52.

As shown in FIG. 58B, status indicator 58002 may include one or more status panels, for example, a first status panel 58002a, a second status panel 58002b, and a third status panel 58002c, which may be arranged substantially longitudinally along a length of status indicator 58002. Each status panel 58002a, 58002b, 58002c may include a different color, mark, pattern, appearance, etc. in order to convey the current status of auto-injector 2 to a user when the respective status panel is aligned with status window 58000. In one aspect, first status panel 58002a may be a first color (e.g., white), a first pattern, or include a first indicator, such as, e.g., a textual or symbol reference (e.g., “Go” or “Ready”). Second status panel 58002b may be a second color different than the first color (e.g., blue), a second pattern different than the first pattern, or a second indicator different than the first indicator (e.g., “In progress”), and third status panel 58002c may be a third color (e.g., green), a third pattern, or a third indicator (e.g., “End”). The third color may be different than the first color and the second color. The third pattern may be different than the first pattern or the second pattern. The third indicator may be different than the first indicator and the second indicator. Additionally, first status panel 58002a may correspond to an initial or unused state for auto-injector 2. Second status panel 58002b may correspond to an active or in-progress state for auto-injector 2, and third status panel 58002c may correspond to a complete or used state for auto-injector 2. Accordingly, the status panel that corresponds to a complete or used state (third status panel 58002c) may be positioned between the status panel that corresponds to an initial or unused state (first status panel 58002a) and the status panel that corresponds to an active or in-progress state (second status panel 58002b). In this manner, status indicator 58002 may move relative to window 58000 via shuttle 58014 (substantially similar to the shuttles discussed herein, including for example, shuttle 340). Although not shown, status indicator 58002 may include four or more additional status panels, which may correspond to additional states, stages, portions, etc. of an injection process. It is further contemplated that each status panel may utilize a combination of color, pattern, and/or indicator, for example, a green background combined with a textual reference.

Status indicator 58002 may include a support structure 58002d that supports status panels 58002a, 58002b, and 58002c. Support structure 58002d may include an extension 58002e, which may extend downward between tracks 58006 along which support structure 58002d slides. Additionally, as discussed below and shown in FIGS. 58F-58H, extension 58002e may include one or more protrusions 58002f and 58002g that can interact with prongs 58012a or 58012b of patient needle mechanism 58010. Status indicator 58002 may also be movable on tracks 58006. Although not shown, tracks 58006 may be fixedly coupled to an internal portion of auto-injector 2, for example, on an interior of housing 3.

In one aspect, and as mentioned above, status indicator 58002 may be moved by one or more prongs 58012a and 58012b of shuttle 58014. Patient needle mechanism 58010 may include shuttle 58014 with one or more teeth 58014a, which may engage with one or more gears (not shown, e.g., gear 360a described elsewhere herein) in order to actuate a needle injection process, as discussed above. As also discussed above, patient needle mechanism 58010 may include a spring connection 58016 and a push rod connection 58018. Patient needle mechanism 58010 may include one or more prongs 58012a and 58012b, which may extend from a portion of shuttle 58014, for example, between spring connection 58016 and push rod connection 58018.

As mentioned above, status indicator 58002 may be engaged or pushed by one or more prongs 58012a and 58012b. As shown in FIGS. 58F-58H and as discussed herein, status indicator 58002 may include a protrusion 58002f, for example, extending laterally from extension 58002e, that may be positioned between the two prongs 58012a and 58012b. Protrusion 58002f may be contacted by one or more of prongs 58012a and 58012b so that movement of the shuttle 58014 between the different stages of injection also moves the status indicator 58002. Thus, status indicator 58002 is moveable relative to status window 58000 during the actuation of patient needle mechanism 58010.

FIGS. 58C-58E illustrate window 58000 and status indicator 58002 in the above-discussed configurations. For example, FIG. 58C illustrates status indicator 58002 in a first position relative to window 58000 and tracks 58006. As shown, first status panel 58002a is at least partially aligned with window 58000, corresponding to the initial or unused state. In this state, second status panel 58002b and third status panel 58002c are outside of window 58000, and thus at least partially blocked by a portion of the housing so that they are not viewable from exterior of window 5800. FIG. 58D illustrates status indicator 58002 in a second position relative to window 58000 and tracks 58006. As shown in FIG. 58D, second status panel 58002b is at least partially aligned with window 58000, corresponding to the active or in-progress state. In this state, first status panel 58002a and third status panel 58002c may not be viewable from outside of window 58000, and thus may be at least partially blocked by a portion of the housing. FIG. 58E illustrates status indicator 58002 in a third position relative to window 58000 and tracks 58006. As shown, third status panel 58002c is at least partially aligned with window 58000, corresponding to the complete or used state. In this state, first status panel 58002a and second status panel 58002b are not viewable from outside of window 58000, and thus may be at least partially blocked by a portion of the housing. As mentioned above and as shown in FIGS. 58C-58E, the movement of prongs 58012 during an injection may also help to translate status indicator 58002 relative to window 58000.

FIGS. 58F-58G illustrate the interaction of prongs 58012a and 58102b with status indicator 58002 during an injection in greater detail. As shown in FIG. 58F, in the initial or unused state, a portion of status indicator 58002 is aligned with window 58000, for example, corresponding to first status panel 58002a. Moreover, prong 58012a may abut a portion of protrusion 58002f at this initial stage. Furthermore, at this initial stage a gap 58002h may be disposed between prong 58012b and another protrusion 58002g. Shuttle 58014 may be biased by spring 58070, as discussed herein, and this biasing may help to ensure status indicator 58002 remains in the initial or unused state until injection. In one aspect, protrusion 58002f may be positioned between prongs 58012a and 58012b, such that movement of shuttle 58104 moves protrusion 58002f, and thus moves status indicator 58002 along tracks 58006 during an injection process.

As shown in FIG. 58G, in the active or in-progress state, another portion of status indicator 58002 is aligned with window 58000, for example, corresponding to second status panel 58002b. For example, as shuttle 58014 moves and compresses spring 58070 during the injection, prong 58012a moves protrusion 58002f. Accordingly, movement of shuttle 58014 moves protrusion 58002f, and thus moves status indicator 58002 along tracks 58006 to the second position during an injection process. In this position, second status panel 58002b may be displayed through window 58000. The gap 58002h between prong 58012b and protrusion 58002g may be substantially maintained between the first and second states.

Lastly, as shown in FIG. 58H, in the complete or used state, yet another portion of status indicator 58002 is aligned with window 58000, for example, corresponding to third status panel 58002c. For example, as shuttle 58014 retracts due to force of pressurized gas acting on shuttle 58104 being less than the force of spring 58070 during the injection, shuttle 58014 will move toward its initial position. Accordingly, prong 58012b will move toward protrusion 58002g to move the status indicator 58002 along tracks 58006 to the third position during an injection process. Because of the presence of gap 58002h in the first and second states, the movement of shuttle 58014 back toward its initial position moves the status indicator to the third position (which is a position between the first position and the second position). The third position may be spaced from the first position by approximately the length of gap 58002h. In this position, third status panel 58002c may be displayed through window 58000. The length of gap 58002h may be substantially equal to a length of any one of status panel 58002a, 58002b, and/or 58002c.

Based on the interaction of shuttle 58014 and status indicator 58002, for example, via the interaction of prongs 58012a and 58012b and protrusions 58002f and 58002g, information about the state, status, progress, etc. of an injection may be displayed to the user. Moreover, the above aspects may help to display whether auto-injector 2 is ready for an injection, whether auto-injector 2 is in the process of an injection, or whether auto-injector 2 has already been used for an injection. The indicator mechanism disclosed herein may be relatively simple, adding only two or three components to an existing patient needle mechanism. Furthermore, the indicator mechanism utilizes the motion of the patient needle mechanism, which allows for real-time indication of the status of the device independent of piston movement shown through another window of the auto-injector. In combination with the smart sense technology of the one or more valves disclosed herein, an improved accuracy or determination of the actual, real-time state of the auto-injector 2 may be obtained. Existing auto-injector systems tend to prematurely indicate that the injection is complete because the plunger rod is used to trigger the indication. In some instances, the plunger rod may reach the end of its travel path before the end of the injection itself.

Other features may be incorporated into the indicator mechanism disclosed herein. For example, a snapfit, stop, or other feature may be used to prevent status indicator from moving back to the first position instead of the third position. In other words, the force provided by expansion of spring 58070, that absent some mechanism to stop the status indicator 58002 as it moves during spring expansion, the status indicator may be pushed past the third position back toward the first position (providing a false status that the injector is unused). A snap fit or stop or stop could be positioned on or in the path of support structure 58002d or elsewhere to prevent status indicator 58002 from moving back to its first position. Alternatively, support structure 58002d may have a tight tolerance, and precise positioning may be achieved by friction levels.

In one embodiment, a transverse (flattened) auto-injector may include a button positioned on a longitudinal end of a top surface of the auto-injector. The button may include one or more protruding bumps, and may have a different color than of adjacent portions of the housing. For example, the button may be teal, green, or blue, while adjacent portions of the top surface of the housing are white. A label including identifying information may be adjacent to the button on the top surface. The button may be a push button that is transversely aligned with the needle opening. The needle opening may be on a bottom, tissue-contacting surface of the device. A contact switch (similar to contact switch 46002 disclosed herein) may be disposed around the needle opening. The bottom surface may be a different color than the top surface and a different color than the button. For example, the bottom surface may be grey and may include a grippy or rubber material, or may otherwise include a hard plastic material. The top surface of the auto-injector may include protruding or etched ribs to facilitate grip. A window may extend along a longitudinally-extending side surface of the auto-injector, and may enable a user to see a container (with medicament) and a piston inside of the container. The window may optionally include paint, frost, tint, or a cover to prevent a user from viewing unused space within the container before injection has started. The auto-injector may include a pull tab that prevents activation of the device before the pull tab is removed. The pull tab may occupy the same space through which the contact switch extends (after the pull tab is removed). The positioning of a button directly over the needle may provide certain users with more comfort by giving such users an impression of greater control over the injection process. In other embodiments, a positioning of the needle opening offset from a center of auto-injector 2 may promote the use of auto-injector 2 on smaller target surfaces, such as, for example, an arm. An offset needle opening enables the use of auto-injector 2 on smaller surfaces, since in such embodiments, an entirety of the bottom surface does not need to be placed on the user's skin for deployment of the needle.

In another embodiment, a transverse auto-injector may have a larger transverse dimension (perpendicular to the skin surface) than lateral dimension (parallel to the skin surface). The auto-injector may have its longest dimensions along the longitudinal axis (parallel to the skin surface). The tissue-contacting surface of the auto-injector is longer than the top surface in this embodiment, and when viewed from the side, the auto-injector may have a generally trapezoidal appearance with rounded corners. A pull tab may be disposed on the tissue-contacting surface, and may prevent activation of the device before it is removed. The pull-tab may extend along the substantial entirety of the tissue-contacting surface. The auto-injector of this embodiment may include a shroud retractable into the housing. Application of force to the top of the housing when the shroud is placed against the skin may cause shroud retraction and needle insertion. The needle opening may be disposed in the radial and longitudinal center of the tissue-contacting surface. A window may extend along the top surface to enable viewing of the container and piston contained therein. Furthermore, this auto-injector may optionally include a flag 54000 as described above. The window on the top surface may be rounded and include tint, paint, or frost, to block the view of unused space in the container before the start of the injection.

In another auto-injector, the transverse auto-injector may be larger in the transverse dimension (perpendicular to the skin surface) than in the lateral dimension (parallel to the skin surface). The auto-injector may have its longest dimensions along the longitudinal axis (parallel to the skin surface). The auto-injector may be longer at its top surface that at its tissue-contacting surface. The top surface may be offset and angled relative to both the longitudinal axis and the transverse axis of the auto-injector. For example, the top surface of the auto-injector may extend at an angle from about 5 degrees to about 65 degrees, from about 10 degrees to about 60 degrees, from about 15 degrees to about 55 degrees, from about 20 degrees to about 50 degrees, from about 25 degrees to about 45 degrees, from about 30 degrees to about 40 degree, or about 35 degrees, relative to the bottom, tissue-contacting surface of the auto-injector. The tissue-contacting surface may be a different color (e.g., teal or any other suitable color) than the top surface (such as, e.g., white) and may include a grippy or tacky portion similar to that described with reference to FIG. 47C. A window may extend along the top surface, and an activating button may be disposed at the intersection of the top surface and a transversely-extending surface. The button may include a divot or bumps as described above, and also may include colored side surfaces to provide a visual indication of the state of the auto-injector as described above. A contact switch may extend from the bottom surface, and a needle opening may be disposed in the contact switch. The contact switch may be generally ovular, and may include ribs to facilitate placement on the skin surface. The needle opening also may be offset relative to a center of both the contact switch and the bottom surface. Optionally, a cover piece, window paint, or frost may block the user's view of dead space through a window of the auto-injector. A user may grip this embodiment by wrapping her palm and second, third, fourth, and fifth digits around a handle portion of the auto-injector that protrudes furthest away from the skin surface. When the auto-injector is positioned against the skin surface, the user's fifth digit will be positioned highest relative to the skin surface, and the fourth, third, and second, digits of the user's hand will be positioned progressively lower relative to the skin surface. The user's thumb or first digit will be placed closest to the skin surface and may be used to press the button to activate the auto-injector.

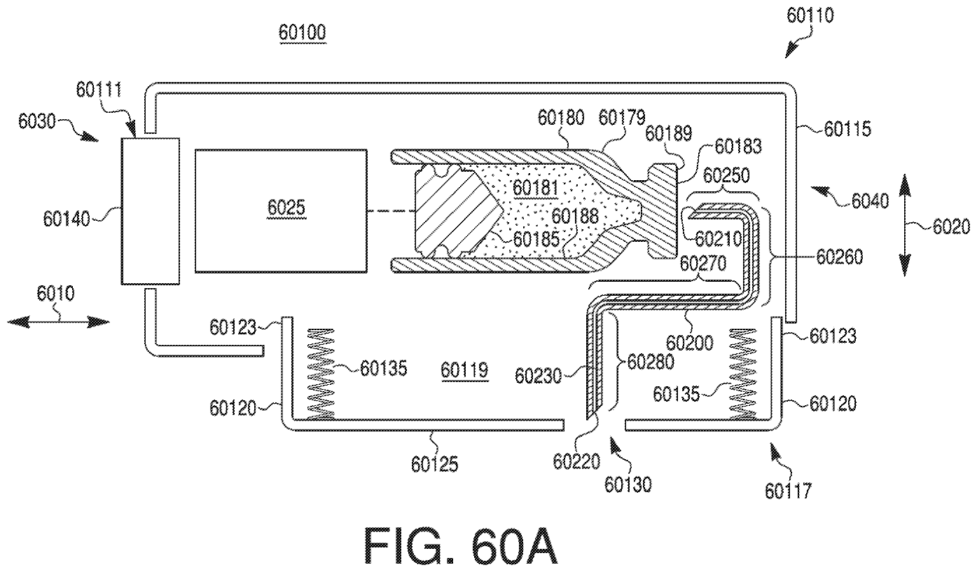

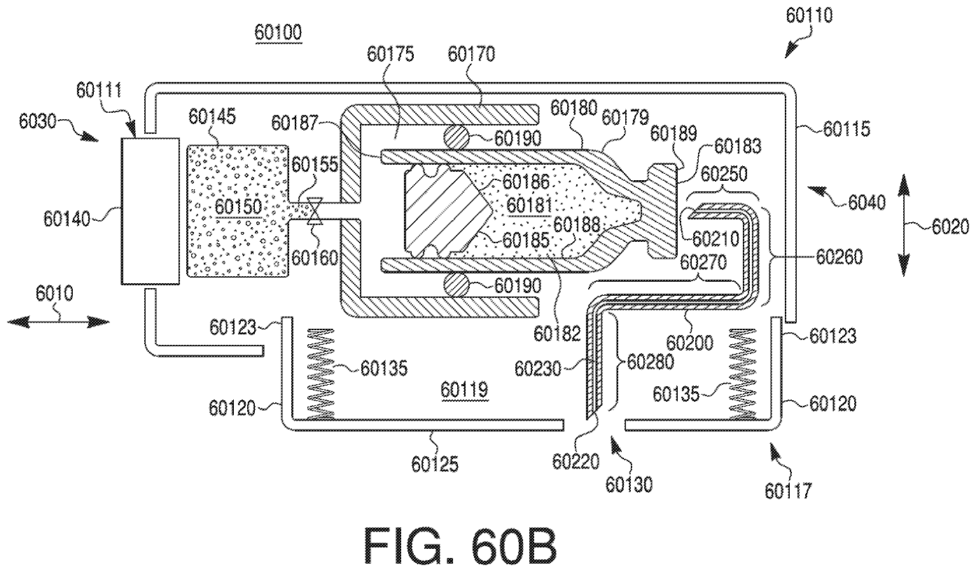

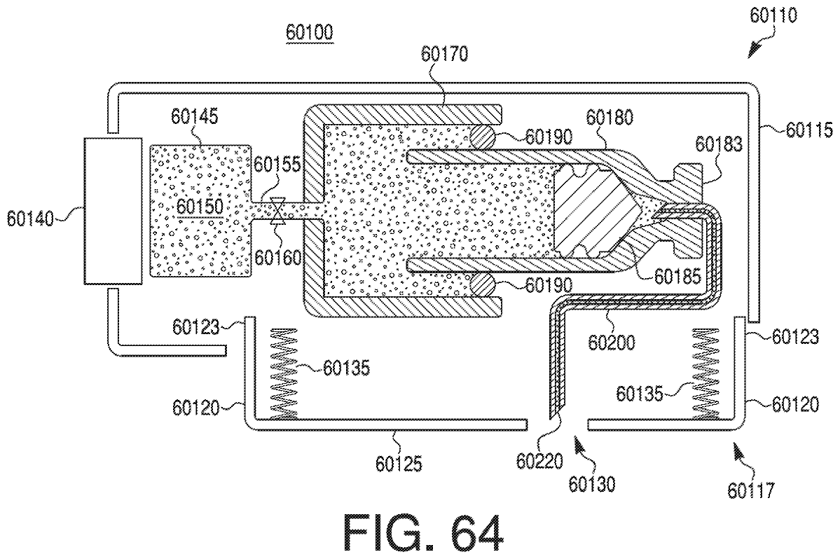

An example of such an auto-injector 60100 is shown in FIGS. 60A-64. Auto-injector 60100 may be a handheld auto-injector, as opposed to a wearable auto-injector. In at least some embodiments, a handheld auto-injector may require a user to hold the auto-injector against the user's skin for the entirety of an injection procedure, whereas, a wearable injector may include features for securing the wearable auto-injector to the skin. For example, a wearable auto-injector may include one or more features, such as, e.g., an adhesive patch, straps, or the like, for securing to the user. In some embodiments, a handheld auto-injector according to this disclosure may be configured to deliver a medicament volume of less than 3.5 mL (or a medicament volume from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL), whereas a wearable auto-injector may be configured to deliver a medicament volume of greater than 3.5 mL, greater than 4.0 mL, or greater than 5.0 mL. Auto-injectors of the present disclosure may be configured to deliver highly viscous liquid to a patient. For example, auto-injectors of the present disclosure may be configured to deliver liquid having a viscosity from about 0 cP to about 100 cP, from about 5 cP to about 45 cP, from about 10 cP to about 40 cP, from about 15 cP to about 35 cP, from about 20 cP to about 30 cP, or about 25 cP.

Furthermore, handheld auto-injectors according to the present disclosure may be configured to complete an injection procedure, as measured from (1) a point at which that the user places the auto-injector onto the skin to 2) a point at which the user removes the auto-injector from the skin after completion of an injection, in less than about 30 seconds, less than about 25 seconds, less than about 20 seconds, less than about 15 seconds, or less than about 10 seconds. A wearable auto-injector may or will take longer than 30 seconds to complete the same steps 1) and 2) discussed above, i.e., from 1) the point in time at which the auto-injector is placed onto a user's skin, to 2) the point in time at which the auto-injector is removed from the skin.

Auto-injector 60100 may include housing 60110. Housing 60110 may be oriented about a longitudinal axis 6010 (e.g., an X axis) and a transverse axis 6020 (e.g., a Y axis) that is substantially perpendicular to longitudinal axis 6010. The housing 60110 may have a shorter dimension along the transverse axis 6020, than along the longitudinal axis 6010. The housing 60110 may include a power source 6025. The power source 6025 may include one or more mechanical, electrical, chemical, and/or fluid actuation mechanisms configured to provide a driving force to a plunger (i.e., plunger 60185 described in further detail below). Such actuation mechanisms may include a motor configured to drive a screw or telescoping rod, spring or other resilient member, other energy-storing mechanical part, compressed or pressurized air, another pressurized or compressed fluid, a chemical reaction, a circuit, or a combination thereof. FIGS. 60B-64 show an exemplary embodiment comprising a fluid-based power source (i.e., fluid source 60145).

Along the longitudinal axis 6010, the housing 60110 may define an actuation end 6030 and an expulsion end 6040. The embodiments shown in FIGS. 60A-64 are merely exemplary, and an auto-injector 60100 may provide actuation or expulsion capabilities at any location of the housing 60110. Housing 60110 may have any dimensions suitable to enable portability and self-attachment by a user or medical professional. Housing 60110 may be dimensioned such that auto-injector 60100 comprises a handheld device that a user may compress or hold against a treatment/injection site. While the illustrated embodiments of FIGS. 60A-64 show a substantially rectangular-shaped housing 60110, other embodiments of housing 60110 may have a circular, cylindrical, curved, or ergonomic shape. Housing 60110 may also include a grippy or tacky coating such that the outer surface of housing 60110 is non-slip or corrugated surface.

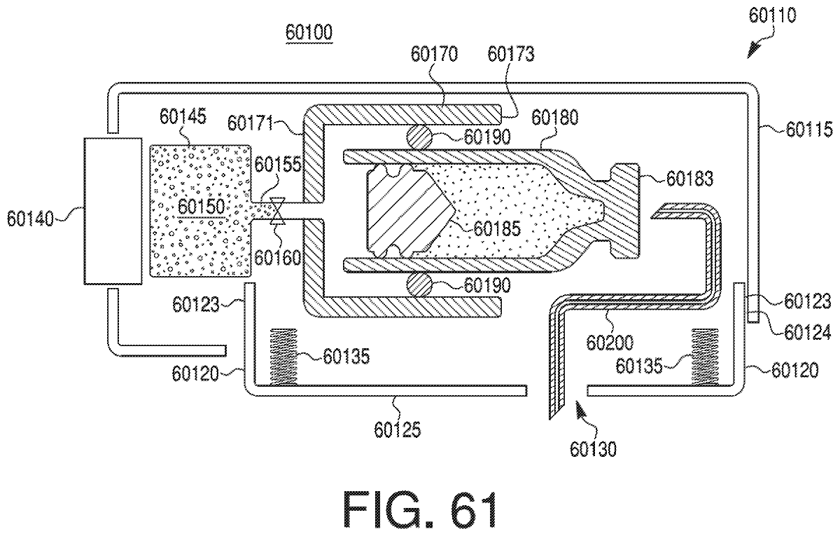

The housing 60110 may include a handle portion 60115 and a retractable shroud 60117. Handle portion 60115 may include transparent, translucent, opaque, plastic, metal, disposable, reusable, rigid, or flexible material. Handle portion 60115 may also include one or more transparent/translucent openings, windows, or portions that permit visualization of the contents of housing 60110. Shroud 60117 may include materials comprising plastics, metals, fabrics, or a combination thereof. Shroud 60117 may retract along the transverse axis 6020, into the handle portion 60115 by application of a force to handle portion 60115 from a user. Handle portion 60115 and shroud 60117 may be coupled to one another to create an inner cavity 60119 of the housing 60110. The inner cavity 60119 may have a first volume at an initial state of the auto-injector 60100 (e.g., as shown in FIG. 60B), and a smaller, second volume after the shroud 60117 is retracted (e.g., as shown in FIG. 61). The retractable shroud 60117 may have sidewalls 60120 and a tissue-engaging (e.g., bottom) surface 60125. The sidewalls 60120 may retract into the inner cavity 60119. For example, sidewalls 60120 may have a portion 60123 that may retract into and overlap with a portion 60124 of the handle portion 60115 (e.g., as shown in FIG. 61). In other embodiments, sidewalls 60120 may be shaped as bellows or folds, which may crease or expand along pre-set pleats. In yet another embodiment, instead of a handle portion and a retractable shroud, a single housing may include bellow or folds near its tissue engaging surface.

Handle portion 60115 and shroud 60117 may be biased toward the initial state shown in FIG. 60B by one or more coils, elastic materials, pneumatic mechanisms, etc. In the illustrated embodiments, springs 60135 may extend into inner cavity 60119 from an interior surface of the shroud 60117 that may be opposite tissue-engaging surface 60125 and may be positioned adjacent sidewalls 60120 to provide resistance to the motion of retraction. Furthermore, springs 60135 may be coupled to an interior surface of handle portion 60115, or to an internal element of auto-injector 60100 that is fixed relative to handle portion 60115 to compress springs 60135. Springs 60135 may be positioned inside the inner cavity 60119 and shroud 60117, as illustrated in FIGS. 60A-64, in the handle portion 60115, at least partially in the shroud 60117 and partially in the handle portion 60115, etc. Springs 60135 may be biased into an expanded position, as shown in FIG. 60B.

The tissue-engaging surface 60125 of the shroud 60117 may have an opening 60130 through which a flowpath 60200 may be deployed (e.g., shown in FIG. 61). Retraction of shroud 60117 (i.e., the movement of handle portion 60115 and shroud 60117 toward one another) may cause a tip of flowpath 60200 to extend out of shroud 60117, where it can be inserted into a user/patient. As set forth above, springs 60135 may be biased to its expanded configuration, so that the flowpath 60200 is contained inside the housing 60110 when auto-injector 60100 is in a resting position. In such embodiments, continued force on the handle portion 60115 may be used to maintain deployment of the flowpath 60200 within a user. Some embodiments of housing 60110 may include a catch or clasp, which may secure auto-injector 60100 into the compressed configuration shown in FIGS. 61-63, without continued force on the handle portion 60115 by the user. For example, handle portion 60115 and shroud 60117 may include interlocking or complementary locking features that interact with one another to secure handle portion 60115 and shroud 60117 in the compressed configuration. Exemplary interlocking features may include a ramp or angled geometrical shape such that the features may both stabilize handle portion 60115 and shroud 60117 in an initial, extended position, and lock handle portion 60115 and shroud 60117 in the compressed configuration. A ramp or angled shape for the interlocking feature may allow handle portion 60115 and shroud 60117 to easily slide past one another before locking. In one such embodiment, interlocking of the locking features may be a prerequisite for fluid 60150 release and/or actuation of button 60140. In some cases, button 60140 may be a component of power source 6025. In some embodiments, flow of fluid 60150 and/or medicament (treatment fluid) 60181 may cease when shroud 60117 is in an extended (e.g., uncompressed/retracted) configuration.

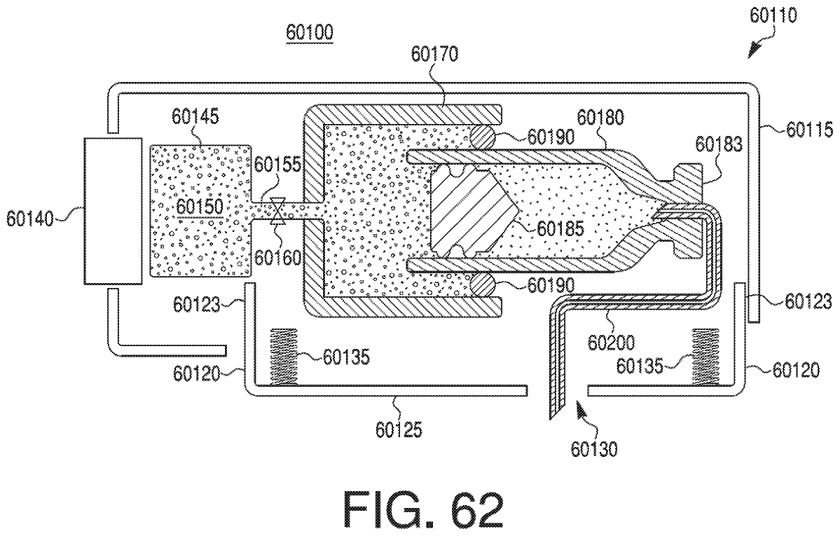

Flowpath 60200 may include a hollowed needle, including a first needle 60210, a second needle 60220, and a lumen 60230 extending from the first needle 60210 to the second needle 60220. The first needle 60210 may be configured to puncture a cartridge seal 60183 to put flowpath 60200 into fluid communication with a cartridge 60180 (described in further detail below). Once the first needle 60210 penetrates the cartridge seal 60183 and establishes fluid communication with cartridge 60180 (see, e.g., FIG. 62), medicament may travel from cartridge 60180, through lumen 60230 of flowpath 60200, and enter a user through second needle 60220. The first needle 60210 portion of flowpath 60200 may be positioned generally parallel to or along the longitudinal axis 6010. The second needle 60220 may be configured to puncture or be injected into a patient's body at an injection site. The second needle 60220 may be positioned generally along or parallel to the transverse axis 6020. The first needle 60210 and second needle 60220 may be offset from one another and/or generally or exactly perpendicular to each other. Flowpath 60200 may be substantially or entirely disposed within the housing 60110 when the shroud 60117 is in the initial state shown in FIG. 60B, but the second needle 60220 may protrude from the opening 60130 when the shroud 60117 is retracted (FIGS. 61-63). In some cases, the opening 60130 may include a membrane or other covering, so that the flowpath 60200 may be kept sterile prior to use.

Flowpath 60200 may include a metal, a metal alloy, polymers, or the like. Flowpath 60200 may be opaque. Alternatively, flowpath 60200 may be translucent or transparent such that lumen 60230 of the flowpath 60200 may be viewable. In some cases, at least a portion of housing 60110 may be transparent or translucent at the location of flowpath 60200 such that a user may observe the lumen of flowpath 60200. Flowpath 60200 may define a 22, 23, or 27 gauge, thin-walled needle, according to exemplary embodiments. Other needle sizes ranging from, e.g., 6 Gauge to 34 Gauge, also may be utilized. Gauge sizes may be chosen based on the quantity or viscosity of medicament to be dispensed by auto-injector 60100. The gauge size of flowpath 60200 may vary along the length of the flowpath 60200. For example, first needle 60210 may have a different gauge size than second needle 60220. The lumen 60230 of flowpath 60200 may be made of a material or coated with a substance to decrease friction in the flow of the medicament.

One advantage of auto-injector 60100 is its low profile along the transverse axis 6020. The low profile translates into a small-sized auto-injector 60100, which may facilitate storage and ease patients' fear of large needles. To accommodate the short profile, flowpath 60200 may have a serpentine or nonlinear shape. In some embodiments, flowpath 60200 may include a plurality of sections offset from one another. As shown, flowpath 60200 has four offset sections, although any other suitable number, including, e.g., two, three, five, or more offset sections (e.g., section 60250, section 60260, section 60270, and section 60280) may be utilized. At least first needle 60210 may extend along or parallel to the longitudinal axis 6010, while at least second needle 60220 extends along, or parallel to the transverse axis 6020. Thus, first needle 60210 and second needle 60220 may be substantially perpendicular to one another.

In operation, the tissue-engaging surface 60125 may be positioned against a portion of a user's body, e.g., at a treatment or delivery site. A downward force may be applied to the housing 60110, along the transverse axis 6020. This force may cause the shroud 60117 to retract into the handle portion 60115 of the housing 60110 along the transverse axis, and extend flowpath 60200 from opening 60130 to puncture the user (e.g., as shown at FIG. 61). In other words, when force is applied along the transverse axis of auto-injector 60100, the shroud 60117 may collapse or retract, while all the components of in the cavity 60119 of housing 60110 (including flowpath 60200) may translate along the transverse axis. In some embodiments, components of auto-injector will move only along transverse axis 6020 during this compression step, and not along longitudinal axis 6010. Because flowpath 60200 may be closest to the tissue-engaging surface 60125 of auto-injector 60100, flowpath 60200 may extend through opening 60130 during the compression step. While not shown, housing 60110 may include one or more detents or fixtures to secure the position of flowpath 60200. Securing the position of the flowpath 60200 may ensure that flowpath 60200 does not twist, bend, or retract into the housing 60110 upon contact with a patient, or deform and twist when contacting cartridge 60180 (as described in further detail below).

Referring to FIGS. 60A-64, auto-injector 60100 may include a button 60140, fluid source 60145, conduit 60155, switch 60160, rail 60170, dispensing chamber 60175, cartridge 60180, and flowpath 60200. The embodiment of FIGS. 60B-64 specifically contemplates a fluid-based power source (fluid source) 60145 and, as is evident from FIG. 60A, fluid source 60145 may be substituted for another suitable power source, including any of those structures discussed above with respect to power source 6025. Cartridge 60180 may be a cylindrical container. For example, cartridge 60180 may be a standard 3 mL container having an 8 mm crimp top, a 9.7 mm inner diameter, and a 64 mm length. In one present embodiment, cartridge 60180 may be comprised of a cylindrical vial arranged with its longitudinal length parallel to the longitudinal axis 6010 of housing 60110. Cartridge 60180 may have an outer surface 60179 and an inner surface 60188. The inner surface 60188 may define a cavity 60182 containing medicament 60181. Cartridge 60180 may have a base edge 60187 at a first end and extend towards an opening 60189 at a second end. The base edge 60187 may be the portion of the cartridge 60180 closest to actuation end 6030 of the housing 60110 (e.g., shown in FIGS. 60B-64). The opening 60189 may be at an end of cartridge 60180 closest to the expulsion end 6040. While FIGS. 60A-64 illustrate exemplary actuation end 6030 and expulsion end 6040, the cartridge 60180, base edge 60187, and opening 60189 may be positioned in any arrangement within housing 60110. For example, a circular-shaped housing 60110 may orient cartridge 60180 to dispense its contents solely with respect to a treatment or injection site, rather than an actuation end 6030 or expulsion end 6040. Opening 60189 may be covered by a seal 60183, which may seal medicament 60181 inside cavity 60182 at the second end of cartridge 60180.

Seal 60183 may be configured to assist with closing and/or sealing of opening 60189, and allow for first needle 60210 of flowpath 60200 needle be inserted into cartridge 60180. Seal 60183 may also include a rubber, fibrous, or elastic material such that puncturing of the seal 60183 may still create a seal around the flowpath 60200, so that medicament 60181 does not flow out from a puncture site around flowpath 60200. Seal 60183 may include an uncoated bromobutyl material, or another suitable material.

The “nominal volume” (also called the “specified volume” or “specified capacity”) of a container refers to the container's maximum capacity, as identified by the container's manufacturer or a safety standards organization. A manufacturer or a safety standards organization may specify a container's nominal volume to indicate that the container can be filled with that volume of fluid (either aseptically or not) and be closed, stoppered, sterilized, packaged, transported, and/or used while maintaining container closure integrity, and while maintaining the safety, sterility, and/or aseptic nature of the fluid contained inside. In determining the nominal volume of a container, a manufacturer or a safety standards organization may also take into account variability that occurs during normal filling, closing, stoppering, packaging, transportation, and administration procedures. As an example, a prefillable syringe may be either hand- or machine-filled with up to its nominal volume of fluid, and may then be either vent tube- or vacuum-stoppered, without the filling and stoppering machinery and tools touching and potentially contaminating the contents of the syringe.

Cartridge 60180 may have about a 5 mL nominal volume in some examples, although any other suitable volume may be utilized. In one embodiment, cartridge 60180 may be configured to deliver a delivered quantity of medicament (e.g., from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL, or another delivered quantity). The delivered quantity may be less than the nominal volume of cartridge 60180. Furthermore, in order to deliver the delivered quantity of medicament to a user, cartridge 60180 itself may be filled with a different quantity of medicament than the delivered quantity (i.e., a filled quantity). The filled quantity may be an amount of medicament greater than the delivered quantity to account for medicament that cannot be transferred from cartridge 60180 to the user due to, e.g., dead space in cartridge 60180 or flowpath 60200. Thus, while cartridge 60180 may have a nominal volume of 5 mL, the filled quantity and delivered quantity of medicament may be less than 5 mL. In one embodiment, because cartridge 60180 is used in a handheld auto-injector, the delivered quantity of medicament from cartridge 60180 may be from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL. The filled quantity and the delivered quantity of medicament may be related to the viscosity of the medicament and the hand-held nature of auto-injector 60100. That is, in at least some embodiments, at certain viscosities, higher volumes of medicament may prohibit the ability of auto-injector 60100 to complete an injection procedure in less than an acceptable amount of time, e.g., less than about 30 seconds. Thus, the delivered quantity of medicament from auto-injector 60100 may be set such that an injection procedure, measured from 1) the point in time at which the auto-injector is placed onto a user's skin, to 2) the point in time at which the auto-injector is removed from the skin, is less than about 30 seconds or less than about another time period (e.g., less than about 25 seconds, less than about 20 seconds, less than about 15 seconds, or less than about 10 seconds). When the delivered quantity and viscosity of the medicament is too high, auto-injector 60100 may not be able to function as a handheld auto-injector, since the time required to complete the injection procedure may be higher than commercially or clinically acceptable for handheld devices. In other examples, cartridge 60180 may have a capacity greater than or equal to 1 mL, or greater than or equal to 2 mL, or greater than or equal to 3 mL. Again, as stated above, since cartridge 60180 may be used in a hand-held auto-injector, regardless of the nominal volume of cartridge 60180, the delivered quantity of medicament from cartridge 60180 may be set such that the injection procedure as defined above is completed in a relatively short period of time (so as to avoid the need for additional features to attach the auto-injector 60100 to the user so that auto-injector 60100 is a wearable auto-injector). Cartridge 60180 may contain and preserve a drug for injection into a user, and may help maintain sterility of the drug. In some examples, cartridge 60180 may be formed using conventional materials, and may be shorter than existing devices, which can help auto-injector 60100 remain cost-effective and small. In some embodiments, cartridge 60180 may be a shortened ISO 10 mL cartridge.

A plunger 60185 may be concentric with cartridge 60180 and seal base edge 60187 of cartridge 60180. Plunger 60185 may close off (i.e., seal) cavity 60182 at the actuation end 6030 of the cartridge 60180. Plunger 60185 may be configured to slide along the cartridge inner surface 60188, from the base edge 60187 toward the opening 60189. In one embodiment, plunger 60185 may have a cylindrical shape, where the axial surface of the cylinder may lie flush against the inner surface 60188. In other embodiments, the outer surface of plunger 60185 may include one or more circumferentially extending seals (not shown). Plunger 60185 may further include a head 60186 shaped to correspond to the expulsion end of cartridge 60180. For example, if cartridge 60180 narrows or has a necked portion close to cartridge opening 60189, plunger 60185 may have a conical head portion 60186 that may fill the narrowing or necked portion of cartridge 60180. Plunger 60185 may include a rubber or elastic material that may deform against the interior of cartridge 60180 and form a seal. For example, plunger 60185 may include a fluoropolymer coated bromobutyl material or one or more rubber materials such as, e.g., halobutyls (e.g., bromobutyl, chlorobutyl, florobutyl) and/or nitriles, among other materials.

Fluid source 60145 may be a non-latching or latching can that is capable of dispensing liquid propellant for boiling outside of fluid source 60145 so as to provide a pressurized gas (vapor pressure) that acts on cartridge 60180 and plunger 60185. Once opened, the latching can embodiment may be latched open so that the entire contents of propellant is dispensed therefrom. Alternatively, in some embodiments, fluid source 60145 may be selectively controlled, including selectively activated and deactivated. For example, in an alternative embodiment, the flow of pressurized gas from fluid source 60145 may be stopped after flow is initiated.

The fluid 60150 from fluid source 60145 may be any suitable propellant for providing a vapor pressure to drive plunger 60185. In certain embodiments, the propellant may be a liquefied gas that vaporizes to provide a vapor pressure. In certain embodiments, the propellant may be or contain a hydrofluoroalkane (“HFA”), for example HFA134a, HFA227, HFA422D, HFA507, or HFA410A. In certain embodiments, the propellant may be or contain a hydrofluoroolefin (“HFO”) such as HFO1234yf or HFO1234ze. In other embodiments, the propellant may be R-134a (1,1,1,2-Tetraflouroethane). In other embodiments, fluid source 60145 may be a high-pressure canister configured to contain a compressed gas.

Button 60140 may be positioned at the actuation end 6030, or at any external portion of housing 60110. For example, button 60140 may protrude from an opening 60111 of housing 60110. Button 60140 may recede into opening 60111 when depressed, e.g., by a user. Alternatively, button 60140 may be comprised of an elastic material, which may be deformed when pressed. Button 60140 may include any actuation mechanism, including a switch, knob, latch, catch, trigger mechanism, etc. Button 60140 may be coupled to fluid source 60145 such that actuation of button 60140 may cause fluid source 60145 to release compressed fluid 60150 from the fluid source 60145.

Fluid source 60145 may be positioned adjacent to button 60140, along the longitudinal axis 6010 of housing 60110. Actuation (e.g., compression of button 60140) may cause fluid source 60145 to expel fluid 60150. In some embodiments, fluid 60150 may be expelled only if the button 60140 is compressed and shroud 60117 is compressed or retracted. In such a case, compression of button 60140 and compression/retraction of shroud 60117 may be order-independent. Thus, fluid 60150 may be released as long as both button 60140 is actuated and shroud 60117 is compressed/retracted, regardless of the sequence of the operations. In other embodiments, compression of button 60140 and compression/retraction of shroud 60117 are order-dependent, and a specific sequence of these two events must be carried out in order to release fluid 60150. In one example, compression of button 60140 must occur before compression/retraction of shroud 60117 to release fluid 60150, and in another embodiment, compression/retraction of shroud 60117 must occur before compression of button 60140 to release fluid 60150.

In some embodiments, compression or retraction of shroud 60117 may be a single prerequisite for expelling of fluid 60150. In one such case, shroud 60117 may include a catch, which may release fluid 60150 from fluid source 60145. In another such case, button 60140 may be connected to a catch (not shown), which may release and allow button 60140 to be compressed when or after shroud 60117 is retracted. In some embodiments, button 60140 may be comprised of a knob or dial corresponding to a switch 60160 comprising a tuner or adjuster. In such cases, twisting of button 60140 in a first direction may correspond to an opening of switch 60160, and the opening of switch 60160 may be reversed by rotating button 60140 in an opposite direction from the first direction.

In some embodiments, release of the compressed fluid 60150 from fluid source 60145 may automatically be initiated upon retraction of shroud 60117. In some embodiments, auto-injector 60100 includes a switch comprising or in place of button 60140. One such switch may be tripped during retraction of shroud 60117. For example, auto-injector 60100 may include an electrical contact positioned on handle portion 60115 and an electrical contact positioned on shroud 60117. These electrical contacts may be joined during retraction of shroud 60117, and thus trigger fluid source 60145 to release fluid 60150. Alternately, button 60140 and/or shroud 60117 may include a mechanical linkage or cover. This linkage or cover may block the flow of fluid 60150 (or be connected to a component that may block the flow of fluid 60150) prior to release of fluid 60150 from fluid source 60145. In such cases, retraction of shroud 60117 may move the linkage so that flow is fluid 60150 is permitted, an element sealing fluid source 60145 is opened, or other actuator component is moved to release fluid 60150 from the fluid source 60145.

In some embodiments, an extent of compression of button 60140 may correspond to speed or quantity of compressed fluid 60150 released from fluid source 60145 (e.g., more compression of button 60140 corresponding to a higher speed of expulsion from the fluid source). In other embodiments, button 60140 may merely initiate release of compressed fluid 60150 and offer no additional control over the release.

Fluid source 60145 may be configured to contain enough fluid so that release of the fluid 60150 may actuate both movement of the cartridge 60180 and plunger 60185, as described in greater detail below. In some cases fluid source 60145 may contain excess fluid 60150, i.e., more fluid than is necessary to complete delivery of the contents of cartridge 60180. Auto-injector 60100 may include, for example, an element configured to help release such excess fluid 60150. For instance, rail 60170 may include an opening for venting after injection completion or dispensing of the medicament. As another example, power source 60145 or switch 60160 may include a 3-way element, a plurality of 1-way elements, a spigot, or any other suitable structure configure to help enable a flow of excess fluid 60150 from within auto-injector 60100 to exterior of auto-injector 60100 (e.g., the atmosphere). Alternately or in addition, fluid 60150 may escape from the auto-injector 60100 absent active venting mechanisms. In yet another embodiment, auto-injector 60100 may not be vented after completion of an injection, such that pressurized fluid or propellant remains in fluid source 60145.

Auto-injector 60100 may further include a rail 60170 having a cylindrical structure extending along the longitudinal axis 6010 of housing 60110. Rail 60170 may have an inner surface which may form a lumen. Rail 60170 may coaxially surround cartridge 60180. For example, cartridge 60180 may be positioned inside the lumen formed by rail 60170. Rail 60170 may be spaced from the cartridge 60180 such that the cartridge 60180 may slide along the length of the rail 60170.

Rail 60170 may include a base 60171 near the actuation end 6030 of the housing 60110, as well as a rim 60173 near the expulsion end 6040 of the housing 60110 (e.g., as illustrated in FIG. 61). Base 60171 may include an opening connected to conduit 60155, such that compressed fluid 60150 may travel through conduit 60155 to a cavity formed by rail 60170. The cavity formed by the inner surface of rail 60170, a sliding seal 60190, plunger 60185, and an outer wall of cartridge 60180 may form dispensing chamber 60175.

Sliding seal 60190 may be disposed between the cartridge 60180 and the rail 60170 to facilitate movement of the cartridge 60180 by preventing fluid 60150 from leaking past the seal 60190. For example, sliding seal 60190 may be positioned along an inner surface of rail 60170 and an outer surface 60179 of cartridge 60180 to facilitate movement of cartridge 60180 along rail 60170. The cartridge 60180, sliding seal 60190, and rail 60170 may be concentric.

In some embodiments, sliding seal 60190 may be fixed to a position at the outer surface of cartridge 60180, while sliding seal 60190 is configured to slide along the inner surface of rail 60170. For example, as shown by FIGS. 61 and 62, the positioning between sliding seal 60190 and cartridge 60180 may remain static. The sliding seal 60190 and cartridge 60180 may move, as a unit, from the base 60171 of rail 60170 towards the rim 60173 of rail 60170. In short, sliding seal 60190 and cartridge 60180 may translate simultaneously together along the rail 60170, in a direction and position parallel to or along the longitudinal axis 6010 of housing 60110. In another embodiment, the relative position of rail 60170 and sliding seal 60190 may be static, while cartridge 60180 translates towards flowpath 60200. In yet another embodiment, sliding seal 60190 may move relative to both rail 60170 and cartridge 60180. In some embodiments, the position of cartridge 60180 may remain static relative to the housing 60110, while flowpath 60200 is moved through seal 60183 to put cartridge 60180 and flowpath 60200 into fluid communication.

In some cases, rail 60170 may include one or more stoppers (not shown) along its inner surface. The stoppers may abut sliding seal 60190 and stop the motion of sliding seal 60190 along the longitudinal axis 6010. Alternately or in addition, one or more stoppers may be positioned at outer surface 60179 of cartridge 60180 to stabilize or stop the motion of cartridge 60180. Due to the coupling between the sliding seal 60190 and cartridge 60180, translation of the cartridge 60180 along the longitudinal axis 6010 may stop once the sliding seal 60190 is prevented from moving along the longitudinal axis 6010. It also is contemplated that no such stopper may be required, and that longitudinal movement of cartridge 60180 will cease once seal 60183 is punctured by first needle 60210, since further movement of plunger 60185 at that point will urge medicament 60181 through flowpath 60200.

The outer surface 60179 of cartridge 60180, the inner surface of rail 60170, and the sliding seal 60190 may form the boundaries of a cavity comprising dispensing chamber 60175. Prior to use of the auto-injector 60100, cartridge 60180 may be positioned near base 60171 of the rail 60170 and sliding seal 60190. Dispensing chamber 60175 may be at a first volume prior to use. After actuation of fluid source 60145, compressed fluid 60150 released from the fluid source 60145 may fill the dispensing chamber 60175. The dispensing chamber 60175 may expand as compressed fluid 60150 pushes plunger 60185, cartridge 60180, and sliding seal 60190, urging that entire assembly along the longitudinal axis 6010. As previously described, sliding seal 60190 and cartridge 60180 may shift towards to rim 60173, along or parallel to the longitudinal axis 6010 of the housing 60110.

For example, fluid 60150 may expand to fill dispensing chamber 60175 and thus push sliding seal 60190 along the longitudinal axis 6010 towards the expulsion end 6040. The longitudinal motion of the sliding seal 60190 may push the cartridge 60180 also towards the expulsion end 6040 such that the cartridge 60180 (e.g., seal 60183) contacts the first needle 60210 of flowpath 60200. This contact between seal 60183 and the first needle 60210 of flowpath 60200 may cause first needle 60210 to puncture seal 60183 and place flowpath 60200 into fluid communication with cavity 60182 of cartridge 60180 (e.g., at FIG. 62). Fluid 60150 may apply pressure to plunger 60185 and thus push plunger 60185 through the body of cartridge 60180. As plunger 60185 moves through cartridge 60180, the movement of plunger 60185 may force medicament 60181 to flow through lumen 60230 of flowpath 60200 to the patient via second needle 60220.

In some embodiments, cartridge 60180, rail 60170, and sliding seal 60190 may be configured such that cartridge 60180 may be replaceable. For example, rail 60170 and sliding seal 60190 may include one or more openings through which cartridge 60180 may be inserted. Alternately, cartridge 60180, rail 60170, and sliding seal 60190 may be inserted, as an integral unit, into auto-injector 60100 and arranged to be in fluid communication with conduit 60155.

In the pre-activated state of auto-injector 60100 shown in FIG. 60B, first needle 60210 may be spaced apart from the opening 60189 of cartridge 60180. As this state, cartridge 60180 may be fluidly isolated from the compressed fluid 60150. Cartridge 60180 also is fluidly isolated and spaced apart from flowpath 60200 at this stage. In particular, there may be a gap between first needle 60210 and cartridge 60180 and/or no direct physical connection between flowpath 60200 and cartridge 60180.

Auto-injector 60100 may be positioned by a user onto the user's body so that tissue-engaging surface 60125 of the retractable shroud 60117 contacts a skin surface. Auto-injector 60100 may be mounted to any treatment or medicament delivery site, such as, e.g., the thigh, abdomen, shoulder, forearm, upper arm, leg, buttocks, or another suitable location. Retractable shroud 60117 may then be compressed against the delivery site.

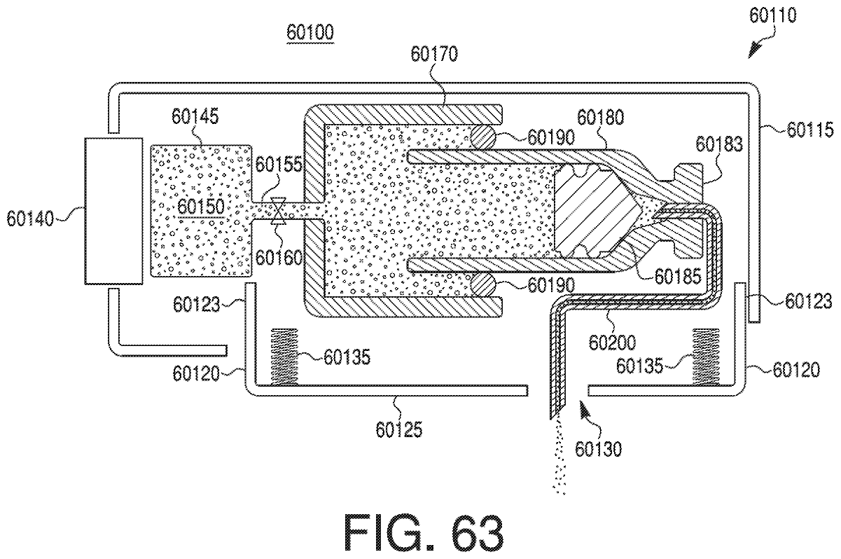

For example, the user may apply a force to handle portion 60115 to retract shroud 60117 and inject second needle 60220 of flowpath 60200 into the skin surface, puncturing the skin. Then, fluid source 60145 may be actuated by any of the mechanisms set forth above, so that fluid 60150 may be released from fluid source 60145 to move container 60180 along longitudinal axis 6010 toward first needle 60210. Because the first needle 60210 is not yet in fluid communication with cartridge 60180, activation of fluid source 60145 may apply a pressure against the medicament 60181 contained in cartridge 60180 as the fluid 60150 fills dispensing chamber 60175. This pressure is then applied to cartridge 60180 itself. This pressure causes cartridge 60180 to translate along or parallel to the longitudinal axis 6010, toward the first needle 60210, ultimately forcing first needle 60210 through the seal 60183 such that the flowpath 60200 is in fluid communication with the contents of cartridge 60180. Once flowpath 60200 is in fluid communication with cartridge 60180, further movement of plunger 60185 toward opening 60189 urges medicament 60181 through flowpath 60200 (shown in FIGS. 62 and 63).

For example, fluid 60150 may continue to fill the dispensing chamber 60175 after fluid communication is established between cartridge 60180 and flowpath 60200. In this way, expansion of fluid 60150 may translate plunger 60185 and thus urge medicament to flow out of the cartridge 60180. Because the cartridge 60180 is in fluid communication with the flowpath 60200, the medicament may be forced out of the cartridge 60180 and into the flowpath 60200, which may then dispense the medicament to the patient. Once the plunger 60185 reaches opening 60189 or otherwise cannot move further through cartridge 60180 (e.g., FIG. 63), the medicament 60181 may be fully dispensed from cartridge 60180 and into the user.

After completion of the injection, which may be visually confirmed or confirmed by another suitable mechanism, second needle 60220 may be retracted from the user. In one embodiment, where the user maintains pressure on auto-injector 60100 throughout the course of the injection, the user may simply remove the force after completion of the injection to expand or extend the shroud 60117 from its collapsed/retracted position over second needle 60220. In other embodiments, where handle portion 60115 and shroud 60117 are held into the compressed configuration, by e.g., a latch, the user may actuate a separate mechanism to withdraw second needle 60220. Alternatively, auto-injector may utilize one or more sensors to determine an end of the injection, and automatically initiate extension of shroud 60117 over second needle 60220, e.g., via a spring, gas, extending of folds in a (bellow-shaped or creased) shroud configuration, etc.

A method of using auto-injector 60100 may include determining whether a drug within cartridge 60180 has been compromised, expired, or is too cold for delivery into the user, determining a dosage of a medicament desired for a user compared to the volume of medicament in cartridge 60180, determining whether the compressed fluid 60150 is at a temperature where it may expand and operate as desired for facilitating drug delivery, determining whether flowpath 60200 has been prematurely deployed and/or retracted, and whether an injection procedure has extended beyond an expected or predetermined procedure time. In some embodiments, expansion of the shroud 60117 over the flowpath 60200 may stop expulsion of the fluid 60150 from the fluid source 60145.

In some examples, a timing of an injection procedure, measured from the initial activation of flowpath 60200 deployment through housing opening 60130 to the plunger 60185 reaching opening 60189 of the cartridge 60180, may be from about 20 seconds to about 90 seconds, or from about 25 seconds to about 60 seconds, from about 30 seconds to about 45 seconds, or less than or equal to about 120 seconds, or less than or equal to about 90 seconds, or less than or equal to about 60 seconds, or less than or equal to about 45 seconds, or less than or equal to about 30 seconds.

Various springs and/or resilient members are discussed herein. In some embodiments, the spring (e.g., spring 370) is discussed as biased into an expanded state, and may be compressed in an un-activated or otherwise new state of the auto-injector 2. Thus, the spring may have a resting, expanded state. The spring or resilient member then may be compressed as auto-injector 2 is placed into the unused state, and then expands as the auto-injector 2 transitions from the unused state to an “in-use” state, and may revert to its original or biased (expanded) position upon completion of an injection, for example. However, it is contemplated that, in at least some embodiments, a spring or resilient member may be utilized that is biased into a compressed configuration (or has a resting, compressed state). In such embodiments, the spring may be in a forced expanded state while the auto-injector 2 is un-activated or new, and allowed to compress as auto-injector 2 transitions from the unused state to the “in-use” state, and may revert to its original or biased (compressed) configuration upon completion of the injection. Furthermore, it is contemplated that anywhere a spring is specifically discussed, that another suitable compressible/expandable resilient member may be used.

Furthermore, embodiments of this disclosure may include one or more features of International PCT Publication No. WO 2018/204779, the entirety of which is incorporated herein by reference.

Notably, reference herein to “one embodiment,” or “an embodiment” means that a particular feature, structure, or characteristic described in connection with the embodiment may be included, employed and/or incorporated in one, some or all of the embodiments of the present disclosure. The usages or appearances of the phrase “in one embodiment” or “in another embodiment” in the specification are not referring to the same embodiment, nor are separate or alternative embodiments necessarily mutually exclusive of one or more other embodiments, nor limited to a single exclusive embodiment. The same applies to the terms “implementation,” and “example.” The present disclosure are neither limited to any single aspect nor embodiment thereof, nor to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein.

Further, as indicated above, an embodiment or implementation described herein as “exemplary” is not to be construed as preferred or advantageous, for example, over other embodiments or implementations; rather, it is intended convey or indicate the embodiment or embodiments are example embodiment(s).

1.-57. (canceled)

58. An auto-injector, comprising:
a carrier,
a needle;
a driver coupled to the needle, the driver being slidable relative to the carrier between a first position, a second position, and a third position;
a shuttle configured to move the driver between the first position, the second position, and the third position; and
an indicator couplable to the shuttle, a portion of the indicator being visible from exterior of the auto-injector, the auto-injector providing via the shuttle and the indicator a first indication corresponding to the first position of the driver, a second indication corresponding to the second position of the driver, and a third indication corresponding to the third position of the driver.

59. An auto-injector, comprising:
a carrier,
a container comprising a medicament;
a sleeve coupled to the container, the sleeve comprising a longitudinally-extending slot and a laterally or circumferentially-extending slot extending from the longitudinally-extending slot;
a needle having a first end configured to extend out of the auto-injector, and a second end configured to extend into the container, wherein, in a first state of the auto-injector, the second end of the needle and the container are not in fluid communication with one another;
a connector housing coupled to the second end of the needle, the connector housing comprising a boss, wherein, in the first state of the auto-injector, the boss abuts against a portion of the sleeve, preventing movement of the sleeve and the connector housing relative to one another; and
a driver coupled to the needle, the driver being slidable relative to the carrier between a first position, a second position, and a third position; wherein
in a transition from the first state to a second state of the auto-injector, the driver moves the first end of the needle out of the auto-injector to the second position, and rotates the connector housing, in a first rotational direction, so that the boss is able to extend through the longitudinally-extending slot;
in the second state, the second end of the needle extends into and is in fluid communication with the container; and
in a transition from the second state to a third state of the auto-injector, the driver moves to the third position, and rotates the connector housing, in a second rotational direction opposite of the first rotational direction, so that the boss is able to extend through the laterally or circumferentially-extending slot.

60. The auto-injector of claim 59, further including a button that is depressible from an unactuated position to an actuated position, a first resilient member that is compressed when the container moves from a first position to a second position, and a second resilient member that is compressed when the button is in the unactuated position, and expanded when the button is depressed to the actuated position, wherein the compressed first resilient member expands to move the container.

61. (canceled)

62. The auto-injector of claim 60, further including:
a fluid source configured to release a pressurized fluid, wherein expansion of the second resilient member actuates the fluid source; and
a pressure restrictor configured to restrict flow of the pressurized fluid, and a valve configured to regulate flow of the pressurized fluid from the pressure restrictor;
wherein the pressure restrictor defines a high pressure flow area and a low pressure flow area, and the valve includes a first valve inlet fluidly coupled to a first valve cavity, a second valve inlet fluidly coupled to a second valve cavity, and a valve outlet.

63. The auto-injector of claim 62, further including a diaphragm that includes a flexible body and a rim extending about a periphery of the flexible body, the diaphragm includes a greater thickness along the rim relative to a remaining portion of the flexible body, and a raised portion at a center position of the flexible body such that the rim extends about the raised portion;
wherein the diaphragm includes a greater thickness at the raised portion relative to a remaining portion of the flexible body, and the raised portion includes one or more indentations extending radially inward from an outer circumferential face of the raised portion, the diaphragm is configured to extend into the second valve cavity and away from the first valve cavity when the pressure within the high pressure flow area exceeds the pressure within the low pressure flow area;
wherein the flexible body is configured to push against the valve outlet to seal the second valve cavity when the diaphragm extends into the second valve cavity.

64.-67. (canceled)

68. The auto-injector of claim 58, wherein the shuttle is movably coupled to the driver via a rotatable gear, the shuttle is configured to move the driver from the first position to the second position in response to rotating the rotatable gear in a first rotational direction, and to move the driver from the second position to the third position in response to rotating the rotatable gear in a second rotational direction that is opposite of the first rotational direction.

69. The auto-injector of claim 58, wherein the shuttle is movable relative to the indicator in some configurations, and movable with the indicator in other configurations, wherein the first indication, the second indication, and the third indication of the indicator each corresponds to a different position of the shuttle relative to the carrier.

70. (canceled)

71. A handheld auto-injector, comprising:
a handheld housing having a longitudinal axis and a transverse axis that is perpendicular to the longitudinal axis, the handheld housing having a shorter dimension along the transverse axis than along the longitudinal axis, wherein the handheld auto-injector is configured to complete an injection procedure in 30 seconds or less;
a flowpath having a first end and a second end; and
a container configured to enclose a first fluid, the container extending from a first end toward a second end along or parallel to the longitudinal axis and being movable from a first position to a second position along or parallel to the longitudinal axis, the container being fluidly isolated from the flowpath in the first position and fluidly connected to the flowpath in the second position; and
the container further including a plunger configured to move from the first end toward the second end of the container to expel the first fluid from the container into the flowpath; and
wherein the first end of the flowpath is insertable into the container and the second end of the flowpath is extendable from the handheld housing in a direction along or parallel to the transverse axis through an opening in the handheld housing.

72. The handheld auto-injector of claim 71, wherein the handheld housing includes a platform raised relative to a top surface of the handheld housing, the platform extending along the longitudinal axis of the handheld housing.

73. The handheld auto-injector of claim 72, wherein the handheld housing includes a button positioned along a surface of the handheld housing, the button being flush, recessed, or raised relative to the surface, and the surface is selected from a group consisting of a top surface, a side surface, or a bottom surface.

74. The handheld auto-injector of claim 73, wherein at least a portion of the button includes a visual appearance or feature that is different from the handheld housing, the visual appearance or feature of the button is selected from a group consisting of a color, a marking, a material composition, a bump, a divot, a rib, or an elevation that is different from the handheld housing.

75. The handheld auto-injector of claim 71, wherein the handheld housing includes a first button and a second button positioned on opposing ends or different surfaces of the handheld housing.

76. The handheld auto-injector of claim 71, wherein the handheld housing includes a window positioned along a wall of the handheld housing, the window extending along the longitudinal axis, and the wall selected from a group consisting of a top wall, a bottom wall, or a side wall.

77. The handheld auto-injector of claim 76, wherein the window is a first window and the wall is a first wall, and the handheld housing includes a second window positioned along a second wall of the handheld housing that is the same or different than the first wall.

78. The handheld auto-injector of claim 71, wherein the handheld housing includes flat or rounded ends, or one or more recessed walls along a top surface or side surface of the handheld housing.

79. The handheld auto-injector of claim 71, wherein the handheld housing includes a slider positioned along a top surface of the handheld housing, the slider is configured to move along the longitudinal axis of the handheld housing, and provide an indication of a corresponding position or state of the flowpath.

80. The handheld auto-injector of claim 71, wherein the handheld housing has a lateral axis that is perpendicular to the longitudinal axis, the handheld housing having a shorter dimension along the lateral axis than along the transverse axis or the longitudinal axis.

81. The handheld auto-injector of claim 71, wherein the handheld housing includes a feature positioned along a bottom surface of the handheld housing selected from the group consisting of a removable seal, a depressible contact switch, a retractable shroud, a label, or a coating configured to inhibit slippage of the handheld housing against skin.

82. The handheld auto-injector of claim 71, wherein further including a removable cap coupled to at least a portion of the handheld housing.

83. The handheld auto-injector of claim 71, wherein the handheld housing includes a shroud having a plurality of colors for identifying an approximate location of an opening along the shroud.
